(19) United States
(12) Patent Application Publication
(10) Pub. No.: US 2007/0249518 A1
(43) Pub. Date: Oct. 25, 2007

(54) Compositions and Methods for Treating Mental Disorders

(75) Inventors: Ilya Chumakov, Vaux-le-Penil (FR); Daniel Cohen, Le Vesinet (FR); Fabio Macciardi, Paris (FR)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Ares Trading S.A., Aubonne (CH)

(21) Appl. No.: 11/575,838

(22) PCT Filed: Dec. 9, 2005
(86) PCT No.: PCT/EP05/56660

§ 371 Date: Jun. 29, 2007

(30) Foreign Application Priority Data
Dec. 24, 2004 (EP) 04293130.3

(51) Int. Cl. A61K 38/00; C12Q 1/68

(52) U.S. Cl. 514/2; 435/6

(57) ABSTRACT

The present invention relates, generally, to methods and compositions for detecting or treating mental disorders, such as schizophrenia or bipolar disorder. The present invention more particularly discloses the identification of human genes that can be used for the diagnosis, prevention and treatment of schizophrenia, bipolar disorder and related disorders, as well as for the screening of therapeutically active drugs to treat said disorders. The invention further discloses specific polymorphisms or alleles of the KCNQ3 gene that are related to schizophrenia or bipolar disorder, as well as diagnostic tools and kits based on these markers. The invention can be used in the diagnosis of or predisposition to, detection, prevention and/or treatment of schizophrenia, bipolar disorder and related disorders.

### FIELD OF THE INVENTION

The present invention relates, generally, to methods and compositions for detecting or treating mental disorders, such as schizophrenia and bipolar disorder. The present invention more particularly discloses the identification of the human gene KCNQ3 as a target which can be used for the diagnosis, prevention and treatment of schizophrenia, bipolar disorder and related disorders, as well as for the screening of therapeutically active drugs. The invention further discloses specific polymorphisms or alleles of the KCNQ3 gene that are related to schizophrenia and bipolar disorder, as well as diagnostic tools and kits based on these markers. The invention can be used in the diagnosis or detection of the presence, risk or predisposition to, as well as in the prevention and/or treatment of schizophrenia, bipolar disorder and related disorders.

### BACKGROUND OF THE INVENTION

There are an estimated 45 million people with schizophrenia in the world, with more than 33 million of them in the developing countries. In developed countries schizophrenia occurs in approximately 1% of the adult population at some point during their lives. If there is one grandparent with schizophrenia, the risk of getting the illness increases to about 3%; one parent with Schizophrenia, to about 10%. When both parents have schizophrenia, the risk rises to approximately 40%. Most schizophrenia patients are never able to work. Standardized mortality ratios (SMRs) for schizophrenic patients are estimated to be two to four times higher than the general population and their life expectancy overall is 20% shorter than for the general population. The most common cause of death among schizophrenic patients is suicide (in 10% of patients) which represents a 20 times higher risk than for the general population. Deaths from heart disease and from diseases of the respiratory and digestive system are also increased among schizophrenic patients.

Schizophrenia comprises a group of psychoses with ‘positive’ and/or ‘negative’ symptoms. Positive symptoms consist of hallucinations, delusions and disorders of thought; negative symptoms include emotional flattening, lack of volition and a decrease in motor activity. Antipsychotic medications are the most common and valuable treatments for schizophrenia. There are four main classes of antipsychotic drugs which are commonly prescribed for schizophrenia. The first, neuroleptics, exemplified by chlorpromazine (Thorazine), has revolutionized the treatment of schizophrenic patients by reducing positive (psychotic) symptoms and preventing their recurrence. Patients receiving chlorpromazine have been able to leave mental hospitals and live in community programs or their own homes. But these drugs are far from ideal. Some 20% to 30% of patients do not respond to them at all, and others eventually relapse. These drugs were named neuroleptics because they produce serious neurological side effects, including rigidity and tremors in the arms and legs, muscle spasms, abnormal body movements, and akathisia (restless pacing and fidgeting). These side effects are so troublesome that many patients simply refuse to take the drugs. Besides, neuroleptics do not improve the so-called negative symptoms of schizophrenia and the side effects may even exacerbate these symptoms. Thus, despite the clear beneficial effects of neuroleptics, even some patients who have a good short-term response will ultimately deteriorate in overall functioning. The well known deficiencies in the standard neuroleptics have stimulated a search for new treatments and have led to a new class of drugs termed atypical neuroleptics. The first atypical neuroleptic, Clozapine, is effective for about one third of patients who do not respond to standard neuroleptics. It seems to reduce negative as well as positive symptoms, or at least exacerbates negative symptoms less than standard neuroleptics do. Moreover, it has beneficial effects on overall functioning and may reduce the chance of suicide in schizophrenic patients. It does not produce the troubling neurological symptoms of the standard neuroleptics, or raise blood levels of the hormone prolactin, excess of which may cause menstrual irregularities and infertility in women, impotence or breast enlargement in men. Many patients who cannot tolerate standard neuroleptics have been able to take clozapine. However, clozapine has serious limitations. It was originally withdrawn from the market because it can cause agranulocytosis, a potentially lethal inability to produce white blood cells. Agranulocytosis remains a threat that requires careful monitoring and periodic blood tests. Clozapine can also cause seizures and other disturbing side effects (e.g., drowsiness, lowered blood pressure, drooling, bed-wetting, and weight gain). Thus only patients who do not respond to other drugs usually take Clozapine.

Researchers have developed a third class of antipsychotic drugs that have the virtues of clozapine without its defects. One of these drugs is risperidone (Risperdal). Early studies suggest that it is as effective as standard neuroleptic drugs for positive symptoms and may be somewhat more effective for negative symptoms. It produces more neurological side effects than clozapine but fewer than standard neuroleptics. However, it raises prolactin levels. Risperidone is now prescribed for a broad range of psychotic patients, and many clinicians seem to use it before clozapine for patients who do not respond to standard drugs, because they regard it as safer. Another new drug is Olanzapine (Zyprexa), which is at least as effective as standard drugs for positive symptoms and more effective for negative symptoms. It has few neurological side effects at ordinary clinical doses, and it does not significantly raise prolactin levels. Although it does not produce most of clozapine's most troubling side effects, including agranulocytosis, some patients taking olanzapine may become sedated or dizzy, develop dry mouth, or gain weight. In rare cases, liver function tests become transiently abnormal.

A number of biochemical abnormalities have been identified in schizophrenic patients. As a consequence, several neurotransmitter-based hypotheses have been advanced over recent years; the most popular one has been “the dopamine hypothesis,” one variant of which states that there is over-activity of the mesolimbic dopamine pathways at the level of the D₂ receptor. However, researchers have been unable to consistently find an association between various receptors of the dopaminergic system and schizophrenia.

Bipolar disorders are relatively common disorders, occurring in about 1.3% of the population, and have been reported to constitute about half of the mood disorders seen in psychiatric clinics with severe and potentially disabling effects. Bipolar disorders have been found to vary with gender depending of the type of disorder; for example, bipolar disorder I is found equally among men and women, while bipolar disorder II is reportedly more common in women. The age of onset of bipolar disorders is typically in the teenage years and diagnosis is typically made in the patient's early twenties. Bipolar disorders also occur among the elderly, generally as a result of a neurological disorder or other medical conditions. In addition to the severe effects on patients' social development, suicide completion rates among bipolar patients are reported to be about 15%.

Bipolar disorders are characterized by phases of excitement and often depression; the excitement phases, referred to as mania or hypomania, and depressive phases can alternate or occur in various admixtures, and can occur to different degrees of severity and over varying duration. Since bipolar disorders can exist in different forms and display different symptoms, the classification of bipolar disorder has been the subject of extensive studies resulting in the definition of bipolar disorder subtypes and widening of the overall concept to include patients previously thought to be suffering from different disorders. Bipolar disorders often share certain clinical signs, symptoms, treatments and neurobiological features with psychotic illnesses in general and therefore present a challenge to the psychiatrist to make an accurate diagnosis. Furthermore, because the course of bipolar disorders and various mood and psychotic disorders can differ greatly, it is critical to characterize the illness as early as possible in order to offer means to manage the illness over a long term.

The mania associated with the disease impairs performance and causes psychosis, and often results in hospitalization. This disease places a heavy burden on the patient's family and relatives, both in terms of the direct and indirect costs involved and the social stigma associated with the illness, sometimes over generations. Such stigma often leads to isolation and neglect. Furthermore, the earlier the onset, the more severe are the effects of interrupted education and social development.

The DSM-IV classification of bipolar disorder distinguishes among four types of disorders based on the degree and duration of mania or hypomania as well as two types of disorders, which are evident typically with medical conditions or their treatments, or to substance abuse. Mania is recognized by elevated, expansive or irritable mood as well as by distractibility, impulsive behavior, increased activity, grandiosity, elation, racing thoughts, and pressured speech. Of the four types of bipolar disorder characterized by the particular degree and duration of mania, DSM-IV includes:
bipolar disorder I, including patients displaying mania for at least one week;
bipolar disorder II, including patients displaying hypomania for at least 4 days, characterized by milder symptoms of excitement than mania, who have not previously displayed mania, and have previously suffered from episodes of major depression;
bipolar disorder not otherwise specified (NOS), including patients otherwise displaying features of bipolar disorder II but not meeting the 4 day duration for the excitement phase, or who display hypomania without an episode of major depression; and
cyclothymia, including patients who show numerous manic and depressive symptoms that do not meet the criteria for hypomania or major depression, but which are displayed for over two years without a symptom-free interval of more than two months.

The remaining two types of bipolar disorder as classified in DSM-VI are disorders evident or caused by various medical disorder and their treatments, and disorders involving or related to substance abuse. Medical disorders which can cause bipolar disorders typically include endocrine disorders and cerebrovascular injuries, and medical treatments causing bipolar disorder are known to include glucocorticoids and the abuse of stimulants. The disorder associated with the use or abuse of a substance is referred to as “substance induced mood disorder with manic or mixed features”.

Evidence from twin and adoption studies, and the lack of variation in incidence worldwide, indicate that bipolar disorder is primarily a genetic condition, although environmental risk factors are also involved at some level as necessary, sufficient, or interactive causes. Aggregation of bipolar disorder and schizophrenia in families suggests that these two distinct disorders share some common genetic susceptibility. Several linkage studies of bipolar disorder have been reported, and several susceptibility regions have been identified. The regions that are associated with bipolar disorder include 1q31-q32, 4p16, 7q31, 12q23-q24, 13q32, 18p11.2, 21q22 and 22q11-q13 (Detera-Wadleigh et al., 1999). Some of these regions, like 4p16, 12q24, 18p11, 21q21 and 22q11 have been repeatedly implicated by independent investigators. Furthermore, some regions that are linked to bipolar disorder such as, e.g., 13q32 and 18p11.2, are also implicated in genome scans of schizophrenia, confirming that these two distinct disorders share some common genetic susceptibility. However, the genes underlying bipolar disorder have not yet been identified.

As discussed above, molecules used for the treatment of schizophrenia have side effects and act only against the symptoms of the disease; and the genes underlying bipolar disorder have not yet been identified. Consequently, there is a strong need for new molecules without associated side effects that are specifically directed against targets which are involved in the causal mechanisms of such disorders. Therefore, there is a need to identify proteins involved in such diseases, thereby providing new targets allowing new screenings for drugs, resulting in new drugs that are efficient in treatment of this serious mental disease and related disorders.

Furthermore, there is also a need for diagnostic tools. There is increasing evidence that leaving schizophrenia untreated for long periods early in course of the illness may negatively affect the outcome. However, the use of drugs is often delayed for patients experiencing a first episode of the illness. The patients may not realize that they are ill, or they may be afraid to seek help; family members sometimes hope the problem will simply disappear or cannot persuade the patient to seek treatment; clinicians may hesitate to prescribe antipsychotic medications when the diagnosis is uncertain because of potential side effects. Indeed, at the first manifestation of the disease, schizophrenia may be difficult to distinguish from, e.g., drug-related disorders and stress-related disorders. Accordingly, there is a need for new methods for detecting a susceptibility to schizophrenia, bipolar disorder and related disorders.

### SUMMARY OF THE INVENTION

The present invention now discloses novel approaches to the diagnosis and treatment of schizophrenia, bipolar disorder (BP) and related disorders, as well as for the screening of therapeutically active drugs. The invention more specifically demonstrates that alterations in the KCNQ3 gene are associated with the development of schizophrenia, bipolar disorder and other mental disorders. KCNQ3, and altered forms of KCNQ3 in particular, represent novel targets for therapeutic intervention in said diseases and related pathologies.

A first aspect of this invention thus resides in the use of a KCNQ3 gene or polypeptide as a target for the screening of candidate drug modulators, particularly candidate drugs active against schizophrenia, bipolar disorder and related disorders.

Another aspect of this invention resides in a method of assessing the presence of or predisposition to schizophrenia, bipolar disorder or a related disorder in a subject, comprising determining (in vitro or ex vivo) the presence of an alteration (e.g., a susceptibility mutation or allele) in a KCNQ3 gene or polypeptide in a sample from the subject, the presence of such an alteration being indicative of the presence of or predisposition to schizophrenia, bipolar disorder or a related disorder in said subject.

A further aspect of this invention relates to the use of a modulator of a KCNQ3 gene or polypeptide for the preparation of a medicament for treating or preventing schizophrenia, bipolar disorder or a related disorder in a subject, as well as to corresponding methods of treatment.

The invention more specifically encompasses methods of treating schizophrenia, bipolar disorder or related disorders in a subject through a modulation of KCNQ3 gene or polypeptide expression or activity. Such treatments use, for instance, KCNQ3 polypeptides, KCNQ3 DNA sequences (including antisense sequences, RNAi), antibodies against KCNQ3 polypeptides, ligands of KCNQ3 or drugs that modulate KCNQ3 expression or activity. The invention particularly relates to methods of treating individuals having disease-associated alleles of the KCNQ3 gene.

A further aspect of this invention resides in methods of screening of compounds for therapy of schizophrenia, bipolar disorder or related disorders, comprising binding of a compound to a KCNQ3 gene or polypeptide, or a fragment thereof, particularly of an allele of said gene or polypeptide that is associated with schizophrenia, bipolar disorder or a related disorder, or a fragment thereof.

A further aspect of this invention resides in methods of screening of compounds for therapy of schizophrenia, bipolar disorder or related disorders, comprising testing for modulation of the activity of a KCNQ3 gene or polypeptide, or a fragment thereof, particularly of an allele of said gene or polypeptide that is associated with schizophrenia, bipolar disorder or a related disorder, or a fragment thereof.

The invention further relates to the screening of alteration(s) associated with schizophrenia, bipolar disorder or related disorders in the KCNQ3 gene locus in patients. Such screenings are useful for diagnosing the presence, risk or predisposition to schizophrenia, bipolar disorder and related disorders, and/or for assessing the efficacy of a treatment of such disorders.

A further aspect of this invention includes nucleic acid probes and primers that allow specific detection of susceptibility markers in a KCNQ3 gene or RNA through selective hybridization or amplification. The invention also encompasses particular nucleic acids, vectors and recombinant cells, as well as kits or solid phase bound nucleic acids or proteins such as DNA or protein arrays or chips suitable for implementing the above detection, screening or treatment methods. In particular, the invention also discloses markers in KCNQ3 nucleic acids and polypeptides that are associated with schizophrenia, bipolar disorder and related disorders. Examples of markers in the KCNQ3 gene that are associated with schizophrenia include the M2, M3, M6, M9, M12, M16 and M24 markers as listed in Table 2, or combination(s) thereof. An example of a marker in the KCNQ3 gene that is associated with bipolar disorder includes M13 marker as listed in Table 2.

The invention can be used in the diagnosis of predisposition to, detection, prevention and/or treatment of schizophrenia, bipolar disorder and related disorders.

### DETAILED DESCRIPTION OF THE INVENTION

The present invention stems from association studies conducted on different schizophrenic populations, using a number of random markers. The results of these studies, which are presented in the experimental section, show that the KCNQ3 gene is strongly associated with schizophrenia and bipolar disorder, and that validated (biallelic) markers located in said gene or RNAs are associated with said pathologies and related disorders.

The present invention thus provides novel means and methods to identify compounds useful in the treatment of schizophrenia, bipolar disorder and related disorders. The invention further provides novel approaches to the detection, diagnosis and monitoring of schizophrenia, bipolar disorder or related disorders in a subject, as well as for genotyping of schizophrenic patients.

Definitions

The term “schizophrenia” refers to a condition characterized as schizophrenia in the DSM-IV classification (Diagnosis and Statistical Manual of Mental Disorders, Fourth Edition, American Psychiatric Association, Washington D.C., 1994).

Schizophrenia related disorders include psychotic disorders, such as schizoaffective disorder, schizophreniform disorder, brief psychotic disorder, delusional disorder and shared psychotic disorder, as well as other mental disorders such as mood disorders (e.g., bipolar disorder) and depression.

The term “mental disorder” refers, more generally, to diseases characterized as mood disorders, psychotic disorders, anxiety disorders, childhood disorders, eating disorders, personality disorders, adjustment disorder, autistic disorder, delirium, dementia, multi-infarct dementia and Tourette's disorder in the DSM-IV classification (Diagnosis and Statistical Manual of Mental Disorders, Fourth Edition, American Psychiatric Association, Washington D.C., 1994). “Bipolar disorder” as used herein refers more specifically to a condition characterized as a Bipolar Disorder in the DSM-IV. Bipolar disorder may be bipolar I and bipolar disorder II as described in the DSM-IV. The term further includes cyclothymic disorder. Cyclothymic disorder refers to an alternation of depressive symptoms and hypomanic symptoms. The skilled artisan will recognize that there are alternative nomenclatures, posologies, and classification systems for pathologic psychological conditions and that these systems evolve with medical scientific progress.

As used in the present application, the term “KCNQ3” refers to a member of the potassium channel family. The nucleic and amino acid sequences of a KCNQ3 gene or polypeptide are available in the literature and may be found for instance under the following accession numbers:
cDNA and protein sequence: NM_{—}004519 (SEQ ID NO: 1 and 2, respectively);
partial cDNA sequence: AF033347.

The genomic sequence of KCNQ3 spans ˜350 kb. The sequence of the 15 exons of the KCNQ3 gene are found under the following accession numbers:
exon 1: AF071478;
exon 2: AF071479;
exon 3: AF071480;
exon 4: AF071481;
exon 5 and exon 6: AF071482;
exon 7: AF071483;
exon 8: AF071484;
exon 9: AF071485;
exon 10: AF071486;
exon 11: AF071487;
exon 12: AF071488;
exon 13: AF071489;
exon 14: AF071490; and
exon 15: AF071491.

The KCNQ3 gene encodes a subunit of neuronal M-type K⁺ channels. In 1980's, David Brown and Paul Adams described the existence of a low-threshold, non-activating, voltage-dependent K⁺ current in neurons, referred to as the M-current, which plays a dominant role in regulating excitability because of its unique activity in the voltage range of action-potential initiation (Brown et al., 1980). The M-current slowly activates when an excitatory stimulus depolarizes the neuron toward spike threshold, repolarizing the membrane back toward resting potential and suppressing firing. In this way, the M-current limits repetitive spike firing in response to a persistent depolarizing stimulus and is therefore a key mechanism for “spike-frequency adaptation”. The M-current is suppressed by neurotransmitters acting on G-protein-coupled receptors, including acetylcholine acting on muscarinic receptors, from which its name is derived. Suppression of the M-current results in membrane depolarization and an increase in neuronal input resistance, making the cell more likely to fire action potentials. Centrally acting muscarinic cholinergic agonists and cholinesterase inhibitors that increase the synaptic availability of acetylcholine are powerful convulsants, an action that is caused, at least in part, by M-current suppression.

Jentsch (2000) discussed the physiology and role of KCNQ receptors and suggests their implication in epilepsy. Cooper et al (2003) discussed the implication of M-channels in certain pathological conditions, such as epilepsy and Alzheimer. This reference, however, admits that further studies are required to clarify the role of KCNQ channels in neuroprotection. Schwake et al (2000) relates to the surface expression of M-type K⁺ channels and their implication in epilepsy. However, the prior art does not provide any indication that a KCNQ3 receptor may be related to schizophrenia or bipolar disorder. For the first time, the invention shows that a KCNQ3 receptor is related to schizophrenia and bipolar disorder, and that this gene is altered in patients suffering from these disorders.

The term “gene” shall be construed to include any type of coding nucleic acid region, including genomic DNA (gDNA), complementary DNA (cDNA), synthetic or semi-synthetic DNA, any form of corresponding RNA, etc., as well as non coding sequences, such as introns, 5′- or 3′-untranslated sequences or regulatory sequences (e.g., promoter or enhancer), etc. The term gene particularly includes recombinant nucleic acids, i.e., any non naturally occurring nucleic acid molecule created artificially, e.g., by assembling, cutting, ligating or amplifying sequences. A gene is typically double-stranded, although other forms may be contemplated, such as single-stranded. Genes may be obtained from various sources and according to various techniques known in the art, such as by screening DNA libraries or by amplification from various natural sources. Recombinant nucleic acids may be prepared by conventional techniques, including chemical synthesis, genetic engineering, enzymatic techniques, or a combination thereof.

A fragment of a gene designates any portion of at least about 8 consecutive nucleotides of a sequence of said gene, preferably at least about 15, more preferably at least about 25 nucleotides, further preferably of at least 35, 50, 75, 100, 150, 200 or 300 nucleotides. Fragments include more particularly all possible nucleotide length between 8 and 500 nucleotides, preferably between 15 and 300, more preferably between 25 and 200.

A KCNQ3 polypeptide designates any protein or polypeptide encoded by a KCNQ3 gene as disclosed above, respectively. In this respect, the term “polypeptide” designates, within the context of this invention, a polymer of amino acids without regard to the length of the polymer; thus, peptides, oligopeptides, and proteins are included within the definition of polypeptide. In particular a KCNQ3 polypeptide also denotes a polypeptide, which is specific fragment of KCNQ3 of at least 8, 15, 20, 50, 100, 250, 500 or 750 amino acids in length. This term also does not specify or exclude post-translational or post-expression modifications of polypeptides, for example, polypeptides which include the covalent attachment of glycosyl groups, acetyl groups, phosphate groups, lipid groups and the like are expressly encompassed by the term polypeptide. Also included within the definition are polypeptides which contain one or more analogs of an amino acid (including, for example, non-naturally occurring amino acids, amino acids which only occur naturally in an unrelated biological system, modified amino acids from mammalian systems etc.), polypeptides with substituted linkages, as well as other modifications known in the art, both naturally occurring and non-naturally occurring.

Fusion proteins are useful for generating antibodies against a KCNQ3 polypeptide and for use in various assay systems. For example, fusion proteins can be used to identify proteins, which interact with portions of a KCNQ3 polypeptide. Protein affinity chromatography or library-based assays for protein-protein interactions, such as the yeast two-hybrid or phage display systems, can be used for this purpose. Such methods are well known in the art and also can be used as drug screens.

A KCNQ3 polypeptide fusion protein comprises two polypeptide segments fused together by means of a peptide bond. The first polypeptide segment comprises at least 25, 50, 75, 100, 150, 200, 300, 400, 500, 600, 700, 750, 800 or 872 contiguous amino acids of SEQ ID NO: 2. The second polypeptide segment can be a full-length protein or a protein fragment. Proteins commonly used in fusion protein construction include beta-galactosidase, beta-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags are used in fusion protein constructions, including histidine (H is) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP16 protein fusions. A fusion protein also can be engineered to contain a cleavage site located between the KCNQ3 polypeptide-encoding sequence and the heterologous protein sequence, so that the KCNQ3 polypeptide can be cleaved and purified away from the heterologous moiety.

A fusion protein can be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences for KCNQ3 in proper reading frame with nucleotides encoding the second polypeptide segment and expressing the DNA construct in a host cell, as is known in the art.

The term “treat” or “treating” as used herein is meant to ameliorate, alleviate symptoms, eliminate the causation of the symptoms either on a temporary or permanent basis, or to prevent or slow the appearance of symptoms of the named disorder or condition. The term “treatment” as used herein also encompasses the term “prevention of the disorder”, which is, e.g., manifested by delaying the onset of the symptoms of the disorder to a medically significant extent. Treatment of the disorder is, e.g., manifested by a decrease in the symptoms associated with the disorder or an amelioration of the reoccurrence of the symptoms of the disorder.

The terms “modulated” or “modulation” or “regulated” or “regulation” as used herein refer to both upregulation [i.e., activation or stimulation (e.g., by agonizing or potentiating)] and downregulation [i.e., inhibition or suppression (e.g., by antagonizing, decreasing or inhibiting)].

The terms “comprising”, “consisting of”, or “consisting essentially of” have distinct meanings. However, each term may be substituted for another herein to change the scope of the invention.

As used interchangeably herein, the term “oligonucleotides”, and “polynucleotides” include RNA, DNA, or RNA/DNA hybrid sequences of more than one nucleotide in either single chain or duplex form. The term “nucleotide” as used herein as an adjective to describe compounds comprising RNA, DNA, or RNA/DNA hybrid sequences of any length in single-stranded or duplex form. The term “nucleotide” is also used herein as a noun to refer to individual nucleotides or varieties of nucleotides, meaning a compound, or individual unit in a larger nucleic acid compound, comprising a purine or pyrimidine, a ribose or deoxyribose sugar moiety, and a phosphate group, or phosphodiester linkage in the case of nucleotides within an oligonucleotide or polynucleotide. Although the term “nucleotide” is also used herein to encompass “modified nucleotides” which comprise at least one modifications (a) an alternative linking group, (b) an analogous form of purine, (c) an analogous form of pyrimidine, or (d) an analogous sugar, for examples of analogous linking groups, purine, pyrimidines, and sugars see for example PCT publication No. WO95/04064, the disclosure of which is incorporated herein by reference. However, the polynucleotides of the invention are preferably comprised of greater than 50% conventional deoxyribose nucleotides, and most preferably greater than 90% conventional deoxyribose nucleotides. The polynucleotide sequences of the invention may be prepared by any known method, including synthetic, recombinant, ex vivo generation, or a combination thereof, as well as utilizing any purification methods known in the art.

The term “isolated” requires that the material be removed from its original environment (e.g., the natural environment if it is naturally occurring). For example, a naturally-occurring polynucleotide or polypeptide present in a living animal is not isolated, but the same polynucleotide or DNA or polypeptide, separated from some or all of the coexisting materials in the natural system, is isolated. Such polynucleotide could be part of a vector and/or such polynucleotide or polypeptide could be part of a composition, and still be isolated in that the vector or composition is not part of its natural environment.

The term “primer” denotes a specific oligonucleotide sequence, which is complementary to a target nucleotide sequence and used to hybridize to the target nucleotide sequence. A primer serves as an initiation point for nucleotide polymerization catalyzed by either DNA polymerase, RNA polymerase or reverse transcriptase. Typical primers of this invention are single-stranded nucleic acid molecules of about 6 to 50 nucleotides in length, more preferably of about 8 to about 40 nucleotides in length, typically of about 16 to 25. The Tm is typically of about 60° C. or more. The sequence of the primer can be derived directly from the sequence of the target gene. Perfect complementarity between the primer sequence and the target gene is preferred, to ensure high specificity. However, certain mismatch may be tolerated.

The term “probe” denotes a defined nucleic acid segment (or nucleotide analog segment, e.g., polynucleotide as defined herein) which can be used to identify a specific polynucleotide sequence present in samples, said nucleic acid segment comprising a nucleotide sequence complementary of the specific polynucleotide sequence to be identified. Probes of this invention typically comprise single-stranded nucleic acids of between 10 to 1000 nucleotides in length, for instance of between 10 and 750, more preferably of between 15 and 600, typically of between 20 and 400. The sequence of the probes can be derived from the sequence of the KCNQ3 gene. The probe may contain nucleotide substitutions and/or chemical modifications, e.g., to increase the stability of hybrids or to label the probe. Typical examples of labels include, without limitation, radioactivity, fluorescence, luminescence, etc.

The terms “complementary” or “complement thereof” are used herein to refer to the sequences of polynucleotides that are capable of forming Watson & Crick base pairing with another specified polynucleotide throughout the entirety of the complementary region. This term is applied to pairs of polynucleotides based solely upon their sequences and not any particular set of conditions under which the two polynucleotides would actually bind. As used herein, the term “non-human animal” refers to any non-human vertebrate, birds and more usually mammals, preferably primates, farm animals such as swine, goats, sheep, donkeys, and horses, rabbits or rodents, more preferably rats or mice. As used herein, the term “animal” is used to refer to any vertebrate, preferable a mammal. Both the terms “animal” and “mammal” expressly embrace human subjects unless preceded with the term “non-human”.

The terms “trait” and “phenotype” are used interchangeably herein and refer to any clinically distinguishable, detectable or otherwise measurable property of an organism such as symptoms of, or susceptibility to a disease for example. Typically the terms “trait” or “phenotype” are used herein to refer to symptoms of, or susceptibility to bipolar disorder; or to refer to an individual's response to an agent acting on bipolar disorder; or to refer to symptoms of, or susceptibility to side effects to an agent acting on bipolar disorder.

As used herein, the term “allele” refers to one of the variant forms of a biallelic or multiallelic marker, differing from other forms in its nucleotide sequence. Typically the first identified allele is designated as the original allele whereas other alleles are designated as alternative alleles. Diploid organisms may be homozygous or heterozygous for an allelic form.

The term “polymorphism” as used herein refers to the occurrence of two or more alternative genomic sequences or alleles between or among different genomes or individuals. “Polymorphic” refers to the condition in which two or more variants of a specific genomic sequence can be found in a population. A “polymorphic site” is the locus at which the variation occurs. A polymorphism may comprise a substitution, deletion or insertion of one or more nucleotides. A single nucleotide polymorphism is a single base pair change. Typically a single nucleotide polymorphism is the replacement of one nucleotide by another nucleotide at the polymorphic site. A “single nucleotide polymorphism” (SNP) refers to a sequence polymorphism differing in a single base pair.

Detection and Diagnosis

The present invention provides novel means and methodologies for detecting or diagnosing schizophrenia and related disorders in a human subject. The present methods may be implemented at various development stages of said pathologies, including early, pre-symptomatic stages, and late stages, in adults, children and pre-birth. Furthermore, the invention is suited to determine the prognosis, to assess a predisposition to or a risk of development of pathology, to characterize the status of a disease or to define the most appropriate treatment regimen for a patient.

A particular object of this invention resides in a method of detecting the presence of or predisposition to schizophrenia or a related disorder in a subject, the method comprising detecting the presence of an alteration in a KCNQ3 gene or polypeptide in a sample from the subject, the presence of such an alteration being indicative of the presence of or predisposition to schizophrenia or a related disorder in said subject.

A further object of this invention resides in a method of detecting the presence of or predisposition to bipolar disorder in a subject, the method comprising detecting the presence of an alteration in a KCNQ3 gene or polypeptide in a sample from the subject, the presence of such an alteration being indicative of the presence of or predisposition to bipolar disorder in said subject.

Another object of this invention relates to methods of assessing the response of a subject to a treatment of schizophrenia, bipolar disorder or a related disorder, the methods comprising detecting the presence of an alteration in a KCNQ3 gene or polypeptide in a sample from the subject, the presence of such an alteration being indicative of a responder subject.

As will be discussed below in more details, the alteration in a KCNQ3 gene or polypeptide may be any susceptibility marker in said gene or polypeptide, i.e., any nucleotide or amino acid alteration associated to schizophrenia, bipolar disorder or a related disease.

An alteration in the KCNQ3 gene may be any form of mutation(s), deletion(s), rearrangement(s) and/or insertion(s) in the coding and/or non-coding region of the gene, either isolated or in various combination(s). Mutations more specifically include point mutations. Deletions may encompass any region of two or more residues in a coding or non-coding portion of the gene. Typical deletions affect small regions, such as domains (introns) or repeated sequences or fragments of less than about 50 consecutive base pairs, although larger deletions may occur as well. Insertions may encompass the addition of one or several residues in a coding or non-coding portion of the gene. Insertions may typically comprise an addition of between 1 and 50 base pairs in the gene. Rearrangements include for instance sequence inversions. An alteration in the KCNQ3 gene may also be an aberrant modification of the polynucleotide sequence, such as of the methylation pattern of the genomic DNA, allelic loss of the gene or allelic gain of the gene. The alteration may be silent (i.e., create no modification in the amino acid sequence of the protein), or may result, for instance, in amino acid substitutions, frameshift mutations, stop codons, RNA splicing, e.g. the presence of a non-wild type splicing pattern of a messenger RNA transcript, or RNA or protein instability or a non-wild type level of the KCNQ3 polypeptide. Also, the alteration may result in the production of a polypeptide with altered function or stability, or cause a reduction or increase in protein expression levels.

Particular alterations of this invention are located in intron 1 or 9 of the KCNQ3 gene sequence, or in 5′ or 3′ regions of said gene. Typical alterations are single nucleotide substitutions. In a specific embodiment, the marker is a biallelic marker.

In this regard, the present invention now discloses several markers or mutations in the KCNQ3 gene, which are associated with schizophrenia and/or bipolar disorder. These mutations are reported in table 2.

Most preferred genetic alterations, which are associated with schizophrenia are disclosed in table 2a below:

TABLE 2a

| | SNP | | | Schizophrenia- | |
|---|---|---|---|---|---|
| Marker | name | Location | Polymorphism | associated allele | Position in sequence |
| 30-113/40 | M2 | 5′ of gene | A/C | A | 20337 in SEQ ID |
| | | | | | NO: 3 |
| 30-31/37 | M3 | 5′ of gene | C/T | T | 37 in SEQ ID NO: 3 |
| 30-28/54 | M6 | intron 1 | C/T | C | 212870 in SEQ ID |
| | | | | | NO: 4 |
| 30-40/45 | M9 | intron 1 | G/C | G | 151457 in SEQ ID |
| | | | | | NO: 4 |
| 30-30/58 | M12 | intron 1 | C/T | T | 73766 in SEQ ID |
| | | | | | NO: 4 |
| 30-63/29 | M16 | intron 1 | C/T | C | 29 in SEQ ID NO: 4 |
| 30-77/46 | M24 | 3′ of gene | A/G | A | 46 in SEQ ID NO: 5 |

Preferred (biallelic) markers of schizophrenia are thus selected from the biallelic markers M2, M3, M6, M9, M12, M16 and M24 as listed in Table 2a, or combination(s) thereof. More specifically, the invention comprises detecting a marker selected from M2, M3, M6, M9, M12, M16 and M24 as listed in Table 2a, the presence of a schizophrenia-associated allele being indicative of the presence, risk or predisposition to schizophrenia or a related disorder.

A preferred (biallelic) marker that is associated with bipolar disorder is M13 as listed in Table 2b below.

TABLE 2b

| | | | | BP- | |
|---|---|---|---|---|---|
| | SNP | | Poly- | associated | Position in |
| Marker | name | Location | morphism | allele | sequence |
| 30-25/42 | M13 | intron 1 | G/C | C | 94383 in SEQ ID |
| | | | | | NO: 4 |

A preferred object of this invention is a method of detecting the presence of or predisposition to schizophrenia or a related disorder in a subject, the method comprising detecting the presence or absence of the associated allele according to table 2a of one or more of the markers M2, M3, M6, M9, M12, M16 and M24 in a sample from the subject, the presence of the associated allele being indicative of the presence of or predisposition to schizophrenia or a related disorder in said subject.

A further preferred object of this invention is a method of detecting the presence of or predisposition to bipolar disorder or a related disorder in a subject, the method comprising detecting the presence or absence of the associated allele according to table 2b of the marker M13 in a sample from the subject, the presence of the associated allele being indicative of the presence of or predisposition to schizophrenia or a related disorder in said subject.

Now that the association between KCNQ3 and schizophrenia, bipolar disorder and related diseases has been established by the inventors, it should be understood that additional susceptibility markers can be identified within said gene or polypeptide, e.g., following the methodology disclosed in the examples.

A preferred embodiment of the present invention comprises the detection of the presence of a marker as disclosed in Table 2 in the KCNQ3 gene or RNA sequence of a subject, more particularly the detection of at least one marker as disclosed in Table 2a or 2b, or any combination thereof.

The presence of an alteration in the KCNQ3 gene may be detected by any technique known per se to the skilled artisan (reviewed by Kwok et al., 2003), including sequencing, pyrosequencing, selective hybridisation, selective amplification and/or mass spectrometry including matrix-assisted laser desorption/ionization time-of-flight mass spectrometry (MALDI-TOF MS) (Gut et al., 2004). In a particular embodiment, the alteration is detected by selective nucleic acid amplification using one or several specific primers, as disclosed in Tables 2c and 7 below. In another particular embodiment, the alteration is detected by selective hybridization using one or several specific probes.

Further techniques include gel electrophoresis-based genotyping methods such as PCR coupled with restriction fragment length polymorphism analysis, multiplex PCR, oligonucleotide ligation assay, and minisequencing; fluorescent dye-based genotyping technologies such as oligonucleotide ligation assay, pyrosequencing, single-base extension with fluorescence detection, homogeneous solution hybridization such as TaqMan, and molecular beacon genotyping; rolling circle amplification and Invader assays as well as DNA chip-based microarray and mass spectrometry genotyping technologies (Shi et al., 2001).

Furthermore, RNA expression of altered genes can be quantified by methods known in the art such as subtractive hybridisation, quantitative PCR, TaqMan, differential display reverse transcription PCR, serial, partial sequencing of cDNAs (sequencing of expressed sequenced tags (ESTs) and serial analysis of gene expression (SAGE)), or parallel hybridization of labeled cDNAs to specific probes immobilized on a grid (macro- and microarrays and DNA chips. Particular methods include allele-specific oligonucleotide (ASO), allele-specific amplification, fluorescent in situ hybridization (FISH) Southern and Northern blot, and clamped denaturing gel electrophoresis.

Protein expression analysis methods are known in the art and include 2-dimensional gel-electrophoresis, mass spectrometry and antibody microarrays (Freeman et al., Zhu et al., 2003).

Sequencing can be carried out using techniques well known in the art, using automatic sequencers. The sequencing may be performed on the complete gene or, more preferably, on specific domains thereof, typically those known or suspected to carry deleterious mutations or other alterations.

Amplification may be performed according to various techniques known in the art, such as by polymerase chain reaction (PCR), ligase chain reaction (LCR) and strand displacement amplification (SDA). These techniques can be performed using commercially available reagents and protocols. A preferred technique is allele-specific PCR.

Nucleic acid primers useful for amplifying sequences from the KCNQ3 gene are able to specifically hybridize with a portion of the KCNQ3 gene that either flanks or overlaps with an alteration, such as a susceptibility marker. The primer sequence overlaps with the alteration when said alteration is contained within the sequence of the KCNQ3 gene to which the primer hybridises. The primer sequence flanks the alteration when the primer hybridises with a portion of the KCNQ3 gene that is preferably located at a distance below 300 bp of said alteration, even more preferably below 250, 200, 150, 100, 50, 40, 30 or 20 bp from said alteration. Preferably the primer hybridises with a portion of the KCNQ3 gene that is at 5, 4, 3, 2, 1 bp distance or immediately adjacent to said alteration.

Most preferred primers are able to specifically hybridize with a portion of the KCNQ3 gene that either flanks or overlaps with an alteration as described in Table 2, more preferably in Table 2a or 2b. Examples of such primer sequences are disclosed in the following Table 2c (SEQ ID NOs: 7 to 30) and in Table 7 (SEQ ID NO: 31 to 82). Such primers represent a particular object of the present invention.

| | SNP | | | |
|---|---|---|---|---|
| Marker* | name | microsequencing primer | primer PCR PU | primer PCR RP |
| 30-113/40/B | M2 | CCACTAGCTTAAGAGGGATG | CCCTTTTCTGGCATATGGAG | ACAAGTGTGGCTCTCTGTCC |
| 30-31/37/B | M3 | AGGAACTGAAGATTTTCCTTGT | CCAGGGAAGGTTAATGACAG | AAGTGCAAGACTTGCTTTGG |
| 30-28/54/B | M6 | GCTCAGCACCCCACCCAC | ATGCTACCACTCCATCAACC | AGATAAATGGGCTCAGCACC |
| 30-40/45/B | M9 | CACCAAGGATGGTATGCCC | TGAGCTCCTTGTGAATTGGG | ATTCCTTACCTGGCTCTTCC |
| 30-30/58/B | M12 | CAGACAGGGAGCTTAGATGAA | GCCATTTCTCTCTTTCCTGC | AACTCTATCAGAAGCCAGCC |
| 30-63/29/A | M16 | GGTGGGTTTCTTCACAGATG | CTCATATAGGTGGGTTTCTTC | GGGCAAATACAATTCAGTCC |
| 30-77/46/A | M24 | GGCACATCAGGAGAAAA | AAGGCAAGAGAGTGGAGAAG | GCACGGACCCCTTATTTCTC |
| 30-25/42 | M13 | CAGGATTAGTGCAGTCCCCT | GCAGCGTGGAGTTTCAAATG | ACCCCTTAGTTCAAGCCAAC |

*A means the mis primer is sense ; B means the mis primer is reverse.

The invention also relates to the use of a nucleic acid primer or a pair of nucleic acid primers as described above in a method of detecting the presence of or predisposition to schizophrenia, bipolar disorder or a related disorder in a subject or in a method of assessing the response of a subject to a treatment of schizophrenia, bipolar disorder or a related disorder.

According to another embodiment of the present invention, the methods involve the use of a nucleic acid probe specific for a KCNQ3 or altered KCNQ3 gene or RNA, followed by the detection of the presence of a hybrid. The probe may be used in suspension or immobilized on a substrate or support. The probe is typically labelled to facilitate detection of hybrids.

In this respect, a specific object of this invention is a nucleic acid probe complementary to and specific for a region of a KCNQ3 gene or RNA that carries an alteration as described in Table 2, preferably in Table 2a or 2b. The probes of the present invention are, more preferably, capable of discriminating between an altered and non-altered KCNQ3 gene or RNA sequence, i.e., they specifically hybridise to said KCNQ3 gene or RNA carrying a particular alteration as described above, and essentially do not hybridise under the same hybridization conditions or with the same stability to a KCNQ3 gene or RNA lacking said alteration.

The invention also concerns the use of a nucleic acid probe as described above in a method of detecting the presence of or predisposition to schizophrenia, bipolar disorder or a related disorder in a subject or in a method of assessing the response of a subject to a treatment of schizophrenia, bipolar disorder or a related disorder.

The detection methods can be performed in vitro, ex vivo or in vivo, preferably in vitro or ex vivo. They are typically performed on a sample from the subject, such as any biological sample containing nucleic acids or polypeptides. Examples of such samples include fluids, tissues, cell samples, organs, biopsies, etc. Most preferred samples are blood, plasma, saliva, urine, seminal fluid, etc. The sample may be collected according to conventional techniques and used directly for diagnosis or stored. In particular, they may be obtained by non-invasive methods, such as from tissue collections. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids and/or polypeptides may be pre-purified or enriched by conventional techniques, and/or reduced in complexity. Nucleic acids and polypeptides may also be treated with enzymes or other chemical or physical treatments to produce fragments thereof. Considering the high sensitivity of the claimed methods, very few amounts of sample are sufficient to perform the assay.

The sample is typically contacted with probes or primers as disclosed above. Such contacting may be performed in any suitable device, such as a plate, tube, well, glass, etc. The contacting may performed on a substrate coated with said specific reagents, such as a nucleic acid array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids of the sample.

The finding of an altered KCNQ3 gene or RNA or polypeptide in the sample is indicative of the presence, predisposition or stage of progression of schizophrenia, bipolar disorder or a related disorder in the subject. Typically, one only of the above-disclosed markers is assessed, or several of them, in combination(s).

Drug Screening

As indicated above, the present invention also provides novel targets and methods for the screening of drug candidates or leads. These screening methods include binding assays and/or functional assays, and may be performed in vitro, in cell systems or in animals.

Potassium channels can be tested functionally in living cells. KCNQ3 polypeptides of the invention and other polypeptides that are required to functionally express a potassium channel comprising KCNQ3 are either expressed endogeneously in appropriate reporter cells or are introduced recombinantly. Channel activity can be monitored by concentration changes of the permeating ion, by changes in the transmembrane electrical potential gradient, or by measuring a cellular response (e.g., expression of a reporter gene or secretion of a neurotransmitter) triggered or modulated by the polypeptide's activity.

Potassium channel currents result in changes of electrical membrane potential (Vm) 25 which can be monitored directly using potentiometric fluorescent probes. These electrically charged indicators (e.g., the anionic oxonol dye DiBAC4(3)) redistribute between extra- and intracellular compartments in response to voltage changes across the membrane in which the potassium channel resides. The equilibrium distribution is governed by the Nernst-equation. Thus, changes in membrane potential results in concomitant changes in cellular fluorescence. Again, changes in Vm might be caused directly by the activity of the target potassium channel or through amplification and/or prolongation of the signal by channels co-expressed in the same cell.

Another approach to determining the activity of potassium channel proteins involves the electrophysiological determination of ionic currents. Cells, which endogenously express a particular potassium channel protein, can be used to study the effects of various test compounds or potassium channel protein-like polypeptides on endogenous ionic currents attributable to the activity of potassium channel proteins.

Alternatively, cells, which do not express a particular potassium channel protein, can be employed as hosts for the expression of a particular potassium channel proteins whose activity can then be studied by electrophysiological or other means. Cells preferred as host cells for the heterologous expression of potassium channel proteins are preferably mammalian cells such as COS cells, mouse L cells, CHO cells (e.g. DG44 cells), human embryonic kidney cells (e.g. HEK293 cells), African green monkey cells and the like; amphibian cells, such as Xenopus Levis oocytes; or cells of yeast such as S. cerevisiae or P. pastoris. See, e.g. U.S. Pat. No. 5,876,958.

Electrophysiological procedures for measuring the current across a cell membrane are well known. A preferred method is the use of a voltage clamp as in the whole-cell patch clamp technique. Non-calcium currents can be eliminated by established methods so as to isolate the ionic current flowing through potassium channel proteins. In the case of heterologously expressed potassium channel proteins, ionic currents resulting from endogenous potassium channel proteins can be suppressed by known pharmacological or electrophysiological techniques. See, e.g. 25 U.S. Pat. No. 5,876,958.

A further activity of potassium channel proteins, which can be assessed, is their ability to bind various ligands, including test compounds or potassium channel protein-like polypeptides. The ability of a test compound to bind potassium channel polypeptides or fragments thereof may be determined by any appropriate competitive binding analysis (e.g., Scatchard plots), wherein the binding capacity and/or affinity determined in the presence and absence of one or more concentrations a compound having known affinity for the potassium channel proteins. Binding assays can be performed using whole cells, which express potassium channel proteins (either endogenously or heterologously), membranes prepared from such cells, or purified potassium channel polypeptides. Test compounds can be tested for the ability to increase or decrease the activity of a human potassium channel polypeptide.

In this regard, a particular object of this invention resides in the use of a KCNQ3 polypeptide as a target for screening candidate drugs for treating or preventing schizophrenia, bipolar disorder or a related disorder.

Another object of this invention resides in methods of selecting biologically active compounds, said methods comprise contacting a candidate compound with a KCNQ3 gene or polypeptide, and selecting compounds that bind said gene or polypeptide.

A “biologically active” compound denotes any compound having biological activity in a subject, preferably therapeutic activity, more preferably a neuroactive compound, and further preferably a compound that can be used for treating schizophrenia or bipolar disorder or a related disorder, or as a lead to develop drugs for treating schizophrenia or bipolar disorder or a related disorder. A “biologically active” compound preferably is a compound that modulates the activity of KCNQ3 or a potassium channel comprising KCNQ3. Modulation of channel activity can be assessed as described above.

A further other object of this invention resides in methods of selecting biologically active compounds, said method comprising contacting a candidate compound with recombinant host cell expressing a KCNQ3 polypeptide with a candidate compound, and selecting compounds that bind said KCNQ3 polypeptide at the surface of said cells and/or that modulate the activity of said KCNQ3 polypeptide.

The above methods may be conducted in vitro, using various devices and conditions, including with immobilized reagents, and may further comprise an additional step of assaying the activity of the selected compounds in a model of schizophrenia, bipolar disorder or a related disorder, such as an animal model.

Methods for identifying compounds that bind to and/or modulate the activity of the KCNQ3 polypeptide and are candidate drugs for treating or preventing schizophrenia, bipolar disorder or a related disorder are known in the art (Cooper E C et al., 2003). Such methods include methods that identify inhibitors such as antagonists or activators such as agonists of the KCNQ3 polypeptide or the potassium channel comprising the KCNQ3 polypeptide.

A particular method of screening comprises determining the ability of a candidate compound to bind (in vitro) to the C-terminal intracellular domain of a KCNQ3 polypeptide, in particular to a region comprising the putative assembly domains (such as the “A domain”) of a KCNQ3 polypeptide.

Another particular method of screening comprises determining the ability of a candidate compound to bind (in vitro) to a region or motif located between the fifth and sixth trans-membrane domains of a KCNQ3 polypeptide sequence and, more particularly, to the pore-forming P-loop of a KCNQ3 polypeptide.

A further particular method of screening comprises determining the ability of a candidate compound to bind (in vitro) to the N-terminal intracellular domain of a KCNQ3 polypeptide.

Another particular method of screening comprises determining the ability of a candidate compound to bind to a potassium channel expressed at the surface of a cell, wherein said potassium channel comprises at least one KCNQ3 polypeptide. The potassium channel may comprise up to 4 sub-units. In a particular embodiment, the potassium channel is a homomeric complex comprising up to 4 KCNQ3 polypeptides. In another embodiment, the potassium channel is a heteromeric complex comprising at least one KCNQ3 polypeptide and at least one distinct sub-unit, preferably selected from a KCNQ2 polypeptide or a KCNQ5 polypeptide.

Binding to the target gene or polypeptide provides an indication as to the ability of the compound to modulate the activity of said target, and thus to affect a pathway leading to schizophrenia, bipolar disorder or a related disorder in a subject. The determination of binding may be performed by various techniques, such as by labelling of the candidate compound, by competition with a labelled reference ligand, etc. For in vitro binding assays, the polypeptides may be used in essentially pure form, in suspension, immobilized on a support, or expressed in a membrane (intact cell, membrane preparation, liposome, etc.).

Modulation of activity includes, without limitation, stimulation of the surface expression of the KCNQ3 receptors, modulation of multimerization of said receptors (e.g., the formation of multimeric complexes with other sub-units), modulation of potassium concentrations or fluxes, etc. The cells used in the assays may be any recombinant cell (i.e., any cell comprising a recombinant nucleic acid encoding a KCNQ3 polypeptide) or any cell that expresses an endogenous KCNQ3 polypeptide. Examples of such cells include, without limitation, prokaryotic cells (such as bacteria) and eukaryotic cells (such as yeast cells, mammalian cells, insect cells, plant cells, etc.). Specific examples include E. coli Pichia pastoris, Hansenula polymorpha, Schizosaccharomyces pombe, Kluyveromyces or Saccharomyces yeasts, mammalian cell lines (e.g., Vero cells, CHO cells, 3T3 cells, COS cells, HEK cells, etc.) as well as primary or established mammalian cell cultures (e.g., produced from fibroblasts, embryonic cells, epithelial cells, nervous cells, adipocytes, etc.) or xenopus oocytes.

In a particular embodiment, the selected compounds are agonists of KCNQ3, i.e., compounds that can bind to KCNQ3 and/or mimic the activity of an endogenous ligand thereof, such as any channel opener.

In another particular embodiment, the selected compounds are antagonists of KCNQ3, i.e., compounds that can bind to KCNQ3 and/or inhibit or block the activity of an endogenous ligand thereof, such as any channel inhibitors or channel blockers.

In a particular embodiment, the screening assays of the present invention use, either alone or in combination with other isoforms, an altered KCNQ3 gene or polypeptide, particularly a KCNQ3 gene or polypeptide having a mutation as listed in Table 2, more preferably a mutation as listed in Table 2a or 2b.

A further object of this invention resides in a method of selecting biologically active compounds, said method comprising contacting in vitro a test compound with a KCNQ3 polypeptide according to the present invention and determining the ability of said test compound to modulate the activity of said KCNQ3 polypeptide.

A further object of this invention resides in a method of selecting biologically active compounds, said method comprising contacting in vitro a test compound with a KCNQ3 gene according to the present invention and determining the ability of said test compound to modulate the expression of said KCNQ3 gene.

In another embodiment, this invention relates to a method of screening, selecting or identifying active compounds, particularly compounds active on schizophrenia, bipolar disorder or related disorders, the method comprising contacting a test compound with a recombinant host cell comprising a reporter construct, said reporter construct comprising a reporter gene under the control of a KCNQ3 gene promoter, and selecting the test compounds that modulate (e.g. stimulate or reduce) expression of the reporter gene.

In another embodiment, this invention relates to the use of a KCNQ3 polypeptide or fragment thereof, whereby the fragment is preferably a KCNQ3 gene-specific fragment, for isolating or generating an antagonist or inhibitor of the KCNQ3 polypeptide for the treatment of schizophrenia, bipolar disorder or a related disorder, wherein said antagonist or inhibitor is selected from the group consisting of:

1. a specific antibody or fragment thereof including

a) a chimeric,
b) a humanized or
c) a fully human antibody as well as
   2. a bispecific or multispecific antibody,
   3. a single chain (e.g. scFv) or
   4. single domain antibody, or
   5. a peptide- or non-peptide mimetic derived from said antibodies or
   6. an antibody-mimetic such as
a) an anticalin or
b) a fibronectin-based binding molecule (e.g. trinectin or adnectin).

The generation of peptide- or non-peptide mimetics from antibodies is known in the art (Saragovi et al., 1991 and Saragovi et al., 1992).

Anticalins are also known in the art (Vogt et al., 2004). Fibronectin-based binding molecules are described in U.S. Pat. No. 6,818,418 and WO2004029224.

The above screening assays may be performed in any suitable device, such as plates, tubes, dishes, flasks, etc. Typically, the assay is performed in multi-wells plates. Several test compounds can be assayed in parallel.

Furthermore, the test compound may be of various origin, nature and composition, such as any small molecule, nucleic acid, lipid, peptide, polypeptide including an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), etc., in isolated form or in mixture or combinations.

Pharmaceutical Compositions and Therapy

The present invention now discloses novel approaches to the treatment of schizophrenia, bipolar disorder and related disorders by modulating the activity or expression of a KCNQ3 gene or polypeptide. Indeed, for the first time, the invention shows that a KCNQ3 receptor is related to schizophrenia and bipolar disorder, and that this gene is altered in patients suffering from these disorders.

In this regard, a particular object of this invention resides in the use of a functional KCNQ3 polypeptide, or a nucleic acid encoding the same, for the manufacture of a pharmaceutical composition for treating or preventing schizophrenia, bipolar disorder or a related disorder in a subject. The term “functional” KCNQ3 polypeptide indicates the polypeptide can form a functional K⁺ channel.

A further object of this invention resides in the use of a modulator of KCNQ3 for the manufacture of a pharmaceutical composition for treating or preventing schizophrenia, bipolar disorder or a related disorder in a subject.

In a first embodiment, the modulator is an activator or agonist of a KCNQ3 polypeptide. KCNQ3 agonists are particularly suited for treating bipolar disorder and in particular the manic phase of bipolar disorder. Bipolar disorder and especially manic episodes are characterized by overactivity, euphoria and running thoughts; agonists of M-channels can be used for the treatment of this phase of the disease.

An activator or agonist of KCNQ3 includes, without limitation, any compound or molecule or condition that causes activation (e.g., an opening), or mimics the activity of a potassium channel comprising a KCNQ3 polypeptide, as well as any compound or molecule or condition that causes or stimulates surface expression of a functional KCNQ3 polypeptide. Examples of such compounds include, for instance, a wild type KCNQ3 polypeptide or coding nucleic acid, an activator of a KCNQ3 gene promoter, as well as any drug that activates a potassium channel comprising a KCNQ3 polypeptide. Specific examples of such drugs include, for instance, Retigabine (Dailey et al., 1995 and Wickenden et al., 2000) and BMS-204352 (Gribkoff et al., 2001).

In a particular embodiment, the agonist is a natural KCNQ3 channel opener, or an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin), that selectively binds and opens KCNQ3 or increases opening of KCNQ3.

In another embodiment, the modulator is an inhibitor or antagonist of a KCNQ3 polypeptide. KCNQ3 antagonists are particularly suited for treating schizophrenia and the depressive phase of bipolar disorder. Inhibitors or antagonists of KCNQ3 enhance depolarization-induced transmitter release and improve learning performance in animal models.

An inhibitor or antagonist of KCNQ3 includes, without limitation, any compound or molecule or condition that causes inhibition (e.g., a blockade) of the activity of a potassium channel comprising a KCNQ3 polypeptide, as well as any compound or molecule or condition that inhibits (e.g., reduces) or prevents surface expression of a functional KCNQ3 polypeptide. Examples of such compounds include, for instance, an inhibitory nucleic acid (e.g., an antisense nucleic acid, a riboszyme, a siRNA, etc.), an inhibitor of a KCNQ3 gene promoter, as well as any drug that blocks a potassium channel comprising a KCNQ3 polypeptide. Specific examples of such drugs include, for instance, Linopirdine (Lamas et al., 1997) and 10,10-bis(4-pyridinylmethyl)-9(10H)-anthracenone (XE991) (Zaczek et al. 1998).

In a particular embodiment, the antagonist is an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin) that selectively binds KCNQ3 and prevents, reduces or blocks channel opening or function.

A further object of this invention resides in a pharmaceutical composition comprising a nucleic acid encoding a KCNQ3 polypeptide or a vector encoding the same, and a pharmaceutically acceptable carrier or vehicle.

The above uses or compositions are particularly suited for treating or preventing schizophrenia, bipolar disorder or a related disorder in a subject presenting an alteration in the KCNQ3 gene or polypeptide, particularly in a subject presenting a marker as described in Table 2 above, more specifically in Table 2a or 6.

The invention also relates to any vector comprising a KCNQ3 nucleic acid comprising a marker as disclosed in Table 2 above, or a fragment thereof comprising the alteration. The vector may be any plasmid, phage, virus, episome, artificial chromosome, and the like. In a particular embodiment, the vector is a recombinant virus. Viral vectors may be produced from different types of viruses, including without limitation baculoviruses, retroviruses, adenoviruses, AAVs, etc., according to recombinant DNA techniques known in the art. The recombinant virus is typically replication-defective, even more preferably selected from E1- and/or E4-defective adenoviruses, Gag-, pol- and/or env-defective retroviruses and Rep- and/or Cap-defective AAVs. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO95/14785, WO96/22378, U.S. Pat. No. 5,882,877, U.S. Pat. No. 6,013,516, U.S. Pat. No. 4,861,719, U.S. Pat. No. 5,278,056 and WO94/19478.

A further aspect of this invention is a recombinant host cell comprising a vector or a nucleic acid as defined above. The recombinant cell may be any prokaryotic or eukaryotic cells as discussed above. The recombinant cell preferably expresses a recombinant KCNQ3 polypeptide at its surface.

The invention also relates to a method of treating or preventing schizophrenia, bipolar disorder or a related disorder in a subject, the method comprising administering to said subject a compound that modulates expression or activity of a KCNQ3 gene or polypeptide as defined above.

A preferred embodiment of the invention is the use of an activator or agonist of KCNQ3 or a potassium channel comprising KCNQ3 in the preparation of a medicament for the treatment of bipolar disorder, in particular the manic phase of bipolar disorder or a related disorder.

A particularly preferred embodiment of the invention is the use of an activator or agonist of KCNQ3 or a potassium channel comprising KCNQ3 in the preparation of a medicament for the treatment of bipolar disorder, in particular the manic phase of bipolar disorder or a related disorder wherein the activator or agonist is an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin).

A further preferred embodiment of the invention is the use of an inhibitor or antagonist of KCNQ3 or a potassium channel comprising KCNQ3 in the preparation of a medicament for the treatment of schizophrenia, bipolar disorder, in particular the depressive phase of bipolar disorder or a related disorder.

A further preferred embodiment of the invention is the use of an inhibitor or antagonist of KCNQ3 or a potassium channel comprising KCNQ3 in the preparation of a medicament for the treatment of schizophrenia, bipolar disorder, in particular the depressive phase of bipolar disorder or a related disorder, wherein said inhibitor or antagonist is Linopirdine or 10,10-bis(4-pyridinylmethyl)-9(10H)-anthracenone (XE991).

A particularly preferred embodiment of the invention is the use of an inhibitor or antagonist of KCNQ3 or a potassium channel comprising KCNQ3 in the preparation of a medicament for the treatment of schizophrenia, bipolar disorder, in particular the depressive phase of bipolar disorder or a related disorder, wherein the inhibitor or antagonist is an antibody such as a chimeric, humanized or fully human antibody or an antibody fragment, peptide- or non-peptide mimetic derived therefrom as well as a bispecific or multispecific antibody, a single chain (e.g. scFv) or single domain antibody or an antibody-mimetic such as an anticalin or fibronectin-based binding molecule (e.g. trinectin or adnectin).

Another preferred embodiment of the invention is the use of a compound that inhibits or downregulates the expression of a KCNQ3 polypeptide in the preparation of a medicament for the treatment of schizophrenia, bipolar disorder, in particular the depressive phase of bipolar disorder or a related disorder.

Another particularly preferred embodiment of the invention is the use of a compound that inhibits or downregulates the expression of a KCNQ3 polypeptide in the preparation of a medicament for the treatment of schizophrenia, bipolar disorder, in particular the depressive phase of bipolar disorder or a related disorder, wherein the compound is an inhibitory nucleic acid such as an antisense nucleic acid, a riboszyme or a small interfering RNA (siRNA). Techniques for interfering with the expression of a protein employing antisense nucleic acids, ribozymes or siRNA for the treatment of human disorders and in particular CNS disorders are known in the art (Wood et al., 2003 and Jaeger et al., 2004).

A particular embodiment of the present invention resides in a method of treating or preventing schizophrenia in a subject, the method comprising (i) detecting in a sample from the subject the presence of an alteration in the KCNQ3 gene or polypeptide as defined above and (ii) administering to said subject an agonist of KCNQ3. Preferably, said alteration is selected from the group consisting of a SNP as disclosed in Table 2.

A particular embodiment of the present invention resides in a method of treating or preventing the manic phase of bipolar disorder in a subject, the method comprising (i) detecting in a sample from the subject the presence of an alteration in the KCNQ3 gene or polypeptide as defined above and (ii) administering to said subject an antagonist of KCNQ3. Preferably, said alteration is selected from the group consisting of a SNP as disclosed in Table 2.

A particular embodiment of the present invention resides in a method of treating or preventing the depressive phase of bipolar disorder in a subject, the method comprising (i) detecting in a sample from the subject the presence of an alteration in the KCNQ3 gene or polypeptide as defined above and (ii) administering to said subject an agonist of KCNQ3. Preferably, said alteration is selected from the group consisting of a SNP as disclosed in Table 2.

Further aspects and advantages of the present invention will be disclosed in the following experimental section, which should be regarded as illustrative and not limiting the scope of the present application.

### EXAMPLES

1—Description of the Schizophrenia Collections Used for the Analyses of Candidate Genes.

The association studies were performed on four different populations. One collection of samples came from Moscow, Russia (the “Rogaev” collection). The others collections came from England and were provided by the University College of London (the “UCL” collection), by the Institute of Psychiatry of London (the “IOP” collection) and by the Burnley Hospital (the “Burnley” collection).

All collections include individuals that are affected (patients or “cases”) or not affected (“controls”) by schizophrenia.

67 random markers that were unlinked and not associated with the disease were used to perform stratification study and calculate the Fst value.

TABLE 1
Description and stratification study of the four different collections

| | Institute of | | United College | |
|---|---|---|---|---|
| | Psychiatry, | | of London (UCL) | |
| Population | London (IoP) | Burnley Hospital | UCLsch | Rogaev |
| Origin | English 1 | English 2 | English 3 | Russian |
| | schizophrenic | schizophrenic | schizophrenic | schizophrenic |
| Cases | 193 (107 males) | 154 (107 males) | 180 (119 males) | 154 |
| Controls | 184 (105 males) | | 295 (142 males) | 158 |
| Stratification | Fst = −0.000174 | Fst = 0.000252 | Fst = −0.000526 | Fst = 0.000386 |
| on 67 | pvalue = 6.13E−01 | pvalue = 3.06E−01 | pvalue = 1.05E−01 | pvalue = 2.52E−01 |
| random | (NS) | (NS) | (NS) | (NS) |
| markers | | | | |

All the Fst values found for each collection indicate that these samples are genetically homogeneous, hence they are ok to be used in association analysis.

2—Association Studies Between Schizophrenia and the KCNQ3 Gene

a—Genotyping of Cases and Controls

The general strategy to perform the association studies was to individually scan the DNA samples from all individuals in each population described above in order to establish the allele frequencies of biallelic markers.

The scan procedure is based on an allele-specific primer extension reaction that allows for the differentiation of homozygous normal, heterozygous mutant and homozygous mutant samples. The reaction can be used to characterize genetic variations that include deletions, insertions and substitutions.

Briefly, a region of interest, containing the polymorphic site is amplified by PCR, using two PCR primers (Primers PU and RP). A treatment with an Alcaline Phosphatase (SAP) is applied to remove non-incorporated dNTPs. The Oligo MIS primer anneals close to the polymorphic site and is extended dependent on the polymorphism. The different extension products and the OLIGO MIS primer can be clearly differentiated in a mass spectrum.

Typically, during the microsequencing (MIS) reaction, the primer is extended by a specific number of nucleotides depending on the allele and the design of the assay. In the reaction mixture, all four nucleotides A, T, C, and G are present as either dNTPs or ddNTPs (for regular SNP assays, usually three nucleotides are present as ddNTPs and one as dNTP). The incorporation of a ddNTP terminates the extension of the MIS primer. Using a DNA polymerase that incorporates both ddNTPs and dNTPs at the same rate, the MIS reaction produces allele-specific extension products of different masses depending on the sequence analyzed. Prior to mass spectrometry, the products of the MIS reaction are desalted with a SpectroCLEAN solution and SpectroCLEAN plate (SEQUENOM), and transferred onto a SpectroCHIP microarray from SEQUENOM. The SpectroCHIP is then analyzed by the SpectroREADER (SEQUENOM) mass spectrometer.

Frequencies of every biallelic marker in each population (cases and controls) were determined by microsequencing reactions on amplified fragments obtained by genomic PCR performed on the DNA samples from each individual.

The experiments were performed as detailed below:

1.) PCR

| | Initial | | Volume for | | Concentration in | |
|---|---|---|---|---|---|---|
| Reagents | concentration | | 1 reaction | | the final volume | |
| DNA | 2.5 | ng/μL | 1 | μL | 0.5 | ng/μL |
| Hot Star Taq Buffer | 10 | X | 0.5 | μL | 1 | X |
| MgCl2 | 25 | mM | 0.2 | μL | 1 | mM |
| dNTPs | 2.5 | mM | 0.4 | μL | 200 | μM |
| Primer PU | 30 | μM | 0.0167 | μL | 100 | nM |
| Primer RP | 30 | μM | 0.0167 | μL | 100 | nM |
| Hot Star Taq | 5 | U/μL | 0.02 | μL | 0.02 | U/μL |
| H2O molecular | 5 | μL | 2.8466 | μL | qsp 5 | μL |
| grade qsp | | | | | | |

| | |
|---|---|
| 95° C. | 15 minutes |
| 95° C. | 20 secondes |
| 56° C. | 30 secondes |
| 72° C. | 1 minute |
| 72° C. | 3 minutes |
| 10° C. | waiting |

2.) SAP PURIFICATION

| | Initial | | Volume for | | Concentration in | |
|---|---|---|---|---|---|---|
| Reagents | concentration | | 1 reaction | | the final volume | |
| ThermoSeq Buffer | 16 | X | 0.1063 | μL | 0.243 | X |
| SAP | 12.7 | U/μL | 0.0237 | μL | 0.0429 | U/μL |
| H2O molecular | 2 | μL | 1.8701 | μL | 7 | μL |
| grade qsp | | | | | | |

| | |
|---|---|
| 37° C. | 20 minutes |
| 85° C. | 5 minutes |
| 10° C. | waiting |

3) MICROSEQUENSING REACTION (MIS)

| | Initial | | Volume for | | Concentration in | |
|---|---|---|---|---|---|---|
| Reagents | concentration | | 1 reaction | | the final volume | |
| ThermoSeq Buffer | 16 | X | 0.125 | μL | 0.222 | X |
| dNTP | 100 | mM | 0.0045 | μL | 50 | μM |
| ddNTP | 10 | mM | 0.045 | μL | 50 | μM |
| ddNTP | 10 | mM | 0.045 | μL | 50 | μM |
| ddNTP | 10 | mM | 0.045 | μL | 50 | μM |
| Primer MIS | 30 | μM | 0.18 | μL | 600 | nM |
| Thermosequenase | 32 | U/μL | 0.018 | μL | 0.064 | U/μL |
| H2O molecular | 2 | μL | 1.5375 | μL | 9 | μL |
| grade qsp | | | | | | |

| | |
|---|---|
| 94° C. | 2 minutes |
| 94° C. | 5 secondes |
| 52° C. | 5 secondes |
| 72° C. | 5 secondes |
| 10° C. | waiting |

4.) Cleaning—Desalting

Prior to mass spectrometry, the products of the MIS reaction are desalted with a SpectroCLEAN solution and SpectroCLEAN plate (SEQUENOM), and transferred onto a SpectroCHIP microarray from SEQUENOM.

The SpectroCHIP is then analyzed by the SpectroREADER (SEQUENOM) mass spectrometer.

The results are presented in Table 2 below.

TABLE 2
List of markers located on KCNQ3

| | SNP | | | | Main |
|---|---|---|---|---|---|
| Marker | name | position | location | Type | allele |
| 30-106/40 | M1 | 64782 in SEQ ID NO: 3 | 5′ of gene | A/T | A |
| 30-113/40 | M2 | 35163 in SEQ ID NO: 3 | 5′ of gene | A/C | |
| 30-31/37 | M3 | 14884 in SEQ ID NO: 3 | 5′ of gene | C/T | |
| 30-49/29 | M4 | 29 in SEQ ID NO: 3 | 5′ of gene | C/T | C |
| 30-107/29 | M5 | 274609 in SEQ ID NO: 3 | intron 1 | A/G | G |
| 30-28/54 | M6 | 248064 in SEQ ID NO: 4 | intron 1 | C/T | |
| 30-32/51 | M7 | 235945 in SEQ ID NO: 4 | intron 1 | G/T | T |
| 30-46/48 | M8 | 209663 in SEQ ID NO: 4 | intron 1 | A/G | A |
| 30-40/45 | M9 | 186650 in SEQ ID NO: 4 | intron 1 | G/C | |
| 30-34/30 | M10 | 160119 in SEQ ID NO: 4 | intron 1 | A/G | A |
| 30-76/55 | M11 | 132375 in SEQ ID NO: 4 | intron 1 | C/T | C or T |
| 30-30/58 | M12 | 108959 in SEQ ID NO: 4 | intron 1 | C/T | |
| 30-25/42 | M13 | 94383 in SEQ ID NO: 4 | intron 1 | G/C | G |
| 30-51/49 | M14 | 66775 in SEQ ID NO: 4 | intron 1 | A/G | G |
| 30-59/28 | M15 | 52318 in SEQ ID NO: 4 | intron 1 | A/G | A |
| 30-63/29 | M16 | 35222 in SEQ ID NO: 4 | intron 1 | C/T | |
| 30-48/53 | M17 | 14570 in SEQ ID NO: 4 | intron 1 | C/T | C |
| 30-42/38 | M18 | 21 in SEQ ID NO: 4 | intron 1 | C/T | C |
| 30-53/77 | M19 | | intron 3 | C/T | C |
| 30-54/37 | M20 | 20 in SEQ ID NO: 6 | intron 9 | A/G | A |
| 30-52/62 | M21 | | intron 10 | C/T | T |
| 30-44/27 | M22 | | 3′ of gene | A/G | A |
| 30-57/63 | M23 | 69537 in SEQ ID NO: 5 | 3′ of gene | A/G | G |
| 30-77/46 | M24 | 27764 in SEQ ID NO: 5 | 3′ of gene | A/G | |
| 30-91/27 | M25 | 8 in SEQ ID NO: 5 | 3′ of gene | A/G | G |

b- SNP frequency analysis

Method

Markers were analysed individually. Pearson's χ2 test (2×2) was used to compare allele frequencies between cases and controls. Data were analysed using a 3×2 χ2 test for the overall difference in genotype frequencies between cases and controls. The Exact Fisher test was performed when the conditions were not respected for the Pearson's χ2 test.

Then we calculated the difference between allelic frequencies in cases and in controls: the larger the difference in allelic frequency for a given SNP, the more probable is an association between the genomic region containing that SNP and the disorder. The “chosen” allele is the allele for which the frequency is increased in cases compared to controls.

Hardy-Weinberg equilibrium statistics were calculated separately for cases and controls data and Observed and Expected genotype frequencies were compared using a Pearson's χ2 test. A departure from Hardy-Weinberg equilibrium (HWE) in case population may indicate that a mutation had occurred, which could be responsible for increasing the risk for schizophrenia.

Results

The p-values in table 3 show the probability of association between a biallelic marker and schizophrenia. A p-value under 5e-02 suggests a significant association between the biallelic marker and schizophrenia [only the significant p-values shown].

TABLE 3
Significant p-values and associated data for SNP located within the KCNQ3 gene

| | | Location | | Allele | | | | HWE |
|---|---|---|---|---|---|---|---|---|
| | SNP | on KCNQ3 | Chosen | frequency | Allelic | Allelic | Genotypic | cases |
| Collection | name | gene | allele | difference | p-value | OR | p-value | p-value |
| IOP | M6 | intron 1 | C | 0.08 | 3.00E−02 | 1.41 | 1.64E−02 | 1.29E−01 |
| Burnley | M24 | 3′ of gene | A | 0.08 | 1.17E−02 | 1.70 | 2.21E−02 | 4.87E−01 |
| UCL | M2 | 5′ of gene | A | 0.06 | 4.67E−02 | 1.36 | 1.45E−01 | 2.59E−01 |
| | M9 | intron 1 | G | 0.06 | 7.21E−02 | 1.29 | 4.36E−02 | 1.23E−01 |
| | M16 | intron 1 | C | 0.04 | 2.99E−01 | 1.15 | 3.24E−02 | 5.27E−02 |
| Rogaev | M3 | 5′ of gene | T | 0.01 | 8.85E−01 | 1.02 | 5.27E−01 | 3.55E−02 |
| | M12 | intron 1 | T | 0.02 | 5.75E−01 | 1.10 | 4.38E−02 | 2.97E−02 |

By estimating the allelic Odds Ratio (OR) we evaluate the probability of having the disease when carrying a given allele (=chosen [or ‘risk’] allele) compared to not carrying it. An OR higher than 1 shows that the probability of having Schizophrenia is higher when carrying the ‘risk’ allele [or genotype or haplotype] than when carrying the other ones. The genotypic OR allows the identification of the ‘risk’ genotype(s) for an associated biallelic marker. The genotypic odds ratio was calculated and Table 4 shows the significant results.

TABLE 4
Genotypic OR for SNP located on KCNQ3

| | SNP | | Odds | Confidence | |
|---|---|---|---|---|---|
| Collection | name | Genotypes | Ratio | Interval | p-value |
| Rogaev | M12 | CC vs (CT + TT) | 1.68 | 0.78-3.59 | 2.0E−01 |
| | | TT vs (CT + CC) | 1.43 | 0.91-2.25 | 1.0E−01 |
| | | (CC + TT) vs CT | 1.76 | 1.11-2.79 | 2.0E−02 |
| IOP | M6 | CC vs (CT + TT) | 2.61 | 1.31-5.19 | 3.0E−03 |
| | | TT vs (CT + CC) | 0.77 | 0.51-1.18 | 2.0E−01 |
| | | CT vs (CC + TT) | 0.87 | 0.57-1.32 | 5.0E−01 |
| Burnley | M24 | AA vs (AG + GG) | 1.94 | 1.21-3.12 | 6.0E−03 |
| | | GG vs (AA + AG) | 0.75 | 0.21-2.71 | 8.0E−01 |
| | | AG vs (GG + AA) | 0.52 | 0.32-0.84 | 8.0E−03 |
| UCL | M9 | CC vs (CG + GG) | 0.86 | 0.59-1.25 | 5.0E−01 |
| | | CG vs (CC + GG) | 0.86 | 0.59-1.25 | 4.0E−01 |
| | | GG vs (CG + CC) | 2.05 | 1.16-3.63 | 2.0E−02 |
| | M16 | CC vs (CT + TT) | 1.59 | 1.05-2.41 | 3.0E−02 |
| | | TT vs (CT + CC) | 1.14 | 0.74-1.77 | 6.0E−01 |
| | | (CC + TT) vs CT | 1.6 | 1.1-2.33 | 1.0E−02 |

Seven different SNPs located in the KCNQ3 gene (M2, M3, M6, M9, M12, M16 and M24) are associated with Schizophrenia, across four different populations. Three SNPs are associated to an increased risk for schizophrenia in UCL and other SNPs—all within the gene—in the other samples. This result suggests that the gene can truly represent a risk factor for schizophrenia in different populations.

In summary, the association results of the single biallelic marker frequency analysis show that the KCNQ3 gene is associated with Schizophrenia.

3—Description of the Schizophrenia Collections Used for the Analyses of Candidate Genes.

The association studies were performed on two different populations. One collection of samples came from Argentina (the “Labimo” collection). The other collection came from England and was provided by the University College of London (the “UCLbip” collection).

All collections include individuals that are affected (patients or “cases”) or not affected (“controls”) by bipolar disorder.

67 random markers that were unlinked and not associated with the disease were used to perform stratification study and calculate the Fst value.

TABLE 5
Description and stratification study of
the four different collections

| | United College of | |
|---|---|---|
| Population | London (UCL) UCLbip | Labimo |
| Origin | English bipolar | Argentiniean bipolar |
| Cases | 315 | 160 (54 males) |
| Controls | 295 (142 males) | 157 (65 males) |
| Stratification on | Fst = 0.000123 | Fst = 0.000566 |
| 67 random markers | pvalue = 3.41E−01 (NS) | pvalue = 1.68E−01 (NS) |

The Fst values found for each collection indicate that these samples are genetically homogeneous; hence they can to be used in association analysis.

4—Association Studies Between Bipolar Disorder and the KCNQ3 Gene

a—Genotyping of Cases and Controls

The same biallelic markers than used for association studies in Schizophrenia were genotyped on the two bipolar collections (See Table 2).

b—SNP Frequency Analysis

TABLE 6
significant p-values and associated data for SNP located on the KCNQ3 gene

| | | location | | allele | | | | HWE |
|---|---|---|---|---|---|---|---|---|
| | SNP | on KCNQ3 | chosen | frequency | Allelic | Allelic | Genotypic | cases |
| Collection | name | gene | allele | difference | p-value | OR | p-value | p-value |
| UCLbip | M1 | 5′ of gene | A | 0.00 | 9.87E−01 | 1.00 | 1.91E−01 | 2.00E−02 |
| | M13 | Intron 1 | C | 0.06 | 4.02E−02 | 1.28 | 6.02E−03 | 1.98E−02 |
| | M18 | Intron 1 | T | 0.01 | 7.90E−01 | 1.03 | 5.02E−01 | 2.10E−02 |
| | M20 | Intron 9 | A | 0.01 | 7.53E−01 | 1.04 | 2.34E−01 | 2.35E−02 |
| | M22 | 3′ of gene | A | 0.03 | 3.51E−01 | 1.12 | 2.31E−01 | 1.74E−02 |
| | M25 | 3′ of gene | A | 0.04 | 1.59E−01 | 1.18 | 7.73E−02 | 3.55E−02 |

| | |
|---|---|
| Labimo | No significant p-value |

The results for the association with bipolar disorder show a consistent association of the gene KCNQ3 with the bipolar disorder in the UCLbip population.

TABLE 7

| | | | | |
|---|---|---|---|---|
| Marker | name | location | Oligo‘s name | OLIGO MIS sequence |

| | | | | |
|---|---|---|---|---|
| 30-106/40 | M1 | 5′ of gene | 30-106/40/A17 | GTTGAGCAGTAACAGGC |
| 30-49/29 | M4 | 5′ of gene | 30-49/29/A22 | AATCATTCCAGAAACATCTAGG |
| 30-107/29 | M5 | intron 1 | 30-107/29/A17 | GCTGGAACCAATCAAGA |
| 30-32/51 | M7 | intron 1 | 30-32/51/A24 | ATTTCATTTAACTGAATTTGATGC |
| 30-46/48 | M8 | intron 1 | 30-46/48/A19 | CACCAGGCAGCAGGAATGT |
| 30-34/30 | M10 | intron 1 | 30-34/30/A21 | TTCAGCCATCACGGAAGAAAT |
| 30-76/55 | M11 | intron 1 | 30-76/55/A17 | CAAGTTACTTGAGCTGG |
| 30-51/49 | M14 | intron 1 | 30-51/49/B21 | CTGTACCCACTCATAGACTCA |
| 30-59/28 | M15 | intron 1 | 30-59/28/A19 | CACCACCACCACATCTCCA |
| 30-48/53 | M17 | intron 1 | 30-48/53/A23 | TATTTCCTGTGACTGTAGAGATG |
| 30-42/38 | M18 | intron 1 | 30-42/38/B22 | TCATTTGGATGAGGGAGAGAAA |
| 30-53/77 | M19 | intron 3 | 30-53/77/B24 | ATCCACTATTTTAAAATCAGCTGC |
| 30-54/37 | M20 | intron 9 | 30-54/37/A21 | AGCACACAGCTAATTACAAGG |
| 30-52/62 | M21 | intron 10 | 30-52/62/B21 | AAACCTGTCACCTTGGTTTCT |
| 30-44/27 | M22 | 3′ of gene | 30-44/27/A20 | CAACATAGTGCCAGACTCTC |
| 30-57/63 | M23 | 3′ of gene | 30-57/63/A23 | CATCAAATATTTGTTGACCAACT |
| 30-91/27 | M25 | 3′ of gene | 30-91/27/A20 | GCTCCCACTCCTTCCTGATC |

| | | | | |
|---|---|---|---|---|
| | | | Polyp- | Major |
| Marker | primer PCR PU | primer PCR RP | morphism | allele |

| | | | | |
|---|---|---|---|---|
| 30-106/40 | GGGTCAAAATCTGCAGTAGC | ATGGTCTGCACAAGAAAAGG | A/T | A |
| 30-49/29 | GCAGTAAATCATTCCAGAAAC | CTCACAAGATTCCCAATGAC | C/T | C |
| 30-107/29 | TATTTTTTTCAGCTGGAACC | GCTTCATGTCATGGACAAAC | A/G | G |
| 30-32/51 | GAGAACCACAGGTACAACAC | CCTGGGGATGTGAATTTCTG | G/T | T |
| 30-46/48 | CACTGCAGATTTGGTGTTGG | CTCCTCCTCCATCTTTGTTG | A/G | A |
| 30-34/30 | TGCTCACCTTCAGCCATCAC | CTCTTCCCAGCACATTTTTC | A/G | A |
| 30-76/55 | ACCTGACACTGACTCTGGAC | GGTGCAGGAGACTTTAAATC | C/T | C or T |
| 30-51/49 | ACTATGTGACATTGATCGGC | AGAACAATGGCTGTACCCAC | A/G | G |
| 30-59/28 | AGTTAGTTCACCACCACCAC | AAGGAAGCAGAGCTCTAGTC | A/G | A |
| 30-48/53 | TTCACTCAGGTTGTTGGCAG | TCTCTGCTTTTCTGGCTTTG | C/T | C |
| 30-42/38 | GCAGGCCTTTCAAAATTCCG | GGAGGAGTCATTTGGATGAG | C/T | C |
| 30-53/77 | AAGTAAGGACCGTGTGATGC | CCTTGGGGAAGTATCCACTA | C/T | C |
| 30-54/37 | GCATAAGTTGCCAAGAGCAC | AGAGTCCAGAGTCAGACTAC | A/G | A |
| 30-52/62 | CATCCTTCAAGTTGACCTCC | CAAGTTAGGAGCCTGGAAAC | C/T | T |
| 30-44/27 | TCTCTCCAACATAGTGCCAG | CTTGTCAAGAGCCACAGTAG | A/G | A |
| 30-57/63 | GTCCCGCATCTAGTTTGTTC | AACACTCGGCACTCAATAGC | A/G | G |
| 30-91/27 | CTAGCAGCTCCCACTCCTTC | TGCTCATGCTGTCTCTCTTC | A/G | G |

### LIST OF CITED REFERENCES

Brown D A, Adams P R, Nature. 1980 Feb. 14; 283(5748): 673-6
Cooper et al (Arch. Neurol. 60 (2003) 496)
Dailey et al., Neurosci. Lett. 195 (1995) 77
Detera-Wadleigh et al. (1999) Proc Natl Acad Sci USA A96(10):5604-9
Freeman W M, Hemby S E; Neurochem Res. 2004 June; 29(6): 1065-81
Gribkoff et al., Nat. Med. 7 (2001) 471).
Gut I G, Hum Mutat. 2004 May; 23(5): 437-41)
Jaeger L B, Banks W A; Front Biosci. 2004 May 1; 9:1720-7
Jentsch (Nature I (2000) 21)
Kwok P Y, Chen X, Curr Issues Mol. Biol. 2003 April; 5(2): 43-60)
Lamas et al., Eur. J. Neurosc. 9 (1997) 605)
Saragovi H U, Fitzpatrick D, Raktabutr A, Nakanishi H, Kahn M, Greene M I; Science. 1991 Aug. 16; 253(5021): 792-5
Saragovi H U, Greene M I, Chrusciel R A, Kahn M; Biotechnology (N Y). 1992 July; 10(7): 773-8).
Schwake et al (JBC 275 (2000) 13343)
Shi, Clinical Chemistry 47:2; 164-172 (2001)).
Vogt M, Skerra A; Chembiochem. 2004 Feb. 6; 5(2):191-9).
Wickenden et al. 2000, Mol. Pharmacol., 58:591-600)
Wood M J, Trulzsch B, Abdelgany A, Beeson D; Curr Opin Mol. Ther. 2003 August; 5(4): 383-8
Zaczek et al., J. Pharmacol. Exp. Ther. 285 (1998) 724
Zhu H, Bilgin M, Snyder M; Annu Rev Biochem. 2003; 72:783-812).

1-19. (canceled)

20. A method of detecting the presence of or predisposition to schizophrenia, bipolar disorder or a related disorder in a subject, the method comprising detecting the presence of a susceptibility alteration in a KCNQ3 gene or polypeptide in a sample from the subject, the presence of such an alteration being indicative of the presence of or predisposition to schizophrenia or a related disorder in said subject.

21. The method according to claim 20, wherein said susceptibility alteration is a single nucleotide mutation.

22. The method according to claim 20, wherein said susceptibility alteration is located within the 5′ region, the 3′ region or intron 1 of the KCNQ3 gene.

23. The method according to claim 22, wherein the susceptibility marker is selected from M2, M3, M6, M9, M12, M16 or M24 markers as listed in Table 2a, or a combination thereof.

24. The method according to claim 22, wherein the susceptibility marker is M13 as listed in Table 2b.

25. The method according to claim 20, wherein the presence of an alteration in the KCNQ3 gene is detected by sequencing, selective hybridization and/or selective amplification.

26. The method according to claim 25, wherein said method comprises selective amplification using one or several primers selected from SEQ ID NOs: 5 to 28.

27. A method of assessing the response of a subject to a treatment of schizophrenia, bipolar disorder or a related disorder, the method comprising detecting the presence of a susceptibility alteration in a KCNQ3 gene or polypeptide in a sample from the subject, the presence of such an alteration being indicative of a responder subject.

28. The method according to claim 27, wherein said susceptibility alteration is a single nucleotide mutation.

29. The method according to claim 27, wherein said susceptibility alteration is located within the 5′ region, the 3′ region or intron 1 of the KCNQ3 gene.

30. The method according to claim 29, wherein the susceptibility marker is selected from M2, M3, M6, M9, M12, M16 or M24 markers as listed in Table 2a, or a combination thereof.

31. The method according to claim 29, wherein the susceptibility marker is M13 as listed in Table 2b.

32. The method according to claim 27, wherein the presence of an alteration in the KCNQ3 gene is detected by sequencing, selective hybridisation and/or selective amplification.

33. The method according to claim 32, wherein said method comprises selective amplification using one or several primers selected from SEQ ID NOs: 5 to 28.

34. A method of selecting biologically active compounds for the treatment of schizophrenia, bipolar disorder or a related disorder, said method comprising contacting a candidate compound with a KCNQ3 gene or polypeptide and selecting compounds that bind said gene or polypeptide.

35. The method according to claim 34, wherein said method comprises contacting a candidate compound with the recombinant host cell expressing a KCNQ3 polypeptide and selecting compounds that bind said KCNQ3 polypeptide at the surface of said cells and/or that modulate the activity of said KCNQ3 polypeptide.

36. The method according to claim 34, further comprising assaying the activity of the selected compounds in a model of schizophrenia or a related disorder.

37. The method according to claim 35, further comprising assaying the activity of the selected compounds in a model of schizophrenia or a related disorder.


SEQUENCE LISTING (6 sequences)

SEQ ID NO: 1 - DNA, 3097 bp, Homo sapiens
  CDS (226)..(2844): <s300> GenBank / NM_004519 1999-05-07 (1)..(3097)
gacccgcgcgggatgtgaccccctgaccccctgcctggcctcccctgccccccagggggcccgcctttgcctgcttttgggggggggtggggaggggcgcgcggatcatggcattggagttcccgggcttgcagccgccgccgccgcctcgttcacgcaccccgagcgccccttcttcccagagcagcagcggagaaggcgaagcggccggcgggggcgaggcagatggggctcaaggcgcgcagggcggcgggggcggctggcggcggcggcgacgggggcggcggaggcggcggggcggctaacccagccggaggggacgcggcggcggccggcgacgaggagcggaaagtggggctggcgcccggcgacgtggagcaagtcaccttggcgctcggggccggagccgacaaagacgggaccctgctgctggagggcggcggccgcgacgaggggcagcggaggaccccgcagggcatcgggctcctggccaagaccccgctgagccgcccagtcaagagaaacaacgccaagtaccggcgcatccaaactttgatctacgacgccctggagagaccgcggggctgggcgctgctttaccacgcgttggtgttcctgattgtcctggggtgcttgattctggctgtcctgaccacattcaaggagtatgagactgtctcgggagactggcttctgttactggagacatttgctattttcatctttggagccgagtttgctttgaggatctgggctgctggatgttgctgccgatacaaaggctggcggggccgactgaagtttgccaggaagcccctgtgcatgttggacatctttgtgctgattgcctctgtgccagtggttgctgtgggaaaccaaggcaatgttctggccacctccctgcgaagcctgcgcttcctgcagatcctgcgcatgctgcggatggaccggagaggtggcacctggaagcttctgggctcagccatctgtgcccacagcaaagaactcatcacggcctggtacatcggtttcctgacactcatcctttcttcatttcttgtctacctggttgagaaagacgtcccagaggtggatgcacaaggagaggagatgaaagaggagtttgagacctatgcagatgccctgtggtggggcctgatcacactggccaccattggctatggagacaagacacccaaaacgtgggaaggccgtctgattgccgccaccttttccttaattggcgtctccttttttgcccttccagcgggcatcctggggtccgggctggccctcaaggtgcaggagcaacaccgtcagaagcactttgagaaaaggaggaagccagctgctgagctcattcaggctgcctggaggtattatgctaccaaccccaacaggattgacctggtggcgacatggagattttatgaatcagtcgtctcttttcctttcttcaggaaagaacagctggaggcagcatccagccaaaagctgggtctcttggatcgggttcgcctttctaatcctcgtggtagcaatactaaaggaaagctatttacccctctgaatgtagatgccatagaagaaagtccttctaaagaaccaaagcctgttggcttaaacaataaagagcgtttccgcacggccttccgcatgaaagcctacgctttctggcagagttctgaagatgccgggacaggtgaccccatggcggaagacaggggctatgggaatgacttccccatcgaagacatgatccccaccctgaaggccgccatccgagccgtcagaattctacaattccgtctctataaaaaaaaattcaaggagactttgaggccttacgatgtgaaggatgtgattgagcagtattctgccgggcatctcgacatgctttccaggataaagtaccttcagacgagaatagatatgattttcacccctggacctccctccacgccaaaacacaagaagtctcagaaagggtcagcattcaccttcccatcccagcaatctcccaggaatgaaccatatgtagccagaccatccacatcagaaatcgaagaccaaagcatgatggggaagtttgtaaaagttgaaagacaggttcaggacatggggaagaagctggacttcctcgtggatatgcacatgcaacacatggaacggttgcaggtgcaggtcacggagtattacccaaccaagggcacctcctcgccagctgaagcagagaagaaggaggacaacaggtattccgatttgaaaaccatcatctgcaactattctgagacaggccccccggaaccaccctacagcttccaccaggtgaccattgacaaagtcagcccctatgggttttttgcacatgaccctgtgaacctgccccgagggggacccagttctggaaaggttcaggcaactcctccttcctcagcaacaacgtatgtggagaggcccacggtcctgcctatcttgactcttctcgactcccgagtgagctgccactcccaggctgacctgcagggcccctactcggaccgaatctccccccggcagagacgtagcatcacgcgagacagtgacacacctctgtccctgatgtcggtcaaccacgaggagctggagaggtctccaagtggcttcagcatctcccaggacagagatgattatgtgttcggccccaatggggggtcgagctggatgagggagaagcggtacctcgccgagggtgagacggacacagacacggaccccttcacgcccagcggctccatgcctctgtcgtccacaggggatgggatttctgattcagtatggaccccttccaataagcccatttaaaagaggtcactggctgacccctccttgtaatgtagacagactttgtatagttcacttactcttacacccgacgcttaccagcggggacaccaatggctgcatcaaatgcatgcgtgtgcgtggtggccccacccaggcaggggcttcccacagcctcttcctccccatgtcaccacaacaaagtgcttccttttcagcatggtttgcatgactttacactatataaatggttccgctaatctcttctaggata

SEQ ID NO: 2 - protein, 872 aa, Homo sapiens
MGLKARRAAGAAGGGGDGGGGGGGAANPAGGDAAAAGDEERKVGLAPGDVEQVTLALGAGADKDGTLLLEGGGRDEGQRRTPQGIGLLAKTPLSRPVKRNNAKYRRIQTLIYDALERPRGWALLYHALVFLIVLGCLILAVLTTFKEYETVSGDWLLLLETFAIFIFGAEFALRIWAAGCCCRYKGWRGRLKFARKPLCMLDIFVLIASVPVVAVGNQGNVLATSLRSLRFLQILRMLRMDRRGGTWKLLGSAICAHSKELITAWYIGFLTLILSSFLVYLVEKDVPEVDAQGEEMKEEFETYADALWWGLITLATIGYGDKTPKTWEGRLIAATFSLIGVSFFALPAGILGSGLALKVQEQHRQKHFEKRRKPAAELIQAAWRYYATNPNRIDLVATWRFYESVVSFPFFRKEQLEAASSQKLGLLDRVRLSNPRGSNTKGKLFTPLNVDAIEESPSKEPKPVGLNNKERFRTAFRMKAYAFWQSSEDAGTGDPMAEDRGYGNDFPIEDMIPTLKAAIRAVRILQFRLYKKKFKETLRPYDVKDVIEQYSAGHLDMLSRIKYLQTRIDMIFTPGPPSTPKHKKSQKGSAFTFPSQQSPRNEPYVARPSTSEIEDQSMMGKFVKVERQVQDMGKKLDFLVDMHMQHMERLQVQVTEYYPTKGTSSPAEAEKKEDNRYSDLKTIICNYSETGPPEPPYSFHQVTIDKVSPYGFFAHDPVNLPRGGPSSGKVQATPPSSATTYVERPTVLPILTLLDSRVSCHSQADLQGPYSDRISPRQRRSITRDSDTPLSLMSVNHEELERSPSGFSISQDRDDYVFGPNGGSSWMREKRYLAEGETDTDTDPFTPSGSMPLSSTGDGISDSVWTPSNKPI

SEQ ID NO: 3 - DNA, 64812 bp, Homo sapiens
  misc_feature: 5′ region
gcagtaaatcattccagaaacatctaggcctttccaaagcatctagtatgttttatcttgtagtcattgggaatcttgtgagcttcatgtttttctctttgttttggccagaaagctaagggctgatattccctgctttcatatatatatgtcttaaaactcttattattcattcattcatttaatagttattgcacaactactatatgccaggcactctactaggtgccagaaatacagcaatacacaaaacaggcaaacagtattgcacttagaaagcttaaaatcagggctgagctcagcagccaatacctgtaatcctagcactttgggaggatgagagggaaagaccacttgagcccaggagtttgaggctgcactgagctatgactgcgctgctgtactccaccctgggtgacagagcaagaccccccaaaaaaagagagagagagggagaaaagaaagcttctgtttaagtaaggaagattaacaataaacacaacagagaagtaaatatagggttaagattttacttgggccaggcatggtggctcacacctgtaatctcagcacttttggaggccgaggcgagtggatcacctgaggtcaggagttcaagaccagcctggcctacatggcgaaaccccgtctcaactaaaaatacaaaaattagccaggtgtggtgatgcatgcctgtaatcccagctactcaggaggctgaggcaagagaattgcttgaacccgggaggcgaaggttgcagtgagtagctgagattgcaccagttcactccaccctgggtgacagagtgagactctgccaaaaaaaaaaaaaaaaaagattttacttgatgttccagttacttatggccatgtaaccaacgccccccaatcttactcgtgcaaagtaaaacatccttctgtctgtgggttagcaattctgacagggcactggggaatgatctgtctctgctgtttgatgtttgggccttagctgggaagattgccactgactccaatggatggggctggatcaggaggggtgagaggatccacttccaacacgacacagcttcttcactgggaatggttggatactgggctcagctgggactgccagacagaaagcctacatgatgtagcctctccagtatggtcatgtcaagatagtgggacttcttatgtagagggcaagtgtccaatgatttaggcagaagctgtacaacctttgaagaactagcctcaaaagtcacctcctgtcacttacaatgcatgccattggtcgaaggagtcaaagctcactgagattcaggaagatggaaaacagactcaatggaaaagtttcaaattgtttgtgagtcgattttaaaactggcacatgacttcagggtcaattcccattatcgtggggaggggcttcatatttagagtcaatcacaccaggagttgaacagcgggtcaggcatgtgcttttatttagcaagcatcattgtgtgttgatggtgagtctatcaccgcgccaggtgctgggggttccacattgaggtagacacagtttatccccttgaatcaggaaaggaaagtggctaaacacccaatgataaatgtgcagtttgagaaatacggtggtggtgaatacacaggggacaatggaaccacatggaaagagaagttaattgagtgaatttgggaaaattatttagcatctctctgtgattagcgtccctatttgtataacaaggctgataagacctgtcttataagactggtttaaggatgagatgagacaagtaatctaaagcagctcctgatcaatcacttctcatcatcctgttgaggtctgtgcaagcaacttctcactatctgagcttttcctagtttgtttatttgtttgtttgtttgtttacttgcttattatctgaactcaccaaactagaatataagatatatagagtctattattcaggctatagtataaaaaatgaactcgtctctgtctagaaagcccttcccttcagatattttgtggccaactctttcaccctttacaaaaaagcactaaatacgctgcctagtcattcttcttaaaattgcatcctagaaccccctctataatcgctggcttcccattctgttctatttttctttttcaaacagcacttaaaacctctaacatactctacataagctttagacttatttgtttatcctcttgccctgctagaatgtaaattgcattagggcagggatctttgtttttttcactgatgcatcacaggtgcctgtaaccatacccttttcataatggaacaaatgaatccactgggaaggggtaggacattgagtcagaagggtggcaggagtcctttgtaagccaggataaacagtttggatttttttttttccaaaataagataaacagtgtagacttttatgagaaggtatgacatgaactaaatgacatttttacgtcattctggctgctgtagagagaatggaatagaagaagtcaagattggacaggacatgagttaggagagagggtgatggcttggaccaaggtgatggtcctggagacaaagagaaatttgcagctttcagatgtgtttttacatggaaggaagaactgaccagacatgctgatgggctgaacaagagggtgagtgagaaggaaatcgaggctaactcataggtttttggtttgagtaacctggcatgatataatgtcatttactgatatgagtcaagactaaggaagaagcagaaagtctgcgtgtgtgcgtgtgtgttggtgtatgtgtacgtagtgtggtgtgtgtgtgtgcagtggtgtgtatgtgtattgtgtgtgttgtgtgtattgtgttttttgtttgtgtagtttggttgtgtgtttttgtgtgtgtagtattagatgtgtgtgcagtgaggtgtgtgtgtagtgtgtatgtgtatagtgtgggatgtgtgtgtgtagcagtgtgtgttcatagtgtgtgtggtgtgtatgtagtgtgaggtgtgtatgcagtggtgtgttgtgtggtgtgtgtgtgtagtgtgggagtgtgtttctagtgaagtgtgtgtgtagtgtggggtttgtgtgcagtgcacgtgtttttggtatagtagtgtgtgtggcctgtgtgtagtgtgggaagtgtatgtgcagtgtgtgtgggggtatggtagtatgtgtgtggggtatgtgtgtgcagtggtgtgtatgtgtagtgtgtgtgttgtgtgtatttttttgtgtgtagtttggttgcatgtgttttgtgtgtgtgcagtatcagatgtgtgtgcagtgaggtgtgtgtgtagtgtgggagtgtgtttatagtgaactgtgtgtgtacactgtgtgtagtgtgggatgtatgtgtgcagtgtgtgtggaggtatggtagtatgtgtgtggggtgtgtgtgtgcagtggtgtgtgtgagttgggtgtgtgtgcctgtagtatggggtgtgtgtgcagtagtgtgtgtatgtgtagtgtcgtgtgtcaatgagtgtagtgtgctgtgtatgtatgtgtgatatgggccaccaggggtggggcagttaagattttttttcttgactttgttcattgtgagatgagtattagactgatgaagtattctccatctagaatccagcctaaggagacagtcagaagtgctcaccaagattcagaggttcttcattgccttaccatatttgtaagtcacaaactggaagctaccacagggtccagccttatgagagtggtgcagtcaatcccagatcttccatatgacagggagctggtcatccttcaaagtgctgcttactaagatgacaatgagactggaaaattctcattaaataaacaaatggctgggtaagtgcttactgcatggtgtattcatgttagtagagaaatatctacaaaatacgctcgtggtaaactctataaatcatgaaaatgtaaaactgcaagaaatctcaaggtcatcgtttcaaaatgcatgctatagttgagcaaaagaaaaaatcagtgaagactaagaggctgaagagttcctcatttaagctgtgacatttgatcctattagtgggaaaagacctgaactgagaggacctgtgtcctcgcccctggttttgttgctgttgctttgctgggtgaccttggagacaccattgaacgtctcttggctcagctttttcacggttaaatgagacagttggtaattccttgtttttcagagtttgcatggtttggatgccattggctcttccttatagcagggagtttttcagctgcaaagagcggtggagttcctctctcccagcccactcataattcggcttatgtgcagaaggcagaagaccttcccaagggtacacaatgcttctgtaaatagcagtttatagacttcagcagaagaaccagaagcaaacagaggaagtgaaaatgaccactagaagaaaaactgatgacagaaaaacaaaatcaattagactttatctttaaaaaaatcaactatgactttaattgatttcaaataaaatatttcgatctttatgagatacattagatcgttgaaagtcatgcttacagaaaatattagtgtctgtgaagaaaacactaagcttctgcaaaagttttgcctctcacttctagtcacgtccccactgctagtccattctctgaaaagtgtacccaaagcaattttcttttcttttttcgtttttctttctttctttcttttcttttcttttcttttttttttttttttttttttttgagatggagtctcgctttgtcacccaggctggagtgcagtggtgcgacctcagctcactgtaacctctgactcccaggttcaagcaattctctgcctcagcctcccgagtagctgggattataggtgcccaccaccatgcccagctaatttttgtatttttagtagagacggggtttcaccatcttggccaggctggtcttgaactcctgaccttgtgatccacccgcctcagcctcccaaagtgctgggattacaggtatgagccaccatgcccggacccaaagcaattttctaaaaatgaaaatgtcattctgtcacacacacacacacacacacacacacacacacccctgccacctgagacatctgcactgtaaatggctcaacgggtaccattttctatgagataaagattgaggtcccctgacctgaccccacccacctcttccagtttcccttgctattcccctccctcccctcctggtccatgcctgttcgctcttaaatatgcccggctgtttctaaaccttctggtctttgacaatcctctcctttcatccttgatcagctctcctcacctggctcaccaggaaaacacctactcatctttccagattcagccgatcatcatctcctccatgaagaccttttctgacctttgctgcttttgtcctttgtaactgattatatagggctccacacaggctgctatcctacccagtggaaaatttctttgcatttacttctttacctgcttagcccctgccccttataggctaaaagctccttgaaaacaagaaatatgttatttcatcttggtatccccagtacctatgatgatgctgccattcatttatttgataaaaattccttaagtggccactaagttccctgcattgcagcgggcagagtagacagcagagactgcgtagtagaatagcagatactgcacttgtttgtccagttccctttattccgtggtggtagatttttaaatctgggtacattgacacccaattaaaggttacatttggcacccctcttgcaattttgtggccatacgaacaagttcaagctaatgggatgtgagtggaagtgatgtgtgcaccttctaggtcttgatcttaaaggggaagggagtccttcctttttccttttcttttcccgttttctgctggtctgaatatgcctgtgacagtgggagcgggagcagcccttttggatcctgagagagaagctacaagttaagcgtggcagaacaaattaaaagtagattaatttcccttcatcatcaagccttcacaccatccctgaactgtgtctaaacttacatgagatagaagaattaatggacagtcgattcaaaccaatgcatttggggcctgtttcttaaagcagcctgccctgtaccttgctcatataaatggtgtctcaagcagagatgatctttttactgatggagcttgtttagcatagacactgaacacatggctaatgaaatgaatgagtgagtaaacagtccaaggtctaaccagaaccagctagaaaaaataaatgagtcatttgtcacaacaggcagatagcatcgtatcctataaggttgaaattatttggagcctgtgcttcagacagtgggtttaggcattgcagcttcacaactctatctgcctcgccctctgcagacagagccactgtggtcttttaattctccctccacgcgttgtctttattctgtttcctcctagcctctcccaaagctgtagctctaatttggatccttgttacctattacctaacccgtggcactagttagtgagctggcctctccaccctcctgtcctcccccagagctgttgccatggcaatcttcccctggcacagtgctgatcaaaaccttccagggctccccactgccttcaggtaacagcccaacctccttaaattatctccaggcattggcccacccccttccctgccttaactcccactcactgtcctcacctacgcggtttcccttcccacgacacacttgccattccctttctagatgcttattttcaagctgatccctttccctggaataacttctttgtccagtttcactcccacctctgcctgttaaaatcctacccttctttcgaagtccaacaaaatgctgcttctttacgatgcttttttatgttttagggtcaaaaggaatctgtcttctctctggacattcctagacttcttcattcgcaatataacatttatcacactaggattaggatgaagtgcaggctgagtagagtagaagataattcagtactattattaacaataaccacaataacagtcataatcaacaagatctaatcattcactgagtgcttactacatgtcatgtcctatcctaagagcttttttttctttgatggagtctctgtcaccagactggagtgcagtggtgtgatcttggctcactgcaacctctgcctcctgggttcaagtgattctcctgcctcagcctcctgagtagctggaactacaggcacgcaccaccatactcagctaatttttgtatttgtagtagaaacagggtttcaccatgttggccaggatggtctcgatctcttgaccttgtgatccgcccacctcggcctcccgaagtgctgctcccagggctttaactgcattaaagcatccacagcccacagcaatcctgtaggtagctccattttagagctgaggaggtcataggcacagagaggttaagcaacctacccagggtcacacttctgggactttgaatcttgacctgaccgtttactaaacatattaacttaggtgaattatttcacctccttgagtccctagtgtcttcatctctgaatggttttgttgagacaatggtatgatatgctgtatgggaagtctctaagccagctccatgtacatagcaggtactcaactcatgctatctcctcatctgcagggctgtatcttatagcatgacttctcatttttctctaggtggcaggtgcctacaaaatgtttgtttacgctgaaaattatgttttgtgttggatgacaaagttccagtcatactctgcccaatatctctcaacataaataaggtagcatagtggttacagcacagagttctgggtgtccactaatcatttctgttttactactatgttagtaacaaaaataatacattttaactcaaaagagtgattcataagcttctcttgctgctaggtatgaccacaatcccagttctggccgatggtatgtaggcagagccaatggaggcaaacagcctctcctttcccttctttccttcttgtcacctgggatgaggatgtgaaggtgaatcttcagggaccatgcagagcgggcaacacgctggagatggcagctgagtgagaagatcaaaggagcctggttcggacagttgacatcaagattgacacatgagaaacaagtaacttccgatttgtttaagccactgtatttgaaccttagttatatccaccaaactgatactccagctagtactgttgtgacagcctaggtgaacataattgcccgcttgctggctgagtgaacttgggaaaggtacataatatcccagtgcctcaatcccctcatcttttttttttttttttttttgaaatggagctttgctcttgtcacccaggcgccatctcggctcacttcaacttccacctcccgggttcaagcgattctcctgcctcagccttcagagtatctggaattacaggtgcccgccatgatgcccagcttatttttgtatctttagtagagatggggtttcaccatgttggtcaggcttgtcttgaactcctgacctcaggtgatccacatgccttggcctcccaaagtgcttggattacaggtgtgagccactgcgcctggccaatcccctcatctttaagatgagatagcatgacacctatgtggcagaattgatgtgaagataaatgagataatgcatgtaaattgcttagcacaatgtttggcacacattaagagctcaataaatgttagggattattgctattacgagtaagctaaaattgcagctttccagagacctgcccccttttctagctggggtgctgattctactcctggaatagctggtgcaatgatctcatcttctcatgtttatagaattcatggcacaaatatgcttctcttcagtcccttctaccaatgtcatccttgctctccctcccttggccttgtgctgaagcatggaatggcttatctcactaccagaaggaaagcagaaagcaaagtcacaccactgtcaccatgacacacttctgaaatggctggtggatctgcaggatgtatgctttgctcaacagaggagccggggggaaaacacatttttactgggcacctcccacagcctggagctcacatgggcactatgcatctcacataagcctcacttctcccctctgtgtggtcattgacacaattgcacagacactaaactgaagctgaaagatatgaaataattggtccaagatcacatagctggacaatgaggggatgctcatctttagatggtaagctggtctgaatgaagtcagagatctcctaacattccaaaggtcataattaagcttctagggtgaaatcaaggaatgttaagagatatctcaaatctcttccgaagatccagatggcaaggagtgttaagagatctctaacttcaatcagaccagcttgccatctggggataagagaataccagtgtgcacaattgatttatttgagctttggggaaagtctccttatgaaaaatatgtctttcctttgctacctgcgttttagcaatgaagctccaggagattccactgctgctgggattccaaattcaaagcaccacagggagacaggggcccatagggtcatccagttttctcacaaagctactgcctttctaagcggcacactatctatgctccccaggctatattttgctcttgggttatgcttgagtttaagtaagactgtctccaaattttagataaggatattttcttaaataaataaaaaagagtgttgctacaaattatgcatctgtacttggggcctatgaggatggtcctagcgcctccatggtttgcaagtccaggggagggtcttaggcctgcagcaggtgacaataggagggtgctggggctcaaagagttcatgcagacaagcaagccactggcctcgtggccctccattccaccttagcagcttaattatgatttctttcatatactggattcttcttaagaccacatcttggacagaaacagggctgctgctagtagaactaagaaacaaaaaaagtgtcaaaaccactggcatattagaaaaattcctgattgatagaggatttcaaaaatccttggacctaagtcctgctctgacactgtgagttcttgtgcaaatttccttacctctcaaagcctccatttccccagctgcaaaatggaaaaaccaagtatcaatttggaggattgttctgaatattgagataagagttggttgttgttatttcactttcagaaatctgtgagctaaccgaagttctcaccctctttcatggcaggatggactttgaaggaagacaaccacaacaactcctagattacacagtaaagatgcatttaagccagcatctctcagaataattccctaataaaaaaagtacttgttcataatgtagacttctgagttccattcagaatgatgaagtcaaaacttcagaatgaacattccagctaattcttatgcatgttaaaatgttaaaatttttaacataggtgataataactaagtggctaaaattaaaaagactgataccaccaaatgttggcaaggaagtagggcaactgaaattattatacatatctggcagtttaaaatgatataatgactttggaaaactgttcagaagtatctactaaagtgtaatatattaataatttattattaataataaatatattatttttatgaactagcaattccactcctaggtacatactgaagagaagtgattgcacatgtccacaaaaagacatagacaaacatgctcatggaagatttattcataactccaaactgcaactaacccaaattctcatcaacaagcaaatggataaataaattgaggtgtgtttgtatcatcaagaaataattgctaggttttagaaataggccagagatccctggatgtcctcttattgtctggcaatactaggtgctgtctagaggactctcaaggttatctgagtatagagagtgtatgcttttctttctattgcctaaacaactttaagttacagaaaactgatagcagtgcaaatgtttcctgttcatagcaatgaaaaggattcctgtccacagcaaagcaaacagattctgtctggtagagaatataaatctcagtaattcaagtacattcttattgaaacctgtttttagtgttctactgattcctttattaatgaatctgttgaataaaatctttgatacatggtcaatttatatttgcataagagtcaagtttttaacttaggaaaattatgatttagatgtatacttgacaaatacacagatcttatgttgattaaaagaagctaaacacacaccaaaaatgcatgctgtaaaattctatttgtatgaaaaccaaaaaagggtaaaactaatgtatggttttggagatccaaacagtggttacctctgctgggtgggtgggtgggtttgatgactgggaaagagccccagggaagcttctgaagggatgaaaatattctacatcttggacttggcactggtcatatggggccatacatctacaaaaaacattaccaggctgtacacttaagctatgttcacttttctctatgaaagtcataccttgattttttaaaaaatcaaacttgaaagtaaaaattaatggatgatacgtaatcctttcagcaatggtgttatcctctcagcaaaagatggtactttatcttcttagtgatggtgtcacattgctgtaactaaacttcccgcagcactttgaagccccagagctgggtgcagtaagaggtgagggagactgacggcccctctagtcccttcctcacagggctgctgctgaaaacagtctccctgacccgagtctccgaagctgtggccagtaacacaccctgtcactctaacaacctcattaaagtggctgagggcagaaataacccaggtctagacagctggaagcagacagatgctcctcattttaaagctcagcaaaatgtattttaaagaagcactaaaaagctttataaggaaagtctgtcaaaaatcatcgcaagcatccatctctaaaatagccctatcagcaacatcttttatacaaaatcaaatcaaaacggaatctttgcctagaaaggaaatttcaagtcttccaagtccttctcatttgcaggcatccatccttgacgtcctctgaagcatccatacaaggggtcattccgcctttactactgaaaagaaagctcattacttctctctgtttataactagaaagtactgaggatttacttggcttcacctggtttcatcctcacaagccagtaattatcattgccattttacagatgggcaaactgaggtttagagagattaactaacttgcccaagaccacacagctggcgagggttagagccagggttccagttcagctctgaccacatgactcctctgaacaattccccttccaaggcaagccagtgccacccttgggaagctctgatatttacaaaattgtccttataataaagacagtctgccttttaatactttcacctcttcagcatctactaagttattatgtaacccttggaactgcacagagcatctatttttttcttacatctgagaaactttcacctatttgaaaatattccctatttccccccaagtcttctcttttccacacaaaatatggacaacactttactttccagtgaccaactatctggattttcaagggatggagggaattcttaggataagggacttccagtgctaaaatggggacagttgcgcaaaccagaagagttgatcaccctacattttttttttgctaatggtgttacctgatgcatagattgtttttattttttttacaaggcctgttctgcatgatgactatatcattcaaaactcttattgcaattgcagaaactacctcaactggcttaagcaaaaaggggaatttctgggttcatggaacaaaaactccaggagttgcaggaacagaagtgtgcagagatgggtgccacctcttgtcttttgttttcatttgcactacctttccagtttgcctgaggaaaacctgtctccaccaaaccctaggcccagttgacaagggcctctttgacataaactgtcaacactgtcatgagggcagcagtcattctctcataaactcatttatttcatagccagctctcccaccacactgtgagtccctgaatatctgtgtctcgcttgtcttcattttcctttttttttttttatttttgagacagagttttgttcttgttgcccaggctggagtgcaatggcatgatctcaactcaccacaacgtctgcctcccaggtctaagagattcttctgcttcagcctcccaagtagctggggttacaggcatgtgccaccatgcccagctaattttgtatttttaggtgagacggggtttctccatgttggtcaggctggtcttgaactcccaccctcaggtgatctgcccacctcggcctcccaaaatgctgggattacagacacgagccactgcacctgaccttgtcttcattttctaaacaccttttacctcaccctttacagagtagttggcatttgttgaacaagttaagactgtcttacttgggacagagactgttcatcaccccccaatatgcacatcttccctttttctcagctacagaacctcaattttattcactgtggcaatgcacccagctaacaagcaacacttctcagtttcccctgcagctagatgaaaccatgagactaacactgggccaaggagatgtaggtaaaagggttacagggaaagctgtttcaaggagccgacccagtggggaggagcaccactttgtcttttacagatgctttggcagcctgctcaaaatggggacttgatgctgaacttccacagacttcttctattatgatccatcttgaggatggaaatcatccactgatgatggcagatcagataagcagttggctcccagaagaaagtgtgaagcttccattatagtccagatttgcctcctcctgacatcttttatgtgtgagagaaaggagctaccctgtttaaaatagtgttatatggggttttcctgctgtaggtgttatgtacagtcgatttacttttaatttatgatcaattgtctgtttgaataaagaaatgtgtgtaggtataactgactaattggtgaaaattagataaaccttcctgttaactagctttaaaaatgaagcttccaaatcccagcactttgggaggctgaggcgggcagaccacgaggtcaggagattgagaccatcctggctaacatggtgaaatcccatctctactaaaaatacaaaaaataaataaattagacgggtgtggtggcaggtgcctgcagtcccagctacttgggaggctgaggcaggagaatggcgtgaacctgggaagcagagcttgcagtaagccaagatcatgccactacacttcagcctgggtggcagagcgagactccatctcaaacaaaaaaaaaaaaaaatgaaacttcctagttcgcctcctataactacaactctttcagtttcctattttaaaaattgagtcataaagaattaacatatctttacaattaagccaaaacaaaatataagtgggtgttcataatgtatcctctcacttaaaagtgctggaaaactttgttggcaagaaatgggtgaccagtgaaccagggaaggttaatgacagacctgactcatcatgacacaaggaaaatcttcagttcctttagtacaaagtctctttatttccaaagcaagtcttgcacttcctggtttgggctgaggtaaaaactgactgtattagtaagggttctctagagagacagaataggatagatgtatatataaaggggagtttattaaggagtattgactcacacgatcacaaggtaaggtcccacaataggctgacctcaagctgaggagcaacgaagccaatccgagtccaaaaacctcaaaagtagggaagccagcagtgcagccttcagtctgtggtagaaggccaagaatcccaaagtggaagaacttggagtccgatgttcatgggcaggaagcatccagcctggaagaaaggtgtaggccagaagaacaagccagtctcgtctttccgtgttagtcggcctgcatttattctggccgcactggcagctgattagatggtgcccacccagattgagggtgggtctgcctttcccagtccactgactcaaatgtaaatctactttgggaacaacctcacagacaccccaggaacaatagtttgcatccttcaatccaatcaagttgacactcaacattaagcatcacactgaccctgcttattagccggaaggtaatgaaggaggctgactttgagaacaactcacatcttgagaggcacccttgcaagagcttcaagtaaggtcttctttatattaatactatgtccttataagaggagactgtttctcttttaagggaaaagaggcttctttgtcagtcagagggttttaggagaatttgagattttaagattctcaagctcatctatccaaatatccctatcctaggtctcatagtcttactctttacctatcatggcccagactttaatataagagacctgtcaagattatatatagtttcctttgaaactatgtctttttaaacaatcagatcctgggctttgttttcagcaaagtctgccctgtgacagcaggagatgaaattatcctttaaaactgccagggagacttagtccatttatgttgctaaaaagaaatacttaaggctaggtcatttatacagaaaagaggcttatttggctcatggttctgcaggctgtacaagaggcatagtgtcagcatctgcatagcttctggtgaaggcttcatactgcttctactcatggtggaaggggaaggggagccaccatacagagatcacatgataacaaagaggaaagagagaggatgaggtgccagtcttcaccagttcgtgcaagaactaagagtcagtgagaacacactcactcctgggagaatggcaccaagtcattcatgagggatacatccccatgattcaaacacctcccaacaggccccacctccaacattgggtgccaaatttcaacatgagatttggagggtcaaatagccaaactatcagagaccaatctgactttcatagaaactttaagttggcaattttgatcttttttttttttttcatttttaaagccttaagtgccatcaaaagtggtcactccataccacactcttcataattatcattactcacaaactggttgtattagtccatttttatgctgctgataaagacatacctgagactgggtaatttgtatagaaaaagaggtttactggactcacagtaccacgtggctagggaggcctcacagtcatggtggaaggcaaaagacacatcttacatggcagcaggcaagagagaatgagaaccaagtaaaaggggaaacccttcataaaatcatcagatttcatgagacttattcactaccatgagaacactatggggcaaaccacccccaagattcaattatctcccactgggtccctctcacaacatgtgggaattatggaagctacaatccaagatgagatttgggtggggacacagtcaaaccatatcattccaccctcattccctccaaaatctcatgtcctcacatttcaaaaccaatcatgtctttccaacattccctgaaagtcttaactaatttcagcattaactcaaaagtccatagtccaaagtctcatctaagacaaggtaagtcctttccacctatgagcctgtaaaatgaaaagtgaattggttacttcctagatacaataggggtacaggcattgggtaaatacactcattccaaaggggagaaattggccaaaaatgaaaggactacaggccctatgcaagttcgaaatctagcagggcagtacaatcttaaagctccgaaatgatctcctttgactccatgtctcacatccaggtcatgctgatgcaagaggtgggttcccatggtcttgggcagctccacccctgtggcattgcagggtatagcctccctcttggctgcttccacagactggcattgagtgtctgtggcttttctaggtgcaaggcacaagctgttggtggatttatcattctggggtctggaggactgtggccctcttctcacagatccacattttccccattgtcttggtgactaacatttggctcttccttacttatgcaaatttctgcagctggcttaaatttccctcagaaaatgggtttttcttttctattgcatcataatgctgcaaattttcctaacttttatgctctgtttcccttttgaaactgaatactttcaatagcacccaagtcacctcttgaatgctttgctgcttagaaatttcttctgccagatactccaaatcatctccctcaagttcaaagttccacagatctctagggtaggggcaaaatgcctccagtctctttgctaaaacatagcaagagtcacctttactccagttctcaacaagttcctcatctccatctgagaccacctcagcctggatttcattgtccatgtcattatcggcattttgatcaaagccattcaacaagtttctgggaagttccaaactttcccacatcttcctgtcttctgaaccctctgaactgttccaatctctacgtgttacccagttccaaagtcacttccacatttttgggtatgtgtatagcagcaccccactctactggtaccaatttactgtattagtctgttctcacgctgctaataaagatggaccaaagactgagtaatttataaagaaaagagatttaatggacttacagttccacatgactagggaggtctcacagttatggcagaagatgaaagaagagcaaagggatgtcttacatggcagcaggcaagagtgaatgagagccaagggaaaggggaactcccttataaaatcatcagatctcgtgagacttattcactaccatgagaatagtatgggagaaaccacctccatactcccaccaggtccatcccacaatacacaggagttatggaagttacaattcaagatgagatttggggaaggatgcagccaaactatatcactggtcaaatgcagctaccatttgatctctcaagttttgcattccactggcacttcattccaattaaatgtaagtgaaactttagaaatggtgattacggtgaaaggttattactgcacttaccataagcaatagagtttgtcttgcctccagactcatgggaaatttacctccaatactttatctggaaggtctgttttcttatatcacttcttaaaccaattattgtagcatgggttcccttaaaagtaaagactgaggccaagcttaattaaatgcgtgtaatttgaggagagcaagagagaggcaggagagaaaagaaagtaaatgcaagctgactttagcttctcaagaaaatacagttggtgacttggtcatgagcgatttccctggggaggttgtatggaattactgcatcaagcaggagaagaacaagtgatatatatatatttgcagacttcttctcatcttatctcttacttcttctcatctcatgtttctggttaaaattcatcccataggggcattaacacattaacacacttaagcatttagtttttattgacttccacaggcatccaccagggaagccataatcaggatgggtctggttggcttgggccagggtggtggagcagctttagtttcctctaggataggcctggttaaaactaagttgatagaaggcagtgttatgacatgagactgtggccgcaggagatttgcaaccaaggatgccatgagcaaggagcctcctggagagacccagagggcctcaagtgttgcataaacagggccagtatacctgtgtgctagctgttctccatttgccccttccacatttatgcattttctttttttactctcgtctgtacctttagaggctggccctaaggactgctttaccctacgtgcttttccctgtctcttctggttggattcagccaatgggagacactaggaagagatcagaaaatagagtaaagagtggtcaagaattgtttctcccactccctccctgactggtgctgtgattctagcagtggttgcatccttccgtgactaagtccctgttggaaatcacttcccccatgattccactaacactggttcctctccatgcccttacaggtgaagggttaccaccaggcttactgtcaatggtactgggttcttcgacatctttcaatggtacccttcacccagagctggcctctgtaaatggactcttccttaaattttcttcaaaatcctagctgattactttctagtttcaactgggacactaagcctacctctgtttatttcattctgttgttttatgaattcttgtttggctttttcattttaaaataatattcctgtttaggtatcctagcaattatgacacatttccttctcctctatgtctaatgctttcttataggttgggatctttgtcttacttctgcatgctgtgttcatttttccagtgtatttatttcttctgtactatatttgcttagtcattgcaccatttatttccatctgtctcctgcctgaacagttctgtacagatgttctatttgctctgatactttggggctgatttattgatccttctctcactgtatatgactccagttaaccttccctttcaggtacagtatgagtgaggaaaggatgttaggtcatcttccatttttcaaaactggagagaatagctgagatgcacatgtatgcacatatgcatatacacacctctacacacccacccatacacataaacacacaaactgtggaaagattcactgagttgctagctcgtttttactaatatatctgaagttttctctctctctgatattttgtggcttgctccgcctggttagggcacatattctaagcctttaaagaggtgctcttgggctttgacagttttctccaagtcactgatgtagaagcacttgcaaagtcatcaactccatgttttgacaagaactcctccagctattcttactagttggctgggttgttagtttcctcttgctgctgtaacaaattactacacatttagttgcttaaaacaacacaaatttattatctcacagttctggaagtcagaagtccaaaactacagggctgcatttcttctggaggctgtaggggagtgcactaggctgagcagcattccccaacctgggaaaaaatgcatgtccatccagaaccaaaatatatggccttatacgggaaaaagttctctgcagatataatcaattaaattaagatgaggtcatattgagtcatattgggttggagtgggacctaaatccaatgactgatgtccttataaacggagaacagacacagagacacagacagagaagacagttatgagaagatggaggtagaaactggactgatgcagctgcaagcccaggaatgccagggatttccaccaagcaccagaagctaggaagaggcaaggaagaacccttcttcagactctttgaagggagtactaccctaatgacatccttgattttgggcttccaggctcccatattggaagagaataaattactgtgttaaaccactttgttgttgttgttgttgttgttgtttttgtttctgagatggagtctcactctgtcacccaggctggagtgcagtggtgtggtcttggttcactgcaacctttgcctcctgggttcaggcagttctcccacctcagcctcccgagtagctgggactacaggtgcccaccatcacatgtggttaatttttatatttttagtaggacggggtctcaccatgttggccaggctggtctcgaactcttgacctgccttgacctcccaaagtgctggaattacaggcatgagccaccgcacccggtgcactttgtgatactttattatgaaagtcataagaaacaaataaggtgaaaatcagtttctttgcctttttcagctcttagaggctatcccccttacttggcttgttggccccacatcactccaatctctctttcctttgtgacatcttctctctgactctgacccacctggcatcctcttataaggacctggtgattacattgggtccatctaggcaatccagaataatatccttatctcaagattattgacctaatcaaatcttcaaggtctttctgtcatataatctaatatattcacagtcttctgggatctggacatgaacatcttttgagcactggattggatggaaattgttctttttaccaaagctgggctggctccttaaagtgtgagatatcccaatggtctataatacactagataacaagaactaaaacataaattaaatatttaaataaatattaaatagagaagaacagctagagggaaggaacctggtctcttgatgacatctttgagttgtacctactgctggacttttcgcataagtcactatggtaggattagctattgctcacggcttaacaccttaacacaatcctaacagaaacagtaggactgtctgtattccttccaaattcccccctttcaagcttcaaggaggtgccctcttgcctaccaagcagggactagcttactgagtgagcaaatccatattttgttctataatcataaccatgttcaagatatttaaaatgttgctcattcctcggcttcgtttttttccttgctttaattatcaggaggaagattgagtgcttatatagtcaaggagaaagtaggactgaaactgagcgcttgctttttttcacatagaatatatgataaattcatcagcagagagtcagtttggggaaatttttatttaataattgtctaatcttgaatatgcctacattatctcactaaccttccaggcggccagcatgggaggtttgataatcttcgtgtacaggcaaacgagtggatgccctgaggttcccagcttcttaaaggctacggagaaagtgtgttacagacctgggattcagacctagtagagaaatccggagcctgcagacagaaagaaagggtcttctgggaaggggaagggactcacgttttttgagtttccgttgtgtgtggtcactgtggtcagcgtttcttattagtggtctcgtatttatagcagccccggtggatcagtattattggcccagcttcagaagcaagaaacgagcctggagataccaggtatactgcccaaagttgaaaagatggtggtaaacgacagaaccggatttgagccaagcttgatttcaaagttgtgttctcacccattaggcaaaggtgccaggcttgggttcaggatgagacccctacctgccttgaggaaagaggacaatttcttcagtatcagagttatttttctctcaggagagctaggaacacttaattcgatactagaagcagtactaggataccctccatccctagatagctgcagcggagacctacccattactccaggggcagtgtcaggagcccatggcctgtttgggcttcacacttcatggtgaagaagggactgcacaccaagccagaagtgtgccccgtttcagacaagattaattcaacctgcctcaaggcttggatgtgcagatggagtctgcatttgggaacaagatcactattccaccaaggatttggagaggtgactcagcagcagactgctggggaaggcaaagtcaaggcagtcaaccctgcctctgaggctggagtgctgtgtgaactcagacaatttccttcccctccctggacctcagcaccttttaatttacctatcaatccctgagttgcttcagctctgacaatctaggacttgcaggaataacacatctggtcatgaggaataaatcagtgtaaagggaaattcagttattcagcaatagtgaagaagagcctacagtgtttatggctgtggggagagttctgctccagctagtgtaatatgggtctcaactttgagcagctctggattttacagagattatactctgatgatttatgagtaaagtcctccttgcagagtagctctggttgcctagagtgtgaattacaaaagacagggaatccaagtgcttttagaaagggtgtgccctcctgatgtgtcacaggcctgctttctcctttgtcttggctcccagtgacaacacgtttgagtttgtatctccaaattagactcttgatttgtcttgcagaacgctagtcctgcctccatttcttcctatccctccctggccactgcaagccgagggggaagctgtaatttgtgagctgccctttcattgcagtgtgagtaacaaaaatgccttaatcctccctcatcctgcagggctgtgcccagcagccaggcccccagaggacatgaaggccaacgtgaacgggattctctcccatgcattattcacaccagtgcttccctttctatttttaatgttcagcatgtagcggttccctctgccagctatttttagcattgttgctgccagctgacagcagagacaaaggcaccgaggcaccttgagaatgacaaatggcgatttcaatgatgaaaatagaaaggctaattttcttccaggaaagaaaaggaagaaaacgtctgtagttgcaaagttgcttgggcttgaattgaatttttaatttggaacatatacactgccacgcacataaaggatttcctttcacacacacacacacacacacacacacacacacactcacatgctcatcattctcccagatcaaatacactctttttttgtgaagctttatctgatgcttctctctgctcctaataagccctcctctctctagatatagcatctccattattgtgatcctcacctggtattgtaaatgtttgcttctctgcaggccgtgagctcctctcactcaaggacttattgttaaatctccattgtctagtattaggaaaaacaagcagcccctgccatctagaagccaaccgggcactcacagttaggcatgtcattccctgttggacataaacaatctcattgaataccagaatcagacaaggttactctgagaccatgagaaagtgagacaaaacaagaacacttcataattttgtctgagcacagacaaaaacaaggtagctctgccaaccacaaaatatcatgtcttctctcttgcctaaaatgaatgactgctacttcttaatcaatccatcaatcaatcacatctttattctttccagtccgcccttcctatagacaaggtttattgagatgttcaataatataattaccccactttctgagagcagccaacctagagaaaatctctgcttctttagacccacccccaagtctccaacaaatatccgaatcccataataggttgtttctagcaccctgttactgagtggtctggaagctttcatggaactacaaaaaggggaagctacaaaaacgtcagaggaagagtggaaggaaggaaataaaaatgtccagggtagtatcttccctttgatcttccactcatagctaagggagctatgtactagccacacctgcgtaggtgtaattttatataaatgggattatattagtgaaaacatgaataagaacattttgagatttcataaggaggtcctcaataataaatttcttttagacctggtgatttttatgttattgctttttccattatgttcacataatagttcagagatttacttgaacctgccttacgccaccttttgccactaggttaacaaattcttcattacaataatctcatatcggcctgcaatttctagggcccagtcagctttggggttgtcatatttctgcttcttaatggttaccttgggctcctggcttctgttccttcattcccgtgatagcaattgtctggtacttccaaagatggatccacagcaaaatcctctgttgtacaacccctagtgcttttatgctctcatttttctctcacagaagccccttctgtgtccactatattctggtcctaaatgggggtggaaagcatgagagccaccttatgaggggctgacatgtgaaagatgattataattgccttcagtgggcccataagatgaaaacctagagattccagagagacacagtccagctatatatagggagaaatttcaatattaggaaaaataactcaaagatataatgggctgctgtatgaactagtgagctgcccaacatgggtagtatacaagcagaaatcagaactggcagaaatgttttagaacaccaacatggggtgtgtgagtctggaggtggagtgtcaacagaaaggaagtgttgttagagtaattgcagagtctctgaatccatggacttcatcctcctccacctttcttacaaatacatattgttaaatgtactataaatgctgttgtgattcataagataaaatttcacttaaaacgattactgagtttgtagtagcataatgtctcaatcaatgaatactaaaaatcaaactaatgttatgcaggagtttgtgaaattttctgaccctgaaaggtgacttctcattcttaaaatattctcattcacacaaggtgggagaaatcagatgacatttacattcttccctgctgtggtttgaatgtttatgtcctgcctcaaatttgtatgttgaaaactcacccctaaggtgataatgctaagaggcagggcctttgggaggtgataagatggtgagtgtggagtcctcaagaataggattagtgcccttataaaagaggccccagacacagtggctcatgcctataatcccagcactttgggaggccgaggcaggtggatcatgaggtcaggagattgagaccaccctggctaacacggtgaaaccccgtttctactaaaaatacaaaaattagccgggcatggtggcgggcgcctgtagccccagctactctggaggctgagacaggagaatggcatgaacccaggaggtggagcttgcagagagccgagtttgcgccactgcactccagcctgggcaacagagtgagactccagctcaaaaaaaaaaaagaggccccagagagatgccttgcccttctgccaagtttggatacagtgaggagacatcatttatgaaccagaaaacaggcagtcagcagacacaaaatgtaccagtgcctggattttggactttccaccctccagagctgtgagaaatatatgtctattgtttataagccacccagtatacagtattttgttagaagctcatacaggctaagatacttgtagaccggttcttggattctaagcattcacctttaatataacattatttcacaaaacaaagaagagacataagctattagcaacaataagatttgtgcgtgtgatttagtttgtcactaagagatgcattatttaacaaatatataagggtcgtgcccattaagaaagaggaaattgtatttagtaattcaattgtccatgtatctgtcactgacatttatttaagacttttttcttaaaaatccatttgcataggccactcaagtgtcacatggccctgaactttagatgtcaactaaggtgaagcatgcatcatgtcaggcagtcttcaggaagagaaaataaaacgcttatggagacaggtggttggtcattcttcaactctactcaggagctgaatgaaacagggagcagttcaatcctgcatcagaagctattcaaattggcccctaagtaggcctttctgaaagcaagtgttgtcaaattttacagctctcgctggataggctgaagtcccttccaaaatatgggcattggcattcatcctcatttttgagcaacttgttttagagaatatgtttaccttcttacctaggaggtcaagtggcatctagagagagaaggataagctcaatgaagccaatttttaaagagtatatttaaggaagaaatgatataacttgcatcttaaggtggtggaagtatccttgggtcacaagacaggtgatctgtgttctggttcagttaatctacatcagagtttgagttctggcctcagtttaccatgagactgaaccatatcacctttggtgagatatggcgaggccagatgcaaacccgaagagaaaaatggaaaagagtttcacagttttgttatactcacagctccttggggaagacacaggatgccacacagggccactggagaagtcagtgttgggcatgaggcagagggagaaagggaaatgtggggaagcacctttatttggtttctgggggaaggaaatgggtgaggtagggtaagccagtttagaattggaaaatttgaataatttcagcagggttaaggccatcagggttttccctggttgtctggtacctagccccagggtgattagtgtgggtgtatagtggttgcactgtgagaggctgatgagggagatgactgggggagtcagcttaattagctacttaggaaggggaactgacaggctcttagccatggtctcaaaactgagtcagacagcacttaaacgatattacatggaccttgaccctttctggcttcagtttcctcattttttcacataggaagaaggaacagaaggtctttaaattttcttcaactctgaagttattcactttggtaaaagtgccttctgaaattatttgtaacatctcccagagacccattgctttccagctagaatatgctgaaattctagtttaatgttaacaatgattttcaagatattgccagcattgtcttggatgcaaaacataccataaaatgaatatgcaatgcaaatttaatttttaatgcagtatttgaagccctggtagacaaatcatccacctcagcattgttctcacataaagtacagattgcagttccaaaagccatgccctggacttgcctttattctcctaactttatctccaaggattctctctccctgtgtaatcttcttggggatcatgaatctcattttctattcattcattcattcgttcaattattcaaccattcaacacacatttactgactcaaatttctaattatcaggtattgtgtaatcattgaagatgcagaactaaataaggcattgcccacctcctcaaggatttcaatgtctaggtcagggagctattcatctcagcagacatgttagggaaagtgaaagaggagtgaaactcaatgtgacaatgtcacagttttcaatctggtctaaaaaaaatgaccacacaaatttctctgtaaatagagaaaaatggggaaaatggctcagccttttctaaaaaaataaaataaaataaagtgaaggcagtgacatgtgacttcctctttgtgggtcttcttttctttattgcttcagtgtctctttacaaaaccccatgggcaaattttatttagatgtgcttttcctgagggtaatctattctctgaagtttccctcatttcctccagccacccaatattttaccaaacccatatttaatcagcaaagtcatgttgtgtgaacagcaacttaattaaactagaatttaagacttaaatcattatgcttgtcttcatcatctgatcatttattttaaaagttttgtaaatgtattttttaaattatgaagtgtgtgtgtgtgtgtgtgcatacactcactgcaacaaatgaaacaatagattacaaagatttataaaggcaaagataatcatcttccgtgtttctcattttaatcctatctccctgaagtaaccaatgttattagcacatctttatcataattcacacaagtgtataaaagcatatataatcaggttttgtctttattttttagcaaattagaatcatatacccattactctgcaacttcctttttttcactttaaaatatatcctgagaatctctataaatccactgaagatacctatctgcttattttcttcatgcatatgcacataaaacacacacaccaacagcaacaaatacgcacacattaaattaattaaggctatatttggctgcatgtgatagacaagatagctgaacaaacaagattaaagtttattctctctcatttaaatgaggtctggaagtcagttgctcagtggtgagctgtccaggcctggtgtggcgaatccataaccagggagccacgcttcttttgtcttgctgcctcaccattctttgtatgctgcctcatggtctaaaatggctgcccaaactctggccattatgtctatcttccaacagaaaaaagaagggtaaaaaaggacacttcctctttctttaaggatgagcatacaatttctcgccttacatctcattgaccagcatttagtcacctagtgcaaggaaggctgggaaatgtctttttctgggcagccatgtgccagctaaaaagtcatttgtttccttactaaaaaaaaaaaaaaaaaaagtaagatagaaactgggagccactcctgagtgtgtgtgtgtgtgtgtgtgtgagatatctcatataaatggaatcatatgagcaagaacatgaataagaacatctggagcttgcacgaggaggttctcagcaatagatttcttttgagtatggtggtgttcagaccaatgttttcctacattgtgttattaatccaaggattttcttgtacctacctttagcctcctttttctactgggttaataaattcttcatcgtaatcatgtcatgtaaacctataattcctgggtctcagtcagctttgggatttccatgtctgtacagggtcaaaagcatgaacctgtgagttaggacattggagttctcagctcagcctttccatgacaagttctgggcttctggatgaactatctccttgctgggctccagcttcctcctttgtactgtactctagtgcctctttcagaaaaagtaaaaagaataaattctttggagtcagattctcatgggttcaaatcttgcctataccattgactgatattcctcaagcaaacggtttaactgaagtttcttcatctatatgactagtacaatagtaactacgtcatacaataattttgagaattaaaagctacatcctgttacctgtttagcacaatgtctgatacattgttgatgctcaataaatgtgagttctcatcaccttgattgttttatctttctaaacaattactaaggcaatctaaattctggttctcccaatgtgtctggactcttaatgacaataatagcaacaacaacaagaacaagagttagtatttgttaagcattatgttcatgttcggtaatactaatcagaagagttcgcttatttttgaatcagtgtatttgatttggaattgaaatatactaagtcactgtgttccaggtactatgaacaggttgctaagatacttcagaggaatatcagaaggcagagatcatcttgtgaccacaagagtggaggtacatcattaatgctcaatacacatgattttcacgaacagacttggggttggcccacgtgaagtggaggatcctgttgagactgaagcttcccattgtttgttgacaggaccccttctcctgttcataacatagtgagaaaagccatttatgaaagggatacatggaaaccagaggcttggtggactcccagggcagagtttgagatagtcttgtacagcagacaaagagggaaacaagggggatgaaatcaggtttctctaaatgtgaatttggctgagtttgtgtgctagagattctggtgtcctgcaattcagagaatctaccatggacttaggtcagagtcagtgtaaataaacacaatgtggaacattacttctgcctttctgatactgtcagcatacgaaaagaagccctagagtcggtggatatgtgaacagcaacatatactgagcatgtgatgtatgccaagtgctatggcaggcaactgatgagaatgatgtcattaatcctcattgtaaccctatgagatggatactttcactttagaaatgtagaaaatgagactcaaagaactaaggaagcaacaagaaaaacttatgaactgggcagatacaaccaattaaaataatgttaagattaaagatagcctcagttcaaccagaagaaagatagcttggataagttggtaggggaaagggaattggacatcattgggaaggactttccctatgggggattcagggttttgtttatgctcgatttttattttcctgtgtcactgttcctgtgtgttttctttcccactggttttcatatttgctactttaacaatttcatggcccaggtacataaacttcacacattgcctctgtgcctggaacttcccttttctgtgtcgtggtggcaaggtttccacctggatgccccgcaagcatagcatgctcacagaaaactaaccttccgtccaccccatcttctgctccttgcttccaggttcaccatctgcccaatctcccaaggtgtgaatgaagagccatccttggctcctactcacaccctcctgttagctgccacttccgtcatctgattctatgggttctatgcacactttctgtgccctccgcctcctcttcatccctccagctttattcctgctcaggttcctattctaccacttggacttctacaacagttttctttcgcctctctgcatccaaggagagggtatttaatgaccagtcgtttgactttggtaaatgcattttctacctaagaaaatcatggttttccaagaagcaggtgctgacaaaaagattcatgtgcagatgatgtgtgtggacatgtttccaggagaacctggaaagggagcaggggaagcactgcttggaaagaagtcaagtaggggtgaaatttcatgccaggtctccagccaaatcctgcaggaaactctggagtttgtaccaactcaagcgagaaagctgggtgttcatattcctgcatcagtcagtccttggctaagaaccaccccagggagaccaaactcagagctacttcctgctcactgcaggtggcttcagtagcctagggcagagaacttcaggtaatggctgaaggcagcaaaggcacaacacacccagaaatcaggcaggggctcatagaagcaggacaggggatctcaggggatctgtgtggagcaccaaaaagtctgctgtagaaattttccagttttaaagataggagagaactaacattttttgagtacattctgtattcacaaccactctgcaagtggacatcattttcctcatattatttgaatgggacaaaaataatatcaccttaactagcttctccagaatttttcaaatggaagaaatattcctcagtaaggttatctaaagatttggatgtccaaagaacaaagagattaagtcattttgcatagaaaataagccaaatgcactcacttattcatgagaaggagaagagaaataagggaagaatggggagtgagtcaggcaagtggtgaaggatagggaacagcatagggtggcattgccagagtaatccccagtgatgggatgaccaaaaggactctagaaagtaaaatgtcacacacgctaatgaccaatgaggtgcctggcgacacagataaagccagggacaaagccagcagaaaactgtgttttccccaaagtcaactgccgctcaaaattactaacaacccttttttttgagtgattgctagtttgttgcaaatgacttccccagccctctctatttctctcacccctaaacgaagagaaactcagttaccaaactgcaccagtttaatggcagtccccagtctggaaccaatccttgcttcttcttcatccctctccattcctccttacaatccttcagcacaagcccaaaccctaagaaaaagaacttcccactccagcctaaagaggtgcccccaggtttctctggtctgtggtttcccttgctgcagcaagttaataaggccaactttgatagtttcaggtacatttatgatggtctgtgtctacgggttctttgctagacttttcacacctaatacattttgtttggttggttggttggttttgttgttttgttttgagacagagtctcgctctgtcgcccaggctggtatgcagtggtgtgatcacaactcaccgaagcctcgacctccatggctcaagcgatcctcctgcagtggcaccctgagtagctggaacacagatgtacaccactatgcccagctactttaatgatttatttattattttagagatggggtctccctatgttgctcaggctggtcccgaaccgctggactcaagctatcctcctacactccctctcagtgttgggattacaggcatgagccaccacacaggtgtattagcccagggctaatacatggttttaaggtgccatgcaatgtggtaagtgatgcttcctccctctttgtaactccttaaagctttgagtgaggggtttgtaggtggttgagtttcaccccaggatcaacatagcatggtatagaacaagacagagtaagaagcccagtgatcacagtaacaagggaatttaaactattcaaactgttgcatgggtttctgatttccatggaccatgtgattgtgatgggggtgggtatcaaatcaatcttatgggaggagatcctgctgctaatcttctgacacttattgtcttccgcctccttttagcatctcccctgagtttattggacccatggctaaccagctagaggttatattttcaatcctcccatgaatctaaatgtgtccataagccttggtctggccaaggggatagaagcacaagaaatctgcataagtttcaggtcaagtctttaagatggaaggctcgttgccttcattacctctttttgcttctgtcccacaggctgcaacatgcaactggtgtaggtgagtcagcttcgaccagggggacaaaagcaacaacctggagaatgctggaggatccaaacagaagggtttggtttcctccccatcctggcggagcatcctcctctgcccgttcatcccctgctatccaccttcctcagtgcattttgcgtgtaagaaagggagaaattaactttaaaaaaacgtttctgagtttgcttatctctttctcagagcatcttagcttatgctctattcaacaaatctagatctgaaaggatttggagacctgagctcataactaggctgtgttacaggcccagagaagctagataatttgtgcaaaatcatttaactcatgtgaccgaacccccaggcaggcacctctgtccccagtctgtttctgaatagtctccaggagtcatgttctccttcagattctcattttctctctctctctctctctctctctctctccctctctcacacagaggcatttggacttctttgccatccccacatcctccactaaaagcatcagctccagaattcagacatgtccatgatgtgttcatgcagagtcctcaactcagtgtggcttgtgaaagaagctctgtggggatcgccctggcttccctggaaatactaggatcactgagatggcaggttccactgagatattacactcagttgcctataaaaacgtcactcttaaaacagaagtagaaaatgtcacactttcaagtcataaacctagttttaaaatataagaaagaaaaaaaaagacaaagaagctccagccccccacaacggagctctgcacctctgtgaatgtgtgagaccaaggcattgtctttggggcaggatgaggacacttgaagaaatgtctcacgatggggaagttaagtggaaaatacaggcttcaatggtttaacaacaatccccttttctggcatatggagctgggcactaaaaagcactccatccctcttaagctagtggacagagagccacacttgtccccacttccaaccttcctctagcaccacgacgaactggatctctggtgactgtatgtgattgttttctctaggaggatgctacagaccaaataatcactttcatcactttgaagttatgcagcttctatgttctatggtcatttcccccaggatagaggagagtgttatatggggttattatgaaaaccctttatttgggtggtaggattgggatcctgttttcaagaagtgagctcctccaagaatgaggcaccaagctgtgcagacccatctttgacatttcttcctcacatcctcttacagggtggcctgacagggtaggcagagcttgagcttgcatcttataacaccagggtttaattctaagctcctgctagctaactcagtgaccatagataaatcagttgacctccttagcctcagcttacacatcagtaaattgagtataatatagtccagggtcattgagaaaatcagaagcaaaactagtcatagccgacatttacaaagtgctccctatttgccaggcacaattctattatttacaggagatctttcctttcttcctcatagcaaatctagaaggtggtgtcatcattctctgtggtgcagatgaagaagcagagcttccaaggggtggagaagaagctgaagtgtaggaagattaagtaatttcctagtggcccccttggtccatgtatccctggcaatctacctcaggaatctacatttttgccccctacgctctagccacttgccttgctgatgtctgtcctgtgtctgccacattgtccttgagtctctgtacagaccagatgtcgtttccttcattagacacaggaggacactgacacccagagccgcctgccaggctcacccagtgataagtggccaataagaatcagtgcccaggtatttccaaagcacttttctgtctattatttcttttgccaaagccccacaaggcaggtactgttatctccaacttagataaaacacttggagtacagagaggccaaatgacttccccaaggtctcatagccaagtggtagcaaagtcacaatgcaacccccagtcacctgaccctgtcctaggacatggcaggaagcacagggtatgtcccaaaggacaatgctgttgtcagtaaaatcacacaccgatggagagggtggacaggtgtctccctttattgtctcctggaaacactacaaaacactcctttctggtgtagggaagtatatctggttaagcaagaattaagtactaggggtggcattacgtatgtgcatgtgaaagggggtggtcatgcctgccttagagagacaaggggcatgtggagtaggggaggacagaatcacatagaggacccctagggagagaaagagaaagacagagagagagaaatcagcctctcactatgggctcagaaaggaccagaagacagccacttgtggggtgtgtgtgtttgtgtgtgtgtttcatggtcagaaggaggtgcacagagtttatgctcaagtctcattagatttctaagcacctacataaaatttattcatttccctggaatgataatttacttatttgcctccccaaccaccctattatctctgtgatgacagaggctctgtttatttatctttgcagtatttattctcctagagatatttgcatttaaaggaaaggaaacccccaaagatgcatttatttcaagaacaacttctaataacatcagtgttttgcttcttaaaacatacctaaggctttatcatgtagagagttggccaaaatgtacctaataagactgaatgcctgggggacatgcaaagtaggccactggtgaggcaaaagagccatggtcagagaagaagttaggaagttcattctgagaataagaggaggaggcgttagccttacttatgagcatgctgactacacagaaaatgcttaaacagaacatacattctgcttccataagtttggtcaaaaccaccttgaatgtcagctgaaaactgatgtgagagaatgtaaattgcctgcctgtgctggtcctatcctcccagtatcaccaaactggacatgcatccatccaaccagtacagcagagccaaatgctggcactgggatttgcagcaagagaaagtgaggcatttattgcagggcgccaagcaaggagaatcagggagttcatgcttaagacccaaactacccaatggcctacatgtaatggttttttttattatttttttagttggagtctcactctattgcccaggctggagtgcagtggcatgatttcagctccctgcaacctccgcttcctgggttcaagtgattctcctacctcagccttccaagtagctggaattacatgtgcacgccaccacacccagctaatttttgtatttttagtagagatggggtttcaccatattagctgggccggtctcaaactcctgacctcaggtgatccacccaccttggcctcccaaagtgctggattacaggcgtgagccaccacgcccagccagggtttttaaaggcagggaggcaaagtcgtaaattaatacatggaggctatccattacttttacctaaaaaggtgggacatctcaaaggggatggggcccgcaggttacgggtggattaaaagaatttgatttgtgtttagttaaactttgtctaaaaacttagcatcagcagaaaggaatgtctctttcacccattcctttgggtatgacttcctccacgcccccaggaagatatttagaacaaagactgaaatttacaacaaaaaatttagtcaacgttcaattctccatttccctttatctgaggtctacatgccagtggatgatattttccatttagtaggggtctgagtttctgaaaagtaactcagggacatatgttaagattttatcttcagtttctataggaaaccaagcattttgtggctctaatatctttagctattgttgtaagctactattacttcctgcttattaggttgctaatttacttctcaaggctagctgggtgcctgaaatttcccctgaaggaactcaagattttcttttttctccatgcctgggggaccacgcaggccactaagaggtgcccctgctctgtatcatcagctatcttacctttgagaaaacttgttaacagtccttccttgctgttcaagttctttccccctcccccgctctttagtttccttcactgggcttgagctggtagacctgtggatttctaattccatccccatcttttttttgacctgtactaacagcaccatatgttatacaagttcaacttcttaccgtggccagcactggtctgatttcttctgcaggctcattttatgctcattattttaccatgagctctctgctccaggcttctgaacttccattatttctccagatctaatgctgggccttcatacatggctatgaacaggtatgcttcccccagcctttacctggttactcctatttaccagcaaatgccacctcctgggaggcaatatagcattatggttctaaatgtggattcttgaaatagcctgggttgtccctagttctgccctcacccaggtgtggaccctccacagctgttctttctctaagaggtccctgttctgggggcatctctgcaggtgtgagagccagcttcccccaccccttttaaaacaataacagctttattgggatatcattcacataccattacaattcactcttttaaaacgcacaattcaatagtttttagcatattcagggttgtgcaaccattaccacgatcaattttaaagcattttcattattccccctcaaaaaattccacacccattagcaatcactcgcctccccactgtcaactctaagtagccactaatcgaccttctgtctttatagatttgtctattgtggacattttatataaatgaaatcatacaatatatgtggtctttggtgactgacttatttcacttaacataatggttccaaggttggcccgtgttgtaacaggtatcagtacctcattctttttttattgttgaataaccattgcatggaaataccatcttttatttattatcatcagttggttggacatttgatttgcttccactttttgctgtcatgcataatgttatttggacattcatgtacaaatttttgtgtggacatatgttttcaattttcttgaatatatacataggagtagaaattctgggttgtacgataaggctatggttaacctttagagcaactgccagattgtttttcaatggtcccaggactctttgttcccagacccactcccagttcttcctctgctagtctctaagtggcaggagggctgacccctgcaggctgtttgtcagctggaagcccggagggtgagaggaaggaagaaaccgaggcatttctctcttccctcttatttccttccactccagcctcaggcagcctctccaacaatcctgtgtctctctcctcatgctggccctctctaattccagctttccgtgtgtgacactagctcttagtttccataacataccctccttttctctccctctatctggggagtgctgggtgctttctgctggtgaaaatcccctgttgggcctctcattcctttcatcactggtgtaactaacctgaattctcttgtaatagttacagctgtttctgttttctagcctatatttgacccttaaacaatacagggggtaggggtgccaaccccccaccccaccacacaataaaaaattcacgtatagctttgggctcccccacaattaattactgatggccttctgttgcctggagcattactgataatataaaatcaattaacatacattttgtatcttatatgtattatatgctgtattcttacaataaagaaagctaaagaaagaaaatgttattaagaaaatcataaggaagaaaaatatacttactatttgttaagtggaagtggatcatcataaaggtcttcctccttgtcatcttcacactgagtaggctgaggaggatgagaaagaggaaggattggtctctctgtctcagggagatggcagaggtggaagaaactctgtataaatagggacccacacagttctaacctgtcttgttctagtatcaactagggatggcagaggtggaagaaactctgtacaaatagggacccacacagttctaacctgtctttttctagtatcaactatatatgcactgcatacaataccaatgcacttatctgtttactgacctgaatccaagcagaatgtaagggtcctgtgggtggggggctacctcgttatatctacccttgtatctggagtgcctggcctagaatggatctttaataaatatttattgaatgatgaaataatgactaaaggggtaaattactagaaaattttcctggcaactgtcaaggattgttcttctttccaactccttttcactcctatttcatctctgcacaatttcaaactcttattcctgtgcacatagcccatttcgcagctcctctaaaagtcagaatggtgcaacccattctccttcctgtctacccttgctctttggtgacttggtccatcttatttctttaattacaatttatgtgtggatgattccaagttcgtatctccagtccttaatgctccgataaactccagactcatatatttaactgccagtgtcacgtttccactaatgcccatctcacacctaatatatctgaaactgaactcaaactccccccacagtctctcccttgcaataaatagcagttctatttcctatatccataggccaaaattgttgaactcattcttggctcctctctctcaaaccccatatttgatctatcagtaaatcataactgacatcttcctacacctccattgctattcccctcgtccaagacgtggccgtctttcacctggattacagtagtcactcttccctggcctccctgcttccactcctgctccctacaagtctactcccaacagagcagccagagggatactgataaaacgtaagccaaatcatgtcccttctctgcctaaccctccatgggtctcccattgaactcagagtagaagccaaatcttcatgaagacctctatgctcctacacaacctagtgctcctccccaccccacccccttcctgacctcacttcctacctcctcatgctcacctggctcctgttaccctgaccttccagctacgcttcatacatgcattcataccaagcattggtatgggccagctacgcttcataccaagcattcctggcccagggtttctgcaccaccattctctctacctggcatgcttttttttttttttttttttatcagacatcttcgtccaggtcattaccagtctcaccttcttagtgaggccttctctgggaaccctatctagatgcaaatttcctgttaccttctgcttctctgctttcatttatatccatctatatcatttccatttataccatagtttacttacgtcttacattctttttttttttttttaaaacagagtctcactctgttacccaggttggaatgcaggggtgcaatctcagctcactgcaacctccgcctccggggttcaagcgattctcctgtctcagcctcctgagtagctgggattacaggcatacaccaccatgcccagctaatttttgtatttttagtagagacagggattcaccaagttggccaggcttgtcgcaaactcctgacgtcaagtaatctgcccacctcggctttccaaagtgctgggattacaggtgtaagccatgagccaccacacctggcctattacttatattctttattatctgtctctactctccaccttgccacacttaaagctccctgaggacagacagataatatttttttctcttgcaatctttggtgtgtcctggtgctaaaatagtgcttttcacaccatagcatttcacagatatttgccatagcagtggaagggctactctaaggaaacgtgccttcggagtttcccaccccacccttgtcatggcagggttaggaacacactctgcaatcagaaagaactaggttcagactctagctctaagagttaatgaccttgagccaagtatttaacccctgcaactggaagtctcgttggtaaattcatatttcatcagggcttgtacagaccaagacacaaaacatggaaacaagatgttccacccaagcctgcaccattatgtgtattccacataaactacagtgtgggtgctgcctgagaatctgggataatttttaaagtcccaaatattcacagacctggatacttagtctcacctctgaaattagttgttaactgtggaagaagctagcttgctggttttgaagcatgagatctgagatgttaccttattatgtcaataaacagataattttagtagacacattaacaacatttaaatagaaatttggggtaattaatagataaatgagaaatagcaggtggaaaaagtttccattttctacgctttcttttccaaaacacctttaaaaatccttttcaaagataattctccttaatatttaatatgtaatttcctcttcaccccataaacgtttacattgatgtaatgtatatattcagtaagttatatttaataaaaagcctgccatatacagggtcatcattgacctctcattcacttatttactccacttcatttattcatcgcacaagtgtttactgaacacctaatatgtaagagactgtgctgggcccttaaggtaatgcacgagtaaataaaagattttactttcataaaatttacaatttaaggaaggacagagattaagacctgcacacgaatatcagtcacctcacatttataaaggtacaaaaatggaaaatgataattcagagaaggagatcaattgcttccatatgggtcatccagttgtactcaatcttctttgaactgctttgcacttaatcctctatgtgccttggttccacagaaccagcaggaaaaaggcagaggttaacaaatagaaaggcctcaggctgctgctactgtcctggctcttctgcctgctcttgctcagatccaccaacctcctacttattacgtctggagccttgtccagttctcatctacatggctatctgatgggactggccacctcctccatggagctacctttccccagtttctcctggttcaatccctatctctttctcagtcctgtgggtacttcactgaatcaccctgctgtgatcatgcctttagataagggtccgttggaccttcttttcctcttagaagagagaaatctccaaggttcacaaaaatattgcctcatccatattgtatttattttagtagcagcaataatatctatagtgtttttaataggctaactataataaactaggggaactgaatgaggtacaaatgatttatttatgacaaaaagacataatataaacttagctctcattgccttattaagtttggggattatacaggagaaaggactctaaagatgcttggtcaagtagggagaaaggtagagtttattgactcagatgtattcctctgaaatacagattttaccatcttgccaaggtctccaggaggtggtgtgaatttgtaatcctggaagtgtggataaaacaaagccttatgctgagcaaagttgaaatgctagcattcctagggtagatgatggaggaaggaatgaaaagtctcatggaagtcaacatgccagaattgacttaccatgttcatattgaggaagacccatcagatgactcatgggaagggtcagaggacaacccttttaccaaaaacataaggaatactctgttgagaagtcaccagcaacacaaaaactggtgaatctccttggtaggccagggatgacgtagaagaggctcttaaagagtttggcttgcagatagcaatggggatgatagaatcttaaagcaatgaaggtcaggcggcagcactgaactgccagaacctggaagttgcaactaccacaactgtcaggcaagagggcccgacaaacagaggattatggagatgattcatagaatatggcatcctttgaggcaaagcacatagagggaacatgggtaatacttagattatatgagcagaagaaatcaagaatgaatgatcaccaaagtggtagctgaacatgtgaagtttttcacactaacttccctcttctaagtttcccttaggaagagaagcccaccagaatcacggaaagcagaatcttccaacgtatttggagaagaagatccaagcagaggaaggggtgggctgtggcatatatgtaagtgtgcttcccagatcctcttcctaggaagaactttctgccagctacagtacgtgtggtcagcatacactacagcctctagtagtgtgttccttcagggtttgactctgctgcttcactcaggttgaagcttttcctagggcagctagtgattgggttggatgggaataatctcacccttactagtctcccttactactccttgttgtttgtcctagcaagtagtccctcttattaaaccctccttgcattttcttaatttgagagtgccttcactctgattgatactataaccactaagactgatacacctatggagacacccataaatacatgatttgggggaggcacattttaagattctgcttcatgtgttgggtcagtgcaaaagccagtaaaagccagtaaattaccaacataaaaacatgggtctggaatatatattccactctaagttttccggaaggtttttttgtgtgtattcccatcagctcatacatgatgcaatttattcttcattccaagtaactgattgcctgtcttcaatgtcacccgctgaatatggcactgactggccaccgtcaatcagcctagactcaatctcttttcaacactgcagatcttgccaggctgaagaaagcatttgttctctaggggcttccaaaaatattgacagtttttggaagagattcaacactaattttggggactttctatttttcttttccatggtgacagctttcctgacagcatgaggctaagcatttctgctatttaaatggtaatttttaaaaagatgatgatgatctcatctctgtagctttgtctgggtaggcatgagtagcagaaacatctcatatgccagactgcataatgagcttttgagaagatgatggtgcattggattgctaacacaactgtggtcttggggtcaggctccttctgtgtacccatggcatctgagttattacatatggggtaactttgcttctaaaaatggaacagctggcattttcttttccagttggtcattggaagctcctctctgtggggattatatttgtggcagatgccttcttctcaaggtatgctagacgtgccattcctcaaatgtgtcctttactgtacagtattcagttctttgcagttccctccacctggaatgcctgctccctcatttccacatgtccaacttctactcattgtagaattgccattgactccataaagtcttcctccaatctctcatgttgaatttagcatttttcttatctgaacatccgtaacattataattctatgataacttagaaataatacggtgtaacattttagttttcaaagcattttacacatacattatttaatcctcacagtttatgacattatccccttagtttgtttcaaggcttgtgtgttatgccagtcattggtattgttcatccaacagtcccagttctcttgacacataataatgttataccttcctgtccctgtgaatttaggcacagccatgagacttgctttggccaagaaaacagaacacatatgtcatttctgggctacagctttaagagccagagtgcaatttgcatcactttctttttctcccatcagaatgaccaatgatgtttgagattgtggctgctctgacagcctggatttttctgctaggtgctagggatacagataggtaagaatgattccagccaggagtgctcgagcctggcgcagaattactggcactagtgttagcacattactagtgctggtgcatgggttactctgataaaattaaatattactatattggaaatattttcaaaataatgtaggtgtatgggaaaaaaaaatcctgaacttgcctggaaagacttatggaaacatttacatttgaactgatacttgaaggatgagtacatgttagccaggccaaaaaagtggaagtgggttgcttcagaacaaagaccacgagaaaggtatacaggtagaaaaactgctttcaaggacagaagagacagattgaatgaatatgagatcatagagattatgatgaaggccaacaccctggtgtattcataaacactcatctttatttttatatggcaacacagtaggcactgagtaaatgattgctcatgtctctcttcacatgtcaagctctatactgttttttttgaggtcagaatagagaacaatttcacattctctgtgtaaattttgtatatgagacccaactttcttttccccaatgcctgacccttgctttggaaaagacatggcttcgttagtaaatgccctgattcaaaactgtcaaagattcttcatccactaccttcaggagaaagttcaaactcctgggaaggcattcaaggtcattcacaatctgtccccaagctatctctctaggatcaattgttctctgttcatgttcatatggaacatgtatcctgaactttcaactctctgagccttcatctgtcatttcgtccaccttgaaggccctttcactccttttctcccaagcaaattcatactcatctttcaagacctaacttaaatatcacctcctagatgaagtcaaattctttcctgattccctttctccaacagagtaaatctctttcctctctgttgctttctctctttttgtaagagggatttttttccccattggtttttggcagttttaatatgatctgcttaaatgtaattttgtttttatttattcagtttaggatttgtagagatttctgcatctttagcttgatgtctttagtcagatttagaaaatttccaaccactacctcattaagtatagcttctgccctattcttgatcttctgtctttttagggctccattaccctctcttctatagttacctgtattttctctgttcccatctcattctagatatttttcaactccaaaattctcctctcagcttggtttaatctgctgttaaagtaatccattgagttttaaatttcagttacttcatttttcagctctaaaattttaattttttgatatttcgaattcactaccaaaattcccaatcctttctttaatcttccagaatatataatgtacatttattttaaagtctgaagttgataactgtattattcacatcatctatgagtctacttccattacgtgttctattttccatttatatcagcttgtctccttgtgggcttggttgttttttattgatttacttacactgtattttaaaaactaggcaaagtgaataataggtgttgcttgactacagtccctgatacagagggattatatgagatgagacctatgattgttccatttttccaccgagggtaactttctacaatatggaagaggaagatgaagcccaaacacagcagagtggttctggtgagctgaagagacagagatcagagcttggaggtgctgaagaggctggagtttgtgggtcagcatgtcagagagacaggagggagctacgcagctaaagagctccagagatatgcatacagtccacttaaatctttgaacaaatactaatctgtgcatgcaaagaatgagacccctactgagcacggcagagaagctgtgaactgaatggagattctgcaggccacacaatgctggggacataggagttccaaccagccagggaaatcttggggaaccaccagtttactcagttgacatccccaaagccctttttgaattgaagtgtctattaaagttattttatgcttatctcagcattgtatgcttggggttggaagggagttaatttgctcatgggttttcagtttgagaagaaccatatttaaaaaagcaaacccagctgggcatagtggctcacgcctgttatcccagaactttgggaggccgagccaggcagatcacctgagatcgggatttcgagaccagcatggccaacacggagaaacccggtctctactaaaaatacaaaattagccgggcgtggtggtgtgtgcctgtaatcccggctactcaggaggctgaggcaggagaatcgcttgaacctgggaggcggaggttgtagtgagctgagatcacgccattgtactccagcctgagcagcaagagcaaaactccgttacaaaaaaaaaaaaaaaaagcaaacccaatgagtccaatgaatctcatctgtacccagaactaatttagatgatgagatcctgaatttcaagctagaactacaatagcatggaatcttgggggtcttgggaagggatggctgcatttgcatgtgaagaaataatttattataagaagatggagtgaggtaaagatgaccacaaatcctttgacactcctcctattgagaagtgggatctatacctccttgacccctgagatcagcttttaggtaaacaagtccatgcaggacctatcttatgtctgtcttgggaatcttgggttcaagcaggcccctctcttcttggcaatttccctaccacagaatcctccttagtggttgttttttctatagtaatacagggataggccaggaaatgtggccaagcagaatccccattaagaaataagtttctggccgggcatggtggttcacgcctgtaatctcaatactttggaaggcagaggcgggaaggtcacttgaggccaggagtttgagaccagcctggccaacatggtgaaaccctgtctctactaaaaatacaaaaattagctagatgtggtagtgttcacctataatcccagctactcaggaggctgaggtaggagaatcatttgaacttaggaggcagaggttgcagcgagccaaaggtagcagtgagccaagattgtgccactgcacttgagcctgggcaacagagcaagactctgtctcaaaaaaaaaaaaaaagataagaaagaagtttctcttgatatgatgaacatctttgagcagaagacacttttatgtgattctgattatttcttgaggttgtatgatcagacgcagaactttgaggtcataggacataaacctatttaaaattcttgctataccttaaaaatgttctttccaaaaatgttccagtttacatcacagtaatagttctgagaagctcagatatgatagatcttgaaggggaaatcaaaggcacatggggttttccctgtccatagaaaatgacataatctcctggtgtctccgccaaaagcaattctctgttgggtggactgtgaagcgtgactgctttttaagccaacgaaaggctgtctaaaaatgggattttacacactttttttcaagtgagtgggagccagttggctgtagatctgagtttagacagaggaaagtttgaactgaactacaattttttttttaaatgggagattcacttggggattttctttttacatctaaaaccttctgcaatagaattggaatgcatttagcactcagagatgatttctaaaccagcttcaaatcctgacttgttagctggatggtcttgaatgtggaacttcacctctgtaactgagtttccctcactgtacaatgaggttaataacacttgcctctaagtgttgctgaaatattagaaagagtatcttctgcccaatcctctgccctgaataccaggctctgtctgcctacataacatttttccttggaaactaaaacttagcatgcccaacactgaactccccacctcctactgccccagacaacgattccagatctctgcaaacattgattcaccttgcagtttctcaagcacttcagtgtcacccttgattccttcctctctctcaaaaagtcacaaatctgcaaatcctgatgtctctctcttcaaaacgtacccaaaatccaatcacttctcccaactttctctgccaccccaaaatccaagccagcagcatgtctggattgctgccactacttctaacaggtccctgactttttcccttgccctgatagagcagctagagcagacccttggaaacaaagtcttccacattgctcccaaccctttagtgcatcaatgttctctcccatagacctccactgggaataaaagcaaagtgctgaccatgctctgccaggccatacaatttggctccaccaactttctgaccacatcttcctaattaacttgcctcccatctccagccatgtggccacctggtcacacttccgaggagcttggctcatgcccaccacagggcctttgctctcattccttaagctacttggaatccccttccctaagaagcccattctggctctgacaactcactgagggctctgctcaaatgcctccttatataaaaagccctccctaaccacaacatttatcattgcagccctcatgaaactccctgtccctctctcacttgttgtttctttataccacacacatcatgtggttggcttgtttatttgtttactacctatctcctgattacaatgtagactccatggccaggcgcggtggctcatgcctgtaatcacagcactttgggaggccaaggcaggcagatcatgaggttaagagatcaagaccatcctggccaacatggtgaaaccctgtctctactaaaaatacaaaaattagctgggcgtggtggcgcatgcctgtagtctcagctactcgggaggctgaggcaggagaatcacttgaatccaggaggcagaggctgcaatgagccaagactgcaccactgcactccagcctggtgacaaagcgagactccatctcaaaaaaaaaaaagaatgtagactccataagaacagagacctttgtcctttttgtttattgttattttctcagcctggaacataacaagtgctgtatcaatatttgcttgaacaatatgaaaggtatattgtagatgttcaataagtaatagttattatgagttttacagtattacattagcctcacttaaccacatatgtcatgaaagggatgtgctggtgagcttaactttctgacttattttgaattaaattactgttggtgaaaattttaaattttaaatcgtatttttaaaaataatgctttcctaaaaaagtaaatagattaaaattactcttcttgcttatttaacccccaaagctcatggaatggccttcatcaggttgaatgaaaaaacaaaggtgtctctgcagacagcagtaagccccagctctgcctctctcctctctcgtcctcctagcaaaatcttgtgatgggatgggatactggcgatgacaggccaagtgagtcctgctcactcactcttctaagaagtgtaatttctttccttaggtacaggcaggacctgggactcttcctacctgctgtgacagctgagctcctctttagctgatggcatgctgaggcctgtgcactgcggaggtatgtgggccaagtttaaaccctgcttcctctgcctatttgctgtgtgtggtccaacatcaatgactgaacttctctgcccaatttgtttatctgtaaaaggggaactttgagacctcctagaatttctcacaggttaaaggtgaggtctgaatactgctggctttatgcctagtgcatagctggccctaaggtcattggtcacttttccctctcagagagggtcacaggcacttcgggcacctcaatgccatacttgtcctcgctctgccagggttctgaccagtggagacatgtctaatgatcactatcccgtgggaagtaagaaagggctccatttatctctgcaaatcctctgtctcccagagcaggcccatctgggaaaggactgtgccctgaatctaaactttgggacatggatattgatagttccttgagggatagaagtgggcgtgaagaacaaactttggggcatggatattgatggttccttgagggatataagtgggcgtgaagaacaaactttgggacatggatattgatagttccttgagggatacaagtgggcgtgaagaacaaactttggggcatggatattgacagttccttgagggatacaagtgggtgtgaagaacagacgatgaaactgttcaccagcccagatgcatggacccaactctaggtctgctgcacagacccagaaccaccccagattttgctcaggatgactcaccatatgggaataagaagagcaggaaattttacttcatggcttgagtgaaataaaagaggaagcagccacccttattcatgcttttaagacatagttatcttgttcaccgttaaaatgtatctcaatgaatgacttgctcaggtggattggattaaccacttggtaaggtgcaagcaaggtgacactgttttaggaggtttctcattggatctctcacattaagatctatgtgccagcttttaggtgtagatttttggattagtaggagtcaagttttatttttccccattcagatatgtgcttgactcagcaccatttattgaaaatgaaaaaagaaagtatcatttccctactgctttctagtctcttgtttattatagattgagtgtacatattgggtcgttctgtttcagaactttattcagttcctacatcacactgttgaaattactttatatatcatgcatttttcttcactatctttacaataactgagtcgtagtcatcaccacaatcaccaccaccaccaccatcaccaccgccaccatcatcatcatcattatcattatcctcttgagagctaatagttactgagtgcttactatatgccagtagttatacgtgcacttagtcttcacatcaaccttctgagattgatattattataaacttcattttgaagaagagaaaactgaggctcaatggtggtaggtagcattcccaagatcatgtagctagcaagtggctacacaaggttctggcccaggtttgtgagagctcagtccaccagggctttttactctatcctagtgtctcactcagctataatcctcccatcaaatatgtgccccatctttgatagagagaaggagatttccaggccatgagatagatagatgctggcttgggtttgctctatttggtaaacaactgggtgagtaaatttcatctcaatggggttattaactctggtgaaatgtctaaacatctgaggttggctattttgggcatcactgaagtttatgctggggaggaaattcacaatccagtcaatgccaaccacagattgggctgtctgaagccttacttgaaggaagttcctcttggacgctccccctttctcctgccacaccaccggctcctctcttgttggcataggggtccctggcaggtaagacccagcattgaaggcagaggttgtgtttccatagggccagggagtggccttgcccttgtcccactggcacccctcatagctgatctatctaggtgcttggagatgccgtgtgctctgtgtaaaagtgccagccagggacagaacagttccgggacagaacagttccaacccagaacagcaggctctggaagacttagactctgagttttgggggctgcagatgcaggggatagtgcaacccaggagaacacagtaattggaaggacactggcgggcaaggaggactttgccttctctgaccctgggattctgctgatggctactacaaacctgctgatatttctgctgagtgcccattcttgtctgaagtcagcagtccagggggtgctaactgcattcaggtaacacttcagatggcaatttaatgcccagccagtgctgatgaggcactattaatttggggacacttcaacaccacttcactgtgccagcttagcaacctggttaattaattaaaagaaaggagtaaaggatttatttaaagagacattgaaataaaaccggaagagtcactgcaaatattttgatgaaaaaagggagtttgtgattctatatatgtcaatgaaagtaaagctatactcttgttgatgcacatatatggaatttcatgctttttattataaatcagtatagtataaaagaaacatattcagtgggaagcacatgggtttgaaataagatagatctgagtttaatccaggctcttcttactaaaacaatgaaacatcacttatttaatctcaatgaaacccaactgtctcacctgtaaaatggggataatattcccaacattgcagggtcattttggagattggaggtggctgtatatgatgtgctcagcatcgtgttcgaccacaataggctccttaaacatgctttatacatgagagtccaacataagaactttatagacattgcaaatcattcttgaagtaggttctgtgcaacaatagttctttggtcataaaacctcctcattatattcctgtatgtagaattggccaaattggtgtggagggggttcctgggtttagctgaagggatgccacccagtgcaggctcacttgtgtgttctacactcagtacaggggcagcaaagctgtttcacacaggaagtcaggcctttgattccagaatgctcagcctttgcaggtttttgagtagggcagtgacatgaaagacagaaactttgaggtaagtgtgcaaaatcactcatattgcctactagggggccaggcttggaaacactcatggttaatacatgccctgctctcagaagagatggagagagaaagaatcagaataattcagaggttgtttcctcaagtaaatgttacagaagtaaagacctagacaaaaaagtcttaatgaaaaagaggatatgaaatattatcaaaagttttggtgttaacatatgtgggagtgtcataagtaacacccctgtttttactgcctgagttcagagtcaatgagaaagaagcaaggggggtgatattaatggatgctcatccacatggaaagggacatcaggagtaggcaacacatgttccctccaacaagaagcttgatagtaacttgaaaataaaatgttgcatcttaattgatattcaacatgatacattgtggaaggcatgcaattggcttcaaggatcactacactgtcttgtgatcaagtaaaaatatctgtctttcaaaggccaaaatggaactttatttcccaaaattatcactatttttgattaattttaggatgcattgctcagtttatttttgtagctaactcttctggttgtctatgcagtatacattctctccttcttcctctctaatagagcccagcttttcttgggtgcaaaacagtacacagttaaaaatactgtagctccagcctccctcacagcaagtggcagccagtgagacagggggagccacaatgtaggtccttgtgtgtgataagtgtaccccctcttactttcacccttttctggggtttcttgctcttctacttcttgtccagaaggttgatgctttgcttgaaggtgcagcagccaatttgtaccatgaagatgaaaggtggatgctaaggatagtgggataagaagattgacagagccagtgtttctgaattagtagtctggccctggattgctcaaccctagactacctagacatcttatgtattagatcatcgtgtgttgggttttctatcacttgcagccaaatgcattcccaattgctgtaattttgaaatttaacattacactaaaggaaggcatggacttttgctgtctaacttataggacatgattaagaaaaggtaataagggccaggcactggtggctcacgcctgtaatcccagcactgtgggaggctgaggtgggtggatcacgagatcaagatattgagatcatcctggccaacagtgtgaaaccccatctctactaaaaatacaaaaattggctgggcatggtggtgcatgcctgtagtcccagctacttgggaggctgaggcaggagaatagcttgaacccaggaggcggaggttgcagtgagctgggatcgcgacactgcactccagcctgggtgacagtgtgagactctgtcaaaagaaagaaatagagagagagagagagaaagaaagaaaggaaggaaggaaggaaggaaggaagggaaggaaggaaggaaaagaaagaaagaaacaaagaaacaaagaaagtgagagagaaagaaaaagaaagaaagaaagaaaaagaaagaaaggaagagaaagaaagaaagagagagaaagaaaggaggaaggaggggaagggaagagaagggagagaaaggaagggaagggaagggaagagaagggaagggaagggaagggagagaaagaaagggtaataagccattaactgacacaggggtctgacaggggcagagttgaaaagctgagtggctagtacttggggaccaagtgttctgctcggagagatagaggcagtaacaatttttatggtcacaagttagaacacaaagtctgaaaagtgatctgactaggaaaacagaatctttgaaacttcctggaatcctgaaggagtaaagcctatgagcagatgcaagtctttgagtgaagcagcgtgggataagacagaaccaaaaagacaatgattctatctcagctctgccacttactgcagtgtactggagtaaattgattaattgcctcaagactcggtttccttctctgtaaaatgaggccataaactctacctggcaggggaaaaaaatccacatcctagtccatgcaaagtcaacccctggagctgggcacaccatacctgtacaaccagacgtggtaggaatgatcctttgctatgggtattcaagcatgggtattatgatcctttgctatgtctttattttacccttacaacaccagtgtgaaaagatacggttatcctttttctataaacaatggaaccaaggttcagagaagttagtaacaggcctgacgtcaccctgcttgaacttggcagtgtaaaagtctctcccatcactgggcacagtggctcacgcctgtaatcctagcactttgggaggctgaggagggtggatcacctgaggtcaggagtttgagaccagcctggccaatgtggtgaaacgtcgtctctactaaaaatacaaaaattagctgggcatggtggcgggcacctgtaatcccagctactctggaggctgaggcaggagaattgcttgaactggggaggcagagcttgcagtgagccgagatcgtgccactgcactccagcctgggcgacagagcaagactccttctcaaaaaacaaaaaagtaaagaaatctctcctgtcctgggttcatttggtttcatacgcattcactacctaccagattcagtagagatcactagccttggttttatgacaggaaaaccaagctgggcaagttgtcatgcagaacaaaaacaaactctccatttcccaccctgtcctggagcacagagtcaccatatatctaagtcttcgccaatgagaagtaagtagaggtgtgtgggaactacaaggaatttccttaaagacatctgtggacatgtacttttctcctttgtctggctggaccatgactgtgatggttggcacttcagcaactatatcaggccctgaggtgacattcaatagagaaactatatgcttgaatgaaggagcagagacttagaaagctactctctgatgacaccatgtaacctccatgctcactgtattagtccattcttgcactgccatgaagaaatacctaagactgggtaatttataaaagaagaggcttaattgactcacagttctgcatggctggggaggcctcaggaaacttacaataatagcgaaaggcaaaggagaagcaggtaccttcttcacagggcagcaggacaaagtgagtgcaagcaggggaaatgctaggtgcttattaaaccatcaggtttcgtgagactcattatcaccagaacagcatggaagaaactgctccggtgattcaattacctccacctggtcccgcccttgacacatggggattatggggaataaaattcaattcaaggtgagactttggatgcggacaaagccaaaccatatctctcaccctatattttctgcctttggactgttgtttgatgagggaaaaataaatgtttctcttattttatccatgtattttattttctattctatgcagtgaaattaatcctaacttataaatcctaactgagataactgatgcacctgttggtgcttttggtttagaagcttagcctggggtagtgggccacacacttatacctctggtctgggatagctggagagtgggccaagggcaaggtctgctgtgaggaccaggggtgtcttcacattctcaaggacaggcaattgtgggacacaagaaagtgccctgggcttggagtccagtctaaatccaactgctggtcatgctcacatgagctgtgtggctctgtgctgggatcctggcctccctgagctccagctcatcctctgtaaagcagagatcagataaatacttctacctcattagtgctgttatgaagaacaagcagatgctgagtggaagcacctagaatgattttgcatcctaacaggtgccgggtccatgagtacaacattgcttaatctgaatctggagagaaacccacacattttccacctttttagctttctcttctcttttcctctctcctcatataatcccaccttccttctcagtgcttctaatccaacttaattgcttccctctatggtcccatcttaatgcttcatgtagttgggatgagtactcacacaatgagttgcaactggaggactcctcaaaccatgagcagccaaggacagggcctggggtttaatgtctttgtgggcccagggtctgccatgtggtttcgtgctcagaggctcttggcatcatttgctgatttcaagggctttgattttctctaccctgatggggaggaatgcctgtggtgagtgccagttgtattttttcctgacacctctcatggttcatgaagttatatttagcttctaaaataaccacagcccagagcaaaggccacctgactgcagaccagactacacaattgatgtgcaggtagtcagagtaaaaagggacggcccttggggccacagaacctggcttcattcccagccctgtttccactaggcctctctgttctcatgggctacatgacgtaagtgacaggtagttattgagatgactaaatgagtgtcaggtactgaggacacagatgcaggaacttgaccccaagctgctctcaagcagctgtcaatctggtgtgggaggcagactcagaggcagccaggactgaggcgggagggcagaggggtgagcatgggctggggcggggtgggagacggccttgtggaagggctgcagggcctcttgcctcactctgccccttttgcccaagtccctggcaaagagaaggtgttcatcaaacgtccactgacaaaaaacaggggcatgaacaatgcagaaaacagaactcaaattaaatccacagctgagtggagtgcagtgttactatcttcagcagcttcaggatatgcaaaatctcatgcgcttttgaggggaggtgaaagatcaagacaaatgggtgtctgatcaacgtttttttttttttttcaattcaatcaacaaaggtttcccacgtggtgctccctctgaatgaaccactgtgccaggcgccggggctgctgaagtgaccaatccagcccctgccttcaagtgacatataatcaagggtaatacagacgaatagagcaattatcgcacactgaggctattagggttaaatctataggtatcctacttttcaaaacaaaatatagaaaaacaggctggccacagtggctcacgcctgtaatcccagcttgctgggaggctgaggcgggtgtatcacttgaggtcaggagttcaagagcagcctggccaatatggtaaaaccccgtctctactaaaaatacaaaaattagccgcttatggtggcatgtgcctgtaatcccagctactctggaggctgaggcacaagaatcgcctgaacctgggaggcaaagttggcagtgagctcagatcatgccactgaactccagcctgggagacagagtgagactccgtctcaaaaaaaaaaaaaaaaaaaaaaaagagagaaaaaaaaagtaatactcccaagaattattgtgccacttctgggtgtgaggagcagtcgtaaagacatgatttgggtactggaatctaagaaatgtggttattttaacaatttgcgagaactggatgagatattttacatggcagctgtagccaaggaccctgagtccacagctgtcagagtcatgcacactcattacaggggcttgttgggtccaaagggaaagcaggcaagaacccacttgtaagcctggtcaggggaaggtgagaagcagtgatgagtgggcgggacttgaaggataaacttcccacccctatgagctcacagcagtctctagctcctgcctcctcagcaaacaagtcaaacgtaaattaggaggatatgggaaggaaaagagaaagaagaaacagacaaatgtcaggtttcctacaaaggcaaaagcgtgcaggaccccatgtgagatgacaagcgattaaaggtattgttttaaagtggagtgagtataaactgagtgctgtggaaatattgccatgaatgtctgcactcgtggaggctggagggtgtctggggctttggtttggggatggagttgacaatgacggctgccctttgaggcagggatttctcaaggtgacctgagaggtgatttttataacccattaaccaataaagaaatgacatgttttcacagcatttctttgaacaattctcaatgggaaatttttctccacagtccacttaactgtgaagggccagagagtaagtatttcaggctttgtgggccatgggaaatctgttgcagctactcagctctgttgctgcagtgagaaagcagccatgggcaaaacgcaaaccagagggactgagttccaactcaactttgtggacaatgaaatttgaatttctataattttcacatatcataagatatgattcttcttttgattttttccaactatttaaaaatgtaaaaccagtccttagttcacaggctgtacaaaaacagtcagagggaagatttggcctgtggtttattgatactcactctcatcttcagagagagtcccaaaggactcatcactgtgacccctcacgtggccaaatgcagcgggcgccccgtctgtcccaagctccagggagaaaatatcaatatcatgcccacaaaaacagtctgagaaaaagtttatttatgtaaactttcaaatatatgtttataaaaacaaatatgtatgtgtgatacatgtgatatgtatgtatatgtgctcgcatatgtattcactcttggtgtggtctttgctgcttgcttccatatatcctttgttttttgggtaaagataatttattaaatgtgtaactaaatgaagtgatgatctaatgatggtatgatggtatgttgtatgtctttccctttaaatgagttcataaggcaggaaatatcctggcaggtcacacaccccatgtgcagtttggaatgtctggcatcttctcagacattctgaggtcacacaccttgcctgcctgtctccttcctcttcctcagtttccttccttcgaccaccatttcaagtgctgtgaaaattaggtacaaaggtacactaatctcaacagccttgtccttgtccttcaatgggaaacatggactatcctgcctcttggtgctctcttcattagtaaggccaggcctggaacactcctatggaggacaacaggtctttttcagagattctactatttcctccttctagaacataaactttcctaggaagcacatttttattggtcatctgtgtagcccaaggggtttagacaatgcttttgcatagtaagcactcatatttgtttaaaaattttttgagtgaccactaatgtgaacttctctgtttcttgttaggtgatttctcattccctaagagaagtataaaaataagaaagcctttgggaggccgaggcagatggatcacaaggtcaggagatcgagaccgtcctggctaacacggtgaaatcccatctctactaaaaatacaaaaaattagccgggtgtggtggtgggcggctgtagtcccagctactcgtgagcctgaggcagagaatggcgtgaacccaggagatggagcttgcagtgagctgagattgggccactgcactccagcctgggcgacagaacgagactccgtctcaaaaaatacataaataaataagaaagcaatattaatttactgatttcctatcccctcacttcctgcttaggatctaagctttactgaagagagatcatcgtattttttcattcattagatatcacccttattacttcacgtgaagcctctgcctggtttatttatgtatttatgaatgcaaggatatatgcattttaattattttctctgtcatcccacgtccccacacccattccactcttccacagacatccaattcactttatttcatttctgtccttcaaattatctttcatacattcattgtggtatatatatgtcctttaaaaacatgtatttcatggggtatgtgatttttaaaatttaccaagattgattcatgctttaattttcacttaatttcttagtcttttttctggttactactttttaaagatctagccatgtttctgtatgtacatctaactcatgatgtctgacaccaaaaagattggcatttgaagcacaaatgtcattttcttcatgcatttccctgaatgctggacatgggtgtcccctaactctgctatcacagacaacactgttgtaaaacccagacccacctttctttctggaactaggtgtgagttgtgctggcatatgcattgcagggtgacttctctgtgattggccaggcatctgttctgattggttgttgcatgggaaactaattattaagtgctttgaccatcaatctccctgcacttacctaaggtcaccaagacctgctgtttctgagagcagaaggagtgttcctgtttgtccacgtcttctcagtacttaataacattaagtttgactatctttgtcttttctgattgccagtgaaatatacgaattagccaattagattttgccttttgtcaatttcctactccttttttttttccttctttttctgatctctgaatctctcaggtgtttttgacacttacctcttgttttagacatttgcacataagttttgatattaggggccttgctgaaaatatcttccccctcccccaagatcttaacatattcatatatttattttctattagcttcataaatgtgtcttttaaatttgcatttttaatctatttgaactttatgtttgcttagttgggaagtagggatgcttattttttctctttcccaatatgatttactaagtaattcacctgtcagccctttactaatgatgatatttctatcatctatcatacaatttccaaaggtctatttttaatttctgtccttttttatttattattttgtcattatcacaataccttcgtttgtattatttagtatgcatttatggtaatggaccaagggaagacctttgtatgtagtaagttgaatctgcagttcattttcgttttcattttaaaatttgtcttagctatttgctgagctcccaaatgctattgtttttattggaaattaattgaacttatatattaattatcaaagatttgacaaagttttaatatttaatcaccctatccacgcacaatggtatgtacaaccatttagaaggacttcttcatgtcctgtaatagaatctaaatttttctcaatatttttgcagtctttggttaatccttaggtactttatgaattttgtttctgaatggcaaatttttgtttgtaaatggcagctaatattctactacacttttaattcaagattactgatgtagagggacactgtgagacttcgtctgctgatctcatatctggaaaatttatcgaacttacttgttctaacaacttgtctttttcctcatacgctttctatgaaaataatcccattatctgcaagtaaggatatcctaatacctcttatgtttgtacttgtatctttggggggtcaaaatctgcagtagcatgttgagcagtaacaggcatagaaaacatccttttcttgtgcagaccat

SEQ ID NO: 4 - DNA, 274720 bp, Homo sapiens
  misc_feature: intron 1
gcaggcctttcaaaattccgttcttctccatcatatgctttctctccctcatccaaatgactcctcctttctactctttaaggctcagctcaaatgtcacctcctctattaattcatttattcaataaaatatttacttatcattcattatatgccaagagttaggaacagacattccattgtcccctaggagctaattgttcatcctctcttgttcaccagtcaacattagaaaaatccttttgtgcattcccataggatttcttcttatctcctcatagaatccacccattccttgacatgcacttaccattctataccctgggcattcaatgatgaacagagcacatcactttgtgcttgcaccccgtgccactgtgttctcttgtaagccaatctggagaagcagagatgctacaattttcctcttgggatcacagtgaccaacccaaaggattggctgaagaaactacacatctcagatctatttccagttctgccatttactcactgtaggagcttggagaagttctttcctctcctcagatttcagtgcctacatctgtgaaatgaaggaattaaagcagatggtccccaaggcctgtcagggctattatcatgggttcagactttcagccaataaccctggtgggagggtctggaaaggctttcaaagataaatagacttgatttcagacctcagctttgccacttagcacctaagggattgtttcctcatctgtaaatagggatgaaaatagctcctaattcagggatgttgtgaaaataaatgctcacagactacacagtactgatatgtcttaactattgcaagagtgaattttagcttagatgggtgaggccaatgactgtgttccaccatgtctcatggattttttttttttttttgccattttatgagacctgggattcctttatttagaatgagcaaatcaactaaaaatttgtagaacccctagcaagatgatatctcagttccctcacacactgtcccttgccccattgattccagtctgagtaaccatctgtctgtttctgaagacctcctttttgtcagtattcccagattcatccaacctgctctacgcatgccatatttgggtgggtgatggttgtggtggaggcttctgcctaggatagagttctcagccttcaccaggccaagcagctctgttccatgcctatgtcctcctccacactaacactgtcatcagctaaaatatacaacagccccattaacactgcctccaacaccttcgctctatccctgttctcgtttgacacttgcatataaaagcaatgctagtgaagctctggcaagtcacattcctttctccctgcaggctagagacaagccattagctaggaacttccaggtccacatgatcctctggccacactaagtgggcaggcacagtgggaatctgagtcaagcaagaagagagaaacagtccatctccagatgttttctctttcttctccaggacaaaccatgtctctgatatatcctggatgctaaggtcaatctggtgacatttaatttttcatttgtggcgtgtattctatgtatcaggcatgctacggactgaagtatttgccttctcttgtggactgaggtctcatcactaacaaagcattttattaaacatccaaaagtttcatgccagggtaataggtactacaaagtgatgtgaggtttcactttaagtatcttcatgttgcaagtacctgccatgtttccctcataaacgggtaccaaaaaggaaaagattaaatatttcagagtccagctgtggcaagtacactctaaaatggctctcagtgatctttgccttctgctcatcacattcttaggtaattccctctgcttaaagttggctgggacctgtgctttgtttccagtcaatagactacagtaaagggtgtcaattctgtgattaggttacaaaaaatttcaacttctgtcttacaagtagactgtctctcttgctggctttgaagttgctgtgctagagaagcccgtgtagtatagaaccaaatgtggcctctggccaacaaccacaaaagaaagcttggaagagaatccttcctcagttgagcctttggatgagcccccagcactggccaacactaactgtagcgtttgtgagggaccctgaacagaagacctaggtaagctatgattggactcctgatccacagaaagtgggagataataaatgtgtgttgtgtttagctaagttttgtggcagtttgttatgcaggaatagataactaatactagttttccaggatctgaatcttagctcaccgcttagcaacgtgatgatatttggcaggtttatttttccaaagttcaatttcctatctataaaacaggagtaacggctgagcacagtggctcatgcctgtaatcccaacactttgggagctgaggcgggtggatcacgaggtcaggagttccagaagaccctggccaacacaatgaaacctcatctctactaaacatacaaaaattagctgggcatggtggcgggtgcctgcaatcccagctacttgggaggctgaggcaggagaatagcttgaacccgggaggtagaggttgcagtgagccgagatcatgccactgcactccagcctgagcaataaaactggacttcatctcaaaaaacaaaaaccaaaaaaacaggagtaacgaattaaggtatctctgttatcttaatattccttatttcagtcttcttagttaagagattgcacaggacagcaaaaacctttataaccaaaggcactaccctaatgaagtctccatgcaaacatccttgacaatatcacagcaacctataccctgggcatgtcatggctttgcaagtcagtctctgcctcaccatcagatacaccattcagtagtacaacatgaaccacttgcaaagcaaaaaggcaaagtttacaaaagatgcaggatttcacaaagttgatagaaatgatgatggaaagctgttttaaatacagcagggtcattgacaggtgacgatcttgcaagaaaaataataaaaaaaaaccaactgatactttaaaggattaaaaacttcaaccaatcctacccctaaatgttgggaaaataaaatgtaaagtcatattgtattatataatttggcattaagagcatatcccccaattaatacctgagtaagtcttgttcattctaaaaattagagcacagttgcaattataacctaactttttgttaagtgtgagatactataaacacatttttgtgttttaatagttttttaaagacaaagtactaatatgatatttttgtcttccctctttggtatgaagtattggttcttgttttcaataggctccccttaagtaggtttttattgattcctattaactgtggaataaaaggtttcttctaaatctacctcgtgggataatcaaatggacttaaagagtatcatttaaagtgtaaatgtgggtgaggtgcggtggctcacgcctgtaatcccagcactttgggaagcctgtgtgagtggatgacctgaggtcaggagttcgacaccagcctggccaacatgttgaaacactgtctctactaaaaatacaaaaattagccaggtgtggtggcaggcatctgtaatctcagctattcgggaggctgaggtaggagaatcacttgaacctgggaagctgaggttggagtgagccgagatcgtgccactgcactccagcctgggtgacagagcaagactcagtctccaaaaaaaaaaaaaaaaagtgtaaatatgtaaaagtgcctaaaaattctagtacatagtgggcacttatcaaatgctagttttttaccttgttccttcttattgtataatcatagaaaataccccaaaattgacttaatacttgtaggaacaaaaataaactgtctttaaaaacttccatattctggaaatggggaaagaaaatcatacctataaataacacagacaaatctatatctatttctagatccatatatatgtttatatctatatcatatccctatccatctataaatccacaaaggtataaaatagaggtaggttttgaccacctaacccaggtgttagcaaactatggcattctgctcatttttgtaaataattttttattatgccactgtatttgtttaattattgtctatggttgcttttgtgatggagcagaagacctgagtagttgcaatagagatggtctggcctataaagcttataatatttactaccttgagcattaagaacaagtttgccaacttctgatgtaaactatgagccaaaggcaatttaattcactcagcaccacctgaggacatgttacgtgtctaatattgtacagaaaaaaaatatttaaaacttaaatatatgggacctaatcttaaaaacacccctgtcgtgttagataatggagcatttaaaatgattagaggaataaaataggggaatatacacgttttatggaggtctctttaaagaaattaatataaattacgaataaaattagcttagggcctgcagtttaagcttctttagtactgtgataaatctgcctctggaaacatctatttaagtttcatgatatgttatcatgcgtttcttcctgactgccttatcttctccttagtggtttttaactaagtcaacggcttgtttgaattcactgttgctgccccaggtgcatgtttacagatagtgtgtcttaatcctttttcggttgctataacagaatatcacagactgagattattttataaacaacagaattttatctaagaagcccatgagtgtggtgtgagcatttggtgagagccttcttgtcatgtcataacatggcagaaggtaccacatagctagagagcaggtgagcaagagagtaagagggggccaaactcacatttataagaaatccactcttgcaataacaaacagacacccctaagataatggcattaacatatttatgaaggcagagccgccctgacctcatcacctcgtaaaggtcccacatttctcaacactgttgcattggggattacatttcccaaacaagaacttctggggacacattcacttcatattgtgccaccctttacttgccaaattcatgtccttttcacatgcaaattacattcattctatcccaatagccctcaaagtctttaactctttccactatcaactcaaaaatccaaagtctggagtctcatttaaatcagacatgggtgagtgagactcacagcacaattcatcctgaggcaaatacctctccagctgtgagtctgtggaatcaaactagttatctgtttccaaaatgcaatggtgggacaggcacaagatacgtactcccattccgaaaagaaaaaataggcaagaagaaaaaaaaataaaggcctcaaagaagtccaaaactcaacagcacagatattaaacattaagataagactccggaataatctttccctccatatgccgcctcctggacacactggagtagaggacagaccctccaggccgtggcccctatgtctttgctgcactcaatccatccagcagctctcccaggttagagcctcatgcctgctagagttgcatgtgggtggtcctatagttctggggtctcaggggctgcctactctcatgacttcactgggcactgccctagcgaggtctctctgtaactctgcacctgtgacaagtctctgcccgggcccccaggctatatgcaatatcctttgaaatctaggtggaggtccccatgcccccacagctcttgcattctttatgtctgcagaattagctccacgtttaagtttcatggtacgttatcatgtatttcttcattaatactgccaaggtccagggcttgcacattctggagcagtgggttgagccacaacttgttgacccaattgggtcatggctggggtggctgaggggtgctgtgctggactttggggagcagagacctaaggcagggaatagcaaatgctgaggcacctctctggaagcctctctggaaaagagaggcacccaggcacctctctggaaatcttgctctcaaagtcctagattttgaagatctgaaatgcctttcagttcattttcccatggttttgatgaatagaactaggcttccttctatctatattaatatctttatcaaactgtggcttgatcacacctttggtattctctcttgaacatgcttttttgttctttacatggccggactgggagcttcctacatctttcaattctacttctcttttaattataaattcctttataatttgactcatttcttttttctctcattttattgcaagaaggccaaaagaagccacgcagcaccttgagtgctttgctgcttagctatttcttctgccagatatcctagttcatggctcttaggttctgccttccctaaagtcctagggcatggacacagtttagccaagttctttgtcactttataacaaggactgcctttactctagttgccaataccttgttcctcattttcacataagacctcatccaaatggcttttaccacttgtattacaatctcggctcactgcaacctctgcctcccgggttcaagcaattctctggatacaggctttttctagcctgctcttccaaattcttgcagcctctgcccattacctagttgcaaagctgcttccacattttcaggtatttgctatagcaacagccccacctctcagttctaattttctgtctttgttttgttttgtcataatagaataccacggactgggcaatttattttaaaaaaaattgtttctcagagttctggaggctgggaagtccaataccaaggtgctggcctcctgtgagggtctttttgctggatcatcccatcccaaggtgagagggagagaaagagagcaagagagggccaaactcacttttataacaaacccgctctcaggagaacaaacccctcctgagataagagcattaatccattcctgagggctctgcctcttgaagtttccacttctcaacactgttgcatcggggagtaagtttccatcacatgcactttaggagacacatttaaaccatagcatgatacgaactgtgtgttactttaggctttgaggcaaacttccctatgacctgaccaagcatcatgaggagctcctgtcaaggaagaccctgggtccctgaactggacacatcagggtctcaaaccagtctcagcaaacgacttctggttcctcatctctaaagtgagaataaagtgtctacctcataagaggattcaataaaataatggatataaaatctttactggcaggcagtcgtgactcagtaaatagtactttctctctgttttgctgacaaaggcatgctcccttgcaaattatttatcatttgaaacataaattcaggagtcagacagggaatgagtttaaattttgactgcattatttattgctgtgtgactctgagtaagtaacttaacctctctggttctccaatcccttgtgtttattatggggataatcatttaaactcaaagggttgctgtgagaactaaatatgcctccatttctccatctgtaaaatagggataataaaatgtttagtctagtgcttgatagatttcagggattattattaatgattgttatttttattatcacataacatggtgcctggggaatttgtaaattcttaataactgaaagcaactgtcactattacaatacatttggatttcgtcaaacatatatctttggggacctctaccaagagacactatatttcattgtctttcagataccatcacttgtaacacacacaataattgtacaaactactaagaaaggaaaactgacaattaccttatgacagccattgattgtgtctcacatcctcattttagagacgtcaaagtgggaaaaatgtgcatcttagaactgatgaaacggaagtaggtaaaagttctttataatcaagtccttgaggacaaacccttttaaacaggaggtgactctgggcatgtctgagctgcgccggaggcagatcctgcatctccaacaccagcaatggcaggtgcatgaaggcagggtttatttggcctaagccttattctatgttttcatattcttccccttcccacaccacagctgtgcactatccctgcctagcctctggcacacctgatccactagcaaatggtgtccatttttcctttgaaataacatctcacatctggtctacctctatgactaaggatggaaaaggctcatctttctccagcctcattctggtcccccattctctgcctaactcctggccttggctgggcatcagggcaattgttctgctccctgtctcctctctctcctcctaacccaccatctcttcatcctccacccttcatacacacagagccacttggcatttgggcaacttgatttattagaaagtgaaggatcttctcaggagtcaggtagatctaggtttaacttctgaatcacatttctaaccatgtgcacttggtcacgttgcctcatctctggaaacttcagactttatttatgaaatgaggctaataaaatgacattgtagatatttacataccagagcgctgctattcaagtgcaaatgaaaagccagcatcagtaaaagttggtaaattcagaccctttctcatcatcctccatgcttgaaatgcagatcaccccctctctgttttcttccttaattttcaccatggtgataagattatgtcctcctacagagcatcttacatgcacagcacagcccttctctaatgtccatccaggaaagctgatcctggaagcttggacaatatacaaaaatgcctgagcttcatttccagttggctcactctcatcaggatcatttttattgatgtcttcctaatttcttcaagagcttgtcataggacacattgtatctgagagctggatggggttgtgtggtactcaccctccaaaaagataagaaggtaaaccttgaacaatcagaagagagaagcaccaagagagaagcttacttctcttcattgtaggaatctgcatgatcttctaaggtgggagaaacaaaagtgttactaaaaatccataaacctcaaaccaaagtccattcagctttgtaaacaaaacactatttgcttgcgctatggatggtatttgttgaggtctgaattcatccttccatggacacccactttgccatgaaaatgatgccaaatgtcaggaggatgtttaatttaacaaacacttacagagcacctactataggcatttatacagcactgtctgtgtacaaggcactaaactaaaagcagtgcagacttggggaaagagcgtgagcttcagaaccagatgctatctttgatcccagcctcttatcctcaagtttttgtgactcaggctaaggtatttaatttaataaggtaaatgatcttagccctagcctccttatttgtaagttggtgacagcagtgtccactatgtgagactatagtagaaactggtgagaaaggatgcaaagtccctggaagaccctcttgtaggtggagatattcaatagattggttcaatgacagtcttgctgtgactgcaggtattggggacagcaccctgcttctaggaagaccagggcaggaaatatctagaacagctgtattaatagatgtgcaaggcagagcatcacctctctctctagcagaagtccctcagctgctcttccctggacccagggagagaagactttggtatgtgaaggtggaaggtgagaaggtggtatgaggaatggagaacaggttgcatggctcccctcgacctctcacactttccagaagaagaggacatatctataacaggctcacagtagatacaggcattttctcgtcagtcctgccccatgtctttccagccataaggatgtctcagaatggcttggggcttctagtccagagcctacgatctagagcacctgctctgggtcatccctgtccttccaccctatatcacctgggtttccacagcctcccagccctctaattctgccctgccaggcttttcttcagcatttcccactgcaagtccaaactgttccaatttctgattggttcctggtcactttgcatgtccactgacagacaccttggtcctatgcatttctagtcctctgggtttaaaagaatcaattcacaaaagacaatttaaagcaaacaatggaaatcaaaataaaagaatctttatattgaactcagaacctaataaatcaactattttcatctgtcccctttccagaccaaggacacagtcatatacgaggtttacaaaattgcactgttacaatgtgatagtccacattttatattattaaagcatatctagaagtttgtccatttcttctagattttaaatttattggcatatagttgctcatagtaaccactaatgatcctttgaatttctgcagtatcagttgttatgtctctttttcatttctgattttatttatctgcatcttctctctctttttcctagtctggttacggtttttcattttgtttaacttttcaaaaaaccaactttttctttcatcgatcttttgtgttattttttcatttcagtttcatttatttctgctctgattcttattatttctttccttctactaattttgggcttggtttgctcttgcttttctagttctttaagatgcattcttagattgtttattattaaagcataaacagtcttccacttagttataaaatattcaaaacaatcatgtttaatgaaaaagcaacattctatgacatcagtgctctaaaatatgtatagccattgtcttactgctagatatttgggtttagattcttccaattgcatgttattataaataatactatgagaaataccattgtgcatacagattttctttaagaccaactctttataataagtctttggaatttctgggctttgaacatctgtatggatttttgctccattctgccagatggcttgactaaagagtgacatgattttacacgatctctagggtaaggtatgaggacctattttcatcccaatttcaccagcattggtgattactatttgctagctgtattgctgctttaacaggtataaaatggcacctggaatttgcattttaatagcgaataaagcattgtgccaaaggactaagccacttcttataagattgccagctgaggaagagaggttctaggaaaaacggaaactagaattctcagcagctccaaaattacctcaagtaggcatttaccttctccacctccacagcagccaaatcagactccagccttttcaaccacttccaggctgaaggcagtggggaaattaccaatgagtaattggcatattacttacatttcttaccatgccctggagacttttttcactcagatataaaaattggtcctctcagactcaaactgaaaggtgtaaacatctcagacttttttttttttatcccattagcacctttctacaaatggccccactgtttcttttgagagccaaacacatagacacaaagtcagatctgagtcctgtgatgtttcccaccagcatgacacaaacatggattattctagacttttttcttttccttttggaaatctcattagaatggaaacatttagtggcttcaagtttccaaaatgagagagattaaaaaaaaaaaaaaaaaaaaagaacgtgcatcacaccagcattagcatggcagagacagcttctgcttgctcagctgtgtttctagagacaatagccatgatggctgattgttgtagacatttccttccctccctgggctccatgttcatctctaaatgctcctggattttatatttactttcacagtcctttgttcaacacatgtttactgagcaattactctgtaccaaaccctgtgtcaaggtacagaggtgacccaagaatcagttctgcccttaagatatgcatggccttataaggaaatcaattacaccaattactttaatcagccttaaggtaggcatggcacatgctactctgctgggagaaagggcggtcaacagtatcctactaagctcttggtggtgaccagctattggctgcaattaaaaaaaaccgcacagcattttgtggctttgagacccctaagttaaggctagtcacttaacttaggggaaagaaatgcaaaaaggttattcactagctgctggtgttgtgagccacatccgcttcagttttgttggccctactattcccaactcctaggaccagcatcaaactttgtgaaatgaggcacaatattaatctaggagagccttgagaaagtaagaacaggctgcatatgttttgatatgtagaaaaagaggggtagtaagggaaaggaatatcagggtcagaaaatatgattttaagtttttgccaccaacttcctgggcgatactggactaggttgactctgagcctcaatttcctcccctgcaaaacgggtctaatcaatagacttctaatgggtctaatcaaaagacttcttagagcaattgtaagatgttgggtgggagaaacaagtgagctcatacacagaaaattctagtaccatacctggcacataggaggctttcagctcaggctcattccatacgtctccctcctaaaacacctcctggatctagagcaggtttgtaacttgtatttgtatcagggtctgggagggggaagcagggagcattttggcctctgggctattgcaaaggaggcagacaagctagtatggctcagcacaggccccaggggttcaattatctgggaatagtgatgaacaggaagcagacaggccccttctcacagactgtttatttgcatgggaagagagacaggtcacttgtgggtatttgggcttctagacacactgggagcaattctaacagggtccatcttctctgaggataatgggtggtcagacttcccattcactagcctctgcctgtctcctccatcagtaagtctccaccctccctgaaaggagccctcctcattctctaaacctgtgctcctcctctgtgcctttcctttatttctctctagcagacttgtcagagtacttggagaagatggttggtgccagcctaacacagactgaatggtgtcttgccctccttaggcaaaaagagctggatgtggaaaaaaatgtttgtgtttcaatggaaaattgtgcctgctctgggttatcaggttctaggcctgttcattgtcattaagccatttttttgtataatgtacgtaattcacaccacacacacacacaaacacacacacacacacacacacgtattgatggtcccctacttacaatagtttgactttgaatttttttactttataattatgtgaaagtgatatgcattcagtagaagccattcagtacagtattcaataacttgcataaaatattcaccattttattacaaagtaggctgtgtgttagatgattttgcccatctgtaggctgatggaagtgttctgagcatgtttaaggtaggctaggccaagctatgatgttcagtagttcaggtgtattaaatgcatcctcaacttacaatattttaaatttatgatggttttactgggacatcaccctgtcataattcagtgggcctctgtgtatgtttgtgcatgtgtgtatatacatgtgtgtgtgtaaattgtcttctatctgtataagagtcatgactgcatcttttctagaaaattttattttaacaaggatattcttccaggatgtaatgatgacttagacttcctgggcatcacaatgtcctggaaccccagtctgcaaagctagctgtgaattaaaatcacctgtgcaccttaagaaaccaaacaccaatgtctatctttaatccacacatcttctaatgtactagctctgggtggggcccatgctggtctggtttttaaacttcctcatgtgatagtcatgtgcagtcagggttaaaagtcactctccagaaggaagaaatcaccagctcacatcgtgggctctggagtctgactgtgtggatgtgagccatgtctcttgctggtcacctgtcaaagaaacacatgagcctgcagtgagtgcctgccatgggcaaggctgtgtccctgcagtcagaaagaatggggtttgaatcatggttctgatactttgagtaaggagcagtaggcaagtcacttgatagctatatacttgctattttcttgacatttttacactgtgtaacaaattaccccgaacatggcagttaaaacaacacacagttatcatctcacagtatctatgggtcaactgactgccacagcttatctaggtctcctatttaaggccacaaagtgttggccaggactgggttctcatcagaggttcgactggggaagaacctacttcgaagttcactcaggttgttggcagatttcattttatttcctgtgactgtagagatgtatggcagcttgcttcttcaaagccagaaaagcagagagaaaaactctagcaagatggtgctgtactttgatgttatatcaagagagaagatcatggaggccaccttaaagtctgtccaccacagtattctcatctggaaagttagtatctacatcatggggtggttgtaaaaattcaacaagacagtttattaaaatgggttggcacataatcactcaatggatgttattagtaaagtcctgcacagaagtcacttttgcataccttcccatagcaaagagcattgggtctccctctgggcccccacagcactctgcacaggcctgccttgtcttctatcacagtgtgtcttccatgtactccttgagcctactgggccctcactgaaatcttgccatgtggccagctttgatcaaagatttgatcaaaatgcaagtcatgcatcattttttgtaatcctcacagcaaactattgatatacaagtattgccatttccaagatggacaaaaatagactccaatacatgtgagaaaaaattgtgcttcagtagaaaactggacattggttagattacacccttccaggtgaactgactcaagttttgtattgtctttgagctattttacaactctgtaaattataaacaactgatagcaatgcaaaaataattcttgcctttagacatgtgaattctctcctgtctagtaaagtttcaattgacctttccccctcctcctgtgtagaatcagaattggctccatttgcctgtttagttcaatactctgatctataaatgtgtcactttttcagataaccttttgagtcctttataagatctgcctggttccctcactgactgatgagttaaagaaaatctttaatatgcattcattgtatatacatatgagagtcataagtctattttttttatttaaaaaatatcttttggacgggcatggtggctcatgcctgtaatcccagcactttgggaggccaaggcgggtggatcacgaggtcaggcatttgagaccagcctgaccaacatagtgacaccccatctccactaaaaatacaaaaaaattagccaggcatggtggcggacacctgtaatcccagcaacttgggaggctgaggcaggagaactgcttgaacctgagaggcaggggttgcaatgagctgagatcatgccactgcactccagcctgagtgacagtgctagactccatctcaaaaaaaaaaaaaaaaaaaacttttaacaaggttatgtatatttcccaagccacggctaataattggtagagctggccttcacctctcacttctcaaccctgaccccctgggcttagctacatcaggattcacattttttgctacttgctgtagtgctaaacacagagcaggtgcccactgatgcattatttgcatctttgctcagctgaccctgattatatttgactcatcacacacagaacataggctcacacactcacttttccaactcaagaatttagctaattcctgcaattcctatggcttcagacaaaaggccttcaccactggcttcagcaccaaaaacctctacaaatccttcccttaaaggagcgggcacctgtctttccataaattcccctcaactgtccttttctctcctctggaaacatatggaataccacctttcttccatgaaaagctcttcaagcattgggaggcagttctttaatttttctctattctttttcagaatcatgttccttaaacctggtcttctaggactgactttcaagcatctttaccatcccgatcattctcttcgggacatgtcccactctccctgtttattgaataagtgatctttaaatctactcaaaggctcatcaggtccatgtcaagcagaaggccttccccatctgtgtgttcagccaggttcatttaagaaggggaatcctggctgaagactcaggcaccccgccatgcttcagtcattcagctgccgactatgtgcttcctcccacagattacatcttccccgcctcactctgcttcacctccactgctgtcactatccccctagcctgtgactttggctttttcccatataggtattaattacttgatatgactccctccaggcctgagccatccctgggatgcccttctgcaaacacagacctgtaggcaggtgccgctgcacttcctgacactacccacggagcaggtgcacttcttccagtcacaccaggagcctacgtgtgcagcctctgtattcactagagttcaggacacctgcatgacactgttgacttttcaaagtgaacttatggaaaactaaaaccaagacttttttttttaaatattattttgaaaccatggatcatttatcaatggtgtctaatggacttcttcctggacctaagcacaggccttcacattgatcttcattcaatttcttcttcttagacccagcccataacgccagccactgcaattattttggaaactcagttctgccaccagagcttaactgtcccttccagctttgtcatctgacagtttgatagtcccaccagtggcaccaggggctccatttcactgaccaaggtgccgctctccttctgcaatttctaattgcttcccctttctttacttcccaaactgcctggggggaaaaaggattctggcttagacttcctactccctctgccccagagttggtctccttgaaggagttttcccagtttggcaggacaggctatttcatttttctgtgtccaagaatggctgctgacaacttcccagggtcaggacccaagacccaatccaatcccagttaccctcagctactgcagagtgcatgtcacccctctcatgctgagctgatggcagagctcacccttgttctgttcctccctggctcaaaggccacaggcttagaggatgaggagagagacagctttctgagtgggcccatctaaaggccagccatcactttagtaaggtattgcaagctctggtttgaaaaccatcgcagtcacagtaacaaagaccccaagtccacttatcaaggtccccttgacctcacggggctctaatgggaactgagcaaatcaatgcttctcaaggctaagttatgaaactgcatgagaaatttgtcatgagtgcagcatttcaaatgggatgtttttttctctctctctgtagaggtgaggacactaagttttctccttgaaatgaggacaagaaaaaaccacggaaaatacacagaatgactcagaacatacctagctaaggcattgtaggtacctatgttcatcttcactataaagataaataaagttctagagcaaggaggcagtaaaaagacctaagacttgggttaggagacctgggttctagtcctggctgtgcccccaaatcacctcggtgctgtaggacaagaaactttacctctctgagctacagttccctcctggtaactgaataatttccataggtcttttctagttctgattctgagatacagaccgcaatttatgcaatagatgtactcctgaaaacaagcaggtaaatgtttcctcctgcaactgaagatgattttaaatgaaccaagccaatatttctctctctctcctttctttccttctttccttcttctcaacttccttcccttctttccttccgttaggagcagaattctttattcaaaataaatcttaggtacaagtccaataactgtcgtataatagaaaaaaggttggtggcgttatggggtggggtggtgaggggcaaccctgcctgtttgttcacccttgacatcctctcctccctcccctcccctctcccctcccttctccatatgcacaaatacaattggtagcacttgcaaagcacagatttaggagaattcctttttcaaagcgtcttcatcttgatgaatacactacctgagaaagcaaaaatgacattgccacatggcaaaacatgtttatgtcattccttcattaactctcccctacaaatctgccaggctgggtaatcaaccgcatgccacaggtgaggagacttagacagattagaggtggaatgtatccattttgatcactgctgtgtttggcacctggggcattgcctgggattcagtaggtgctccaggaatactttttgaatgaatggtaggagttttagaccttcctcaatcaactcctgccagggctggggctccaaaccctctgacttctgcccatccacagatgccggactcctttctgggacatgtgatcacttccaaatgtcaccttgtaaatgtggattgtcagtcaggcttccatgaagtggagtgcacctgtcgttccccctggctggagaaggcagatggagccatgcactcgggcagcttgtagctgcagcaccatcacccacacctgagtctcaattcagccaatccatagctgtgggaccttaggccagtcacttaatctttctgaaacttaattccatcacctgaaaaatgggattaataacaatatttatctcatacaatgtttacatgggtgccctgagattatgtatgggaaagaaatatggagttaattctaagtgtatggaagagagaaaaatatttaaagtcaaaattaactatagctatttatgctgcagttaaccacagggacaacagcaacataatatgtaagggcagtgccataattcacagtagaagatgcatctctattctgttttcttctctccataaagacgattaaggaggaaaagagaaagaaatgtgttctaaaaccttgggcagttctgttttgccctgaaagactgtagcagatgctgtccatgctctgccccatttctctcctcaaggatcttttactgtttcccacgcttctgcctctgaggagcctgcacccactgctgtttgttgaaggaagtagaccatgagttactggaattgctttctctgtggtcaccagcagagggcagatgtgcctgccaggtgagggttgggcttagcccatgactgataggtgagggtgctggtgaaggcacggcttccctgccctgggtgggtaattcatgttccagagttcccctgtgggcagaggctcaagttcccttccatggggtgggactttgctaaagttgtcccttagactgtgcctctctccttctcttctctgctccctcttgctcaggacgcctcatcttatagtccagtttttgggaactcaacgtaagataaaggggagacttggacctgagagtctatgattatcagtaaatataaatgagtctgagaatatgcagaaaagtaaaaagagccacatttctctgcataataatctcctctcataatatatcaggagctgtattatgcagtacttggaagagccacagaataggggcctagagaaagaaaaaatggaaggctttacagagaataaagggccaaaccacatgggaatgtaaaacaacaatgatgacaacagcaacagcaactatgaccacaacaacagcaacaaaacacaactctgaagtagcccaaacctcagcgaaatgtgctacatgcgcaagtgttttgagcgcgtgatattctgagggacatcagagagaaagtgctgtgcaccagggctggatgccacaggcactaacaaggacataatgatgattcctttctgggataggaaggagtcacctgcagggtttcacagtcaggggacagcaggcaaacctgatccctccccagccctgtctttgggcagaatggatgggagggagggacgcagcctcccaccagtggcacaatgcgttggaaccaattccacttggatcccaaagacagagaacttcaattgacatctgggtttcgaaattttgattgcatagaccccagcctaggagacaagtacacaaatcattcttaacacacataggaaaagtgaaaaatgccacaaaaaaaggggtgcagagagttggaggggctgcacagagggatggtacacacactatcatttggttatttgcatgttaggaagcacttattagctcatagtcctctctgacaacacagagagggcagagtgggcacctgttagagtcatgttactcagagcttgcatatgttcaatgggcaagaagaagtgtgctggtgccaattcctaaggagtcctgtgaggccactcttaagagtctctggaattttgcaaggctggtaaacttattaaagaacaaattatatatatttacaataaaataaattatgttaaaaacaaaggcaataaatgctcaaaactcatcagcacctaatgatttttcatttcattattatctatgaccttaaaattatatcttttgcatctgtatcttggaattactatacaatggcgcacctctctgcccaactctatgttaagtgaggtcagattggcaggttgaaatttaccacggtgggagtatttataccagagaaaccggcaaacatgacaggtgattgttaaacattcctcagcacaccacgggagaagagtccagacagatggcaaaggaagaaaatcaaactacttcattcattcattcaacattttattgattacttactatgtgcaatttacatttagtcaccatttcttcatggctagccttcaaagctgattcatggaatctctacctcattgaggtaaaggcccatataaaacactgagagtcggccggacacggtggctcacgcctgtaatcccagcactttgggaggccgaggtgggcggatcatgaggtcaggagatggagaccatcctggctaacatgatgaaaccccgtctctactaaaaatataaaaaaattagccaggcgtggtggcgggtgcctggagtcccacctacttgggaggctgaggcaggagaatggtgtgaacctgggaggtggagcttgcagtgagccaagattgtgccactgcactccagcctgggcgacagagtgaggctctgtctcaaaaaaaaaaaaaaaaaaaacaaaacactgggagtctgaactattgagtatctttcaataagtgcattttgatgactttccagctcatgtgcagcagctccaactaatagctaacaaagagctgccaaggagaggtctggccaggcctacttcaataaatagataaggatcatttaaaagcagcccattaagtgtttgcatgcaaatggccctgccaggctcctcgaaaaacagtttgttctcctaaggaggttaaatattttcagtcttgtttatgttattaatttgcttagccaagtgtgcggttgtgggtgtatttttaatagaggatggaaaaaccttgcaaaatgagctttatccctgaaacataaatgaaggaggctttgtgaattctatacaaatgaccatggtccttgcctgtaccccacggagaaaaaaaagctctgctccattcatgtcaggtagccctggcataggtgcaacccacaggggaggacataaagtagaaaaataatacatcccacatgtgctgcacccctagcacctgggacactacctagcacaggtgtagcccacaggatggatggcgcataaaacagaaacgtaaatggacagagtcatgctacatcccaagcacccagaatggcatctggcacatgtgggcacacagtacattttggttgaatgaataaatgaatgagtgaatgctcctatgctataattctaaccacagttttgaccaaaaaggatcttctaataacaatagacagaaacaatcatagaataaaagaagggagagatggagatttataaagagaataaggctttttttatatcacatctcatttaaccctcacaaatacctggaaagttttattatccccattttacaggtgagaaaaactagaagtaggcagtccactcagcatttgcacagataggaagtgcagagggaggtcaaaatctggtatgtttggattcaaaatccaggctcataccttgtactatgtaatggtagcaaatattcctggaaatagtcaatttaaaaggagaaatcttttctctgcttccaaaaaagctatgataaatttcccttactgtggaagatagaaaattgaaaacaaaaaattcatagcagaaacacatggaggcccagtctcccttcctcacttcccttctctccttggccacagaggaatggagggatgactgtgcatgctttgttactgtttttcactattttctacaacattgacctttggctaggctcagggctaaactctactcatagcatctctgtcccacaaaactctctgtaaggtgaacactatgacctctgtggtgtggaagagaaaattcagaaaaggaaagtgatttggctaaggtcaataccggcaaatggagcatctcagaatctggtttatctgtttctaaagtgtttttccactgacagttttctggaactccagtgtgcactggaatccttggggaccttgatgagtgcagattttgattcagcaggtctcaggtggggcctggcaagctcctgggtgatgccaaggctgtaagtctcagaccacccttgatgagcaagtccctgcacacgctgccttattcatgcccagtgagctataagcaactcttaactcttctccttgtgtagagtcagttctcagttcttcccgctttgatgaaggtccagctaaccaactacactggtgacttggtaaacccaggtagaccctcactatccccactggaccctggctagctaccttcttctctacccgctctctgtccctgggacatcatactgcttaccctggagcccccaaacttccctgctgttctctctcagtctggcttaaacaataatacctaaaaatggtctttgcacctgcaattctgcctccttgaatctttttttttttcacaaaacaatcactttgaactatttaaatataaatgtaatgatgtcactctagttcttacactccttcaagaaaggacaagccactaatcctcttgtcctgcctcaaatcctctttaagcagagcttattttgtttgtttgtttttatctgctgtggatattgtggtgcaacacaagctttaaaagaaaatacaagattttgctactaaagagaagtaagaatccatctgatcaatatactccatgtcttccattttacagatgagaaaaatgagtcccagagaggtcatacctcattcctgaggtcccccagaaagataactgtggcgtgagcatgaatcccaaaaaccttgatgatatatttttttaattctgattctctttccatcgcaccatgcttctcataaacaacattctttctattttttcatctacgtcttccctaagttccattaggtgggtttatttattggcagagagcaaagtactcatgtctgcatgccccctccaaacctaccccctgcaattccaagaataaagaaaggcatgcagactgggtgagttcttacaagcagtctgcagcagactagactgactacttggccatgagatcctaagtgtgggggcagttacagataaataagaaaaactgaccacagccacctctgcccctcaaaactgaaggaatactctttaaggcatcatggccaacttaagctggttctccagacatgataaaatgaatgcagaagctgatcaaaattagtatattgagcctcgagctgatggactttttcccaaataattattcttttttaaaatatggccaggtgtggtagctcatgtctgtaatcccagcactttgggaggccgaggcaggtggattgcctgagatcaggagttcgaggccagcctggccaacatggtgaaatcctgtctctactaaaaatacaaaaattagctgggcatggtgatggatgcctgtaattccagctactcaggaggctgaggcaggagaattgtttgagtttgggaggcagaggttgcagtgagtcaagatggcatgactgctttccagcctgagtgacagcgtaagataaataaatatatacataaatacataaataaaaaattaaaaaataaaaataaaaatatcaatggcatgaaacttttttttattttttttattatactttaagttctagggtacatgtgcacaatgtgcaggtttgttatataggtatacatgtgccatgttgtgtttgctgcacccattaactcatcatttacattaggtatttcttctaatgtgatccctccccctgctctccaccccatgacaggccccggggtgtgatgctccccaccctgtgtccaagtgttctcattgttcaattcccacctatgagtgagaacatgcagtgtttggttttctgtccttgtgatagtttgctcagaatgatggtatacagctgcatccatgtccctgcaaaggacatgaactcatccttttttatggctgcatagtattccatcgtgtatatgtgccacattttcttaatccagtctaccattgatggacatataggttggttccaagtctttctattgtgaatagtgccgcaataaacatacatgtgcatgtgtctttatagtagcatgatttataatcctttgggtatatacccagtaatgggagcactgggtcaaatggtatttctagccctagatccttgaggaactgccacactgtcttccacaatggttgaactagtttacagtcccaccaacagtgtaaaagtgttcctatttcttcacatcctctccagcatctgttgtttcctgactttttaatgatcaccattctaactggtgtgagatggtatctcattgtggttttgatttgcatttctctgatggccagtgatggtgagcattttttcatgtgtctgttgcctgcataaatgtcttcttttgagaagtgtctgttcatatcctttgcccactttttggtggggttgtttgtttttttcttgtaaaattgtttaagttcttgtactggtaccaaaacagatatatagaccaatggaacagaacagaggcctcagaaataacaccacacatctacaaccatccgatctttgacaaacctgacaaaaacaagaaatggggaaaggatcccctatttaataaacagtgctgggaaaactggctagccatacgtagaaaactgaaactggatcccttccttacaccgtatacaaaaattaattcaagatggattaaagacttaaatgttagacctgaaaccataaaaaccctagaagaaaacctaggcaataccatttaggacataggcatgggcaaggacttcatgactaaaataacaaaagcaatggcaacaaaagccaaaattgacaaatgggatctaattaaactaaagagcttctgcacagcaaaagaaactaccatcacagtgaacaggcaacctacagaatgggagaaaattttgcaatctatccatctgacaaagggctaatatccaaaatctacaaagaactcatcttcattttatgatgaggtaaatccgtctctaaatcagaaaatcaactgaagaaaaagcttatctttatcaagttcatgtgagcaaacaatgatacttggtatctcacatccacccattgatctgaaaaccattcaagacagtggttagaaaacatattcacagcaggttaataaatattccttgagtgagtgcgtatatagtaagaagcttactgttctcagagaaaataatggatacttggcatataattccttgctaagttgaatggtttagttagattctgagaagagtgagtttagggtaggcttaaggcaagggaaggtattctaaggggactttgggttggtggaaaagataacattccatggagattctaagaggagccaattcagaggcagggagatgtgtctatttgttctgggtacactaaagaggacatcaggctggaagagaaagccaagaaccagatgtagaggagcttaaaatcagaactgaacattccagtatctagtgcagtcagtcacaggaagatgaaggacagtcagtttcatagcgtgggacacacacgtgggtgggatgcaagggcacatttagtagtgcttacatagttatgtgcttaatgtggatttgaagaaagcataacttcacacatcagatacatgacttcacagatagtattgcttacttaacgagaaaaatattaaatcaatgccagcttggatgtgacaaaatccaggggagcaagaatgattggaaattaacaaacaatttggctaaagctataatgtatccataatcattactggttgtcccagcaaaggctacagtgtgggtggaagtgcttttttattcattctttcattttcaaatgtgtaaattcatacattcaacaaaactaatgaagtgagataatacagagaataatgactaatttctcctcatgaaaggaaggaaggagggaagaaaggatggaagaaaggaagaaaggaaggatggaaaagataagggagggtgagagggaggatttttaaggacctaataagaatctaataatgtgctaggtctggggactcagacaggaatgaagcacagtcctcctcctcaaggggcttgctgtccagcagggaagcagacagctgagcaaatcactgtaatcccacgtgagaggaggcacaggtataaaaatgtgcacaaagtacaaaaacttgtgggagcctgtcagagcaggggtagcttgggagaggtgctggtatttgagtttagcacattttgccaggtggggctatcaatttgtattagttataattaagatttgtaaggagttacagttcatggatcctaaaatatggcactattgggaaagcatgagtgcaagctattactattatttagtgcaatgaatggcattaaactgcatgagatgatcagtaaaacttatttaaaaacatgcattttgagacactcccttacggggccatatagtatgcgtggagtatggttggattaatcttaaatttttataaactctgggaggttctgatgctgtagacaggtactcatagagagactaaattagagaggtcatggatcttattgactttggattccttagtagggggcacaatacctggcatatgggaagtattgaaataaatgtttggtaaacatgaacaaataaattattaaaaatccaataacaaatctaaccaccaggatcatatttttgagacagaatttcactcttctgcccaggctatagtgcagtggtgtgatcatagctcactgcaacctcaaactcatggcctcaagcaatcttcctgcctcagcctctcaagtagctgagactacagacacacaccatcatgccaggctattttttttttttttttttttttttttttgcttttggagagacagggtgttgctatgttgtcagggctgctcttgaactcctggcctcaagtgattctcccacctcagcctcccaaaatgttggaaattctagatgtgagcctctgcacctggtcaatattttgttttcaaataaatttaatttattgatttaaaatttatacttgcatccttccagaaagtgatggagttagcctgtaattaaaaatctaaacaggattaattgctttttaaaatttaattttattaattgattaactcaggctcattctgatgggtgcctcctggtggtggaagcactgagtgtctatatgtacttgaaagtaaggaactgtccttaagaacattcttccttgttactccctaattctcactggtcaccctactgattggtctccactggtgagatctgactcacaaacttctccatcagcaggaacataatgtactctcaagaaccaaacccaaggcatgctttcacattttgcagtactctctattcctgaaaagcgttaaaattagatttattatggctcattaagttaaagggaacaggacaattctctttctaagcagctgaaaaataagccaaattttctagtccatattcaactcaaataccttgattttgatacttttttttgagttctcagcaatgctctgtcctaactttccattctgctcattggaatctgttgtactagcatcagctacataatggttattcaactaattggaagaagaagggaaaatgggagagggggcagggaaagggtcgggtcttgttactgagtgtatactgtgttccaagcattatactaggcaccagtagaatgattaacagagcagacatagccccttccttccctgtgcttacaattgacagaatgatgctacattaatcaatcatttatagtaaagaatgagacagaatagacccaccagtttgtcccctgtttgcaaatcaggaaaaaatctggaagtgatttccagcaggacttgccccagtaggaaaaactgcactctgttggcagaataacagtaaagaatattgtgcccaaatgaaaacggcttttaaaaattagcttcataattctaatagggatgtgataagaccaatacaaaagtagcttgagttgagttcctgctcaaaggtagagggcactgagttcttagccagagttgaaagtattcaagggaaaatcaggtagtccacctggaaagatcactggaatcatttttagggagtgagaaggagattgtggctgggaggcagtatagagcttgaagttcttctaaaaagtcatagaaagcaactgggatcaaatgtgaattcagatacatcagaagacaggatgggtgagtacatgggatttggaaccaagaactgggacaatatgaaagatttttatcctacttatatatgctaccaagagggtgacagtgatgtgctgcagcctgctaataccatcttgcaaaagccaattatggacacctcttccaaatccaatttcagtcacttcacatgggcagcttgaaatgggttatcatgggagtatttacaccacagaaagtggcaaatgctatgaatcaggacttcgctttaagagaagttatacaatatttaccagcaggtcactacttacagagtccaggctaacagcctccaggacctgcatgcaaccagaaatttagaactggatctgtacccagttagcattggttgccttggaaataatgcatttctcctcaactgatatgaacaaggagattcaagtgtcagagaggtgcatagaacaaatgagcttgaagcctactccctactttgagattctgcattcttattctaaaaatatttatggaggattttggccaggcatggtggctcaagcctgtaatcccagcactttgggaggccaaggcgggtggatcacaaggtcaggagatagagaccatcctggctaacgtggcgaaaccccgtctctactaaaaatacaaaaaattagccggacatggtggcaggcacctgtagtctcagctacttgagaggctgaggcagcagaatggcgtgaacctgggaggcagagcttgcagtgagctgagattgcaccagtgcactccagcctgggtgagagagcatgattctgtctcaaaaaaaaaaaaaaatcatggaggattttaagatcatctcatggtgataacatacacatgagaattaaataccacactgtataatgtttctatcccctgctgatggaaaaaggtgtatatgtgtgtgcatgggtgtgtgtaagtgtgggcgcgcatgcgtgtgtaggggagtggaggcaggtaaagggattcaaggttttgtggtatatctaaaataaactaagaaattgacaagtagccaaggtccaaattataataatttccaagaaatacagagccagaatgacataatattacacaccaggaatggtgttcttaaaaataaacctgagttgtagctattaaggaccatgctattttgcagaagtatgaagaaaggcaggttgaggctagcatgtggtcaccccgggaagacagcagggtatcaagtgtcagtgagctcctggagctgaagtctgactgagcagaagaaaatgggtcttcttgtggatcctgccaaaacgaaatcaatgaacatggcctggctggggaggatatcactgtgtccacattttggcaatactctctgagggataaaaacaagaaatgacactcatataaaggtttagaaatatcacatccattatcctgttgaatcatcccagcatttttggagaaaaatatcattgacttcatttttcagaaggcaaaactggatctcagagagcttaagtcacttgtcatagttacagggaaggaagtggcacacacaagacaatatataattaagtacttaaaacatatctcactcttttttttttttttttttttagatggagtctcgctgtgttgccaggctggagtgctgtggcgcgatctcggggctcactgcaacctctgactccctggttcaactgattctcctggctcagcctcccagtagctgggactacaggcacgtgccccaacacccagctaatttttgtacttttagtagagacggggtttcaccatgttggccaggatggtctcgatctcctgatctcatgatcttcccgcctcggcctcccaaagtgttgagattacaggcgtgagccaccacacccggccccactggtgtctttataagaggaaatctggagacacaaagaggcactggggtgtgtgtgcagagaggaaagggcatgaagacacagtgagaagccggatgtctgcaagtctaaaaggaaggcctctgggaacccaacaccttgctgacacactgatcgtggacttccagcccccagaattgtggaaaaataagtttctgcagtttaagccactcagtttgtgttattttgttatgacagccctagcaaactaatacagagggcctctttggccctttgtggaaacctgattgaaggtgactacactggcggtgccgggggcaaatgacactctcccggggctccaaggaaaaatccatagtccatctcagttcctcatttgttctgttcttgcacttccagtggtcacaccagttatacgagttcaatatgaatgcagagaaggaaccaaatggcaattagaaccacgatcaatgggaagaggtagtctttcacacagagaatctattgtgtgctagaatgtcttcacagaacctgagttctgtgaagttggtgctgttataccaattttttcagataatgctcaggctcaaagaagtgaagaaacacactcaagttcaaacagaagttaaaaaaaaaaacccaataaatccaacatgattgtgtttagttccctcaacagtaaataacttgcttttgatgaaaagtcctttgctcatgcaaaatgatgttaataaaaccaaagccgctgcaattagtcagatggtgctcatattaatttgtcatagatgtaagacccaaacagttttgcctctgggtaaactttcttgatttacagaaataagtccatatgatttctcaaatagtgggccctttttttcaagaatccaaaatattcaggcatcaaagtgacaaatgaataattgtattaagcagtatggacagaaaggcactgtattacattgaataactcactgattcatccatccatctatccattcactgagttattgagagagagagagagagagagtgtgtcccttctatgtgtctggtttagaattaggcactggcaatacctaattgcgaaagaaacaatccacagtccctgcttccaaggagctcaaaatttggtacctacttcagagaattcttgagagttttagattaattactacacggaaatacttagaaaaatgcccagaacatagtgaacactcaaaaaatacagttttaatttttattgctatctgatatataatcagacaacaaaacatgttttcagtcccccgtagtaaaggttcagtatgtttttgggggagggtgctgggaagaagttctatctcagtctgggggtgcctgaaactcttcctccagaagtagcccctcagctgactcttgaaaaaaaaaaaaaaagaggcaagcgaagaaaaggagaaaggagttctatgagatagagccaggaagatgaaggccagagcttaaagagcttcatgcattgtgttagtgagttgagcatttatcttgcagataagagggaaccattgaaaggtttgacacagggagagagggaacaatcaggatcacatttgtgtatcataaaaatctttctggaggctgggcatggtggctcacacctgtaatcccagcactttgggaggccaaggcagatggatcacgaggtcaagagattgataccatcctggccaacatggtgaaaccccgtctctactaaaaatacagaaattagctgggcgtggtggcacatgcttgtagtcccagctacttgggaggctgaggcaggagaatcacttgaacccgggaggaagaggttgcagtgagctgagatggtgagatcatgccactgcactccagcctggtgacagagtgagactccgtctcaaaaaaaaaaaaaaaaattctggatatgaggtaaagaaagccatgaaaggtggtaccagtgcagtttttcccaggagaatgttgatgactcaaccaagccagtggcaatggacagcaagaggctgtgggcaactcaaaatatactgaggacttcaagtcaacttgacttcacaactccagtaaggagaaggaagagtcggaggatggcatggctagctggtgcccttgtctgagttttggtacatgagcaggttcggaagaaagttgatgaaattcattttgaaggtgtgttcaaggtgtctgtggggaggcttctaggtggtgatgtctaatcaacaaacacaggttggaactcctaaaagactattacctggggctagaaatttggagtcataagccaaaagctgggcaattaaaccacaggagccaagcaggggaccagagacaagcagagggcggtgagtgggacaccccagggccttccaagaggcatgttcttgtcaataaatacactaagcaagggagcaactacagcatccccaccaagggtgaaacataacaggtggagagaagccctaggctgaatgcattaggattctaatgacatgccacaaactcctatcaggagacgtggtggaagggagctggcaaatatatcaactcacataattggcatttttgcagcacacttctggtgcagaagagctgctggctttcagaaggaagggcaggatggggtctgatcactcactttttcaagtctactcttaggatcttctgtctgggaagccagctttgtcaataactgtttttctctgcctcagggccatgttggatctacctcaacagttttcagatgacagcaccaaacaaccctcccagcttctcccaaaggctgcctccaatttggatgtaatcagaatccaaactggctcctttccatgtctcctagtaggaagtgaccctggaagctgcaaagtcctcatttgcaaactgacagtcactttttcagtttcctaggccaagatgaaaaaggctccttaccttccaagataaagcccccccgcccactagagcaaagagaaatcacacccacacccagctgacattcattttccattttcccagcctgtcactgctgagaaagagacagctgctgctgccaccacaggcagagagagagagagacagagagagagagagagagagagagagagagagaaaatcctttaacctcttaccatattaagtcacagaaattttcttgcaatgtgtcttcctgtcatttactcaggaaggtccctatgacatagttttgaaagattttacagacagcagaaaaacaagagtgttatcccaaacctcaatcaactaagcaatgtaaagacataattcctgaaattcttgcccacaatagtttttggaaagaaagcactacatttcactggttgtttccaaactgattttgtgtgtgtgtataacactttgctgtggccatctccagaacattaggttgtctcccatagtgtcaatcccttctgtccaaagaattacatccacttcttacccccttacttagtgtaataagctcaactgtgtcctcctcaaaaaaatttatgttgaagtcctaatccacagtacctcagaatgtgaggttgtttggaaacagggtcttaccagaagtaattgagttaaaatgaggttatgatgggcccaaatctgatatgactggtgtccttataaaaagggaacatttggacacagagacagacatgcacagagggaagattatatgaagacactcagagaagatggccatgcgaccagaatgatgcattcacaagccaagcaatgccgaggattgccagaaaacagcagaagctgggaagggccaaggaaggattctctcctagcacagtcagagaaagcatgaccctgtcaataccttgatttcagacttctggcttccagggctgtgatataataactgtctgctgttttaagccactcagtttgtggtaagttgttatagcagccctgagaaaagaatacactcatataggtgggtttcttcacagatgcctttctagcttttcctttgatttgtgatgttttctctgttaaggactgaattgtatttgccccaaaatttatatattgaagcctgaatcctcaatgtgactggatttaaatatagggatttggggggcagttattagggttaaataaagacacaaaggtgatcctctaatctgataagattagtattcttataaaaagacaaagaaacaccagagattgctttttctctacacacagaagaaagggtatatgagaacatagcaagaaggtggccatttacaaaccaggaagagagtccatcagaaatcaaatgtgtattttgatttccagactatgagaaaataaatttctgttgtttaagccacccagcctatgatattttgttatggtagccaaaactaactgatacacatgccaatcctctgccatgttctaagccacattcatgctttgcatgatggctctaagtcacaatgatcattaggtctttcctcgaatccttggcaagagtgacttcagaccctaatagttgtacgacagttctaattttgagtattcttagaaatgtgcattgattgagcccccacaacattccaggcactgtgcttaacacttcagatattctgttccttatacctccctactggaatagtgactaccatcccagtaatttccaggagagttaacggagcatcagaaactaagtaattggtcacccaccaaggaaatggagacacctggatttaagtcctagacaaatgactcctctgaatatgctcattcccctatatcagaggagcatctactcttgctcttgtccaagtttatagtcttggttctgcaaatatgatcaaggcacttcaagcattactgtagcaccagagcttcttaatctttaatgggcacatagatcaccaaggggtcttgttaaatggcagactccaattaagtaggtctagagggaggtgcctgataagctcccaatggtgctgatgctgtaggttgttggaccattccttgagtggtgaggatttacacacaggactcaatcgttgataattttatgctttcttttctaattgtcatgtggatgaattctttatcacacacattcagaatctactatgctcctagctctgtgctaagtaatgtggacccaaagaataattaagaacttaattttaaattccttaaaagcattggctgttattttgactattttttatttgaacaactaacacagtgggcacatagtaagcacttggtcaacatggctgtacatccttaacatctagaacagagcattcaaataacaaaatcagattgagatgtgtcctgtgacagatattcttagtcaaccactatgactcattcacccttttctcctttactaatgaagaccggtttcattaaaaaacagccccaaatactcacgccccagcttcccttgtggattggggtgttcgtgtgactttgttttggccaatgaaatgtgctaggaagtcttttggtttttcctcataaaaaggtctccatatctttcttcttctttctcgtatacagatggattaatgcacacagatgccgtgtttttatgatgatgaggcaactaaggtgagaggaaagctaagataatcatttagatattgtctctggcattgctaagctgctgaacaaatatcagcagctgcctgcttcaaatatcttgccacgtgagaaaaataaaattctttcctttagccaatcttattggtgacctatacatgcagctcaaagtgattttaaccaatagtaacccttcctaattaggacatttccgtcatgacaccagctgaggtaagagtagtctagtcaaaagttggaagcaaaactgttaggcatgaaaaacatgttttcaaaattaattccagccaaacaccaaccagaagcatattccatgacttgatccagattctgttagatgcaaatcttcttttctgtttttattttcccttgctatctaatccaaattctataggtccattccataaaattatttcaactcaagtgctggtatgaaaaatgctaaatcccaagttgacctaatagttgccaccttaaccttgatattccaccagggtgtgtgggaagcctagtgcttttaggatgcattttgggttatctttgagctgcagccttaacattagcattgactcatggctaatatatcaccaggacagcctactctaaaagcatcaatggaagtttggtttcctccttagactttcaatgccagtaagaagaaaaagcagagacctacatcctgctttcttccattcatctcacacacaaatacatcaattttcttcactgcttaagacagacatcagcctaaacgggtccctcatttcactttgtttcagttagaggacatagaaaaaagtttgttttcattaatattttacagaaaccttatataatggaattaatcacatcttaatgttaactttaaattctgaaggtggagaagcgggcaaaaaaaaatattcaaagctctttggatatttcagctgaagcaataaaggaaaaggtctaatttgacctgtggggaaaatgacaaagtaaataaatcaagtgaacacataatttattgtccaaaccagaccactttgaacagtgaaaggagaagctattaataattatgctggggcagtaggcgtaaactagaacataccaggacaaaccgggacatgtggccatcctgtaaataaagggttatgtctgtagtttttttgcaatcgagcctacatagctcaacagtgcacagaagaaatgactctgctctgtatttacagataggaaatcacacaaacgcccctctgcctttgcacagccccttgtgttttttatggagcctcatatcaggtctcacttagtctttgctctgtaaagtgggtgttcagatcctcttttggtaagaaagagcaaaaactgtgcataattctgtccatgaaaagtttttttttttttttttttttttttttggcaggtgaaaggccaagcagttcttccaaaggattctttccaaatttgggaaattggctcagggaaagcctgcctgcacagagttctggaaagccagtcctatgatggggctaccttctgcgggtgggtggaggcagggggcagagaggaaagaagattcaacctgaaaaatctcaaaacagaagaaaaacaaagtttatgaaacacacactatatactggacactgtggtagacacctatgcattctatcttacttgattttcacaacaaacacacgaggtaagtattcgcagtcccatttttcagattgagaaactaaggctcagaccaattaagtgatttgactctggttaggcagctagtaagtggtagacagaaagggaagttgtccagccccatagcttgtgctcttgtcaccacatcaggttgcctgacttggataactgggagtttctggactatgcactcctctccatcaccacggctgccctggcccagcccccatccaatattccctgggctgctgcagggtcttcattgccttgcctcatacagtccagaatgtaaatcacattccgtggtgatggttgcccaaccttatgaatatattcaaaatcattgatttatatacattaaaatggtgaattttgtggtatgtgaattatatgtaaaaaataaaaagatgcttggcagccaacacaataacaaatcataaaagtttttaaaatgtgagtcagatcccaccattctccttaaccctctctggtgtcttcacatcacagtgcatctggaaaaacacatagccttctgggtgtcctaggaagccctaagtgatttaagaaacttcctggccgggcgcagtggctcacgcttgtaatcccaggactttgggaggccaagggtggggggggaatcacctgaggtcaggagttcgagaccagcctgaccaacatggtgaaaccccgtctctactaaaaatacgaaaattagctgggcgtggtggcaggtgcctgtaaccccaactacttaggaggttgaggcaagagaattgcttgaacctgggagggaggttgcagtgagctgagatcacaccactgcactctagcctgggcgacagagtgagacgccatctcaaaaaaaagaaacttcctgtccagccactagctgaccaccaagctaactggagagagacttacgtgaccacacatgaaaaacaatacagacattacagaattaatccaggaaattcactaaagccagttacctgcctccttgactagccttctctccttcactcagtcggtgccagtcattccctgaacacaccatataacctcccaggtcggaacgtctgcctttactccactttgtgtctcaaatactctgctcacttctggagggctggctcctttgcatcatttagatctcagccaaaatatcattttcttagaaaggccttccctgaccaattagaataagttagcacctttctaagcggctttcagtaacatcaccaagtccatttcttgctagcacttaaccctgtctgaaatcatctgacacatgacttggcatgtgctcttggcgggagtgggctccatgctgggcttcttcacagctcagtctctaagacccagcacatgccctggcaagaaacagttgataaatgggcttcttttctggataaatccatcataaaaacttgcttctcctccacacagtcacctataccagcatggcagtgggactgggtaaaactagaaagagtggagagctatctattctactcttgttaactccaccactactggaaagagttttctgagcccagttatgggatccaaattgcaaagggcaaacctttttcatttggaaaatatttaaatcttccagggagtgaacattttaaaaacgctttcctaggaacaattgttttccaacaattgcagcaaacatgacctccatcaggttattctatccaaacacacgaagcaaacaacaaatctttgaagctcgcagggattcaaatgccttcttttgtaagttaaaacaaaaggcaaaggaggtagtgttgacaatgaaaagacagaggcctaattgagaatgactgagattaatgctaataattaaaacaataaaatatatatggacactgtcaatgaatgtctgccatgtgccaagtctcatgctcggaagttcacctaagttatctctttataaacccatggtggcaggcagaattctaaggtggaccccaagattctctgtccctgcataattccaagcagtgtggttatgatggatttcactccagtgactgtgtttccttagatggcacagttgactttgagtaagaagattaactgggtgggtctgactttatcacaagagccctttaaatctgagtgtagtggtcagagacagaggagtaagaaatttaaaataaaagatttgacacaagtgaagctgtccattgctagctctgaagatgaaggggaccatgggtcagggcacatgggtggcctctaacagctgagaatggcccctagctgacagccagcaaggaaacagagacctcagtcctacaaccacaaagaactgaattctgccaacaacctgaatgaactcggaagaggatgccgagattgaggtgagaatgcagcctagctaacaccttgattgcagacttgtaggaccctgcacagagaatctagcaatgttatccccagatttttgacctatagagcatgagctaacaaaagggtcttttttaagccactaagtttatggtaattagttattgcaatggaaaactaattcaaccacaataaccttacaaagtaaattcagtctgatgacacatggagactggataagagtaatgatgatgatgatggtagctatgatgatgatgataatcatgatgatgatgatgatggtacaattgacatctactctgtgttccaggcattgttttcagtgctgtgcatataatatgtcagttgattctcataccaacatttacaagacaggaactagtattttcatctttatttgcagctgaagatatttaggaacagacaccttaattatgtcattggcccaaggttacagcatagccaagattctaactcagaatccatctggccctagttttttgcacttagccagtgcacagcaacccacttggtgctggcccaggcttggcaacaggcaagggcttcacagaaaaggaagaaactcaggctggcagcacccacgagggcagtccaggatattgcagcagtgtaaacagaggccaggaagcttaaggctggatgaaaggtgtgggaaattgaaagctgggtggtttgttggaatttgcgctttaatagtatttcttgattgcaaattacagaaactcaaagtaaaatcacctaaaatataaaagaaatttgtcattaagataaaaacaagaacaaaacaaaactagagcatcccatggtattagtgagttgagtctaaggtattaatagaaccaggtttaaatgtaaagagcaggagcaccatcatctcggacaaatactaccactttaagatccagctccctttctaacctcatgcatttcaaggaaatcacttctcttctaacaacaagcagccagaaagagaacacagtaaagcacagataagacagctctggcacagaaagaggggagaagtctcttgggtaaccaccaaacttcacacttgtacaatgggccccagtaaaacagggggtcccaataagcacattcctttccctttcggtgcactaaattagggaagctaagaacagactcggcggagggggtatgcttgcagctgcaggaagatgtatgggaacagacacaaaaactctccctcctagataagcaagacaaagagacaaagactaaaagttggcctatgtggtcttgaaatggggtgagagctgataaaaactctgctctatacagatagcacacctggtcctaactgaaccattgggccctagaagggtaaggcatcctctcctcatgagcccctgaaccctgaagtacccttagtctgtaagagagcaggctcctgacctaactgcccagaagcctctctcaggtttattctttaaaataaacctgtctttgactgtagatccgctttttgtgtttctttcttccttcttcaactcttatgcaaacactatctctctcctttttggtccttcctgtgcaacattctcattcttctctcatttctatttttctctctccctatcctacttctctccttcttgtcgcctggcttctttgggtaacattattgccaataacttcccaaatatgtcatactctaagcatgatgaatgatggggtctctgaggctcacgagcaaattctcagtgaaagagtctgttttcccagcttgggttaggggtccatccccagcccaatgctgtgtacatgaagtcatatggtcctaacctagacactcagactgcaaccatatgaatgagggagagaagaaaaaagttaatagaaggataatgagaaggctaccctgtaagtgtaaacaaccgtgacatggagcaagagaaggctctggaggtaaagacagaggcaaagtcatgaaatactttatgggctggcttacttaataactcattaattgatggacccaaagtcttggcagaatcttatttggacattgacgtattaccaagaaattcctgggccccttaattcatacaatttgtaggatcagacactcttctgtaattaagcccaactctgaaatgaagctaggctttggaaatgctggaggaaccaaagctgcaaattccaacacatgccctactccagccctccctcctgtttaatttgattcgatgggctgatagctcctgcaaatacatcctgctcctctttggcaaattgctaaatccaagaagatgtaatcaaccacattctcacacatgaataaataaaagctgagtgagagattgaggaaatttaacacctctgtaagtaccactaaggttactgcttggagaattaaagtgcatttcatgacagccactgatttaggaaggtggtgatgtagaagaagacaaaatgaaactcaaagtaatttgagaagggaggagaccagaagaagattgttagtactccaggaagccttgtatgttaacgttgaaaccaacagactgcaaaacctggaagatcacgcccatgctgcaatttgccaatgctatgtgtaacccttgcaatgatttccagtgaaatgtgacatttaagacacacttgcatgatggaaccagaccccatatatgaccaattttagatagattgtagaaaatgctcagttaaagatagcagagcaattcctggatttgattcttagctgtatcccacgatagttgagtgatcgtgcacaagtcatttcagctctctgagacccagtttctatgttcggagaaaaattcccaagtagttgtgaatatttaaaggtccgatgacaacctttgacacaaagtagataatcaaatgtatgtcagctttgctatcttccatttcactttcaatcacggcctttgaggcactcagacagtatttaaatattgattctgctacctattttattaccctggaaatgctctaggtctaaaggaaactttgcagcttgcgtctaatgcatcatttccagatgggttgggaaacactgcaggaacgctatcttcactaccaactcctcatgccactaaattttgacacacagcttgggaggatggcatatccataggcctgtgccatgattctttttttttttttttttttttgagacggagttttgctcttgttgcccaggttggagtgcaatggcacgatctcagctcactgcaacctctgcctccagggttcaagtgcatctcctgcctcagcctcccgagtagctgggattacaggcatgcgccaccatgcccagctaactttgtatttttagtagagactggatttctccatgttggtcagggtggtctggaactcctgacctcaggtgatccacctgcctcagtatcccaaagtgctcggattacaggcatgagccaccatgcccggcccatgccatgattcttaattccagctacctctatcctctgccatgctcacctcccctgtgtagttatcctagaaaataaagcagaatttactcctggaaggcaaccaacacctatctggatgccagagacatcacaagaagccaacggggctttgataagtcagagtgagcctcgcaccacctccccatgagggcagagatatttgtcacttttgttcactgttgtatcccaagcacctaactttgtgcctggcacctagttggctctcagtaaatatttattgaacaaatgaaagaataagtgaactagtcaatgagagtctattatgcacctggcattgtattagggattttatgtatgttaactcctttaattctcaaaacaacccaatgagctagctacaatttttttttttctttttgagacagagtcttgttctctcacccaggctggagtgcagtggctcgaacttggctcactgcagcctctgcctcccgggttccagtgattctcctgcctcagcctcctgggtagctgggattacaggcacacgccaccatgcctggctaatttttgtatttttagtagagatggggtttcaccatgttgaccaggctggtctcaaactcctgaccccaggtgatctgcctgcctcagccttccaaagtgctgagattacaggtgtgagccaccgtgcccagcctagctagctagctacaattcttataaccattttacagatgagaaaatagccttagaaatgttaaataagttgcccaaggtcacatagctggaaaattgtcagagctccttccattgttttcatgtttttgagtacaatgtaaaattcctgataccttataaaggtgttatgcaatggaattatttgaaataattttgctatagttattattatttttatttactttgagacggaatttcgctcttgttgcccaggctggagtgcaatggtgcggtatcggctcactgcaacctctgtctcccaggttctcctacagttattattatctattgcctactttcgtccttagaaaaacactcattgggctgggcacagtagctcacacctgttatcccagaactttgggaggccgaggcgggcagatcatctgaggtcaggagttcaacaccagcctagttaacatggtgaaaccccgtctccactaaacacacaaaaattagccaggtgtgctggcgggcacttgtaatcctagctactcaggaggctgaggcaggaaaatcacttgaactggggaggcagaggttgcagtgagctgagatggtgccactgcactccagcctgggcgacaagagcgagactccgtctaaattaaagaacaaaacaaaacaaaacaaaaaaacactgagtggtagaagggtaatggattattcttattttttgcagaaatgaaactgcaagtggaattggactaacttactgaaaactgtatcaagcattataccgtcaagggcatctaggtagatcaaggatattttaatttgcttaggaacttattcctctgcacaaccacttaacagcaacgaaatttcttctcaggcaggaagtaaatttagctcattcttttgggttgcattagtgggaaagtaatatctatttgagtcatgtcgaatggcaagtacctcatatgtgggtaagtgagagttgctgacctgtgaggtggttaatgcatctgcttgctggtatctaatagctttttactattcaatcaacagacacatattgaacacctactgtccataggacactgtgcagggaattatggaggtggggtgggtagaaggcaaccctgtgtaggaattggagggatgctcatccagggaccacttgtgatgtgctggcaaccttcataccttgccttatctgatcattaaaacggcttcaaaagattgacaatatttatgcttagaggtgtcaatgcacctgcccagggtcacacaactggtaagcagcagagttgcaatttgaacctagagctctctgattccagagcttaagttcaacatctgtactacaccacttctatcatttagttttacaataatctatatcctctttctggactatgtaagtgataggaacctaaattcatagatacactaaaaatataaattccattactatttcattcactgctgtatctccagtacctaggacaatgccaggcacatagtaggtaccaaatgaatatttatcgaatgaatgaatgaataattgaattattttttaatctgaatccccagggcttatcatgatacctgacattacatttctgtgaaacagagcaatgtttatgttcatttataggcactttgcctaaatcctgtggagtagacttcattttcctagttttatagataagcacataaatctctgagagaataaggaggtagctctgggtttaatgaattttagacagagaggctacctctgttcacttccgttctaccatgatattttacccaaagaaacagtggcaatcttcaaatatttttgaggaggaataagacaataaaatccccattgcagaaggaaccctagcagcagtgggcaaaagccttcaggatgggaagacagtagaaaggagaaagagaactatctgattaatatcccagctcaattctggaaactgactatgagatttagctctaataatgttgagcccattctacagatgagcaactggaggcttacaaaggttcaataatttgcccaaacttacaaaactaggaagtggcagagtcaggatttggattcccacatgtgagatctgatgagtctctagtctcactactgacactatcaaaaagggaaagaaaatgtgaggtggaattatgcccaatgcttcctggaggaggtgacaggtgactctcgtttggaaagcagagaaggtgacaaggagggaattctagacagagaaactgccaaggtttgtggtcaaaaaaggaagcatatgtagggcccagaggggcttcagtctgatgggagcagagtaaaaaatagaaaattaaaagttaatccttttcttatccctgtatgaagattatattggaatcctcaccatgtaagacaagagtgcctcatggaaacagcaatggctttggtgtcagagacttgtcctcagatcctgattcccaacttaacagctgtgtgtctttggataaatgacttaatctccccaacctcaattttgtagcaaggtaaagtatctaatggtttgatgtacacagtaagtactcaacaattgttcatttccctttatcccttcctttcactgatggacatgagggtagaaatgcctagcaattacccttgcatacagattctaagcttttagaataatatattgacttcatgctctagatattatcattcttggattgtgcatgtgtgcccatcctttaccaaaaaaaggaggaagatcaatggcaacgagcattcactgagccttcagatgccaggtgctcaggaaagctagtgcttagcagagaaggtgggtgtcctagatgtcattgtgcagattcaaccactgatgaaggactgaggcccctggtcctcctacaccccctggaatctcagaactggtcagggtcagagaccagatagttcagagttctgaaactttatgatgatttatcctccatatttattcaccacctgagccaacaatgactatctttcatgaacttgaattatagtttgacataaaatatttttgtatttatttttaaaagaaaactttatatcacttgctataaatagaaggaaattataaaaattagtaaaatgaaaaaagagaataaaaagagcctaggtcttgctctttttttaagaaagaaaaagagagacagagagagaaaggaagtgagggagagaaggaaagagaaaagaaggaaggaaggaaaatggaaaggaggaggtagtaaaggcaactagtagatgatagagaggtgttaaaaaatattctgtccctaaaataagattttctgtttttacccagaaagatcaaaagaatgaaaaggcaaaaaccttcatcctgtgtgattcagtgttatacagtgtcctgtttctccatttactttctaaaaacatctcatgaacaaaatctatgtttgggaaaatgcagacccagacacacattttgcacatgggagactgaggtcaggtttgaggatctccaattcaagtccagctgcacagaaatgagctgatgtcctctctgctccctttccaagtaagcacagaagcctaagggatacttctcttatttctgcctgtgtcatcccataattatttactggcagctctggagccttgaggtaaaagtcttttcattatttatagaagcacagtttttattataaaggtaacataaccacagcacctcacttgaccaaaaaaaaaaaaaaaaaaaagatgaaaaagctacccacgattccactgctctggctcattggtatattttcagtcagcatttcttttacgcatttatttacataagtgcaaccacaatatacatacctttttttagcatcttgaatggtcttgtccaatctcctgatataatttgtgcctgttcatagcaaaagccttgggaaagaactgagtttcctctgtgattactttcctgtctgtccctcagttctttcatttgcaaaacgtgagaaatactatcatacttgcaccccaggcagttgtaagggttctcagagagagagtatatgtgaaatacttggaaggatctgacttgtacctaccttttgcacatcatcccctgcagtttcttcctatttgtaggcctgttgttcagtgtcatcattctgcaaaactcctatctatttgtcaaagccctattcagatgtgctctcttacaggaaaccttcccttcttcctgaaatgctctcctatattatgcctgaagcctatccatgcatctaatgtgtttagcatatggactctaaatatgcaagtgtgatacaaatacattcattggtcaaaaaattacagatgaagttaaaacttctgaaagttttaaccacccttctccatgcccaccaagaaaatatcttagttgtttccactactcaacttccaggttaaccaccaagttgacacagatttataatgtctctcacacaactgtgtcacactcaacgtatcattttcccattccgtgtatgtctcagaaacctaagcatattagaacatttagattatctaattctatctactgcttgtcacaatttatttagctacttctcaacatatatgctgctacatccttgtacatgccttcctaggcaattttcatttgtttgcttatccactagttagaccgtgagtccttcggggataggatctacatcttatttatctcagcagtcctttaatttagcagagtgcctgttacaacaaaatgcctaataagtagttggtgaataaatattcaccaacttattaaatgaataaggagaaaatcaagggacccttccagaatctagacacagaaacgagaaggctttcctgatctttcttcccaaaacattaaccttgaagattcggagcatggttcgttttccacacggggcatggccaggggccctcagttatttcagaagagtacagctgcttacagaaaacagactgcctgtcccatttacctattcttgtgcaagtgcccagagcccttaggcacaggcaaaggggatttaaataaacctaggaaataaataaataattgcttcgaaatagccaaatgggatatttccaataaaccatttccctacatgagagaaggaacatgagggggaacaatgcccaaagcagcaaatttatgaagaagctacaaagttaacagaaataggaggtttgcagagaaatatgctgctttagcatcccatcttcagggtattgctggagttttcgtttcttttatgaggcagtcatgaagttggaaacataaacaacactttaaatcattatttagcaagagaggagcaatttatttgcttagacaagcctgattgctgccatgataatgtcccttgttcatttgttctctctctccaagaagctggaaatatttgatagattgttacagtctgtgcatttaccatggcacaccgttatatcagattatttaatggaggattccacttcagggttgagacagcacttcctgcctttatttgtgctcgctcttcaactccgtctttcctaaccagctctactgatttctcagctctcatcccagcattacgtcctccagaaagtcctcagcaccatggagaagtatattgtatggactatcagcaccatgcactgcatgtgtgagactgtcagctacacaagactctgagtctcacttttggaattcataaaatagggagaatactggcagccccatcataaagttcatgtaaagatcagacgtttataaagtacttgccaatagccactgcccaagaaattacagtcacagtaataataattgttattatacttttcaacacctgatagtctctctatcaaagacaagctgaggtctcttgtctctatacccacagcatcctgtgactgcctcattgtgacttttattatattgccttgttgtcatctatatacctgcatttccccctagctgtgctcttccagagagcctactttctccatgaactccaacaaaattctgacctttagtaatgtctatgcattttctcttctctattttattttatctgcacaatgaagctagagattaagctgggtaactttttaactttctttagatgcaagagaaaatgaagttcaaagaggcagagtgactcattagggtcccccaggtttgatgaagtccaagtcctgattgaatctctctacaccacaaaataaccttgttattatcccttaacacactggccatcttaactccttgtttgctaaaagggcaagagaaaaccctattaaaaactgagaatatggccgggtgcagtggctcacgcctgtaatctcagcactttcagaggtcgaggtgggcggatcacctgaggtcaggagtttgagaccagcctggccagctggccaagatggtgaaaccccatctctactaaaaatataaaaaaaattagccgggcgtggcggtgggagcctgtaatcccagctactcgggaggctgaggcaggagaatcgcttgaacccaggaggcggaggatgcagtgagccaagattgcgccactgcactccagcctgggcaacaagaacaaaactccctcttaaaaaaaaaaaaaagacagaatatttcattcagcctgatcggaacaaaaagggagaaatgaagttagcagtaacaaacacttacgtatcacttaccaggtgccaagcactgttttaagcacttgacgaatgttagctcacttaatcctaatcaaagccccaggagtgagcactattattatccctatagtatagaggtggaaacagagacactaaacactttaccaaagtttacgctgccaggaagtggcagagctaggattcaaacccaggtgtctggatccacagcctgttctattaactgccattaacattacaactccatgaaatgtacatatcaaaaatatatatctgcatgtaagagaaacacttcctagctctctcttgggtttctgagaattacagctactattctgggaaagggtactgatgaaattttagtcattagatacttttcttcttttcatcagaaaagaatccagaatccacattagtggcttccaaaagctgatccatggaccttttttagtctataagtatatatatatatatatatatatgatgagatagtattctctttccaagtaagtataggaataagcacataaatatcctattaaagttagttcaccaccaccacatctccagttgaaaatgaaaagtgactagagctctgcttcctttgtattagcttctagggaaagctcaagaccctctctggcccactctgaatttcccaatttaacacaatgagcaaaaccaagtgagagctcaaattctaaccagatttgttaagctatttcatttaatagcagcactccaatatcctttacttgtctttccactcccatatgcgcagtccctgtggttcacctggggctgcacctcttccagtcccaccagaactgggcacaggctcctgccccagccaatcagcagattacttctactccctgccacggtgattggctgaagaacaagatccagctgaattactttgtttagaggcaataaagagacagatgtttgctgggactgttgggaaagaggtatttgtcttcagtggaatggcttatggctaggccataatcccgggtgtgctggtgtgtccggaattggtgggttcttgatctcactgacttcaagaatgaagccgcagaccctcgcggtgagcgttacagttctgaaagatggtgtgtccggagtttgttccttctgatgtttggatgtgttcggagtttcttccttctggtgggtttgtggtctcgctggcctcaggagtgaagctgcagacctttgcggtgagtgttacagttacagctcataaaggcagtgtggacccaaagagtgagcagcaacaagatttattgcaaagagtgaaagaacaaagcttccacagtgcagaaggggacccaagcaggttgccactgccagctggggcagcctgcttttattcccttatctggccccacccacatcctgccgattggtccattttacagagagctgattggtctgttttacagagagctgattggttcgttttgacagggtgctgattggtgcgtttacaatccctgagctggacacaaaagttctctaagtccccactagattagctagacacagagcactgataagtgcatttacaaaccttgagctagacacagggtgctgattggtgtgtttacaaaccttgagctagacacagagtgctgactggtgtatttacaatcccttagctagacataaaggttctccaaatccccactagactcaggagcccagctggcttcacctagtggatcctgcaccggggccacaggctgagctgcccgccagtcccacgccgtgcgcctgcacacctcagctgttgggcagtcgacgggactgggcaccgcagagcagggggcggtgctcgttggggaggcttgggctgtgcaggagcccacggctagggggaggctcgggcatgacgggctgcaggtcctgagccctgccccgcaaagaggcagctgagtcctggcaagaattcaagcacaacgttggtgagctagcactgctgggggacccagtgcaccctccgcagctcctggcccaggtactaagcccctcactgcctggggccagcggcaccggccggccgctccaagtgaaggacctgccgagcccatgcccacccggaacttgcgctggcccacgagtgcactgcacagccccagttcccacccgtgcctctccctccacacctccccgcaagcagagggagccggctccagcctcagacagcccagagaagggctcccacagtgcagtggcaggctgaaggtctcctcaagcatggtcagagcggacgccgaggccgaggaggcactgagcgcgagcgagggtcaccagcacgttgtcacctctcactggtagcagtcttgcctccaggggaagagagtctgacagagaatggagactacatggagaaaagcagagccaagggacaaggagacaaacagattcctgacaacatcacttgagcctctggactcaacctggctggaagggtcttcagcaattctttaatgaagaaaaacggaaaggccaaccccatggaatatcttctataaatgggacatgtatttagccctaagttaatgaactaggaaatagtgtgtcgttattattttatactgtggggtcataatgtattttaaatatttcatagtcttaattttctgggtcaggctaaaacaaggctaaagctaccttaaaatacagagtcgaattttgtgggtctcagtggcctagttgtacagcacatgaaccgattataaaaacaatctctcaggagacttttggctcatctgtggaccatttctctatttccaagtggattgggtggacctcgggttgagacactggtgtctcacaggatgcatgggctcctcccccagctcactgggcactctgtcatgggctggcttcaacctggcaaaacgtaatggctgcccttaaggtactcaaagagggtgatctcaagctctcccctacccactatctcccaaaattttgaaagaaggaaactgtattgaggaaggattttgtgaagtgcagttcatcaactctccccagtagtcaaagatcgacctcttagaccagataagatcattagaaagacatgcatttgtggctgggcaccatggcttatgcctgtaataccagtactttgggaggtcgaggtgggcaggcagatggatcatttgagctcaagtgtttgataccagcctgggcaacatggtgaaaccctgtctctaccaaaaatacaaaaaattagctggtgtggtggtatgcatatgtggtcccagctactcagtaggttgagatgggaggattacttgagcctgggaggtggaggaggcagtgagccatgagtgcaccactgcactccaatctggatgacagagtgagactctgaagaaaaaaaaaggcatgcttttgtattgccctccaaaaaagctgccttcattgaactgcaagaaggggagcagcatacaatgaaggtctggatcaggaaaagctgtaaaaagatccattcactgtgaggatgaccccgttgaagatgctgtggaagatactactcctcctgatatcattttaaaagagaactcctgggaaaacttctgaatggttacggttttactgagcaatgggatgctttgtttttgtggtctcttccaaacatgatactttgtgtttctgatgtgttcctgttgttgtgttgccagcttaggcagagagcttggctgagccacctgcagagaaagtgtgttggtcatgggaggctcactcctgctccatttaggtttccattttagttttcctttcaggcttggctcccaccattaatgttatgtatcactataggctactcaagttctttctggaagaaggcctcctataaaacagctcagtcagagaatgcttaaaaacatagttatagagagacagctgggccctgtgggctagtgacaagcaaacatccctctcggagaattttatttctatctggacctttatatgccccactatgatctgaagggttatcatcactatatctaatacaagtgggaaggacagatgaaagtattcttctcttaagtccattagtcctttctcatcttctggatccttcttagacaattctgtagaatctttgtgttggtgtggatctcagagttagcttgccctttgtttttctcctgatggggaggatcatggccactatgtatgatctcagttggcatcatctgctgtttccatcatgaagcctctggggatgcccttcagcacactcttattggacaggcctgtgtccggaattggttccttctggtcggttcttggtctcactgacttcaagaatgaagctgtggaccctcatggtgagtgctacagttcttaaagatggtgtgtccagagtttgttccttcagatgttcagatgtgtccggagtttcttccttccagtgggtcatggtctcactgacttcaggagtgaagccgcagacctttgcagtgagtgttacagctcttaaaggtggtgcatccggagttgtttgttcctcccggtgggttcatagtctcgctaacttcaggacagaagctgcagaccctcgcagtgagtgttacagctcataaaggtagtgaggacccaaagagtgagcagcagcaagatatactgtgaagagcaaaagaacaaagcttccacagcacggaaggggacccaagccagttgctgctgctggattgggtggccagcttttattcccttatttggccccacctacatcctgctgattggtccattttacagagtgctgattggcccatttacaatcctttagctacacacagagtgctgattggtgcatttttacggagtgctgacttgtgcatttacaatcctttaactagacacagagcgctgattggtgcgtttacaatcctttagctagacagaaaaattctccaggtccccactcgacccaggaagaccagctggcttcacctctcaggcctagtgttcttctatccatcacagcatatagacattcagagagtgacactactctgggtaaaacactgccctccttctcacttttgccttcattcctagctgttaagaggtcagccaatccatttcaccacactcatcagtagaacatttccaccatttctggaaaagcacctttccacttcttcagagaggattttacaacggcacttctgcatcccttttcaaaatctacctacctatccattcaaacttgggcatatatttatggaaggtttctgtgtgtcaggaactgtgcaagggagatttaaaaccccacttactccccaggagtttgacttacagcagaaaatgaggttcgtttccaaaaggcaaggacagaaagacttggagattagatgtgggcactactcctggctctacatctagctatcaactgtagacaggtcacactttcttggcttcagtgactgagtctataaaatgagggtaataatgattccttcactgagttaagataaactaacattactaatcacttactatgtgccaagtactggtatgtaggtactgatgttttccccatattacaaaagaagaaaccaaggcgcagaggagacaggcccaaagtcatgacaccaataaatgtgagctcgaggatttgcacttaaggctatgtgactccagaaataatctactctcctgaagctcctggaacacagtggccctcaagcacatgtgtttgcattttccctctgcttcgttttcctctgctttatacatttattctcttcccctctcctctcctcagtgtgcttagagaaaaggtctatgaaagacacattgcatgtttctaaccattcagttttctcttggattcgtcttcaccggtactaatagcacacagcactcagagaacccaaccatgatcgattttacagtgacctggaaatttcctccatctattcctggccctggggcagaacctcacaaaaatttactgtagaagaaaagcttctcctctactctgaaagcccctggaaaatcccataggcttcatctgttacccacattctaaagttcaagtgcataaacagcccaggtggagaaagcagatgggagctattatctattaagaagatgtaggaggtgggctttggaaggccaaagcacctgaatctttaggactctgctactaagtgtgactcagtgatcaacatcaccttggaacttgttaaaaattcagactctcaggacttaccctagaccgactgatttcgaatctgcatttaaaactgactcatgtgcccatggaagtgtgagcactgttgctttaggaagaggtaggaggatgtgtgaaacgagcaggaaagtgcatgttctccctataaactggtgatagagatagatgtgctcgactttacacaaatgagtgattggtgagtaaacagtacagaaattacaggggactttaagaaacattaacaatttaaaaatactgcttgcatagacaaattaaaaacacaggcaaggcataaactgaagtatatcaacctgcaccctgaggatgctactaacatgatcatgaaactgttggatatattaatgtgtttgtcatatgatattttgtttatatgtgtttgtctcttcaagattgtggatctccttgaggtaaaagactatattatttctctttgcattcctaaaggctgataataaatgttttaataatgaatgaggccatgtatggtggctcatgcctgtaatctcggtacgttgagaggccaaggcaggatcacttgaggtcaggagtttgagaccagcctggccaacatagtgaaaccccatctctactaaaaatacaaaaattagctgggcgtggtggcatgtgcattcagtctcagctattcaggaggctgaggcaggagaatcacttgaactggggagacagaggttgcaatgggcagagattgcaccattgcactccagcctgggtgacaatgagactgtctcaaaataataataattaataaaaaagactggttcagcaatggtgaagtaaggacctcctaaagtcctctcttccataatagcaacagaaagcataataaactttttcagatctctaaaagttaaccacaatttggaacaagctgactgtttttttttctttttaaattggctgaattggtaagaacaatgagctttgttgcattttaatttgcttaatcctattcctccaatgccccctcccccccacctcatgatagccttaaaaaccaatggctccaaaaatcacagtgaaaataagcagcctataagtctttggaggagacagaatggagctcctccaaagcccacttcccaggtagctgtcatcagttggtctgtctggtgattccctgaaaaaatttcacttgcaggacttgtctatatttgacctgactcagaactttcccaaagagggcagacttttccctcagagaagtttgttgaaaacaagcagtggcaattgttaaacactgcaggtgcctgagctagcaatatgagttggggaaaataagaagttaagcaaaaaacttaaaaggaaaatctgtgctacagggattttcctacatattcctgggaacctagaaaaccaagtgcatatcttgtctagggctgggcacatgcccagagataagtgcatacccaataaagacccgagagagccctaagctctcaccttttgatgattttaagaccctaagtaagatgtgaagacaaagatgagttataaactagctgcctgagcattaaagttatgccctgtcatgcacacagagacctttatcaatgactgaaagacttattggtcccaggaatttaagacaattcctgtccaataattagctgaccattaaactaactgaacacagacttaagtgggcacacatgaaaaggaatacagaccttacagtgttaattcagaaaattcatttaaaaattacataaacaacgaaccctgggggatctaatttctgaagttgacacattttatttaaaatgtccagtttcaaaaacaatgagctatgcaaagaacaaaatgtattacctatacccaagaaaaagaaaataaacaaaccaaaaaaacagtcaacagaaaattgtcaaaaaaaattgtctgtgaggaagcccagatgtagaactttctagacatagattttatgcctactatcttacatatgtccaaaacactaaaggaaaccacatacaagaactaaagaaaatatgaaaacaatgtcataccaaataggttatattgacaaagacagaatctatatgtaaaaaataaaataaaaaataaagtgaaatgaaaaattcatgagaagtcaacattatgcttgagctggaagaataaaaataagccaacttgaagataagtcaattgagattattcaaattgaggaataaaaagaaaaaaagaataaagaaaaacgaatagagccctagagacctgagagacaccatcagtgtagcaacatatgcacatggaagttccagaggggagaggagatagaggaagggtcaaaagtatttgaagaaatatggctgaaaacttccaaaatttgatgaaaaatattcatctaaacacccaagaaagttaactaaatccaagtagataaacttagtgaatcagagctagataccacataataaaactctcaataggcagaaactgagaattttgaaagcagcaagagagaaacaactaatcatatataagtggtcctcaataagattaacagttgattacttatcagaaaccaaggaggccagaagagtgtaggatgacatatttaaagtgctgaaagaaaaaatactgtaaaccaagaattctacatttaggaaaactattcttcaaaaatgaaagagaaattagggcatttccagatttaaaaacaaaacaaacaaacaaacaaaaaatttgctgcttgcagaccttcactacaagaaatattgaaaggagtcttttaggatgaaaggaaaggaaattagacagtaaatcaaatccacataaggaaataatgacatcggtgaaggtaactacatagtttacatatacaagatattagaaatgtattttttgtttgtagctttttttctatctgatttaaaagacaacccatgaagaaataattataaatgtttgtgagcatgcaatgtataaagacacagtttttatgataatggtagcacaaagaagagaagagagaatggagctatataggagcaaaatttttttgtaaactattaaaatgaaattgatattaatctgaactagactatgataaattaaaagaattaattttaatccccagaaaacaactttaaaaagtagtttaaagaaaggagaattaaagcagtgcactagacaatctatttaatacaaaaatagacagtaatgaaagaatggaggaccaacgaagatgttagacatatagaaaacaaatagcaaaaagacagatgtaagttttataatatcagttattacattaaatctaaattgattaaacactccaatcaaaaagaagaatttagaaaaataagagacacagtttaggccgggcgcggtggctcatgcctgtaatcccagtactttgggaggctgaggcaggcagatcatgaagtcaggagattgagaccatcctgtccaacatggtgaaaccccatctctactaaaaaaaatacaaaaattagctgggtgtggtggcacgcacctgtagtcccagctactcaggaggctgagacaggagaatctcttgaacccaggaggtggaggttgcagtgagctgagagcatgccactgtcctccagcctggtgatagagtgagactccagctcaaaaaaaaaaaaaaaaaaaaagagagagagagacagtttagatttaaaaacataaaaagattaaatataaactatagaaaaacatgtaccatacaaatggtaaccaatagagagctggggtggccataataatatcaaacaaaatagacttaaatacacaaattgttacaccaaagggcattttatgatggtaacagtcaatccattagaaaggcataacaattataaacatgtatgcaagtgataacagagccccaaaatacatcaagtaaaaagaatcaaattcaagggagaaataaacagtttaaaaagaatcactggagatttcaatatctcatatttaataatgaatagaattactaggcagaaatcgataatgaaacaaaagacttatacaacagtatagttagactagacataacagacaactatagaacaatccacccaacaacagcagaatgcacattcttctcaagtacacatgaaacacttcccagaatatagtatatgttggacaataaaacagatttcaaaaagtgaaaataattgaaataatacaatatgtgttctctaatgacaacgtaataaaattagtaatcaaaattagaattttgagaaattcataaatatatgaaaataataataatacattcctaaatcatcaaggggtcggaaataaaaatcacaagggaaattagaaaatactttgacatgaatgaaaacaaaagcacaacatatcaaaacttatgagatacagcagaacaagtgcttagagaaaaatttacagttgtaaatgtctatattcacaaagaagtatctcaaataaattacctaaacttccaccttaagaaactggaaaaaaaatagcaaatgaaccgtaaagtaggcagaagaaggaaaataatgactacagcagaaataaatgaaatagagaacagaaaaacaacagagaaatcaataaaaccataaatttactctttgaaaagatcaacaaaattcacaagcccttagctagagtgacaaggaaaaaggagaagatgactcaagttactaaaatttggaatgaaatagagaacattactagtggtcttaaagaaataaagagaattataagaagatgctatgaataattgtatgtcaaaaaattagataacctagatgaaatggacaagtttctaggaagacacaaagtattaaaactgagttaaaaagaaacagaaaatttgaatagactttagcaaataaaactattgaattagtaacaataaaccatccttgccccatgaaaaagagtatgatcaaatgccttcactagtgatttctaccaaacattaaaagaagaatcaatactaatacttcaaaaattattctccacaccaggaaaaagaatatctcccaactcattttatgaggctagcactattctaataacaaaatcagataaaaatatcagaataaaaaaactacagaccaatatccattatgaatatagatacaaaatgccttaacaaaatactagcaaactaattcaataacaaataaaaaagtttatacaacatgaccaagtgagatttatcccggtaatgcaaagttgattctatgaaatcaatcaatggaatacatcacatgttaatagaataagaaacataaaacctcctcttgagtatttcaataaatagaaaaagcactgacaaaacataccacttttcatgagaaaaatactcaacaaattagaaatagaaggaaacgtcttcaatctgataaaagatatctattaaaaacctacagctaacacagtccttggtagtaaacgactgaattctttgccccacaaaatcaggaacaaacaaagcaaggatgtctgctctcacatttctattcgactttggactagaagttctattcaggacaatgaggcaagaaaaataaataaaaggcatctgaactacaaaagaagaattaaactatttctgtttgcaactgacatgatcttttatacaaataatctcaaggaagtcactaaaaaactattagaactaataaaacagtttagtaaagttacaagatacaaggtcaatattcaaaaatgaactatagttctatatgctagcaagaaacattctgaaaaaattaaattaagaatataattacatgtacaatagtaacaaaaacatccccaaaggaataaatttagtacaagacgtacaaggcttgtacctgacaactacaaaacattgttgacattaacttttaaaaagttaaataaatattaagacatcctgtgttcatagactagaagatttagcattgttaagatgaaactacttcccaaatttatctacagagtcaacataatcaaattcctagttgtagaatttacaaaaattgaaaaccatgatcaactggcttcaattcgatttgtattctcagttttctgcccaaactacacagatgggtttcaaatataatgagaaggttgtagaaaattcatgataatgttttctgtattttctcttctatctaagctttcgggctagaaggggtatgtcattcctacccacaaggaatatgaatatgtattcattcacctaacaattatttactaaacaaggttctacgtgtcaaactttgttctatgagctgggaacacagtcatgaacacagcagaaaataaatctttcttcttgaggagcttacattccagtggggatacaggtaattaaaaaaaataataataaatacatagtatgccatattgggataagttctgtagagaacaattaggctagataagaagaggaagagaggaagagagtgtctggtagatggtgaaggcattgggagtgggtggctggggtaggcattagctgaaaaattgaaattcatggtggtattttcttgctctccaatctattcccatccttgccaatacagcagctgaaattaagtgttctctgcttggaaccgcctcagttttcatttatcaacttctgctttgatccatccaacactccatccatccatccatccattatccatccatttaattattagtccatccaatcctcagtccatccaaacttccatccaaccatccatccaaacatccatccatccatctgtgcatccaaccatcatccatccatccatccaatcacatttcatcctcttactcattcttttattattgagatataattcatataaaataaaattccctcttttaaagtgtaaaatacagtggttcacaaagttgagcaaccattaccactatgtattttcaaagcattttcatcactccagaaagaaaccccataccaattcactatgctcaacagccctagtatttaagtccctacttaggaggccagagaaagaaaagaaactgggactaagaatatgcaggcaattcagctctctatctggcattgagaaaagtagggcattgggtgggatatcctgaaaccaggatctgtttggatgtattggtttcctctatgaagactgagttgccttctttttgttttttcattgtcatgcttcctggtgcagctgccttcatatgtgcctgcctctctaccccagtcaaggctgcgtgcctcacccagtggggctctgcactgctaaatgagcttgcaggattccactcagctgtcccattctcactgtgccaatgtggcctgagtctctcatgcatcttttgttctctgactggctcacttcctcatcctcagctctggggagcttcccacaatggtggggaatgttttgaagcttggaatagaaaggcaatatgcagtcttccctcggctggctgcaggaaagaggcaggtggcagaatgtgtgggtggcaagctcagtggggaccaagctggcaggagcagacgtgactgagaggacaaggaggagcaacagcagagacagtctgtgacagaggactttgaattttctgcccacactgtgcatctccatgttttcagccttcacctgcgcaaaccctggatgctgaggccaacaccctgcttgggaatgaggcttctctgcgggtttgcatctaggctatagaataaaattattgcgtgtagtaacaaggctggaactacagtgaggtaaatgagacactcactttgggcacaaagtttaagagggtacttgtgcacaaatgctcataacagcattagtcataatagcaccaaagtggacaaaacccaaacatccataaatggatgaatggataaacaaaatgtggtatgtccatatgatgaattcttacttggcaattaaaaagaataaagtactgatacatgctacaatgtggatgaactttgaaaacattatcgtaagtgaaagaggtgggacacaaatggacaatttcatcatattgtaccatatgatatgatagcatctatgcaaatgtccataataggcaaatctacagagaccaaaagtagattagaggttactttatgacgccactaattcaggggatggaggagtgatacccaaagggtatggaggttcttttgggggtcataaaatgtctaaatttgactttagttatggttacacaactctgtgaagataatttttaaaaacattgaattgtacactttaaatgggtgaactgtaaggtatgtgaattatatctcaataaagatgtaaaaaacaaatgtaaagatctcagcaatcaaggtaaataatattttaatgcaataacatctcgatgaaggtgtcagggagggctttaataagcaagaaacctctgagctgagcggttcagaatgacaaggttagggaaaaggcatcacagttagtggaaatagtttgtatacaggtaaagaggcaagaaggggtaggccacagtgaaggggtggaatttcttacagcagttttttttttttttttaccatgatatactcttgaggatggagacatgagctgaggagggtgggatcaaaaaacagaaggcactttgggtcccctatgacaccatgacactgttacacccgtcttggacagacataagagaaaaagaaacatctgtctgctttgttagttgggttttctgttttatatgttgaaaactaagtctaactgaaacaccgccactcaccatttactgagttcttactatgtgacattgatcggctaaatcctttagatgaattgactcatcagtgagtctatgagtgggtacagccattgttctcatttcagtcaagggacctgaagctctgaggaattgagcaacggtcccagattccatggttactgggaggtagatctggaatttaatctccatctatctgacccgaaagccaaaactcccaccactgctctgtaactgtctcccactgagcgcaattaagctggcgtcacacactctttcctgttggctggtgctggcctcactgtagtttagatggtaacctcgtgtctgattgcagaagaccactcatcctggatgctccagctcttacagctctgtccctggcaactgcctggtaacctatccactcctaggcagtgatctcagactctatgtgctttctttgagccccctcttaagggctgacttcctggagcctgaatcccatctctgggcctggggccctgattcccctcgacaggcttatctgatgctgacggctccaccctacagccccatttctatccagttccctgaagtgtgggcaagcgctgtcccaaggacaccagtccatcaggctactgctaaggttgcctcctgaggctggggctggatgagctggtccattactcaaaatggccagaagcagatgaagccagagtccacctaagcagactctaaaatttaaatacaccataaggaacaaagaaaaacttgtggaccaaagtcaaaagagataagtgactgcagaaacaaagaatgatgggggaagcaaaatcaaaaagaagtaaaacaacccagaacttttgcagtggggatgtagaggctccttcttcatgagtggctcaggttagaacagcagcctcagtgcttggccagcctgtgggaacatctatgacttcatcttgggctcccagtgttgatccttcgctctgtctcactccttggacacagtgtattcttacttgactgaagatctccagctcttcgggtcaccctgagccacctcttgtctcaccctgtgaccccagcctctttactccacccagttctccttatgtaacctgagagcttactaggtgttaagcacaaagtccagcaccttcatagtagctaatgtagccctcactaggatccaacagggtaaatataatcatcccagccttggacactgaggtacagtccagtgacttgctcaagttgacccagctagcaagtggtgaaattgggattcaaattcaggtttgtctgcctctaaaacctgtgcttttgatcattagggagtacatgctatttcattttagtcttcagatctcccccaggccagccactgcatgacacacttagttcctgcccacagctacttggccacaggttgttcaaaggtctgggctggaatggccccaggtaactgttcactactgagctgtgcagaatagaagcacagccccatttcaccagcccccagctatcaggaggggccacgtattggggtcatctccccaaaccctcctggccatttctggttgtgaaaatacctggagctcactaaggctgttgtgcagtgagtgtagtgggcaggcatgagatgggcctattgggagaatcaacaggaagaaatggaactttcagacagacagtggcctccaggcaggggttcctccttgaaatccatatcagaatcttctgtaggacttcataaaacacgacatgtgccaggagtcccaccctagaaattcagatgccatgcaggtctgggcgaagagcatttaagtgaaaggacagcagatcccatttgtctggctttaaatccgtgctctatctctgtctagtggaacagccttgggtaagttatctcacttatctgtgctcaggttcttcatctatatttcaggataataacttacagggtttttgtgagggttaaagaaacttagaaaataacctgaccctaagtaagcatgtaagggggtgagttgttataatcacttactagctctagctgtaaaattcttcataagttagttgagaactatggcaaaaggaaaaagcaggcatagtagaaaaacccaggcttgggtttagacagacctgtgagttgtgtgaccttgggcaagccacttaacctctctgagccacaactgtcccacctgtaaaatgggaatagctaacatgtagggctgctaaaaggactgctaaaaaggtaataaaggtacaggtcctggcatagcacacagcacatagtaggtgctcaacaaatcacaggtaatatttttaaggcattctagaatagcagcttagctaatttcaactgggcctggtggttttaaaatttgagtaaccatttattctctttgcctagatgaaataggtcctctgcatttccaaacaggaaattcatatttgcttgtaatggcaaggccatccaccaacaaccagtatgcttagaacttcaggtgggaagtgttaatgatacaactttaattccttggcaaagagtctacccatcacttccaggttttcccagcacactaaaccgcaagggaaggaaaaaccacggcattattcacagaacccactgcttctacaaggtttgtttcctgatgtttctagctgctcctcctaagccctagacaatgtctgggacacagagacagttgctatggagatgggggttttcccacagggacagccaataataacctcttattcagagaggcttatgtgttacacattctgcatcagtatggcagattttagcagtgaaacacgtaaattgttcaaattgacgtcatagcacatcaaggctggcccctgtgtctaccactcatatagaactaagctcatccagctttaaatggccttatgtgtcaatctgtcttaaataccccatcccttttttcctccctagaacaaagggacgaatgaaacacccatatacacactttttgtctgtgtgtttttctctccatcttctgtcttcatttaaatggtaagctccttgaacttggggaattcacttttcacttccctcgctttcctacagtcccctggtggcaggcaagatagagcagagagagttaggcagcctccttcagctatcacatagcagctgtgaaccactgccagacaattcatctcttagagccatggatttctcctgtaaaaggggagttaaatgtagtacttcccaaagtgtagttaggaatgaatgataccatttatgcaaagtgcctttcagttagtaagacctcattcaatgctgtctcaaaatcattatttacagtcaaagcatttgttgattgacttgattggtagacagatttctcaggatatgttatttgcagaggcagaacaaaaaattatcaaacaacgtaaatgatggaagactaatagtctctcggccacctcaggggattgagggagatgtgggtaggggacaaaacccagcatctcttctaagctaggagccaagagcacatcttttctagtaatatgtcacaagttactctgtaaacgagatattatagtctctgttttgcagacgggaaaatgggaaaaggctagacagcaagctcatcacagcctgttttttatactttttggacactcatactgctaatcccagcctgccttgcataggtgtggccaaagggatgagagggaatgtgctctgtttggatccagtgcatcagacttgctacacacacctccccactctttccccttcagacagaaatcagagggtcccagaggccgagagtagggtttctcaaaccggagctgcatcacgatctctgaaggccatgtgaaagcccagatttctggaacccactccagactttctgattcagcaggtctggagtgaagctgaagaatcttcctgtctagcaagcttcccaggtgatggtgttggtccaggaccacagttggaaaacttctgttctagggcatggcagagccacaagatgaatactgggtctctgaatcaccatacggtgtaaagactcccactgagtttcatatctaaaaatttttcttacatatcagaaatttcaagatttatttattataactgctagcaggactctagaaaaggaaggctaaagaaaacattttctccatgtgaaaatgggtggctttatagaaaaggctctgaagggaaggaaggaaccctgggtttcatatccagacctcatcacctcaaatccttcagtaagcaccaatacaatttccattgactgcagtcataataccaagtgccaccttcaaattccactaaacataaaaaaagtggctataatccattactagaaaaactggcaaagaaaaaattccccatttaaccaaggcccctaattatgttaaaccttcaacactcaaattcaggcataatccttcctcctgattcccattgccattatcttgactctagagtaaacattgtgactgaaaggtattgcccacgtcctctgtgccaggcactatttaaggtctttgcacgtatcctggcacttaatcctaacatgtatttcctggaggtgttgcaatggtttctgctgacggcttgcagattaatcttctcaaaccaacactttgaacaagtcactctcataatcaaaacctcagggcctctttatggcctacagtgcaaagtctaaactactcagcatgatcttaaattctctccaaaatctcatacataactacttatcaattttaaaactcttactacagtctgcatctgtgactcccaaatattaacgtatatatcagaactccctagaaggctgctgaaaggcggattcccaggcaccactcaaatctcctgaatgaaaatatttggtgatagcccaggaatctgcatttttaatagactatattctagaacattttaagttaacaaacagcaaaaataaccagaaaatacagagatcttccatatatccctgtccccaaacatgcatagcctcccccattctcaatatcctcaactagcatggcacaattgttacaactgatgaacctacagtgacacatcattatcacttagaatctatagttcacattagggttcactcctgctgttgtgcattccacgggctttggcaaatgcataatgacatggatccaccataacagtaccatccagagtagatatattgatctaaaaatcctatgtcctctgcctattcatccctccatgctccctccccaccagctactgatgctctttttagtgtctccatgttctatctcttctagaatgtcatatagttggagtcatatagtatgtagctttttcagattggcttctttcacttagtaatatgcatttaaatttcccccatgtcatttaatggtgtgatagctcttttttgtttgtttgtttgtttgagatggagtctcactcagttggtcacccagcagcgcgatcttggctcactgcaacctctgcctcgtgggttcaagagattctcctgcctcagcttcccgagtaactgggagcatgtgtcatcacacctggctaaattttgtatttttagtagagacgaggtgtcaccatattgaccaggctggtctcaatctcctgatctcaagtaatctgcctgcctcggtctcccaaagtgctgggattacaggcatgagccaccgcgccggccagctcatttctttctagtgctaattagtagtacattgtctgaatatgcccacgtttatttatccactcatctactgaagaacatcttggttgcttccaatttttaactattataaataaagctgctataaacatccatgtgcaggtttttgtgtgaacatcagtattcagcttccttgggtaaatactgaggagcacaattgctggatcatatgctaagaatatgtttcgttttgtaagaaactgtcaaactgtcttccacagtggctgtaccattttgcattcccaccagtgagaaatgagagttcctgttgctccacatccatggcccatttgatgttgtcagtgttctggattatggccattctaagtgtgtagtggcattttatttttatttttatttggatttccctgatgcaatgtatgcatatattatcatgtggaacatcttttcatatacttatttgacatctgtatatcttctttggtgaggtgtctattaaagtgtttggtccattttttaaatgagttgtttgttttctttgtatatttggacaacagctttttatctaatatgtcttgtgcaaatcagacagtctgtgcttgtcttttcattctcttgacagtgtctttctcagagaagaaatgtttcattttaatgaagtccagcttatcaattctttttcatgggtaatgcctttggtgttgcattaaaaaatcattaccatacccaaagtcatctagattttctcctatgtttcctcctatagttttgtgtttcacatttaggtctgtgatccatttttagttaacttttgtgaagagcataaagtctgtgtctagattctacttatttatttatttttttgcatctggatctctggttgtttcagcaccatttgttgaaaagactatcttttctccattgtattgccttttctagaatctgcatttttaaccagctctctcagtgattttggtgtatttagtctaaagatccacacattgaaagacatgtttttttacatatcaagttgttgtcactcatgagcattgaaacaaccgagccccctattgccaattagacttcatatcagagagaaattaccgtgtagtttaaaccactgctgtgacagctagcattgtcttaactaaggaacaaatcttgtcttccccactagactgtgggcctctaaagacaagggcccagtctcagtagcccttatatatttctcacctcctcctgacctaccctctttctatgacaaccagactttgttcaagagctggtagagggcaggggctcataaatatttgttgaaataaaatttctgtctttttgcattcagccaacagccagatgggtgggggtagggcaaaaggatgcagatcccaatccagacatatctatattactagaaaatttacattttgatcaccagctccaaagtgattaattgttgagtagttttcaagtggcccattttctgtgggaacggctttctctaaccttcccgctttcttattaaacacctgcagcttccctaggatttctaagctttatgattttcctgaaaaaaaaaaaattaagcagactttattattcagctttgccaaagtaatcaaagaaaagtttaacttgatgctgcgattaaagctttggcaatgggcgttgacgtcgccagattgaaactagtggtggggaaaaggctgtttttctaaaaataccaaattaagaattctccagtttccaactacatgcaagagaatctccatacgcaggaagtcctagtcacaggcattttattgcagaaatgggtactttttgttttcctaacatgctcatctaaggctgtccttctaagaactgtgtgtaacagaaggcaaaacttgcaccacctccctccttttatctcttactctggacaggactttgatcctctttgcgcctcagtaccttcatatgtaaaataaggagtctggattcgaaaatctggtgtgcttcttgcttttataaataaagttttattggaagacatacccattcctttaagtattatctatggctactttttttgttctctaccaagtcagagctgagcagcagcaagagaggcccagcaagtctgaaatatctacaatatgtatttttacagaaatatttgccagaatggttttgttacagaatatttttatagaatatttctttacagaaacaatttgccaacccttagactgcaccgttttgattgtaaaaatttaaaagaaagttcactcattcatttattcatttatactgaatttgtgtatttgggatggggcccagggatgtttactttaacaagttccacaggagatttgtctgcagtctgcacattaaaatttgggaagtgccaatccagtcaattgtgttacagcaattcatgagcctcttagtgactaagaaaagacaaggaggagtgatgtggctccacattccagcaagcatgtattgtgccctgatgatgtggaaggctaactcttctaggcactgcagatacagaaaagacaacctggacaaccccactgctgtcatggagctcatgttatttccatgaaccccttgggagtctgggcatcgcaaggcctcaagcaagagccattgctccaggacttgggccagtgcctgccacagggtggtcaagggcaaagggcaagggctttgtcacttacattatctaaccacagcctgaaacccacatcattgtaaagtggctgaccagagggctaaatggccatgggccaaaatgaaagctgccatatattacattggtcagtgccaagaaaaagatgggcttcttctatgaactgctttgtcatattttatttatttatttaattttcaaaaacacctcttgaactctgcaacccagcttgtttccatagtagccttggctgccatgcatttcacttgtagtaacttcccttaacctacacggaccactgagacctccctgcccttcacccttccttttattattgtactaattaatgttgttgttttaccagtttgtggccttatcaaatctatttgggaaagtaaaacttgaaaaggaatataataaatgtactgtagtgatgacagtgaaagtattctgtaaatattaacacttccaccatctctccaccctccatagctctccacccccgggaagctgattgcctttccttttctggatgtggaaagaaggctgagtgattcactgcaccgtagtgagtcaaagatgaaaacatgcaaatccggccctgtgacgtctgctgtcagcaggcttgagattggaatgggcatggtctttgttatcatcttaacctggctgatccttttgtgtaccctgttagatggtagttcttctcatttagtatctaaagaaactgacctgacagagggctcagttcctggcctgaatggaagcagaaccgagacattttgactcttctgcctagaaagtggctacagacatgccttgcacatgtagactttgcttcctctctcttactacgtctaggtaactactttggacatgatgcttaacctctctgagcctcagtttttaaatctgtaaaatggagagaataacatgcaaaatgcctactgcagtgtctgggatgaagtagattaacagtaatgtgatagttctcagaccttccctaccctgccattggcagtattcattgggagctgctgggactccaagggcatggctgtctatggaggtgaggctggaatctgggctgtgatcaatgaactcacaattggttcatgggcccacatggccaaagctaccctttctgtagccactgtctagctagaagagtcggaatgcctgggctttgcttcagttcaggccaggaactgagccctctggcaggtcagtttctgtattagtccattttcatgctgttgataaagacatacccgagagtgggaagaaaaagaggtttaatggacttacagttccacatggctggggaggcctcacaatcatggtggaaggcaagaaggagcaaatcacatcttacatggatgggggcaggcaaagagagagagcttgtgcaggggaactcctcttttgaaagccatcagatctcatgagacttattcactatcaagggaacagcacaaaaacacccgtccccataattcaattaccttccactgggtccctcccatgacatgtgggaattgtgggagttaaaattcaagatgagatttgggtggggaaacagccaaaccatatattccaccctggccccttccaaatctcatgtcctcacatttcaaaaccaatcatgccttcccaacagtcccccaaagtcttaattcattttagcattaactcaaaagtacacagtctgaagtctcatctgaggcaaggcaagtcccttccacctataatcctgtaaaatcaaaagcaatttccaggcccacggtgcaagctgtaggtggatctaccattctggggtctggaggatggcggccttcttctcacaactccactaggtggtgccccagtaggaactctatgtgggggctctgaccccatattttccttctggagtgccctagcagaggttatccatgaggttacccaaccctgcagcaaacttctgcctggacatccaggtatttccaaacatcctctgaaatctaagtggaggtttacaaacctcaattcttgacttctgtgcacccacaggctcaacatcatgtggaagctgccaaggcatggggcttgcatcctctgaagccatggtctgagctgtaccttggccccttttagtcatggctggagtggctggaatgcagggcaccaagtccttaggctgcacacagcatggggaccctgggcccagcccatgaaaccattttttcttcctaggcctctgggcctgtgatgggaggggctgccgtgaagacctctgacatgcgttggagacattttccctattgtcttggggatcaacatttgactctttgttgcttatgcacatttctatagtgggcctgaatttctcctcagaaaatgggattttcttttctaatgtattgtcagggtgcaaattttccaaacttttatgctctgcttcccttataaaactgaatgtctttaacagcacccagttcaccttttgaatgctttgctgcttacaaatttcttctgccagatactctaaataaactctctcaagtttaaagttccgcaaatctctaggggtaaaatgctgctagtttctttgctaaaacataacaaaaatcacctttgctccagttcccaacaaattcctcatctccatctgagaccacctcaacctggatttcattgtccatatcattttcagcactttggtcaaagccattcaacaagtctctaggcagttccaaactttcccacattttcctgtcttctgagccctccaaactgttccaaactcttcctgttacccagttccaaagttgcttccacattttcaggtatcttttcagcagcaccccactctactggtgggctatcaatttactgtattagcccattttcacgttgctgataaagacataccagagactgggaagaaaaagaggtttaatggacttacagttccacatggctggggaggcctcacaatcatggtggaaggcaaggagaaggaagtcacatcttacatggatggcagcaggcaaaaagagagagcttatgcataggaactcctctttttaaaatcttcagatctcgtgagacttattcactatcacagagcagcacaagaaagacctgtccccatgataaaattacctcccactgggtccctcacacgacacgtgggaattgtgggagttacaattcaagatgagatttgggtggggacacagccaaaccatatcagtttctttatatgctaaatgagaatatgtactctctaacagggttcaactagcctgccacccctttctacagaatatagtgctcccctccttgagcctccactatatctgaaacttatttctatattgtccatgttatatttcattcattcatcaaacagtgattgcatctgatgtgacacatgccaggctagggtctagacatacctgtcttcaaggaacatgaccttcagtagcagagatatgtcaacacataaataagtgaaaattgtgaagttcccagtggggtgctgggaagtgggcagctattaatatttctttaagaggtgacttcaagctgagatctgaaggatgagaaggagccaaccttacagagtgtggggaggctgaggagtttcctgcacaggagccctgaggcccagaacagagcacaggactggtcaaagaaagcaaagaggtccaatcagctcgggtgaagggggagagggaagagcaggaggtcactcaccccatgcaggaggtcatctaccaaagcaaggtgatgggtttaattggtagtgaaaaagtgggaggggcgtggggaggtgtcagcagagggttttaagtaaagtcaggacataattttcattttaataggacccttttggaatggcatttgttgaggacagagtaagtatctaacttttgtttacctccccagagaatgcacaatggccaacagatagtagtcactcaataaatatctaggaaatgagggaactaataaagagaaaaaaggatatatagaaaaatgtggaaaaatgctttcaagttacaacacattataaatgtgaatgaataattgctattatttaaagattatttacatgccctttcaaatcaaatagaagcaaactcaatgaatttattgcatgatcccttgaagagaaaaagaaagctgtaaaatgacatagaaaggggttccaagcacaatccagtcatcattagcaacagcaaaaccccagcctttggaggctggaaggagtatgagggtcatttgccccaccctagaggcccaaaacgcacctcatgggatcatgggatttttatgagcaccagtgaaacctttttttttttttttagacagagtcttgctctgtcgcccaggctggagtgcagtggtgtgatcttggctcactgcaagctccgcctcccaggttcacaccattctcctgcctcagcctcccaagtagctgggactacaggcgcctgccaccaggcccggctaattttttgtatttttagtagaaacggggtttcactgtgttagccaggatggtctcgatttcctgacctcgtgatccgcccacctcggcctcccaaagtgctgggattacaggtgtgagccactgtgccagccagtgcaaccttctacatgtgcactcattcccaccggcctggagccctctgtggaatcacctgctgaacccaagtaattaaactaaaagactcctagtacaaactatgatttactgaccacttactaggggtcgctacacatcaagcacttgcacccaggaccttgggtctttgcacctcatctgatttgcacaatagtctcagtgactgatggggaccacagaataggcctttgattctaggactgctgtttcggcaaaggagccaaatcgtacaagaactcaatttttatttttataaaaatgaaaataatcctagcagctacatttattgtacccccaacgcaggttagaaatggttcttgacactttacgtggtttatctcaaggctcgtgcctactccaggaggtggacaccctttgtgggttttacagaagaggaaatgaaggtgctgaggtgtaagtaatgcaggagaattgcagaaagaggagtgacaggggagctgtcagcagaaccacagaaacatccagggcaggctctgttcctaaccagggaacttgctgtcacctggcttcccctccccagcccctgcaatgggctctgagactttctgcttagacattcagtggtgggagctcattacctgcccccacccccaacaaggcagcccattctaattccaggcagcgatgggtatttaaaactacttcctcatgttgactcacaatgtgtctcctctcacttccctgatgtatttttatcctactctgttctgcggctgcacctgcattgttttgaaatatcataacaatcctccatggtcagcaccatccaccccaagactgaatgccatgaagagtatgtgcctcaggaagaaccaggcagaagcaactgagtctccaccccaacaaccagagctaggcgactatggaaaatcattcgaccccttagaggtcatgtcctagtctgtacaatgggataatgccagtaataataacacctatttcagagcaatgctgtgaggccaagatgtcataataaatatggaatgctgaaacacattaataaatgctccttcaactttctcatcttcttggcacagaatgagcccaccacatcaaagtgacaaaaattatgattgaagaaacatgcaatgaaactggattaaagatggcaaaaacataataaacaataatttttaaaaaaaaaaaggaaaaagaaacttgattaaaacttattatgtgctagactatatacttaccacttttgtgttttctattctaagttgaaaatctacaaggtagatgccatcagcatcatttcacaaattaaaacaaaacaaaacaaaaactgggattcagagatgtcgaacaacctgcttaaagtcacacagcttttaagtagcacattcaaactcaggactcttgacccaaatccagtcctctttccactcccagtcttctctttcctccaacatctggccaagcccaggttattatcagtgtttgctcagccctggaataggcacagatcatgctgagcatcacagatagtgtggaggagctacacattcaagtttgcagagtaaatcctaaaaagccttagagaaggaactgcaaggatgccaaatcaatctgaaggaggcctcagtagacaaggaggcatggggaagaccggctggggagacagatttttgagcgtcttttcacaaaatctaaaaattactaaccttctacatgtcaaaagatatttttttgtcaacacatgcaagaacaccctgctaagaagcccaaatgagcagatggatgagttcgcagactcgcagatgctggagttggaaaagtccctgctggcagtccccaaaggacggcccagaaaccccaccctgccctagcccctcaccaggctgagttcaatacctttcagagagccttcaagcttctggccttagactgaatcttgctctctagaaaccaggttgttggaaggttttttcttctttttataatgatccccctgtctctgcttcctgctcctacctccctctccttagcttctcactgctttccatttggcattccacaagcatctcaccttcacccccacaggacagaagggatcatccttctgtgataggcaggaattcaaggctacagagctcagattcaaacaagtcaggaggacacaattacaccaaaatctgtccatcctcaaagaccatgagatttccacagtaccacattgcctctaactaactagagaatccccattttgtactctggaaatttacaacccagagatgtcatgtaaattggtttcacataggatggtaagaccaaagacaaaattagcatacaaaagtagaggcataaatctcaatatcatttctaatttgggttgcaatttagactgtgcttcctatggccaaaggaaaaaaaaagaagaaaaatgaaaagaaagaaacaagattaatcccacaatcagcattgtcctcaaggaaacatctaccagtgctgcgtcgacacatgcttcctggtattcctgtctgaaagagcagcttcctatgggctagtgatccttgggcagtaagaccaactctcaactttcccacgacagtttattttgatgtaagtggaaagggacccttgagtcagaccttctgaacacagttcgatgggactgaaagtccataagtaggactctggggtgttcccgcctaagttagtgataagaacaagatccaccttcttactactccaagtgtggcctgaagaccagcagcatcagcatccctggaggtgtggctagacatgcagaattgcagacctcctgggctaggaatgagaatctccattttaatgagaacaacagaaggttcctatgcacattcaactttaagaagtactgcactagggcagttgctctaattagcccctccttaagaattggagtgttcactaaaaatataaattccagcctccccaaaagctagatgcctggtccttaaccttgactgcacattagtatcagtagaggggcttcataaaaaagtgcacatccgtcaggggcggtgactcacgtctgtaatcccagcactttgggaggctgaggcaggcagatcacttgaggtcaggagttcaagaccagtctggccaacatgatgaaaccccgtctctactagaaataaaaaaaaaatcagctgggcttggtggtggatacctgtaatcccactactcaggaggctcaggcaggagaattgcttgaacccggaaggcggagtttgcagtgagccgagattgtgccactgcactccagcctgggtgacagagccagattccatctaaaaaaaaatgcagatgcttagatcctaaccgccagtgattctgatttattaaatatttgatctagggtgtgaactaggcttcaggattctaaaagtctcctaggtgattttaccgtgcagcatggttaagaagccctggaccagaggatcaacattatgtgtagctctgctaaacaacacatggtaagatgtcagtggacactactgtggtgagacttagtttagggtcagttagatctgggttcctactccatatgaattcagtgagaatctatggacgtgaagacaagatttcgaatgtttttaaagctctgcagtggactctacttgcaggttgaggatcactggttaaagcatactgcatagcacctggcacatagtaggtgacttatgaatggtatatattttttcttagaagccatgatcacacacatcaacagatacacacgcacacacacgtgtatatgtatacacatgattgtatgtctacatatatgcacacatacatatgcatgtaaacacaaacacacatctatgatcatatctgcatatctacatgcagacatcaacacatacttatgtgcacatatgtgcatatatatgcatacatatatgtgcgtgtatgtgtatatatgcgtgtgtgtgtgtgtgtgtatatatatatttgagatagttctgggcttctcaagggagatcctggatttgagaagctttgacaaattccataaagaattagcaattaggatatgagtactgatggagagtcaattgtttattcagcataatttactgagaactcaacttatgacagaccctcttccaagtgctggtgataacagcaatgaacaagaaaaatcaagtctttggtcccatggaacttacagtttgttggggggaaagacatgtaatgaacaataagcaaatactcaagatgatttcagagagaaaaattcaataaagaagatgtcccaggctgccatgattgaagggggttagctactttaacttgggtggttatattagtccattcttgcgctgctataaaaaatatacaaggtagggtaacttataagaaaagaggtttaattagctgatggttctgcaagctgtacaagcatggtgctggcatctgctcagcttctggggaggccttagggagcttttactcacggcagaaagcgaagcagcaggagcaggcatctcacatggcaggagaaagagagagatagagtgggtgcagagatgtgcctcattttacaacaatcagatctcttgagaactcactcactatagcgggaacagcaacaagccatgacggatctgcctccatgacccgaacacctcccatcaggccccacctccaacaatgagattacatcttggcatgagatttagaggggacatccaaactctatcagtggtcacaggagaccttttggtggagatgacatttaagctgagagatgagggacaaaggagccagccatgagaagatatacagaaagcatattccaggcagagggagcagctggtgcaaaaatcagaaggtggaaatgagttaatcatgttcaaggacgaatgtgatttcattcctctttttgtttgcgatgtagttgcatttgtcacgtagttggcagagaacatggtaggaactgagcatagagagctacactggggcttgttccacagttaagtgtcacacagttttgaacacgggagaaagaagtgacgcaattcctttattttccgaaaaaagcttggattccaggaagaccagaacaaagcaatcatcagggttattgttcagcttggggtgggaggagaaagctgagggtgaggagctctcatttattaggcaccacctgtcttcagtggcaggcagatgtgacttcatttaatagtccacatcagccctgtggataagatatcattaactctcctggaccaatttggaaacagagattaaggctgagcaccttgtttggcactatacgactaataccatgtgcccttcctttaggaaagctctgtctcctgaaaacacagttaactgggaacttacaggcaaacctcaggcatgtggtggcactccccaagaggtgctctggcacctcaaaagcacctgcccttcctcctttgctaccttatgacaagaaggcagcactgaactccatagaaaattatatagacacaaaaacatacacacatatattatatatatacttggaatatatatacacatacacatggagtatgtatatatggaatatatgtatacatggaatatgcatatatatggaatctgtgtgtgtgtgtgtgtatatgtgtgtgtatatatatatatatatgaaatgaatatggacctacttcattccatctccacctccatctccatcctagtacaggtttccacatggacaactataacaatctccgacctagcccctccacattcgctcaactcctgattgcatgcccaccggcacccccacaccccaaccaccaccatgtttactgcagcctgggagaactttacaaagtgaggttcatgtagaccttcagatgctcaaaaccctgcagtggctttcactgcctacggggtggagccccaggtcctgtcaaagatcaaggtgctccctgagcacttctcttacctctcatctcactgctctcactgatggtcttgcaactctggaaacttcaacgtgacctctcttccctgggtcttcacacacattcttccctctgtatcaatcaccttcctcacattcttcatctatctcactccaactcctccttcatgtggtggcttcaatgtacttgcaccatgattttgctaactatactggcccatcacatatgctcctaaagcatcctatacccaaagcaccctgtaagactcaacagggatccctgctctatcaacaccatgagataaagaactggttttatcttccagactccagcagagtttcaaaacactatctgcctaagtgaattacactatgacctcctgaatagtatggcatgagatgatcctctgagagtttctcttccagtcttgtatcctattctcaagtaggttaaaaagagccatgtccttctcctgaattttaacaggtgagaaacttcagtccaaccttcctttgctcggctaaagaatcacagggggagagtagcccaccatattgttcccagtcccatctgtcccccttgctaagttttgggagactgaacacaattttgattttcgtacactctccagcatctcctaggctccctctttgtctcttctttcttcccacagtgcttatccccccttgctctgctcccgccccctggaaccatctttttgtgtttgcattttccctcctgctcctccgtgagaaggatcccatttctaagactcatgctttgccagggctgcttggcatagggctgccttctccccaagatgcctgccagaccagcttaggcacaaaggcaggtgccaccagccctccctttccctcagcatcagttgtcaagagctctgcttccatgaagctaccaaaatagaattgtgctctgacctctgcttagcagggttgccctcctgatggggaaacctgcatgttacatgccagaagcctggcccaatgaccactagggtgctcagaggccacagggtcccaacaaagtgtggtgagaaaaaagccggcaggagagagccaggatacgagagagtgaaggaaggagctcagtgaggaagcagagagaagcctgggcagagaaaagaaaggaagacagtggctgggagggaatcacagatgggaaaccgaacatacagagggaatgaggcagaaaggagatgaggatctctctctctctctctcgtgtgtgtgtgtgtgtgtgtgtgtgtgtttggtggaagatatctatagaaattcttgttgagtatctgctccattgctcctaaatatctcatcaggactttcctgagccgccattattatttagttcccattttccagaggagtaaatggaggctcagagaggcaaaaaaaaaatttatctttggttctccagcatatagtccagtatcagggcataaagtagactctattcagcaaagtattgcatgaaaaaggaatacattaatagatttagcatgtattattacaaattctctccttttaacattatattttcctgtatttctctcaacaggaaacacactcaaaggaaagaccagcatactcaagtgaacaaatgtgatgtatttgtttccatatgttctgcattttgctggcagagattccagtgctgggtggaaggtcctcttcctggtctcatttgcatcatcagcaatggaggtcagcatgaccagcaggctgctgcctccatgaacatcttagtagattcctgatcccccttgtcatcggtaccctatgccaggttagacaatgggaaaaggtatcagtgacacatcatgtggggtttgaacatgctctaaaaatacggagaagtatacgcatgtagaaggtaattgtttttgctgttacacacttagttctataaaataaaagctgggaatttctgacatcatctttcataaactggttcctgatagcaccaagaaaacatggtcacaaggattacaagtctagcaagatcaggattggaaccccaatctttttatttctattcattcaattgaaacaaatatttgtcaggcatctacttttgtgtctgacagattctgtgtttggccctaggtataaagatggaactcctgtctttagaaggctagagccagatgaggaaccatacatcaaacaagggcaaacaaagaagcacacattgaattacatgctatgaaggaaagaagcgagatgccacaaggagatccactctgctgttaataatatacatatgtaactaacctgcacattgtgcacatgtgccctaaaacttaaagtataataaaaaaaaataatagggcttgagagtgtctttctaagaaattgaccctggggcatgaggaggcagattccagatgaagaggagcaaattgttattcctggtccagtgaccaggggtaaatataccttgtactattaagttatacctcagaaagaagaaactcaagaaaacaccagattaattttgattcaataatgaccatttgttaaaacccactatgtgtcagtatctgtctcaggtaacacagctaggaagtgccaaggtggtatttaaattaagatctgtctgtttgtgaaactctatagattttttttttatctactctttggatatgagtgtcccatggagaggaggaaaggggagcaactgtcagccttcagttgctcgggttaccagcccaaaacaagtctgaaacacagcaacctgcatatggagaattttctagagagattttataagtatctttccaaggaaaagaagttcaggtcatatttactagccttaggattagacacttaaaattagacacctagggtggtttagatttttcctcgccaaaactcatgtcaaaatttaattgccaatgtgacaatgttaggagctggtgccgttaaggggtgactaggtcattaacatggattaatgtctttctcataagactaaattagttctggcagaaatgaattaattcccatgagggtgagcatgcctctcctattttgctctctttgcatgcacttgcttgctcttcaacttttctgccttgctattaagtagcataagtgcttcaccaaatgcagctgcctcatcttagacttcccaacctctagaactatgagtcaaaataaacctcgtttctgtataaattattcagtcataggtatttttttggtagcaacataaaatgtactgaggcagaaaattggtactgatgagtgggatgacacctgaaaatgtggaagtggctttggaactgggtaatgggtcggggctgaacaaacttgggaatagcaggctggaaaaaagcctgtgttgcatgaatggagtattaagggtgattctggtgagagcacagaagacgagaaggttacagaaagtctggatctttttagaggttggttaagtggtcatgactagaaagctcatagaaatatggacagtaaaggccatttttatgagatatcagatggaaaatgaggaatatcttactgggaacttgttgtatagccacaaagaacttggatgcactgtgtccatgccctagggctttgtagaaggccaaacttaggagcaatggaatagcatatctggcagaaatttttaagtagcaaagtgctcaggcagatgtatggttacttataactgtttacagtgagctgctagaatacagagaagcagagtgaaaagatttgaaaaatccacagcctgagcacatggtagagaatgaagaacgtcttcaggaggaaatcaagggtgtggcccagagactgtttgccaagaagattaacatggttagaagggagctaggtaatactcatcaaaactatgggagaaagatgccaaggcatttcagagatcaaggttgtccctcccatcacatgcccagaagcctaagagggcagaatggttttctgggattgacccagggtgttcccaactggctgactgcccaggctacctagggactctgctccttaaattccagcactgggccccttggtcactccagtcaaagttccagtggccccaggtgtggctcttcctatcactctggaatgcaaattgcagaccttggtagtgcccatgtggtgctaattctgcaggtgtgcagaatgtaagagttatggaagcatggctttctctacctagatttcaagggatgttacagaaagcctgtgaacccaggcaaaaatttgcttcatgggtggagccataaagagcctccaccagttatacccaatggaaatgtaaggttggggccactgtcagggccccagaatacagccactagcagcctgcaatgcctgcctaagaaagccacagacaccaggatgtaactctacccagagacagcagccacgtggactgcacccagcaaagctgtagaggaggagttacctgaggccttgggagcccagccccattccagtgtgctcaggaggcaacacatagagtgaaagattatttgaggcctttaagatttaatgtccttccctctgtgtttcagatttgctttgggctggtaactcatttcttcctccctatctgttcctttggagtgagaatgtttaccctatacctgttctgccattgtattttagaagtagacaacttgtttttaattttacaggctcacaggtggaaggagcttgccttgagtctcagatgagactttggactttagacttttgagttggtgctagaatgaattaagacttttgaggctactgggatggatgacttttgcacgtgagaaggacacgaattttgggggtccaagggaggactgctatggtttagatgtaatttgtcactgtcaaaacttatgttgaaatttaattgccactgtgacaatgttgggaggtagttcttttaagagatggttaggtcattaagatgaattaatggctttttcataagactggattagttttcacaggaatggattagttcccatcccacccatgagagtgggttgttatgaggagaggttacctctcccattttgctcactttacattagcttgctcatccttccacttttccaccaactgatgaagtagcactaggcccttgtcagatatagcagcccagtcttgaacttgccagcctccagaattacaagccaaaataaacctcttttcagtctcaggtactctgttatagaagcataaaaaccaactaaggcagatgtttagattaaaaaatactttagatacaataacctgatctttctcctctaactttgtaatggaaacatttcaccatgttattgagattttatagaattattttacattgccgtgtagcagttcaaagactggctgagaatgatcacatacacaaataagatgcctttaaggggcagtgtaatttaaatgtaggaatataaaagggttgggtataaaaccagagagagtggttaaaactgaaaccaagctgaatatgaggcccaactaaagacattcaagttgaatttgttttaaatttgacaatccagaaattttactcatagatatgtactctccaagttgaaaacaggtatttaaacaaagtttgtacacaaatattcacaaaagtactattggcaatagccaaaagttgaaaacaacccaaatatccatgagtggatgaactgatatacaaaatgtagtctctccagataatggaatattactcagctatgaaaaggaatgcactactgatattttattccttgctacaatgtggatcaaccttgaaatttggtggatcattatgctaagtgacaaccaagacataaaatatgtctggtttgtatgtatgattttatctatatgaaatatctggaatagaaaaattcatagagacagaaagttgattagggattaccagggactggggaaaaatgagatgggtaataactgcttaacaggtacagggcttctatttgggtgtcagaaatattttgtaactagatagaggtgatggttgcacaacattatgcatgtagtgccactgaaatgtacactataaaatggtgaattttatgttattaaaaaaaccctcattttaaaaaaattgacaaagtcatatttctggctttggtcctgggttgccaccttgtgagctctagaaaccatgattttgttcactatttccttcttcttgtatgcataagttattttttgctttccacattataaacatggctgcaatgaacatctttatgcatgacatctttccatattttgtgttactcctttaggatagattcttgaaggcagcattctcagtgtaaaatatatggatatgtttatggtttttaatacatattgcaaagttattccctaataaaattggattaatttagccccccacaaacactacatgagaggtcttaatcacctcattctcatcagcattcccatgttactctttataaattgcttgtatggtacaggacactatgaggcatctcataattttgcctttgatcttgtaaaagtcatttctcaatattggcttcctttaggaacttcttctatataaacatgagatgctctgctgcttttggactacctgttcttcactaactccataagtcaccctcatcacatttgcccatgctgtgccagctgtttaaaatgcctctccttccccctctcggagctacaaattcatcaaggctgagactgtcatatttctctatagctttctgactctctggcattgtattgtttccacttctctactcccaaagccatttgtccctacctctttcatagggcagtgcattgtatgggtgtttttttttctcttacatgtctattttgttctctggatcttgagatccttcaatacagaactaatacttacaaaactaaaagctgaactgaccctactagcattgtccctgacccactcttccactggactcatctgtctaccgatgtgtccaggacatctcaacagcatggcaacatggtctgaatactcaagagccattcacagtgaaggaagagtggtattatttatgctatcagatggcaaggccttgtcaatgagacatgattatactacatgtgatacaagtgaaaaggtaggagttccaagtggcagaactatggttggtgttcaagaaattgaagtggatttcaaagtgattctggaagcaaaaatgtcaactgagctgcaaagacattgaatataagaggtaggttaaaaaatgcatcactaagacatgttctcaattcaaggttatgtggaaagtacatagctgggtcctcattcaacaagaatacccctcaaaaaggctggagggatagaaagaagctttgagcatcattgtatggcatgccaatttaaaatgtatggattttaatggttagcacttctcatacatcatttatatgatcaataatttcagtataaagtttcctatggctgctgtaacaagttacccaaagttggtgacctaaaatcagtatccttgtgctgaaaagagttgccagcagagccagctccctatggaagtctaggggagaatccttccttgtctcttccagcttctggtagctgctggcattctttggtttgtggttgtatcattccaaactctgcactgttttctcattgcctttttctcttctatctgtgtgtcaaattcctcaatgtctctcacctacaaggatacataagattatatttagagcccaccaggataatctgagataatcctcccatctcaagatccttaactagggtaacattcacatgttccatgggtaaagacatggattttgtttggtggggaggggaagagaggggattattcattctattacaaatatataaatgaaaatattctcagtcagtttgctgtaaagctccaagataagtagtgcatttatattgtttctctgcctcttttttatattacagacttcccaaaggccagaatgtgaccttctgagtctcttatccaacctgtgactgcatccttggtagcaagaagaaaaaaatcccaaaatcttcaaatagagctaagtcccagtgacacaattctacttgctgtctcttttgtagactcttccctgcattctcagaaggagcttcaacctccacatctgtagaaaaatggttttgggtacagcagactttaattcatttagctgatatccactaatgtagaggccaacacaaaagctcatccatgaaaattatctcatgtcctttcatatcaaatctgaaatcctaataaagagcagaacattcacaactaatactcctgacatggaaagcctgcccactactttgattagtcactaagttcaaacagttaaattatatctcatgtcttgtattccttgaggtctagccaaagactaaaatctagtagacaatggaggagatggtaggggatgagacaagagaagaaggtggaggccattccaggtagaagctgcttatatgtattggtaaggatgttgtgcttcatctcaagaacaacgaaactcactgaacgttggtttggagagagggttagggaaaagggtgagacaagaccctaactgtattttcacatggattactttggttgcagcgtggagtttcaaatgaatgaagcaagaatggttggtcaggggactgcactaatcctgacacaaaattatgttggcttgaactaaggggtggcattggagatggagaagtaaagacatcaggaaatatatgaagatagagacaagagggcttggaggatgtggagggtgagaaataggtttcgtggatgttttaaatttctggtaggaaaggatggaagtgccaccatgctgagttagaaaacaccggaacaggatgagatttttgaggaaagatgacattacctaaacagcaataacaataaggcaagtggatattcaggcagggagcagacacactactgtgagaaatcagtacctggaagaatactcgagtcctgcagatgggtgagatttcctaggcaccgtgttctgtctggaagggacaggcaccctcagatgataagggaccaacagttagagaggaagaaaaccaggagatacagtgccatggaagctaaaagaagacgagatttcaagaagtgctgaatactgttgaggtatcaagtacaattgcgactggaaactgtccactggatttaatgccatgaattgaggatctcagcaaaaatcatttttgtacagtaagccagactagggtgggaggaaagggagacagcaaattgagacaatagttttggggattcacagaatccagtaatgcatataaagcactcagcacagtgttaacacctagtggctgaacaataaatgttaatttccttgccttcctcattttaaacaccaattgactttcccttttggatactgacagagatgaatgcaaatgtttcttttcatggctttgcaaaagtaccaataaaagtgattttctatcaccaagatacagcctgctgatgtgaaatatacccaggcctgcttattattgttgttgttctccaggacattgccgatgtcaaatcatgtaaatctatattatcataaaggcaattaattaattgtatatctaaaagtgcttaatttttatgcctctgctagacttcttggacatatttctgaaccacatgcaaccacgcttagctcagacaataatattcccaatgtttatccattattgtactcaagattcatgttatctattttacttatgataaaatccacatgagatttcacttagaaaaaataagggcttttgctattcacacataaccagatgcagagaagcttttgagaccccagagccaaggcctccaaattgttggggtgatccattcatttatgccatctataactgcgtattaggaaccagatgagaaccttatgggacaaaataactttaaaaacagcaacattaagcctgtcttggaatataaaatgtaaaaagattacttggcctccataagttgcagataacagaagagaaataacaaaggaaatatcctgcagactgacacagagcaggagctctccataaggagatttttaataacttctgtctccaaaacagcccaccccacctttatcctatctgaatgccagaaattgttcattgcccacacatttaagagttggaatctctttttcacatattgagttataattcccaaatgatagcttcactgacctattactacttgaagtgatacatgctaaaataatcaaatatataaaaggcagaaaaagcttgagtctcactggggtgaagagctccttgcagatggctggacctcaagtgtcttgatttccccactctctatagtagggtattaataactgataacagtggttgaatgtttacctatgcatcaagaagtgttcttgagatttatatgcaccacctatgtaattctcatccaatcttaggagtatgaggtaaacattattcatccccactgtgcaaatgaggaaaaagaggttcagagagatgaagttccatggtaataatttgaagagctagaatccaaactcatggaatctgaccacagagattaagcacattacaaatgtttgtgggtggatatatggaaggaagtagcgtataagtggagatatggttgaataaatgcatggatggagagacagcaggatggagaccaacagaggaatgaatgggtggatggagagttggatacatggatattcatctgtcttaggttcagtaaaaaacacaaacttccaagggcactcgtgtggtgttatgatatataattggttttcatccatggttcctggctcctaactcccatagtccttgttataatattggggcactttgggcctcaggaaacagaatctctctctgaccttcttcatgtccttctttcacctgcccagtgccagactctaatctgattgtggatcagaagaccctaatccagagagattcctgccccatacctagaggaaggaatgctaaacagagaggccaagaaaagtttgaatggacaagccttgctggtgtagattatgcgctttttgtccaatcacatttccacagggttgtcaatcatgcctaagtaatgaagcctccatagaaaacccaagagaactgtgttcagggagcttccagacagctgatcatgtgaagatttctggagggtggcggtgcatccagggagggcatggaagctccacaccccttcccctagacctcaccttacccatctcttcatctatatcctttgtaatatccatataataaactgtaaatgtgtttcctggagttctgtgagctattctagcaaatcaatcgaactcaaagagggggtcatgggaaccccaactggcaactgcttggtcagaagttctagaggtctgaacttgagactagtgtctgacgggaggcagtttgaggactgagcccccaacctatgggacctgacactatctccaggtagatggtgtcagaattgatttgggggacacccagctggtgtctgctgcctggtgtacagtgaaaaacccccatacctttggtcacagatgtcttcttctgtgttaatgattgttgtgctatcagagcagaggaaaaacactttaagagagtttttccctacacgactagaaatgatgattgaaagctgactcttgttctttaatccagtttcacgtgagaatgagtcatcagtccctgcatgctatgcttcttattatgtgatgctgtcagactataggggaggcaggtcttcagactgagatgcaggggatgagagggaaggtgttgggtggggaggcgtgtcatatctctttgggtcttgattactgaagagtcaggcatgacacgtgaaatagtgtccagaaactgggtgataagttgttatctggaaatggcagcaagcaaatcacacaggcaagggtcttgctcatgtatcttgggattgagccagggagtcctgaaaaggactagaactctcctccatcaaccatgttactgaaattagattcctcataggttctgagattactcctctaatgttctctctcagggttcactaagggttttttgtgtgtgtgtggttttttttttttttttttgagacggagtctcgctttgtcacccaggctggagtgcagtggcgtgatctcagctcactgcaacctccgcctcccgggttcacgccattctcctgcttcagcctcccaagtagctgggaccacaggtgcccacaaccacacccggctaatttttttgtatttttagtagagacagggtttcaccgtgttagccaggatggtctccatctcctgacctcgtgatccaccggcctcggcctcccaaagtgctgggattacaggcgtgagccaccgtacctggcccactaaggtatttttgaaagacagtgtttaccccagtgatgatggagtgaatcagaggcaggggactgggggtgttgcagacattgttatgaggagcagggcttcccctgggaccaccttaaggagaaaaggagaatcaaagagagaaagaaaatctgaggctagagttaggaagaacagaatcaaggcttcttggtcctataggtcctcttcagtgggaaattctcattctggggctaaacctgggagattttgagatttactttctttacccagggacacaactttcataagttcttgtggaacgtttggtgagatactctgtattttgtaagctgcagatgtgtagacttaaggctgaccacattgcatcaagctggtcacactggcagctgctcacccagagtgatgcccaccccataggagtacagatcagataaactgctagcaaaaggaatacttatcatgttgagcaactatcctaggtcaagtattttacagagatcctctctactacttaaagttattgggcatttggcatatgcagaacattgaaactggacccctcccttacaccttatacaaaaattaactcaagatggattaaagtcttaagtgtaaaacccaaaactacaaaaaccctagaagaaaatctaggcaatacaattcaggacataggcacaggcaaaggtttcatgacgaaaacatcaaaagtgcaacaaaggcaaaaattgacacatgggatctaaactaaagagcttctgcacagcaaaagaaactatcatcagagtgaacagacagcctactgtataggagacaatttttgcaatctgtccatctgacaaaggtctaatatacagaatctacaaggaacttaaacaaatttacaagaaaataaaacaaccccattaaaaagtgggcaaaggacataaacagacacttctcaaaagaagacatttatgtggccaagaaacatgaaaaaatgctcaacatcactgataattagagaaatgcaaatcaaaccacaatgagataacatatcacaccagtcagaatggtgatgattaaaaagtcaagaaacaacagatgctggcaaggctgtggagaaatagaaatgcttttacactgttggtgggaaagttaattagttcaaccagtgtggaagaccatgtggcaattcctcaaagacctagaagcagaaataccatttgacccagcaatcccattaccgggcgtatacccaaaggaatataaatcattctattataaagatacatgcatgtgtatgttcattgcagcactattcacaatagcaaagacatggaataagcccaaatgcccagcaatgacagattggataaagaaaatgtggtacatatacaccatggaatatgatgcagccatgaaaaggaacaagatcatgccctttgcagggacatagatggagctggaagccattatccttagcaaactaacacaggaacagaaaaccaaacactgcatgttctcacttataagtgggagctgaacagtgagaacacatggacacagggaggggaacaacatgcatcggggcctgttgtgggggtgggaagggagagcatcaggataaatagctaatgcacacggggcttaatacctaggtgatgggttggtaggtgcagcaaaccaccatggcacacatttacctatgtaacaaacttacatgttgtgcacatgaatcccagaacataaaattaaatttaaaaaataataaagttattgggcacttggaatgtggctagtcatctgaggaagtgaattttaaattttatttgcctactttaatatgttccaatttgaatgccacatgtggccattgcgtaccatattgggcagtgcagaacagaacacttccatcatcacagaaaattctcttggacagtgctgccttagggcggcttttcagcgaggtcctgtaaaccctgttttgactgaagaagaaactaaagctgagatgtcaaatcatttgcccagcatgacaaaaagtgagggtagcaaagccaggatttgagctttggtctttctgactccaaagcctctttttattttattttattattattatactttaacttttagggtacatgtgcacaatgtgcaggtttgttacatatgtatacatgtgccatgttggtgtgctgcacccattaactcgtctttagcgttaggtatatctcctaatgctatccctccccactccccccaccccacaacagtccccagagtgtgatgttccccttcctgtgtccatgtgttcccattgttcaattcccacctatgagtgagaatatgcggtgtttggttttttgtccttgtgatagtttgctgagaatgatggtttccagtttcatccatgtccctaaaaaggacatgaactcttcatttttcatggctgcatagtattccatggtgtatatgtgccacattttcttaatccagtctatcattgttggacatttgggttggttccaagtctttgctattgtgaatagtgccgcaataaacatacgtgtgcatgtgtctttatagcagcatgatttataatcctttgggtatatacccagtaatgggatggctgggtcaaatggtatttctagttctagatcactgaggaatcgccacactgacttccacaatggttgaactagtttacagtcccaccaacagtgtaaaagtgttcctatttctccacatcctctccagcatctgttgtttcctgactttttaatgatcaccattctaactggtgtgagatggtatctcattgtggttttgatttgcatttctctgatggccagtgatgatgagcattttttgtgtctgttttttggctgcataaatgtcttcttttgagaagtgtctgttcatatccttcacccactttttgatggggttgtttgtttttttcttgtaaatttgtttgagttcattgtagattctggatattagccctttgtcagatgagtaggttgcgaaaattttctcccattttgtaggttgcctgttcactctgatggtagtttcttttgctgtgcagaagctctttagtttaattagatcccatttgtcaattttggaaaagtctctttttatttcatgacctattctatttaaaactctgatttcatgaacagtgtgctaatcaataattttccctgttcaagaaggtttaatgtggcttttcctagagcctttcccactacagcacaaataaaaactaaccctatgtcataaatgtactctaaattattttaaatgtatatataaatatgaatgaatcaataatggtagtgcagtttacattacaataagacataaccctctctgtggctgaatgtgatgaagggtcatttctgcctcgttcacatgacaagtccactgtggatctgagcagggctctgctggagaaggctacatggccaactctcctttcgcagccacccagactccccggctcctaaagctgctaaccggaactcatgtatcagttccattcccattcattagtcaaagcaagtcacagtgcatcttaaaggagaggaaataagtggtgaaagcactgatatcttctacaagcaactcattatcttggcatgtgtcagtttcttttaaaagtgtgttttaaattacatgaaatctttctttggttgttcagattctaaaatcacaaatattgatgcttttgttcccttaagaggctttgtccggcacccagactgcagagtgttctggcctttgtgttagagaagaggaggatgaaacagtatgtttcctgtctacagggggatgctggctagtagaggacacatgaagaaatcaaccatcccagtttgcctgggactcagaagtttcccagaacatgagactcttgagtgttaaaagtagaattcctgggcaaactccctctgcattcatggggtatgagatttgatgaacatcctacacaaataaacagaccactatactcctgtttgagagaattatgatggggagcaagagatgttttgaaaacacagagacgggccatctaactggagaaggtagggtgggggtccttaatcagggtgtggggatggaggttgctggaggggaagtatgtaccaggcaaaaggaacttcacacacaaatgtgtggtggccagagagaatgtgcctcattcagagaactgtgaatgcttaattatgcctggtgtctagaatttaacatgaagatggggtgggggagccttaatataccgattttagaatgtctgtaagataagtttatgatttacacatgtactcggccgggcacagtggctcatgcctgcaatcccagcactttgggaggccaaggcaggcgatcacctgaggtcaggagtttgagaccagcctggccaacatggtgaaaacccatctctactaaaaaagtacaaaaatttgctgggcatggtggcgggcacctgtaatcccagctactcggaaggctgaggcagagaatcctttgaactcaggaggtggaggttgcagtgagccgagatggcaccattgcactccagtctgggcgacaagagtgaaaccccatctcagaaacaaacaaaaagaattactcacttaaaaaaaattactcacttttaatttaattaaaattattttcaggtggaaatacattcatatgacacagagtttaacagggtctagagtagtctgtctcttattctgctatttagataactgttcctattcccataggcaaccaaggtttttagtttcttttttttttcttggtttataagtaaatgcatttttaaaaatttttaatttttgtgagcacatagtacatatatatatatatatatatatggtacatgagaagatattttgatatgggcatgcaatgatgcaatgcataatagtggtggtttttagtttcttacatattcttccagacatactctgagcagatacaagaaaatatgcacattttccccccaaaaattgtttcccacactaatgctactggattacacactctcctacatttggctttcttcacttaaaaacctaatgtggaggtgttttcagagcatttgtattcatatgaaagctggatccttgagcaattctgacaaacatccagggttgagacccacgggggatgtttgctatttcatctgtgtatcccctgtgcctggcaacattcatggcaaattctgatggaccagaccaagggaataaatgaacaaataaataaagtgagagaagggaaaatgaatttcaaaaagtgataagaatgcaaaagggggaaggaaggacccgattaatatttaatcctttggaggcatcttgccccaaattaaatgacatgccctggttttggaaaatatcacacaattagggaggtgccagtctgcttcagcctgtagatcccattgcctcttccataatcagatatgcaacttttctgagggcggtattttcctgcccttctgaaaaatgaagcaatttaaattctgtttataaggtcacagacttgcgctcaatgccaggcaatgacagaaatggtttcctgctggcagaaaccaagttggcttctaaagaggccatgtgctcctcttctctgctctcttagaactccctggacatctttctatttcagcacctctggtgggagattgacagtatctatttatgtgtctgattccctgactagaggaagtccctgactgaggacaaggattaggtctcattcatctttgtctccaataacatgcctgcaacacagcaaacacagtcaataaactgaggcctactgcaatgacaggtgaattgaaagaatgtgcctattaagggaaagaaaggacaagacaagataagaatggaccatggatcattctgtcatgggaggtgacaccatttttttttgcattttccctcctgcaagggaaagccctttgctgccgtttcatgtgcagaatgttggaagtgttggcctcccatatgacaggagtgccctgcacagggacaagaatacatgaacttgtttcatggtttctatttttattcctggtaggaagggctatttctgtttcaactctgaggggctgctgaaggaaggtacattgcagttttcgttggctgtcagaatgtggcacattttaaaaaccagaacctgctgagaggtaccccaaaaacctttcagggttttgagatggcagcgggaagtccagaactgtgggacacatcagtgtgtggcaccaggactctgtacccatagcatcagaaggagggtgtgtctcatgccctagcaggtgaggaatagctatgttttccaaaactacctgggcaagttagccccacgcaacatgcactaaaacagaacctttgggttttaacccaataaaagatgttttaaactgtatctcaggtcctggccatataaagggactccacctgcccaactctggcaggttaattcttttccatgaccccttgtgggctcatctgtagagtatcagtaccaccgatcactgggcatgatgtgagaattagggcagatgatgaatgagcagtttgtcatgccgaaagcaattacgagggttcactagcatcaagatggtgataattattattattaacttgatgccataacaagggattaagccagccaatgaagaaagagaatatcatccgtttagtcattttccaggtgccttctggctggctagtttccactgaatcccaagcccatcacccactctttaagcctaccccacgattcatcactcttcccttgccttaggccttttattttctgttctgtggcaggtaaacacagatttccttctcaagaatcagcgaatgtgctctatttctgatgcataaactctgaataacttctccacacttccagattcttcacacctcgctctggctcacccatccgactgtacgcccatctcatcatatcccactcctatcaggtcagcactcacctgttggctatgaaatgaaccctgtccactccagccaccatggccaccctccctgcctgtcataccttctttattatttacctagacacccacctggtcaataaagtcctactcaatcccatttccttatggagtctttcctgactgttgcagtccttactgtgcactttactctgaaatcgtaaataataacaggtccataattaaacaactaattaaagaagcatttactgaccatctactaagtggcaacaagggtgaagtgagaatgatggtgactaaatgggaatactgaaccaaggtcttgctgggtgattgatgggatcaggaaggtaaggaagatgaaggaacctaagatgatcccccagattctgccctgagaattggatggggtgctgccactcactgagatggggatacagaaatgggatcagactggtgggatggggatgcgagacagttgcaagcagggcacattgagactgagactgctgtgatgcacttggagagaggtgtccaacagaaagttggctctgtgaattttgtagtcctggagctcaagagctctctgtctaatgaaggaagcccacaaataagcccagaaattataatagcaagcagtaaatactacagacaaagtggaaacaaccaaaggagaccaaaggaaggacaaatgcctggggatattgggaagcttctaaaggaagtggcactcgagtggggctggaagaccagtccaggtttaacatgtgaatgggtagaaagagcacacccagatgaagcaacagcatgtgcaaatataccaagtggggacgaggatgggctagtccacaagtggcaagtgggtcacagtggctgaagagtagagaacgtgaaggaatccatggcaatgaagctggagcagatgaaagacctgccagtaatgacctctggaccatatctgacacaaagtagtttaagacactagatattttacactcgaatccagatttccctcttctactgaaagaaaaaggagggccagagctttcattttcatggtgacaatggtcaggcagagcagtggctactgccatcgaacagtcaggtctttcttgttggccacaactctcaaaacaacactttactcttgatatgatccacctcgcccctgttggcatttgcagtttgagccactgctggaactggaagacaatgccaaggaatctggacttatttccaagggcaacacaggcctgctgagggcaaggatggtccggctggacaacataatgaagacatgtggataaggtcatttcttttgagttccttctcctgaaagggttgaccccaaactaggcctgaatagaggccatcagaatgttctctggtcttgaaaagcagtttcctggcactcgatttttttctctctcctgccctgttaacaatgctgtatcctagatgttgagtcataatggctctacctaattttactaggaaatccacccatcccaagacctctcagcatggtggtcctttggcccacaggtgatttggatgtctagagatgtatgtgtctgtccaggaggcagctggctgttttataagctgctcaggagaacctattgaattgtatcacaatcttcaggcacttctgatccaccaccctcctggggagcatgctgggaaatgcatgactgctgagggaagaaagactcaagtcaaagggtagtaggcttaaaatactcccagccagagctattgatcatgcaagaacaggaaagggaaggagcccaagagggcagcaggctggcccaaattttctctcagcagagatcttgctgatctatagaatttaaacctataaaggcaatgaaggagattggcaccaacaggcccatgctacagcattactatttaggtgcttatggaacccattaaaacgatgataaagcactcatttgcaaatctttttctttgtacctactatgtgtctcccacagtgactcagttcctgtcctcaaagacctcacggtctatttaaaaatgtatacaactaaatgaatcactacaacacaatgtgtaaagtgcatccattacatatttaccaaatgttacgacattcacaactatggttgggtattatggaggcttcatggagaaagggagagtttggccgaattttgaaggatgagtaggaatttggccaagaggctgagaagaaggatgtctaggcagagggagcacttgtgatcagataaagaaaatgtgatatatatatatatggtgtgtgtgtatatatatatggtgtgtatatatatatatatatatatatatggtgtatatatatatatatatttatggtgtgtgtgtatatatatatacacacacacagccacaccacacacacacatatatatacaccacatatatgtacatgccacacatatgtgtatatatgtatatatcagcctcccataaaatattgcataaaatattacatgtgtgtatagatatacacacatatatacatatgtacacacagaaaatattatatgtgtatatatacacacatatatatacacatataatattttacatattatatgtaacattctttttatgtctgagtaatatgcagccataaacacacacaccataaaatattactcagccataaaaagaatgaaatcctgccttttgcagcaacatggatggaactggaggccattatcttaagtgaaataacttaaaaacagaaagtcaaatactgcatattctctcttgtaaatgggaaataaacctgtacacatggacacagagaatggaataatagacagtggagactctgaaaggtgggagggtggaagggaggtgaagaatgagaaatcacctattgggtacgatgtacactattggggtgatggttacataaaagtctagaattcaccactatgtaatacatccatgtaatgaaactgtacttgtaccccctaaatctacagaaatgtttaaaaatgccataaaataaaatgtggtatagccttaaaaaaatgaagctgcacacgtttggggccagtgagaactgggtccttgttctggctccaccaataataaccattgtaattggtgtccatggtcatcttaacttctccagggctcagttttctcatgtctgaaatgcagatagtaagagcagccctatagagttatttagagcaacactgtccaatacagtaaccctaaccacctgtggctacttaagtataaattaattaaagttaagtaaaattttaaggtccaattccttattcacaatattcatagtacaaatgttcagtggccacatgtcccaagtaggaccatattggacagtgcagcttataaaacattttcatcattgcagaagtttccattcgacagtactgatttagaggatgagacttttggctgtaaaagtcaccttgcatattgtttagtatataaacaatgtttaatagcataattatacctattattaattataataattataatttgtaaaggcctagagtcataattctcaaagtgtgaaatgaatgaaggaaagaaggagacacagataacaaagaattaatatcgattgggaaatgaggcaattagggagtaactgaaggaaaggaggaaggaagggagaaaggcaggcaggccaataagtgtgctggttcttctgcatttgttccccagagccatttctctctttcctgctctgctcagtgcctgggaggctgatctctgcagaccacttcatctaagctccctgtctggctggcttctgatagagtttggttaatggaagtgagaaataaaacagtcaccaatttgcaaaataaagtatgacctgcaaaagggataaactgatccagccatgacatgtaacacaaattagctcaaaacattcttaaataccagctcattcagtcccacatgttcccagctaatactacaaattgatgtaaccataactctgtatgaacttttatgctccacagataagaccactgactgtcctagaaaattgcttcattcataaattctgaccaatccctgtctagacaatgcatattaaaaccatcttaggactaaccccacaatcttacaagtaccttccctaactccccttttttgagacattcttgagattttgttgcaggactactcttccttgcttatcatatttaataaatccagcttagtaagataaccagtgttccctggtggtggttgcttcaacaggaggcaaccacaggagccaggagagcaggaagtagggtgaggcattgctttccatgcctcactctgtttcagtactgactctggctgtagctgtacctgcccaacccccaacagtagcttctgcttgggggccccttctttatgagctccaacaacatatcttcctcctcttgtcccttcaacttgggggcagtaatagagtttgaaattgctagcttctgccgcttcaatattcctcatttgttcccttatccttgcccattttctacacaacttcccctcattaaagagttttttggggggttttttgtttgtttttgtttgttttgttttgttttgttttaatgtgtgggcaaactgagtttctgaatgacagatagtgcaacttattatttattgtcaagcagtgtgctaagctcccttccctgggcatcttcttagacacacgcatgacaggcactcaaccccaatttacagatgcagaaacctaattcatcaggttaggcaactcgcccaaggccatgtcaatccatgtttagtgtgggaacatagacctttcaaattcagatatctgtgtgctttctactactctggactgaactcaaacgtgggcatttgaaaaatggttctctccttctctcaaatgagtgttctgttagctcctggaggttgttctcagtgtttatgtttttgctttcttctctctctcccaatcctggtaacattctgactttgcattaagcttttacaacttcccctcaggaactaacgtatcttctttactccctctaagaaaaacttggccaaaaaatcatctactgtccttgcagatctctgatcatccatctctggcttcagccttggaagatgccaatgtcctgaaacccagcatcaaagagaccagatgatgacttttaaggagggtggggtgccacccggaaacctcaaatggatcttcctatccaattcacaaatctgtttcccagcagtgtgtgcataggtgtagcacagctaaattgcatcagtatatgcacatctaaatttatacaaaaggggacaacactataggaagcaggcaagctagcaagagtcagtaagagttggaagttagtgtaaaggatagcaaaatcttcctcgaaggatgtagcggtaaacttagaaagatcaatatttaggggagaatctcttgagtaatagtccttaattatgcaaacacacaaatcatttctgggggaaaagtaatggaatattccctagcttaacaaaggcagactcttgacagcatttttttatatcttaagttgagaggtggtggaggaatccacataaggagtttttggggattccagatgggaacagaccaagacagaatacaaagtgactcagaagctgtgggagtcttggctagtgacattaatttaatacctgggccacagagtttgggggtgaaagctggtcccatcattacttatagctgaggctgattcagccacctagctatggaattctttaccacaaatgactgagcatataaagctgaaatcaaagagatgaaaacttggagcctgagtttgtggtaccttgggagcaagtgttagaatctgggccaaaagtagcttcagcgcaggggaaataagcagccactgaactgtgcaggacctgtgttccacagtgacaccatgtggcagtgagacaagagtggactaagtacactttccaattttcttcaaaggagcattaaaagcaagactggctcctgttttgcactacatacgccccttaggtttgtggctgattcaagagggagctagtggtcaccccagtgataagtcataaactggaaaagtagaggtaaagtaagctacaacaactacactgagaaacgtggggcttagttcttgggttctaacttgctgtgtgtgacttaggccgaatcacttcatttctcttggctttatcatttcatctttaaaaagagcctttctagattcaagacttcccagaggaccatgagatccataacaatagagacaaagttttgttcactgcagtatccccagagcccagtggacagcacaaagtactcactcataagtgtttgtttaattgaatcagattaatgatctcaggcatgaactattccccaacaagcttagccttgaggcaaatgcaaaagaaaaaacagatttgggccaggatattggtcactaaaggagtcacagtagtgctgagttcatggaagatggatgataaatgttcatcccattcatatcagaagattagaaaggttatatgagtcggggagttgcacagctccccacacttccatagggttctctttatgttccctggtgacagaaatgtgtccaccagcttcttttccttttggaccagctgtctcatcctctggtccccggaggatggcaacccttgcctcctgtcccagaaacaagcactaaagcttcctggggcttctctgagagttttcagtgtcaggcccagcagggagctgcaggcagggagaaagaacaaaaaagtaaatgatatctcctatctcacggggctcacggcagaagccttcccaggtttgaggttgaataaaaacagagctgggctatgaggttccacctgactttgagtctaggtccctctcctcctccatgggctcctgagtccaccggctttcttctctatcagtagctccaactgcaaccctgccttcccgaagcctgaacaagtgagaaagagagagcagataaggaagccaggcagaatctacagtgacaagtaccattagagagggatggatggagggcagggggagcccagagaagaagataacaaactcagcctggaggggaaaggcttgacaagggacctgcatctggaggcaatagaagcattgaggagccaagaagccatatcagtgggaagtacaacagcaaaaagaaaagccatgttctcagagttatttatcatgcttcacatgaggggcacacgtaagggaaatcagacactgtgattcaatggctgcatgcgtttgtttttctaattattattattaccatcaaggggagccataataaccacaaagggtaagacttgtcaagcgctgacactggggcagccgctgtgattgacctggtttatttgctcatgtaattctcacaataaccccatgacagaagaatgttatcatccccactttaacaatggagatactgaggatcagaggggttaagtaaactgcttgagctcccacagctagaaagtggcagaactagaatttcagaccaagtctatctgactccagagtctaaattgttgctatttgtgaaggttaatattgagggtcagcttgattggattgaaggatgaaaattattgttcctggttgtgtctgtgagggtgttgccaaaggagattaacatttgagtcagtggactgggagagacagacccaccctcaatctgggtgggcaccatctaatcatttgctagctcccctagaataaaagcaggcagaagagcatgaaaagactagactggtttagacttctgacctgtatctttctcccatgccggatgcttcctgcccttgaacatcagacgccaagttcttcagctttgggacttggactggctttgtgtgagtcaatactccttaatatatactccttaataatgtcccctttatatttacctctatctgattagttctgtccctctagagaaccgtaatacactattattatctcctgctgctcattaattcattattccacaaatatttattaagcacctactgcatgccaggggctattctctgccttattatgctgatttattgacatttgagttagaggcaaatttccagggtcacagctactgtatggagcattatgcacactggaagatatatgtgagctaatgaatggctcaactccctttcaggaccggcaccccaaacaccctggactacatccatgagtgtgcccccagaaaatgaggcacctcccagcatcgctgacaagcgagctgcaacaccaaatagcaaggaccacacactagaaatcccctcaccacatgtcagctaaaagaatgaactagtaactgcatgaccaggatttcaccaacacagaactgggttgaagaagccgacaaagacaggacaaatccccatccctttaatcacaaaatcttacaaactggtcccctctgtgagagtttgaatcccagctatctcagagagaactctgagctgtgaaatactaaacttggaagggccctaaacatctttgagactttcccaaatgtcagttggtcagataccccgtcaccatctttgctttgtttaggcatgacttgtactattaattactttaggtattgttttaaatcaactttcttttccacttcaatccaattacagcataagagaaggagactttatatcatgacctttaataggaaagccagaccttctcatataaataggttaccataggaatagatctactggcctcagaatgtgactgagttcaaagcaatttaatatatctccgtgtaccccatattcaagggagcacactgaccagctgagtgggaatgagccctgctcagatccctgggtggatgctggtttactgagactggccgtgcagacccaggatgctttcactgctgttttactgcttggtagctgcctgtgtgaatagctgtgcaacctcgggtccatctccatgtaacataaagatcccccagctcccttttccattgcaggtttctctctctctctgagcatgaatctattgaccacagcttcttaagactcctctcactccagtctccacgaggagtgcaggagtgcggggcaaagcgccccactgggaggacctgggcaagggtaacctacaggaggagaggtggggtgggcagcagaggacgagttccactgcctgggctggtcttccacttctgtgactgactgagatgatgctgccctccctgtaaaatgagccgtcaaagagcacaggtgcttctcaatgtgggcctgggcaccacaggtggcaccacaggtgactggacgccttcacagctgaacatcttaaatatgtttaagttaaactcatcactccacccctagaacatctggaatcctaaaagtggtatgtgagctgagtggtctctgtttttaaaagggttaaaagagaatttgccaaactatatccgaataaatcttggaatggattctttgaaaaattctgcgtggtttattaaagtgatgggtttgaggaaaaatactggtgaccactgggaagcagcagatgttcactataaacaaatggtatcagagtcaccttacgacttttttttttcttttttttttttgagatggagtctagctctgtcgccaggctggagttcagtggcacgatttcggctcactgcaacctctcctgggttcaagcgattatcttgtcacagcctccccagtagctgggattacaggcaagtgctgccacacccagctaattttctattagagacggagttttaccacgttggccaggatggtctcgatctcctgacctcaggattctcccacctcgccttatgactatttttaacaaggcaagcctggtagattaaagtgatttgtttattctgacaacgtttattgaacagctacaataggtcagagagtttgttgggtgctccacataagccatttcatatcatttgctttggtagggcatgacagcaaaaagataccaccaaatcatttagaaactgagaaaagagattgtcccgtttactaatttattgtaaaagtaagcaaacagaattggaggacagatgtgtaaaccgtccttaaggaacagaaaggtcaaaatgatttagcagctccctgtcctgtgtccttccacttctcattaaaatgccaccaaggacacacacaggaagaagccaaaatctcctaacttacaaacaccctgaaaaccaaaagtgataagccaattattaccagaattagggagaaattttcattgactgtatttgaagaattcacatatgtgaagtgcttagagcagtgtctggcacacagaaatgccaattgttaattattattaacttaaagtcaaaggaactgaactgaggaaaaaaattggtcacctctgaagacactgtcctatgaaagagaggccctaccaggcagagaggtgctgagcagatgtagttgagctgaataatgtcccccaaatttgtctattcagatcctcagaatgtgaccttatttggaaagagggtctttttagatgtaattagttaaagaggaattatactggagttgcgtgggaactaaatccagtgactgatatcgttataaacagaagaggagaggagacacagaaactcagaagaagtccatgtgaagatgggggcagagatggagtgatgcatctacacaccagggaatgtcgaaaactgctggcagccaccagcagcaaggaagagtcaggagaggacttttcccaagagccttcagaaggagcgtggctctgctgacactttgatttcagacttcttgtctccagagctgtgagagaatacatttctgttgttttaagtcactcaggttgtggtgctttgttatggcagactcagaagaggtatctgtccctctactgcactggcagaggcccagggtctgcattcactggtaaaatgtgccatatttctgctttttggtgctgctttatgaatcagcagatactgttttcagtcatttccaatcttttcagctccagggaaaagtcaaaacagagaacagctatgacagactatgtctgggggatgcagctaccacaggcagaaccttgccttgggggtgaagacagaaacaatatggggggcttcaagtgcagggggttgaggtctttcatgtgctccaccactggatcaatagaatggtgatatggtttggctctgtgtccacactcagatattatctagaattgtaatccccataattcccatgtgtcaagggagagacctggtaggaggtgactggatcatgggggcggtttcccccatgttgttcttgtgatagtgagtgagttctcacaagatctgatggttttgtaaatgtttgacagttcctccttcacactctcgctctccctcacctgctgccatgtaagacatgcctgcttccccttctgccatgattgtaagtttcctgaggtctctccagccatgtggaactgtgagtcaattaaacttcttttctttgtaaattacctagactcaggcagttctttatagcagtgtgagaatggactaatacaaatggacttcttagaaatggaagctcccaccaccattcatcaaaactgcacaaaaccattgtctgtgaggctctgggacctgagctcagcattatgcacatcaaccttccagccggacgtctgagagaaagctgtgtctattaaggtggcagtgagcaaggccaacagcaatccacgtaaccctgcctgttggcaaatgctggcctagaaagacactccctgtttacagaaaaattaaggggagaataaagaatattgtggtatgagtggaacagaacattatcttgaagtcttaaagagaatacctatgaaaagaatgtgctacagaagcctgaagaatagtttagaaaaacagaaaaggcttttgttttgcaacaggatagagaacctggtctctgtgaaacagaaataaagagttacatgaagacaattcacaaggaaaggagaactgggtatgataaaaggcaaatatctacatttataacatgaatgcaaatttttttttaatgttaccatatggattgtggaaagatattttagaagactaacccacaatgacttaaatagagatttttcaggaatgctacaagggtttagagtcagcaaatcacttaattatttgtatcaataagacatttgatcaaagtcattatctattcttgaaaaaaatctagtaaactgaaaatggaaggttattttgctgacatgaggaaatatcttacttctgagctatgagatgggatccaattaatgttattgacaagataagtacctatattatcattacagtaattcaatattatccttttgaaaaatatagtcagaataattagaaccagaaataagagttctggcttttgcaaaagcaaagaaaaatatatcattgttttcaataatatgactgtctacctccagtaacacaggtaacatttactgaatcgtcaacaggcccatgcactggttaacacttaataagttcccagccaaatctccacagtaaccttatgagataggtgctatgattaaacacttaatgaacagataaggaaagttaagaatagagaggataattttgctcagaataatacaatagtaagcacagagtcaggtaagttggctttaagatccatttctgaatgggtacttccaagcaaatcaactgaaaattgaacaaaacaaacagcaactcaaccaagccaaaccaagcattataactagtaagacaaggctgtgaagtggctggatataaaaataaatactcccaatgcaaaacatttcctaaatgtaaacacttaggaaagataaaattttaaaaatttctatttttattaacaaaccaacaacaacaataactccagcaaccatatgatgagggaataaaagtcccgacattaagtcgtcaggtctagaggaagacagacaaaggaaaactatgcttcaagaaaaaaagactaactgtgaagattttgtgaatgtcccagttaattttaggcttcatataattcctaacaaaacccaaagattttggtgaagacatggggaaactattctaaaatacaactgaatatatcaaaagttcagaaaaacagagtaataagaggggccttgccctaaaataaaatggcagttattaagtatctttaataacaaaaacaataaggtcaaaaatggttatctatgtaacaggaattattgagtagaggcaataaagtagaatacatggaaacaggtcttaagaatataaaatttagtttatggtgaaggtagcagcactaatgggtgaaggaataagaatgatccaataaatgttttccaatataattagcaaaagtaggcgttttctctaatccaaataccaaaaaacactaagagtcctaatagaggttatgatgtttgagagaagtcttaaaatatgagcaggattatatcagatgaactagcaaggtaatattcacaataggcgggaaaaaatatgcaacgcatgctgtgatagaaaaacaacaacaaccatattcagacattcgcaagaaattggttagatggtgtgacagggaatgtcaagaaagaagaccataaaggtaaattggacctagttatgaactttagattctatctcacaagggatggagaaccaaccgctgaataattttaagcagatgagtagcatgttctgcttagtgttttaggaagatcacttagctggcaatgtggaagagaagtgggagaaatgagaaattgaatatatttaggaggctgatataatggaattatgcaacagggcaagaagaatgtggggaaaacgaggggaaggaatggagtcgagagctgcttaggtggtagaaagaagtagactaatggcaaactggtaaaagtggagtgtgagaaaaaaggtgagggtgaggtgtgagaagaggtcagtgtgatagtgaagaggaggggaaagtcttgatttctgcctggggggccgtagggtcactactggagatgaattcggggagcagaagggataatggttaggatatgttttatgggtcacgtagctgaaggagctgagctatgaccttggctcggggaagggaggagtgtaaaagctcatactcccagggtttgatttgtccaatgactactttaaaagaaaagtttaagtgttttttttccaccccctccccaactttcgggttgagatatagggtctcttctataagtagtttcttcaaattgaaagataacttatttcccatgacatcccagtttctgtttgtttttccttctgcaagggcaatccacattctttattttccttgtgtctgaaatatttctaataaattagctaacttatccctgaagtcatatccttcactttaataagaataaaatgaggccactctatgtattattaaacttcatttcaggctgaaattgaattataaactagccatagttcctctcatgtatcacaagttattgccctatgattcatgcttctgaatggttttcaatcttttagaggcagtagggaaactgtcttaaaatagcctgaatatagcgcaatgaatatttttgagaagtctgtaattttggaaacttttatagaaacatttttactaacaacgaccctggaaaagggaatctgcggtaggcttttaacgtgtgagttactgttgtttccttgcttatttttttttcctgaattactggtattttaaaagactacatttgtccccagataagccatcgtgattaaatgggacccttttatccaaaccctaagatctcagtggtgcactgtggcataagctctcttggcatcattctgtcctgaaggattacatttacatgttcaacttgcctcctctccgctgaagagttcctatcttctccccttctgagtcactctgtcacaaattactcaccacctcacagagtgaggtgttaggaagaataaagtccctaaagccagatagacccggttagaatactggctctgccaattccttagttctgtgacttggggcaagatcctgaggtccccaagcttctgatttctcattagtaaagcaggaaaaacaggagttccttcaacactggccactgtgtggctcagtttgtgatcctggagaagtactagcacagcatctagcccagagtgattgtgaaattctgcacacatctgcacatatgcatgggatcattgagcccaagatcaagtgctgctgcagaccagctgtgcagatttgggcaagacatccaccctctctgaagagatcaccagttgttttcctcagttcagttcctttcactttaagttaataataattaacaattggcagttctacgaaccagacactgctgtaagcacttcacatatgtgaattcttcaaatatagtcaatgaaaatttcttcctaattctggcaataatggacttatcacttttggttttcagcatgtttatgagttaggaaattttggctttttctcatgtcgttggtggtattttaatgagaagtagaaggactctggacaggggctgctggaaagaaactatgttaatctttctgttccttaaggacaatttacacatctgtcctccaattctgtttgcttacatttacaataaattagtaaccagaggacaatctcttttttcagttcccaaattatttagtggtatctttttgctatcatgccctaccaaagcaattgatatgaaatggtttatgtggagcacccagcaaactctctgacctattgtaactgtttaataaaggtttatggaataaacaaatcactttaatctaccaggcttgttgtgttaaaaatagttgtaaggtgactctgaccccatttgtttatagtgaacctctactactccccagtggtcacctatatttttcttaaaacatgcctaaaaaccatcaattttcatgcagaatttttcaaggaatccattccaagatttatttggatatagttttgcaaattcacttttaactctttttaaaaacaaagaccactagctcacatactacttttaggattccaaatgttctagtgggtagagtgatgagtttgacttaaacatatttaagatgttcagctgtgaaggcgtccagtcacctgtggtgctacctgtggtgtccagtcccacgttgagaagcatctgtgctctttgatggctcattttacagggagggcagcatcatctcggtcagtcacagaagtggaagaccagcccaggcagtggaactcgtcctctgctgcccaccccacctctcctcttgtaggtcatccttcccaggtcctccctctggtgacccccgtgaagtgctttgccccgaactcctgccctccttgtggagattggagtgagaggagctttaaggagccatggtcaatagattcatgctcagagagagtgatatctgcaatggaaaggggagctgggggaactttatattaaatggagacgggcctgaggttgcacagctattcacacagatcagtggctaataaatacaatgggcagctaccaagcagtaaaacagcagtgaaagcatcctgggtctgcacagccagtcccagtaaagcagcatccacccaggcatctgagcagggctcattcccactcagctggtcagtgtcctcccttgaatatggggtacatggagatttattaaattgctttgaactcagtcacattctgaggccagtagatctatgggtatggtaacctatttatgagggagtctggctttcctattaaaggtcatgatataaagtctcttttccttatgctataattggattgaagtggaaaagaaaattgatttaatacataaagtaattaatagtacaggttgtgcctaaacaaagcaaagatggtgatggggtatctgaccaactgacatttgggaaagtctcaaagatgtctagggcccttccaagtttagtatttcgcagctcagagttgtctctgaaacagctgggattcaaactctcacagagggaatctgtttgtaagactttgtgattaaagggatgggaatttgtactgtctttgtcagcttcttcagcccagctctgtgtttgcaaagggcttgtcatgcagttactagttcattcttttagctgtcatgtggtgaggggatttcttgtgtgtagtccttgctatttggtgttgcagctcgcttgtcagcgatgctgggaggtgcctcattttctggggcacactcatggatgtagtttagtgtgtttggggtgccggtcctgaaaaggagttaccatcataccaaaatgctaaaaggaagggaacggatgtgatgtgttcactcactgaacaatttgctgagtgccaactctatgggaaacactgcgttaggcactatgggagcatcagtcgaccctgaatccattttcctcaggaatttttacctctaaagatgtctagatcccatgaccttggagaagtaccttgaattggctagagaccaactggaatgttagtgcaggaagctgcccaaggttggagacttaaaaagtactgaattctgaagactaagggggaaggtttcaggaagactgtgctagagcagggtagaactataaccccaaaaatgttggctccagagaatttaaaaagtgggataaagcaaggcaatacagaacttgggaaaagtaagtgaaagggcatgtctgggggaaaggagggcatgttaagggagtgatctcatttctccatggatcccagtgttaggaaatatacaaacaacaatcacttctgtgcatcaaattgcacagataaaatattttgggggcaaactgaggctcagagagattgagtagcttgtttaagctcagatcactcatctgacccaatgggaatttgaccctaggtttgtctaattttttgtattcccacagtgatgcctgtttgtcaacaggctcacttcaccctctataggactctctgaaggcaaggtctgtgggcttattcttcttgtcttccagcactggcaggctcaatacttatttgttaagtgaagtgaaataataaatcaacacgcaggcgcctttagaacaagcctatgatcagaacagagaccaaaggcagatttctcaattatctgagacaggctgcccaagactctgcagaccccgaggttgggctgaccctcgtccgatggccccgggaataaaagagtcatcgagctgccacgacgaagcagccgcactgccagatgatgtgatggatgatggccacaagtccctctggactcttgcatgctcccccgcccatctataatccttggatcactgacgcagcatgatgtaatgggctttccatcgctctaatagtctggcctgtcagtgtggatttatctcaacatgtacagggacatggggcctgaagcctattcaggactatatgatggaagagctttaacaaaatgaagtgtctactatggcagacctcaatctaaccttctcagctccccagatgaactccctcccaatccccacagcacctatggtggctagccttggacaaacttagctcccttcttgaactacctggtgaatgaatgaatgaaataaaaaatgaataaatgctagatatgaattccaataaacccatagattttatttccaataaatagatggtacacacatctgtcaatttggaaagtagttctttgggaaactggtatgtggttggatttgtacagttcccagtttctagaggattttcatgtattttaaccagtaattaagactgtgggctctggaggccaaatgtgaagactagaatatcaggcttataatttaccagatatgtgaccttgcacaagctactaaactctcagacctcagtttatccatctgtgaaatggggttgacacaagaaataacagagataacacagataaacagcatgatcttcaccttcttatctgaattctggccttctatggagatggtctttcttgcctgttttgccgatgaagcaactgaagctcagggggattatgttatttgccaaaggtcacagtgtcaccaaaacccaggacagctagctcaccacagtccatcagccagagccacttccaatctgaaatgttcccaaattctttgtaattcaggtaattcttcctgtccctattaccccattcaattctgagggctataaacagctcccactctactcagaggagaagctggaacttgatctcctggaaggaaatctaatacatactgacagccccacttgctcagagccagagcccctgaaattacatttaacaccaagcagaaaagggaaggaagggcaggcttaacgaatccaaggctgctgttatttataacagaagagaaaacaaatgaatgctggtgcctgatcatgtttgccacttctcagcacctctgtccaaccagcagaagccctctattcacgtgcaacagtgagaacttccttttcacccacagtccttagggtttttcagctctagctgcccactggaatcacctgggtgcctttaaaaaacacataagtatgcagaggtacatgaaaactacccatagagagttcatattcaaatggtctaagacaggacctggtcttcattgctttctgaaagctcccaggtgatttgaatgcacagcccaggctgagaccacagctgtaggttgtggttattgagtagttgattggtttggaacatagagtcatctcatgttgtagaaattccatttcagatgaattcagtcacagaaactcatgatgcccaatcataaggtgttgtttcccaaaccaatccctcacccacttccagttcttgccaggttcttagtgccctactaaaatctggataaagccctccattttgcctgcataaaacctagccttggcacagccatctcaccctgctaaaactaggccatgacacaaccatattgtagaagcccttggtggatcacgaacactattttctcctccaacctccaacctgacacggaatacatacctgaaacacgcttcctccctctgcttcctcattgaggtcctccatgcctgtgccaccatggccctcctgcttctgtttgctctcccttcccacacagacacacaaggatgcttctcagaagtagccaagcatactgggtcatttcctggttggctctgagtcatacgggataaaatgcaaacttctcagcaaagcacttcacattttcacctagtttcctaaccacatttttctggggttactccatgtgatttacaccctagccaaactcatctatgttgcatttccagaacacaccattcactaccacacccctgtgctttggcccaggctgttcttttcaccaaatatgccctctacaccagtgcacctggtgaaatctgacttgttctttaagatctctgacacctcagggcccacctccccacctgtactggccgggccctgggcagagtctcttccagcattactgttcacagtcccccctactgcccaatggaggaggaggtgctatttttatgcccacatgagaagtgtttttttaaagcacattttcttgaaaatgacttggatgtggcctgaggacaaggtgtggcagaagaaggagagcggagaaaagaagtgaaagaagaggggcggagagagataagacggttaagtcatatgacgggtagtgggagcaaggggctaacaaagagactttagcttgcaattcttcggtggcatcttcaacgaataacagagaggccaattaaccagtttgaattgtttgctgtttagtgcttgctttgcttctctcatctctgctaattccctaaactctcagcaaaggtgactctatatgcaaatgccccagagagtcatttggaaagaaatcaatgcaaggggcttggaggacatccctgctggctcagagcagagtgatgaacaggaacagcaggtgaggcagaactgagatgcagaatttactggggcaggaaactcagagcccagtgacatggagaagcccatctcctctccaccccacacctcctgagctgtaaggacatgggcagaattgaaaaatagccctctggtattccccgcatgtgtgcatatgtggaatagagggaggtaggtcctaccttttatttaaatctcaaagatagagtgaccccaacaggtacttggctgatctgccagagcatgagtaccacacaagccaggctttcagacaggcgaaactgggcaggcagacgaatgtgcctctatactcacttgctcagctcaaatatgtttgcatgggaatcttggggcagaaagctttgtcctcatccctcagatgtcggctcaagactatacaggcttttaagcctccaaataaccaaggaaaaacaatcaagtcatgaaggcctttgaacacagttggtgatgacccatctgagaatagagagctccaaaataattacatttccccatgcagcccacagaaaagtgccaagtgcaccacttgcaaggttactgcgttggtgctgagtcgagcagtttgggccctttgtttcagagagcagtgcttatgtctgcaagataccaagtactcccagcagtgatccagctggacgtgcttccctgcccatcacggatccgggaagcttcctccaccccaaggaagcagggaaggcagttcacatttttaggagaccctatgccaagcactgaggcccgcactctttattaacagctcagtgccctctgtcttcttcctccaaggccaaatcttgattatgatcacttaaagatgagcagaggatgaggaagagatctcctgacctcaggataggggactttagccccatgggtccaatccaaggggcttgcaggccagatccaaccaatccagcctgccacctgcatttgcaaagttttactggaagacggccatgccattcatttacacattttctgtggctgagttcatgccacaacggcagcactgtgtagctgtgatacaggttatatgcggcccacaaagcccaaaatatttacttattctttggtcgcttacagaaaaagtttactgacctttgacatgacttgacatgtcccaaattgtattgttttcccccaaaacctgctcctagcctgttttagaaaatggccgtaggtgctaaaaccacgattgggcttattgctcgtttccatcctctctctgacttcccatttccattaaattaatgtgtcaactgattctgtaccaaaatctgtatttctactatctgtcaccttttttcatagccctgctgtagtggagttgagttctgtccccattctcaggtaagccatcaagacaactgtgggtctcggccttgcgcacctcaattccactgcccctctcctgcagaagtagtcttaaaaaaaacatatctgattttgtcatttcctccttacgatacttaatatgctttcatcacctacaaaaggaagcacgcgttctgaccttggcaatcaagaccctccagtctgacccttgacgatctcttcagcctcctctcccatggctacttctacatcagcagtgtagggttcagaaattccacactgcttacggtttctctgaagccatcatactgtttcatccctccatatcttagttcttcatgctgttccctcagtctgtaattccctctggataatttcaatctgaaatatccttcaagccattcaataccatgttcaaatgtcaactcaatcccaggaagacttccctgatgtcctgtcccgtcagcatggtcagttcacctattctcctcctttagcttatacatgcttcagtttttgtgcctttacttcattctaatgtccttccttggatcctctcttctcagcaaaactatagaattattgagaccagggaccctatttatatgcctctgcatccctaatctctaagcacaacatctaaaactcagtagatatttgttaattgaattaagtgaagaaatgtgtgaataaacaaatgggcgaatgcagacacagtcattatccccacattgccatctaagaaaactctttccactctcaccctttctttaccccagtaacttgtctctaaaaaagaaccagaagaaaaggggagtggacacagtttaatatgaggactaaggctaatgaccaagcagaatttccttttccattaagcaaccatttgttgagcacatactatgtgccaagcatgtaccaaattatttacatataattatctgaaaccatcgcatggagaggggtaaggacaatgagtagatcagccccatttttataggctaaggctagatagtgacttcccagggtctcccagtgagctagtgatggctccaaaggtaggacccagctctgctggctccagagcccggcggttgccatcataatgtgctgccttcctgagctttcctgcaactcagctccactgagttctacttgttatttaattttgtgtgaagcaaagttagaggctcgccagcctgaaaacaacccagaataattttgtattcgctgtcctagttagttttttttttttttctttttaagaaaaaataaatcagggcagcttgttccctggcctcattcccactgtttctaggattgccacacaacacagcttggtaggagccctggagtgggcagagctgacgcccacatggtgataggctggctttctcgagccttgtcctggggtcctggcggtgtggatggggaaagacagttagtcagtcaataaaaggaaagaaaaccacctaatcatgagagctgcagatggacttcataaaatgttggactcacgggatgcaggagagagaagtgtgttgggggtcaagagagctgggttccgtccagggtctaatataggcttggggcatttgtcaagacctttccatttctgagccttgatccacacctgagaaaagagacgactggctggtgtcatgggctgaactttgtctctctaaaattaatacgttgaagtcctaacccctaggacctcagaatgtgactgtatctggagatagggtttctaaagacgtcattaagttaaaatgagttcattagggtgacctgctccaatatgactggtggtcttataagaagatatttgaacacagacataaacagaggaaagaaaatgtgaagacacagggagaagacagccatctacaaggcaaggacagaggtcccagaaaaaccaccctgttgacaccttgatcttggacttctagccttcagaactgcgggaaaatacattcccgttgttgaaatcacccagtctgtagtactttgttttgacagccctagtgaactaatgcagctggatgcatcagacatccagcttctgctccaagctatgtcacccaagtggatgtgtctaattctgggatctcaactggagagaacaggatcatgagaggccaaaaaaggtagtcaaatacagaactgaaactatttaggtctgagaagagaaaactctgaaagaacaaactttccttcgtatgtcgaatgtaaaagggacaatatttgttgggctttgtgggaggcaagtggccaccctcaatggctactgccattgagagagatatgttgtctcagcatgaggcttggatgtgcattactttgaacttcctgtctttgggggcattgaagcaaaaacccaaagacaatgtggtgaaggtgatgctgagggctcccactgtgtcatggtggaaggaggaagtggcatgagtgcagaaggtggcgtgacatacaaaaccacttcacaaaactttctcaataaggagacaggctaccaaataaaggcataggattcctgtgcctggaagtcatcgggcagggttccaaggccaccaatagcaggtggagattctgactcttggtcaaagcctggaacttcatccatcagacaaatcttttgagtgtccattgtgtgttaggcacagtgcaagagaaggaaagatccagtccctgcccccaggcagttaatactcactccctcatccacagctacagtttgaatgtgtctccaccaaagttcatattgaaatttaagccctaatgcaacagtattaagggatgattgagtcacaaaaggtaccaccgtcatgaatgggattaatatcctgataaaagagctctaggctgaagggaactatcttttgatctcttccctccccactgccaggcaagacacagtgctcctctccagaggactcaacagcaaggcaccatcttgaaagaagagctaagccttcataaagcaccaatcctacccatgacttgtcttgggcttccagcctctagaaccatgagaaataaagttatgttctttataaacaacccagtcccaggtattttgttatagtagcacagagggactgagatactcattcatttattcattcatttactcatttgccttatcaaagagactacctcgaccatctcacctaatacagcaactctcagtcctctttccctgtttcattttccaccctaacacctacagcctccggccatattctatatttactgctcatgtgttaacctccttcactgaaataaaagtaccaagacaaaggaagcttgactgtttttttcattgctataccctcgagcctaaaaagtcaataaatagttggttctgtctatgtgcaaggcatggaggcaggtgctgaaaaatcagaggtgaatttgacaaatttgttccctgttttatggcctttaacacatgcaggatctcatgtgtgagaagaaacatgccaaccaaagaaaactgagaaaacagaaagaggtaataggtactgagggaagcactctaagatcagacactctaaaaccttttttgccttatttctttgaattcccatagtgtctctatgaagttaggtgctatcattatccctactgtacagatgaagaaaccgaggctcagagagatcaagtaacccgccccaaaccacatggtaacagggtttgatcccagatctgtaggtatcagagtatggcccttaagtctggatacccagggaccctgtcctggctctggcttttggggttccactagaactctcattcagctgtgcccatctgcataggggaaggaatctgcaagaccaaggggatgtgcctacctggagcccatgtgccccctgctgtcccacaactgagcacccaggaccctgcaattccctgcctgacaccccaagtacctgctgggctccatgggcctcatcctgttctctgctcctagaaaatcccaggccctgtatgagcatgaagaggcctaaggggaaacccagagggctgttttgtggaggatgaaaggagcttaaatgtgtgggctgggggtccacagcaggcatgtgcatgaagccccccatggtgtgggaagggaaggggcagtctggggtacagaggaccccacctgtacttgtactacagctccccactcacagaaattaggggcaggcctctctgtgggatctaggacctggggccttaatctccagactctatcccacaaccactatgtcccctctaccccagagccagcatgggggcatgggtgatgagcaggatgcctgctctctgcagtcacacaggatggctgggctgtgccatgttcacctgaaccagtgagggaaggtactgaatccagatggcttggaggaggagcccacatgatcaaggcaaacacccaggaataggaaagtgacataaggcacgggcttcccatcagctggccctcaggactcattgtctgcacactcccctacacacctcaccccactcagtctaaccaggttccctgctgcaggggacaacaacacccccagtgtcatagtcatgctgcctaagagtggggatagaggggcaaatcgactgaggctatataagggagaaccaaatccttcaccttcggcaggccaggccagcctcataattagagagacaagatggtgaggtggctataggtgtgtgctcagggaatgatcccctgagttccacctgacactgactctggacagctgggtgaacacgcccaagttacttgagctggcgcaagtgatttaaagtctcctgcaccccaatttccttatctgtagaacacagaaaattatactctccacctcaagtcttgtggtgacaactagagagttaatatatccaaatctacaatgagtatatagaacagtgcctagtacagagtaatgttcagtaaataccattttttacaatactccgtatctcttctcttcccctcaactcgagcacctcctttgtctttgggtattatagtttctggttcctaaaagtatccactctaccactctttctcatttttacctgctccttcctaggccacatcctctctcctgaattactgcaacacttcctaacaagtcacatgcctcacattgtatctccccaaagtattctcaggcccaggccacagagacctcctgataatgtaaatctgctgtcacatctctacccgcccattcagttactgagctctttactatcacgttcagcaggtacgcgctgagtgcctactaagtgccaggcattctctgaacactggaggaggaggattgagcaccagtaaacaagaacattgcccttatggaatttctcatggaggaaggcagacaatacacaaataagcaagaaagtagcaggtaacgccgggagatctgcagagcatgaaaatagggcaacaggcaggacctttgtaagccctaagatgtctccatgtaattgagaaaatggcacgccttcctcctgggtgaggcacccccaagtgctgttcagcccccactcacctcctccaccagccccaccatgtgctgcaacccaggtaacgaggtgctgtaacagaaaacacttaaatgctagaatcctccagtgctgaatttgcagctccaaccaagtcctggacggatttctaattctgtatatatagcaaaacgtctgctagtagaaaaggatactggattctgagattctcttgaaataaaccttcattgccatcaaaggcgacacatgtctttctggatttcagaggccatgcacaccttctcagctcccatcacaacatacgacagtggtcggtgtagctctggtccttcccacatcccaaggcacccccagatagctctaactaaccatgccaagcctctgcgagaaaaccttctgtggcattgtattaccaagaggctggcactcggtccctcccagatctgctcttgttatctctctagctttgacacagtgttccccaaactctagaggacaaacaaagcagcttgattatccaggcctaagaagacaaggaaccgctctcatcctcaggactaccccacatggttgtgcaaggtgtgccctcgcaactcatgaattaggccccctggagttgttcagtgcacaccctgtgcagctgtgtatggcagacctgggtagttagctgaatggtggcccccaaaatataggtctacaccctggagcctgtgaacgagatcttatttggaaaagggtctttgcagatatattaaagttaaggatcttcagatgaagagatcatctggattatctaggtgagccctaaatccaatgacaagcatccttgtaagagacatccagagcagaggtacatagaggcaaaggcaaagtgaagatggagtggagactgcagtgatatggccacaagcccaggaatgctggggcaggcacaagaacccaaacgaggcaaggaatggattgtcccatagagctcccagagtggacaagaccctgctggatttgggacttctggccaccagaactgtgagaaaataaatttctgttgctttcagacaccagattaatggtacttcattatggccatctcagggaatgaataatgcctgcaccaggtcttagtttaaatgccatttgctcgaggaaaacctccaggggatctatctctctcctgcagctgtgctgagtgctcccactatggggctctcaggcacatcaccttcctctcgacaccagcccttttgccatttgggggactctagtctgtgactttgccctccactcctgggcttgtctcagaagtgggagatggacatgtcagcaaaaactcacccgtgtcctccaaatgcaaggggttctttttcaaaatcttgagaccccaaaggcaaaacattaggagctgggaagtaagtgagagttccctagtccctgattttctgcagccagaaacatgtgtggacactttataaggcttatataagatgatggtgatacacacttgcagctggaggaagatgcagcagctgtcaggccacctcagccctggtggttttcccaggccacactgggcagtctgtcagttgttgtgtcccagaattgagagccccaggagtcagtggcaactgccgggtctgatggacataactttcctgccctacatcttctccatgatcagcacatcatagtgataaaggcagcgtccactggggtggcttctgataagccacgaacctgaatgttcccatttgtcccatttttcctggcttccagtccccttgggtggctggatgctgtgcccagcaggggaacaggagaagcactattgctaacactcatggagcaccttctaggaaccaggcactgtcctgtgccctttaccaggagaactccatctaacctcaggacaaccctgtggtaacagctctctctgtacactgcagatgaggaatttgaaagtctaggtggagagtttcccaaagtcacagagctcgtaataaaggccctgagattcaacctcaggctctctgcttccgaaactcatgctcttaacttagaaagccagagttagcctcagtcagggtctcagcctccaggaggatcacccttgagggctgactcagtctcacctgcagaggacccagctgagactcaggggtgccctcatatggccacccgtggggcctctggtttggaagaaatggcatcccatggttaacatctggcctcttactctagcaaacagactagggaaagtcatagcctgggcagctgtggctgctgcttcctcccctccctgtcccctcccctcttctcttctgatttctcctcttcctcctcctcctgctccttgttctctcaaaaaatatctattgagcacccaccgtactgaaggacctgtgtccactgggaatttagcattgtaaaggagacacagcatagtataatgggatataaataagtaaacaggaaaaatgaaataaatattatacccattgctgctgcaggagaggaagagatttccttgatgggctactttgtttatggcactgagaatctgtctcacaaaggctgaagatgaagagtgactggagaactccgaagcagtccagaagtctcaagagatctgagtcatcagggcatgccagtcagaagttctgtggtagacagtggttggcagtctctaagatgctctgatcatgcttgcctcctgacagtcacacccttgtgcaatcccctccccttgaatgtggtctggatttagtgacttgcttataatgcatagagtaggacagagtgatgggatgtcacttctgattaggatataaaaagactttgatgtccttcttgggggcctccttgcatgctctctctcactcttgccagcagggaacccagcagccatgccatgaggcagcccatggagaggtgcctgtggggagggaccagagcctacccaaaaccacgagtgagcctggaagcagactgttcctccccactctagctctcagatgagactacagccccagcaatagctccggagaaacacataagagactttgagccagaggtacccagataagccatgctcccatcagaggctggccagaaatgcattaggcattattctcctgcagaaggcacccactgcacacagtaggacagttctgacatccaaactgtgccacctctaccccgagcactagttctatcttccaggaacacacagagatgatgaatcaattcctttgccatatgatagtctttcaggtgtgggaagttggtgacctaacagtgggttcccctattctcagttttcttcactcctagccacacttttctaactggttctcaaatccttctctgtccttgtgtttccccacaacacaatatcccaattatgttcccataatgtcaagccaattgagctgtcttgtttctggctagcaaagctttgctcaagatgtgccctccacatggaattcccttcctctttcttgtctggcccaacttaccacgactgttccaagaatcctttctggagcctgccccctcagtctagctggacacctccctggtgcagaacctgtccttcctgctgcactcacctctcttcattgcatcgctgtgccatcatgatgtgtttgtgggtttgctcctgtaactgtgagcaccttgagttctgggaaggacccatggttcatctttgaatccacaacacttgttcagagtgagaggatggatgaatggattggatggatggatggatggatggatggatggatggatggatggaagatgaacaaaggaagagaggggcatcctccttagtagacagggttccattggcattcatcctcagtgctatccttctgggtatgtgcactttgtaaggaagcttctgtgcccttcaagataaagcctggacaattcctcatggcctaaagcagagcaagccttaccaacccctctaagtcctgcaggtacaagtgtcatgacttcagtggtaagaaagttcagagagagagagagagcaagcgaagagaaaacaagagagaaaatgagcctaggttaaaatatttaaaacatttccatacttcctggacttataaatatttcattagtaagatgctgtcccacataaatgcaaactcaggggaaagggtcttaggaaagctgggagtcagcattttaaggaagccgatttccctttataaacataataccttctcactgtaatgtggaaaacagaaatacaaagaagaaagaaaaattgatgtattacttaaagtcccatcactcaaaaccaaccatttttagtattttataaatttttgacctatggattttgtatatttggggtggttgcatgatactacatattctcttgtgtgcctggattttcttattttccctaaacatagtaatgtaaactttgtcctatgttactgtgaactctttatgaaacaaatttttttttttttttgagactgagtcttgctctgtcaccaaggctggagtgcagtggtgtgatttcggctcactgcagcctccggttcccgagttcaagtgattcccctgcctcaggctttggaacagctgggattacaggtgcccaccaccatgcccagctaatttctgtactttagtagagacagggtttcaccatgttggccaggctcgtctcgaactcttgacctcaagtgaccctcccgcctcagcctcccaaagcatgtgagccaccacacccagccatgaaacaagttttaacaggactacataatattccatccaggacatgtaccagctgggaagtcatcaaggcctctgtgatacagtggcagagacaggtgtagttgatttaaatcttcccatattgttgggcatttaggttgcttccaattttcttactatcagaaaaatgtcaaagttgtggccgggtgtggtgactcacacctgtaatctcggaggcggaggcaggcagatcacttgaggtcaggagttcgagaccagcctgaccaacatggtgaaaccttgtctctactaaaaatacaaaaattagccaggtgtggtggcatacacctgtaatcccagctactcagggggctgaggcaggagaatcacctggaccaaggaagactctgtctcaaaaaaacaaaaatagaaaaaagaaaaatgtcacagttaacttatttgtctaacctcaaattctttccttaagtaagttcctcagaaatacaaataattggcaaagaagacaacctttctatggctttttatacttattttcagttgctttctaaaaggactgtacctaaatgatctccccacagtgggatttgagtgcttgtctcccatgccatccccatctgggggccatgtcataatgcttccagatgagaatacctcagagataactgatcctggcaaggaggcaggtgaaacccaaaactcattgatacattcaagagcgtttcttgtagggaagtttcgctccttgatcttcagcccagacaagaccctcttccctggacactgaggctatttaacggcccctcctcttgaatcccactgcctgacttcagaccccatcctttctggcttgggtcatacaacagcccccacacttctgacttctctctgtcccatttattttattccccctctttcagccaaagtggtctttctaaaatcaaaaatctaaaaacaagtcatcttagacatttttcttccttccttccttccttccttccttccttccttgttttcttttcttttcttttttcttctttttttgaaggagtcttgctgtgttgccaggctggagtgcagtggtgtgatctcagctcactgcaacctccacctcccgggttcaagtgattctcctgcctcagcctcccaagtagctaggactacaggtgcgtgccaccatgcccagctaatttttgtatttttagtagagatggggtttcaccacggtggccagcctggtctcaatctcttgtcatcatgatccgcctgcctcagcatcccaaagtgctgggattacaggtgtgagtcaccgcgcccagcccatcttagacatttttcaatgactgcccattatcttgcacccaagtctttccatgatggtccccaacttctctccagtctcactgtctactctgcaactctatccccctcactttgtaataagtattcactaggtgctggtgacaggccaaagagtgtgataaccgcccagctactcaccctgggggttcttggtctgacagaggaattagagggtgacggtacagggtgacagagcccagcaaaggttggggatgtcaggtctcctttcttttcattcaaagctttcattccttacaactgaaaaaaggcaatttcaaaagagaatccaaattcaaaatcagagaaaccattcaagagttttgtgaaacgatccaagagttagtgattttcagaaaagcctgtatcaggctatggggtatgctgtcgggggaaggggtggaagaagcaaagttggggaaaagaatctacttcctccttgttctgtgtcacctctttttgtaggttcctttttcctcctctccactagtggttccatttatttacctgaaggactcctgcacctgcctgtgatgtctaacatgataaacaccaggaggcccttgtgctggagccttttcttccttccctctgcagctccaagagactaatttgtagagaagaggatccttctccagcagaactcaagtgtagaagggagctgagctggctttttagcagcagtgggaaattaatctgtgttctcaccatcacagtcccacacctgagctgccgtcctgttttcttgcctctcccacctgactgtgcattcccatagggcagggtctagggctgggcactttgggacctaccccaaccaatgccagcagggtttccagaagcaagtcacaggggtctcccctgcaccattggcatggcctccagctgtgcctccacctccacatctgtggaaacctcatgggtcagtgggccacacaccatctaacaccttccaagtcagagatccactttcaaatggtggctctatcccacatccaaacacaaggcccactgtagtgacttaatctcttagggtttctgcgtcctcttctgtagaaagtacctgcatgatgggggaggcagggttggaggataatattcatggagctccagacaaacagtaggtgaccacaaagagtaaaaggcacagcggtgttataaggcccataagatgaataacggggatctgaagccagggagcctgcctttgggatccacgtgcctaaacattgcaccaaccacctcctctctgtctagccaagtccccctgccccttgcagtgtcatatagttcttcccagtagtagtttatagtgctcactccctctccctcctcataattattctgtatgttgggcacccatcatcttataatgccccagtgcctattgaatcttttttggatcagagacatccaatacaactcatagctctctcactctaaaatgatcataggcacaggcaataattcacaggccctcttataggattgttcaggttgtgcactgcataagggtacctagccaatgggcctctacaggaatctagaatccagcccacattccattcaggagacttgagtccttgagaggaaagccaggaggaagaagtccattttggtaacaggtatgcccagagaggaggtcctttgtgcattttttccttctaaaaatgttgggtgccacctttgtctaatttgtataaaggtcccttattgcctaaagttggtcatgacacttacatataattccagaggagtggatgccccagaaacctgtccatggaacatcctggggcataaaggtttctcaagtcaagaactgtaaggctgagcatggcggctcatgcctgtaatcccagcactttggcagtccaaggcaggaggatcgcattgcttgagcccagaagtttgagaccagcatgagcaataaagtgagagcaataaacccagtctctataaaaattttaaaagttagccaggcatggtggtgtgtgcctgtagtgccagctactcagggggctgaggcaggagaattgcttgagcccaaggaggtcgaggttgcaataagccatgattgtgatcgtatcactgtactccagcctgggcgacagagcaagaccttgtcaaaacaaaaacaaaaacaaacaaacaaacaaacaaaactttagggtcagcacccaccacatgaatatatgaatgaagaagggctggtaccaacaggtcacatgttgattaacggtttgggactcatacagacatggcattcacaggtataaaagactcctcagaccccccagacctgccgaccttgcagtttggaaatcaccatgcacacaccattgctacaggaaaatgcatttgccctcaatgtgaggataaaaatcaatgtgcctaggcccatgcctggcatataataaatattcaatgcatattgccccaaaaaccttttgtgggttgattcggtgccataagactgaactttgaagtctggcagtatagaaatgagctttgcaaaccacttgccaagagaggaacatcataaatgcacatgatattcttatcatctaaatatttttccaaatcaattcaacatagtaaagataggaaccatcttggggctacatagggatgggctgggtaagagggaaaacttatgggcaaatctcagatggtcgaaggtttggagaaacaaatggcttctataatttcctttctttttggatttattgagtgccttcggtgtgctgggcagagtatactgggaagcactttgtagatatccattttaattctcacaatggccctgtgagagtatcagcatgcctaggaggaccttggaggtgaggaggcttaggcatggcccaaggtcacacattagtgtgtaaaggcagaactgaaattaaacacccaggtctgacttcccagtggtcctcctgcacaagggagtagctgggatcccagacccaggagtccaaccattgggatttaaatcccagctttgccatttcctacctgtgtgagcttggataagctccatgtcctctctgagggataataaaatgggggtgattgtggtactgacctcagagggtcacaggataagtaattattactatatgatagcacttagaagagtgtctgacacatatgtgctgttcatttcaatatcatcactgttgttttgatcatggctctttggaagctggtggtttgaccgtggtgttcagagggaagccgagggtgtgagaacactcagtagcgtcaccctcaaatggcgcccgccagtccagaattcccatctgtgctctcaggaacacaccggttgtttgttagcaaatgacctttcttgctgaggggatgctgacacagaaaaaattaagctgcttctggcagaaaataggcaggaaactggtcttcccagtaagcaatggttcagaaagaagggatgttgagacctcatggatatgtgaactccaagacctccatcacctcctcagtgagccttctcttaatggggcttgtacaacgtgggtcagggggagagggaggtcagctagcttggtcttgtgaaacctcaggagaaaccagtgttgagaatgaagagagaccacagggcaaaaccattaaagttgcatgcatactgaaaacttcctcttcccgcattacaaaagttcttgggataagatggcatgggaattggaattgagggtcctgggctccagctcttgttctaccattaatcaggcagctgtgtggcctcaggtcaatctattcccctctctaggcctgaggcacctcctctgaacaaagtgggggctggaacagagccaggagcagggcccttccagataagaagcaatgagttttgttaaaaataagaatctatgttgtatatcatttttaacttcttctgttcattaatccgttactcatttattcattcattccaccaactcgaactgggcaatctagaggaccccctcacccttcaaaaatctaaattatacaagtttgtcctctgccaattccaaatgcctaggaatgctttgctctttaccaccatgtaccatggtcagcttcttcactggtttctttaaaaaaaaacaaaatgcactcacactaaacattttatgagtttggatccttaagatggggcaggggtgaaaccccatttctactaaaaatattttaaaaattagctgggcatggtggcggatgcctgtaatcccagctgaggcatgagtattgcttgaaccaaggaggcagaggttggaatgagccgagatctcacctggagagggcttatatgacaagcaaacagcccctgtatcctgcctgcactctgtttatgcctcccagtgctttgccatcactatgaagtccacatttttctgacagtattagaaagattagcttcccccattccccaaagcctggaatcctggcgacacccagaccctgggaacagagatggggtagcttttattggaagatttataaacccaaacctatgaggacatccagcaacacaaaggccttctctgtaaagaaatttactcttctgagtgcgggcaacaaacataaaaaaaaaaaaagctcatcaaaaagttagataaggagtttcatccatgcagctgaagtcatcactggtcattagagaaatgcaaatcaaaaccacaatgagataccatctcacgccagttagaatggcaattattaaaaagtcaggaaacaacagatgctggcgaggctgtggagaaataggaatgcttttacactgttggtaggagtgtaaattagttcaaccattatggaagacagtatggcgattcctcaaggatctggaaccagaaatactatttgacccagccatcccattactggttgtatgcccaaaggattataaatcattctactataaggacgcatgcacacatatgtttattgcaggactatttacaatagcaaagacttggaaccaacccgaatgcctgtcaataatagaatggataaagaaaatgtggcacgtatacaccatggaatactatgcagccagaaaaaagaatgagttcatttccttcgcagggacatggatgaagctggaagccatcattcgcagcaaactaacacaagaacagaaaaccaaacaccgcatgttctcactcataagtgggagttgaacaatgagaacacatggacacaaggaggggaacatcacacaccagggcctattggggggtggtgggggatggaggcaaggggaggatgcgttaggacaaatagctaatgcaagtggggcttaacacctagatgatgggttgataggtacagaaaaccaccatgacgcatgtatacctatgtaacaaacctggacgttctgcacctgtatcccagaacttaaagtaaaataaaataaaaaaagaaattcattcttcttaaagagcagcctagattttaagtagtaaaaagtagtttccttcattgcacctatattgagcacgtactgagagctaggaactgagctggatccctcaaggagttctctgatatagttgtcactagctacatgtgatcattcatctaaattcaaatgaattgcagctaaatcacatttaaagtttggttcctcagtcacagtaaagacatttcaagtgctcaatagtcacatgtggttggtggctacaatattggatagtagagatatggagtatttccatcatcaaagaaagttctaccggacagttctggtctaacaggagcaaagagaggaacagaaaactatgccagccagaagccaaagcgtcaaacactgaggaagaggtctcctcttgagttcagcattagtgcgtgactacctcgcaggcaggagtcattcacaaacctcagtggaagaggtatctactggagagctgtcatgttcaatctgtttcaaccttcaagttagataaggagtttcatccatgcagctgaaggcacgttttctccgtctcaagaggttccagtcaaatgtcccctctttctgggccttctcctcttcacttctgagctcttcatcttgctcactcctgccacacccatggacattctaccttgcatcacaatcatatgcatgtaggtcttatggcccttccatctgtgagtgccttctcgtcccttggttcctagcagtgcaatttgataactgaacgaatgaatgaatcagtgaaaaataagtgaataactgaataaacaaagaaacaatgaaaacatgcacaactgtccattccactgtgggcttaatccagacctggtttctctgtcataaacctatccaatcactggtgaacttgaagaggttaagggtctgtgtcagctaccagcttattatctctctaatcttagaaacctgtggaaaaaagagacattcagagcatatccacctctaatcaacctgataactgcctatacaaaactctcaggcatccatgcagtttttacctggcaaaaataattcatgagtcattgtattttctctaataaaggggataattttgcatgcacagaaacatccccatttgtccacacagctctttcttcctattcctcctcttttcagacttggaaaagtgttctttctgcttgttaagcaaaactctctccatgtgtccttggcagtaacctcttagttccactgggtattgatccatcaatcattgccctgtccccttcccaattcatgtccactgttgctcatggtcagttttctgtaaatgtggacggaggatgatgatctcttaccatctctctaacactccctaaactcatttgattgcctctagctcaaacccaaaccatgaccctctctctttctttcaccatatctcctttaaaaggcaccatgtcttcacctccccctccccctgaaatccaactctgtccccatccattgatttcttttttcttttttctttttttttaaggcgcgtcatccccattctcattacccaaaccaccatcaaatttccccaattctcgccttctcaggtgcggtgccagagtagaaagggcaataagtaaggtaaaggtggccaacaggcagcccacgggcaaaatttggcctatggatgtgttttgttgggttgacaaggtatgatagaaatggagaaagttaacagaaaaaatccagattctccctcttgtgaaaaataagatctgataaattatcatccctactcagcgtcagctagcagaagcagtagttgctctctttagagaaactagagtttgccaccagtttcctgcaagcctcactacttcctattgtcttgccctagtcatgcctttcatttgaaatacttgctaatccctgtaggcatttgagtctgtaagtgctggactacagtttcaaatttataatattttacaataaatgtgtttcaatagttctgaagtatgtatagtcataaacatctaaacatcactgaataaattgggcatagattatgtgtagttcttcatcatgagatatggcttgtattctcccccatcacctactgcactataaacacattttcttacacatgtggtttttgtttttacaggcatgtcaaagaaagcatagggccacttcttcagggctaacgtattgggcattgtgagctttcctgcaagctgatgcctctgtccaagtacactgaatccacagcacacagcgcttcaatccaaccctgctgtaacactgaacatagtgtaaggactgctgggactgctcacctcttcattcttgaagcatctgctactctcgtggtgaatcacagtgttcaatggatttgcttgctcactccatttcatccatgcactcaccatgtcacttattcattctttattacagttttattgagagtacactgtgttccagaccctgtgccaggcacatggaagacacatctttgtttatacccttcccatggtgtctacatgacactgcctgtattatatttatctgtgttcatatcttgcacaagaggttgaaaccagtgcccaggcctaatctttctgtgtcccaagcaccaagctggggaaatacccaaggaagcctccataagtgcttatgaaaattgaatttgacgtgtatacccaaagctggcagaggctggagaagacagaatcttgctaaactcacaggagtcaaccccaaggctctagctgtctcatcaaaggccatctgggctgaggcatcatggtcagttcagcaaggcagaaggagggccaccaccaaggtgctgctggtggcttctttagggaccttccaatgagaggtttgttttccagctactcctaccacccaagagatactcgctccctgatttcccaagctggaggtggcatgcttgtggagacctccacctggtgtcctgggtggctttgcttgtggctcttccagtttctatagtgtgcagtcttcagttccatattttctttgccaccctgaaatgaagggcgtatctgtgccaaatctgctcaacgaagaagacaggaaatgaaatgagatttgaagccaagttactactcctttttccaattttaagactgtggctgaacagatgaaagatattcagataagtggcacatgttctttgtgtttaaatagagcttgttgctgggcaataattggcctctgcatgcaggtgcctttggtgagaaatccaggcttctttggagggatgatctgagtaagtgcacaagcatattatgggagatatctaaggaggaaaagggattctcccataatacacggtgatctcatctgattatatccaggttgcaaggaaatctccacaacttacagagtattatagggtcagtaccctattctctactaacataaatgtataaacagcccttacatttactacacatttaacatctttcccagtgattttataaatgcatcatattcaaagacattgccaaatctaaatctgtgcatgtgtctactgggaggctatctaccaggtgccatttgaggaaactgcacaccttccttcccctttgcctccacttattagacctgaataagacagacatttagtgagtttgttctaagtgctgagcactgttctgagaacttgctatgtattaactctgactcctcaagaaaaacctatgaagttgtgtaattatgatctccactgtgcagatgaggaaactgaggcctggaggagtaaagaaacatggccagagtcacacactcatcacggtggaatcagaatttgtatcttcagcacctaggcaaagaaaatagggcaagtagctttgtttatattcctgggagagatcacaacataggctacaggttcagggtatagaaagctggtggggacagttgggtgaaggcaacaggtaaggtcgaggccagtgcagaaggagtaatggagactggagaggaaccagttggtggagttacctagagaggagggggctgggtgctttgcgtggggcaacttacgagcagcagtttccagaagcttgggagaagggagagagaaggtgtcaaaagacaaaggatgtgaagtaacccaaaggagaggacaaagagctgagatagagtgaccagtctctcaaggtcaactttcagctccatgtgatcaatggtacagtttcactagagcatgcccccctgtaatttgctgatccaggggcatagattggtggccctaacttgctgagggattttaagtgaatccttgctcctctggggccctcaggacactagaatttatgactctgatgaaatttagaataatattgattcacaagtcagaaattaagatcggtgatctggttaagtcatggagcagctacattcatcacctactttctctcacgtatgaaatggaaccacccacacccatcctgccttccttagatctgccttaggatttaaaaagtgcaatgaaggtgaaatacatttggaaccttgaaaagagtacataaaactgaggcagagtgatggtccaggaacgtactcaagcaactggtaggtagtgagagcctactccgtgcaaggcctgcatgaggcactggggatgcagtgaacaagatgagcttcaaccatgatagtaacaccagatcaggaagacagccaccacacaaataattcctggagtacagttgtaataaacagtatgaaagaacaatcccatatacagtgagagcacatactttggaggaaggcaacatctggtttgaatactgtcttgataatttccaactggggaatgtttttctgcccattgtttaaagcactctcagtctcattacccctatctctgaaatgcaagtcttggagggtggtggggaatacgatgattatcacagagctgtgcacataataagagctcagtaaatagttcttgtatgtcatgtagaagactacagctacattaacaatgatgatgagataatgaggatgatgactgataatgatgatgataatgatgaaatggtgatgatggtgaggaggatggaatggtgatgatgatgatgatgacttaatggtaataacgtgatgatgatgatgatgatgatggtgatgatgatggaatggtgataatgtgatgagtattatgatgggatggtgatgatgacaatgatgatgatggtgatgatgatgaaaaggtcatgatggtgatggtgatgatggaatggtcataatggtggtgacaagatgacgatgatgatgatggtgataataatggtatggtgacaatcatggtgattgaggatgctgatggcagtgatgctatgtcagggcatgaccatcacttttccctttcttctcttcccagagggctcatatgagctaatctacaagtgggtctggaactttaagccacatatgggtaccctgcttgggctgtcaggttccaaaaaaggcagattggcatttccacactggttttctctggctacatttcttctgcctctgctcatgcaagtgccacttcactctgagaaaatgccagcctctttgaggtgagctggagcctagagaggcggaggcgggagcatttccaaaggtcgggatgactcagcagtcctctgaaaagcctctggtgatagggaggtgatgctgtcagaacaaggaaatactagaaccctctccaccactctccatcacagctaaaagcttgccttgccatttctttgccctcttctgcaagtctaatgagggaatatagatgcttactatatttgagctgcaactaatgcttcaactctggaaactctgagcaaactccttcatgtttgcctggacaaatctcccaggagtcccaggctggaaggtaaactggtaccccaaagccacagggtctcctaccagacttgtttaacctgcaggacagagtctgggaggggagaagaggaggtctcacctcacagctgttccctctagtgtgcactcctcagaggacttgctgatggtagagctgccccaaatacaaaacggctgttttgtgggaagggaagctggcaaagcttctggagacactagggagggagaatatagtatagactaggctaaagtggagggcagcagtccagggccctgatgttgtttcgacggcgttgaatggaactccttctaggtgaagatgctggtagttttgccatcaccctccattgtcttgtgtgggcggattcacgcaggtaggatacctgcctggcccctgtagatgtctgagtctgtacccctgggctggatatttttaaggataccttctgcccctggagtctgtagttctcagaaataagttatggtaaacaaatcacccaagaatgaatagccattgattctaatcctgactttgccaatggaattcctgccaagtccctggccctctctttactcatcaagtatttcaatggcctggaaagtacccattcttacctttccaggcatgatgatgcgatggcaatgatgacagtgatgatgatggtgatgatcttataacactgcatttgcttactttgctctttgctaatgctcccattcttggaatacaatagtgaacaacacggttgtgttctttgaccttgtggaattatttgctagcgggggagggagtaaaaaagggtatatataaatacttaagacaatacacaatggcaaatggtgacaaatgctgtgaggtgaaaggacaggcccctcagagcaaagggcagcagcaacctaacttacataattcatcaaagaagatcttgttttaaataactactctttaaactgagataaaaaatattagataaaagttatccaggtaaaaagaggaaaaccggccgggcgcagtggctcacgcctgtaatcccagcattttgggaggccgaggcgggcagatcacgaggtcaggaaatcgagaccatcctggctaacacagtgaaaccccgtctctactaaaaatacaaacaattagccgggcctggtggtgggcgcctgtagtcccagctacttgggaggctgaggcaggagaatggcgtgaacctgggaggcggagcttgcagtgagccaagaacacgccactgcacttcagcctgggcgacagagcgagagactccatctaaaaaaaaaaaaaaaaaaaaaaaaggaaaacccactattccagataaggggagcagaccttgaggatgctccaaggcagggaagagttggacagttctctgagtctctattgcatttcctacattgtaatttataattgaaattcttcattatgaagactgtgatctgctcctacaagttaactctaagtcccaccctaggagaagtgaattactatttgcagctgctaatgaaaaaaaaaatgctgccaggatcataagccaggatatgaactgccctaatttggaatcagcaccttcagaggtggttacttagcccttaaaatatctgtgtaaatttgcaacagacggtttgctggacttggataagggctgttagagataatttttcatgggtgaaataaatagtatttacattagaatctgacaagccctgcaactgagggtttctctgctgcatgggaaatccagtggggcactgtccaacagaactttctgtgatgggagaagcatcctacatctgtgcaccactatggtgtcactggccacctcaggctgtagaacacttgtaatgtgcataaactaaaattttaattgtattccatttaaattagtttcaatgcaagcagcagcatgtggctagtggctaccatattccacaggtgcagaggctggcggatccaccttctctcctcttcacagagggctctgtcatctgattccacactaaccaggcagtcctgcttcaaaagatcatcgtgttcacctgaaagtgactccttcttgactttctaggctagtaaatcgtttgcaatactgttcattcctttgccttgcttttcacatatgtggtagtgaatgtccgcattttggcagaatttagtcataaacttgctttcactgtaacataccatgtgcacacaagattctggggaggaaacagatagtctcccacctgtaccctcggggcccacctatcactagctatgagaagctattaatattcaccaaaataatctgactctttcagaaaagccaagttgaactgagtctgtttccaacagccttggtttcctcacctgtaaaagcaagaggctgagctagatggtcctggagagcccttccacacttccgccctgggagcttgtcacagtaatgcagattgtcagagctggaagagactcatccaggtccctgatgctcagggaggtgaagtggcctacccaaggtcacacagcaagtcaacgcctcaggcagcatgtgtggctaagtaccctggtttctattccactttaatggggagactgtgaagacagatttactttacactaaactagtatctaacgagcgacttccaaatggaatggaacaacccacaaaatcagaagcacagatgagagatacgctgcatttttccttactttacagatgacggggggaagaacagaaagagaagagagaaagaagtctgctgtacttggtgactcatcactgacaaagcggacttccctgttccttcaatctggacttgtcacgtgacttgcttttgactaataaaatattataagatgttagcagatgtgatgcaagaaggggcttgggatatgcatacgcatttgggtttcccctcttacacctccaccaagccctgaaagaacatgccctaattactccacatatggaacagattcagcccaggagacctgcagagcagacctgcaggtagaagaaaagccatccagccaagcccagccttgtttaagcagctatattcttcctgaaagcatgtgagcaagcccagccaggatcagcagaaccacccaagccaacctgcagatctaggagaataaaaggttgttttgagccactgacaattggggacagcttgttacacagcatttttgtgacaacagctgactgatgtactcagaggtgaaatgacacgctgaaggctacaccactagtaagagcgtagctggaattcttactctgcatcaagcgagatcatgcttcatggatggggaaacatgtaatgaatcatttaaatggagcctcttttgacagtgaaaaggagcagtgaacattactttggacctgtgaaatgatactgaagtgtaaggtgcctccccactcccaaatggcacacttagtgaggtgaacacaaacattgttggcagtccggatacatcttgcagacactgaagagttcagaacgtgtaatatctgcacaggttgacacacagacccaatcaccccattgcaaacaagttccttccaagagaaaatgtccgggtttcagattcatggtggggaggggctaggagcttcagccctggagatgctggctatactttgctctaactcgccctagctggtgccactgccaaatgtttcagcttccctttgttctagtcttcctaacatcccagggacagaagggtttacaggtaagatcccgaagtccaccgaggccaagctacctgttgtaatcacctagccaatgagcacccagatggcactgattccaaagcaggggccactgcatctgctcatcccagtaagggtgggcactacaacatcctaccaaggatcctgccaaccaggcaagcggggctctttcctgggagcacctcagagtgcaccacagagcaaagaagggactgcattttccatctccttttccttgttcttcccttcctgacaaacagtctagggcatctagggtggtcagtcagcattcctcacgcacgtgagtcttagacaaacactacctggtattggtttaggcagccatggaggaaatgttaccaaaccctcaccccaagtgcccatgcaattctaccagccttctcttctggaattctaagtcctatttttctttattagaacttaaaattgaaaactcaagagagaggaatttttttttaacgaagcaaatctattcgtgtggctaatgacaggaagctcaggggttggcctcaaactccaggtagcgacgcttcacccacctgacctactcgcaccccaccactaataactaggggcctggcctccctcaccaccccttcccttccccacacctccactctcacacatttctgcttcccccacagtgacagatcccattaatacaacttgaatatctgccaagcactaagcattctctaggttacagtaacacaaatgatcccatttatgtccaatcctcaatccccaaggctgggatggattattcccattgttcagatgaggaaagtgagactcacagacataaattgccaaatatcatcgagcttagccagtgttagatccagggctgaactggtttgtgtgtgacctcaaagaccattccccttcttcctttctgaaatttactcaatcaaaaactacatccctggggatactgtggtgaacaaatccaaatggaaagcaatctcctccacccctgccccaactctctaatttctacgaaagtcaacctcactcagagactgacgggctcaccccttagtgagcccattgaagcccacggaggaggcccactaccagagaccccagaatccttcaaatataggtgttccctcctccacagtggctgtggggctggaagtaggccctcccagtggatgaccatgagggtcacctgctacagctcaggctgtacagctcagggcacagggcagggagctttgcccttcccgtgggtggcactgtgaccacttggaagcaggtgactgtccttaggctgtggcttgagaagcatcccactcactgaaccagaagcagcctccttgactcagttttgcttctttccctgagaggcactggctatagggataagacaatcactttgggttccaacagatgtgaatttaaattctggacctgccagctgacagtgggcaaatcgcttctgctctctaagactcagtttcttcagttccatctggggtgacttcaaattacccccaccgcacccctctcagcagggaaggaagtcagcaggcttagagtcggcattatagctcacaagtgtgcagaacataccatttgccaagccatggtttgggctcgttttatgaattaactcaattaatcctaataaaaatcccatgcattgtgagggtggcactgttttcaacccacgtttagacgaggaagctcagagagattatgtaacttacccaaagttacagagaagaaagcagctgatctgcattttacatccaagcagtgctgtaagacagtggttctcaccaggggcggggtgttaggagcagaaataggggacaaagggatgatcttgaccccccaaggagcattcagcgatgcctggaaatggttttggttgtcacaggtgggtggggtgctgcgatcgtctagtgtgcaaggatgctgctaaacattctacaatgcacaggaacaagactcctacaacaaagactcacctggccagaaatgtcaacaatgctaatgctggaaaactctggcgagttcaggaacacagagcaggactcgcttaggaaactcactgccccgagctgctgtgagggctaaatcagaggttgcctgtgggcaccctggcaccacagtgcttgcagggaggtgttccttagctctgggggtgacagatcggaccgcaaggtggcacaagtaagtgatagcaacagttatgccaaagggctcatccttttcctttgattttatttttcattttttatggagattacgctattttaacactgttgtggcaaaaatactcagtgactcatgtgtcatgattttcatagctgatttcatgagtttttgacagttgatggtggttcctatagttagggttattttctgtctttgggacagtgtggggaaaagaggttggtgctcaaggcacactgccctgaccccagttccacactgggcatgatgtcccaggaaggctggtttggattcatggaagggatggcatgagagtgaagcacacaaagtaggattttaatcccagcactttgggaggtcgaggcgggtggatcacgaggtcaggagtttgagaccagcctgcccaacatggcgaaacactgtctctactgaaaatacaaaaaatgagccaggcatggtggtgggcgcctgtaatcctagctactcaggaggctgaggcaggagaatcgctggaacctgggaggcagcggttgcagtgagccgagatcgcgccactgtactccagcctgggcaacaagagtgaaactccacagcccccgacccccagccctccgccaaaaaaagtaggattttaactggcagacctgggaggaggcagagggtggaaagaagggaagggaaaggaagggaaggaaggggaagggaggggaatttcaggcagaaggactgtcagaaaaaaaatattgatcgacaactaaggacaaccattgggcacatggaaacggtcaaaactgggaagttgaacaaatgtctgttcaaactttggctctttcactgaagagggactttgtgcaaacctgctaatcacttacggtctcattttctcaactgtaatttgattaaacaatacttgctttcaaaaatgttggttttttctttgggcagctgaagcaggcggatcatctgaggtcaggagttcgaaaccagcctggccaacatagtgaaaccctgtctctactaaaaatacaaaaattagctgggcgtggtggcgcacggctgtaatctcagctactcaggaggctgaggcaggagaatcgcttgagcctgggaggtggatgttgcagtgagccgagatcatgccactgcactccagcctaggtgacagagtgagattctgcctcaaaacaaacaaacaaacaagcaaacaaacaaaaactttggatttttgtagcaacaagatgaggtcataggcaagagagaacactgtaagctctaaaacggctgcagatgaaggttaactagagccttgtttgtggtcatgagtgtgtgttccctacttgtctactaaaaagggaaagaagccctgagccaggtactgtggggaacacacagagcaggaatatagcacctggctcagcactttgaccaaacaggagcttgaatagcgaaccttctgtgaaatgtaggagcttgtctgatttccctcctagtgcccaccttgggctgaccactcacagaacccccatgtgtttgaagaaaagctaagacagatatctgcaggagaggcagctggctcctgtgtgctctcatgggatggaattcaccattagtcaatcatttattttcagggtctccacccccataggtagactcacattaactaaactgatacatacatacactagcatgctaaatgattgctatatagtgagaacaacggccaacgatatcagacaatacatccataggccagtttatccctgcagatcacctgtaattagaaattggcttggtaagaaaggaatcaaaacagggggcattagttatattatctgagtgtttcatggccctgaacttctagccagcctgacattttcatctccatttcaaggatacagagccactaagaccaggcaggtcgtattctggggtcagcacaccctctcccctgaacaatgctcagaggacacttcccacctgcttctcagactccccatatccatacttccatggtgagccaatgctacttctgatgggcaagttattgcctctaagatgttcctaccttccgtgagatgacagcttggtcttgctctggactgagagccattctgccatatagatcttggatctcctcctctggaggctgaattcctggccagtctcatttcctgtactgctgggcttgtggagtcagtggggcacccccaataatccctggtagcctctgctctttacagtcctcagtatacagccactggttctggggatgggtttgagggacatgggttcaacatcccagcagccttcctagatcatcaccctggcattgtctctgcgactgccctaaggatatagccccagaaagcagtcagcaacaggaccagttctataacagtcctcatatcagcaaaaaacctctagggaaagagagaaatgttccttgttctttatgctagttgactaagagcttatctatttatgaaaatgtcacaaagcacctctagaccagattgaggtcaggatttaggatgctttcctttaccttaatgcagagctgtaggctcaatgtgcataaattactggtataaaaacacctcaaggctatttgttaatcagtggtgagcaaggatcacatatctagtagggcctaagatgccctaagatgctgtccagggcctctgtgcctcccctccactccagcaccatcacagccaccctgatgactgaaggagtcctccagtccaagagaagctgtccctccgttatctgtccagggagaggcccctgacccaagctgagccaatcagaatagttcactctctgtcacagtgattggtccagggattgacatgtaaccctgagtctgccaatcaggaccctttcctggaaattgatgagagtttaagaggaagagcccaaaacaatcagaaggtgagtttcagtctggtagcaatcgtgtttccatgccctcagggaagctggtctaaaaaaataaagctggcaagcaaagagaaaaagggatgagaaggggaatgctggtgggatctgagtgttcccctggtttcttttgaacctgcccatcacatggtctgctgacatgagctcatatagtcccctttgtgcctagtcttcggaaaacacagactttatcacttaacaaccacggagtctaggcttactggacctctgagatatattcacctaccagagtccctcaactacctgatcctgaatttgccttcagaaactccaaagattgcatggattctccaatggtccttttactcggggagagtcaaataacaaaaggcaaatgtcatcttgatgccaagaagtcaggtaaacatggctttgaggccaggctcacctgagtgtggatgcggttcatgaaggtgataagtcagataagcatgctacttctctgagcctcagttttctcatctataaaaatggtgaaaatcacagggccattggtggatctcctgagactctcagcacagagccaggcactgagtggaggctccatgaatattagttccttgttattcagaaagtagcagaagacacaccactgctcgcaggctgctcaccttcagccatcacggaagaaatataagtgttcattcaaagtcaagtttctacaagaaaaatgtgctgggaagagagctggactggggcaggacagcagggactgattgaaaagataagatagcagcagtgtgacagctttgatgcccactactctacgaagcaggaatgatgtttttaagactacacattcaagtatcttcctactttgaggctctgggccctctggatatgagagctgtcaggtagacctcaagcctccacagagtggctggatgtgtgtccactctgcagactcatctttcccctcaggaataacacacactctagtcttggaaacagaatatcaggaagctcttgagatcaggaggaagccccatttcctgatgtataattatcggcaacaaagctggcatctacgagaccccatctaactgttgtgctatttcttaattgctttacaacccagagggaaagggactgtgattaatgcccttctcaaaaactccagcacctggcacttagtggatgctaaataaatattcattgagttgattttgttgagtgatggcccaggaatgggatggtcaatctggaaagtagtgagatccccatcaaagagacaaaaaacagagagtgacgactacatgacagaaaggctacagaggcaatttcaatatgagttgtgaattggattagatgtcttttaaaatatgtgccagacttgaggttttacagtcacgtggctcaggagagactatagtaaatctaaaactattttattaacaacaacaacaacaacaacaacaacaaaaactaagggccttggaattctggaagttgagtcacttgcccagggaggaccaaaaacatcttgaagatgatcatccctttctaaatgagccagagaataccatgctactcacccagtcagaccatgtggcattagattcatttgacataaaacaaaaataatgcccctatcttagcttgggcttccccaaaagcagaacccaagaaaagggctgggagtggttcctttggaaggtaattcagcgaagcaagagtgaagaagtgagccggtagaggaagacaggcagagaagtgcgtcaatgtgagggtgtgctgtggagaacaggggctcgattctcctgagaccacatgagatactgaaaaatcttccataattgtctgcacgaaaggcaaaagactggcacatttatccatgtctcctcagacaatgattgtgctggcaccagggtcgctctctgccctgcacttgtggaatgagtttgcctgcacacagtgatgtgaggtcagtctgcaagtctgagctgccccagccagtcctagccaaaaggagatatgggatgagcgcaggagacatgggcaccacaggcagctgcagccctaaactcatcactccatgtcgtccagatctccaaagtcacagccctgtgtgccagtctccaagctgttgcttctgtgatcttcctgagactgtcttccttgctcaaaacccacaggactacaagacaagtctaaaacccttctccatgggatcccccactccactggtccatctcaacctatggctgctcctcctccaatcagaaccttccccttgcactccaatgagtcacctgccattccttactcatgtccttccctaaaggcctttgtgctctggcgcaaagagctctgtctggaacaccatttagtttcattcccatccatcaaactccatcccgtcctcgacagcccagctgaaacatttcttccagggaatttgctcccttgtgagtatacttactgagttgcattgtaatttgtgtaagtgttggtgtcctcacaaaaaaggagcttctttaaggtcagggataaagttgtaatctaacttcagggccatccataaaggagatattcagtgaaaggtggctgagtaaatgaatggatgactccagaaaacttctcccttcaaggcctcagcttcttccactttagaatgaagaagtgggaggagctgaattagagtttcctgcagcattttctgagaagtcctagcactgccagatgcctctaaagaacatattctgaggcctaatgggtttgagagatgctttagaccccctctgggagtcactgtggctctacagagaattcctttagtctctgtttaagtcaacaatgcccagatccattcagccacagaatatctagaggaagaagtcaaccaaagaaggctcattgggtaatgttagctgagacatgactttggctccttccaactcaagaattctataatcatcatcaaactctgatgaagctgaacattacaaatatatgcgtttgattcagaaaaaaaataatatcccagagctctccaaaggtgaggaggcacgaagcttgccaccatgactggggctgttcaaaaagaatggatggtccccagcacttcacgtggaattcaaatatgggcaatatcagggggtcttcatttggggtccacagacatgcttgtgggatgagtatgaacccctgagattatacatgaatttgtgtggatgtacactgtcgctgaataaggactcaatactttcaatgcattctcaaaagggactgtgagtacctaatggccaaggatccctggcctacataagggttcagaagggtagtgcataagaaacgaaacacaaccaaaccccatcagctcgaggatgagggaatacaacaagtcttggaaccatccctctccattgctggaattctgcctccaatactctcactaaagaattatctgcattaacactcattatgcttgttacctcttcaccttggccccatacttgacctctccatatcctcatttccttatctgtctattgggaacagtacagtataattgggagggtcaaatacagtgatgaagcatcacataacttgacagtttaacggtagtggggtacccaaccaaatcacctgccatttaaaaaacaggaaaatcctcactcagtttaaatctaagagggagaaatgtttcatgtttgattcagatcaggggtgtcttgctgtagtctacattttatatctaaacacttgccaatttcccagcattgttggtgcatggattggcatgaggctccttgctccttctctctcataggcttatcattcctgaagccctaccagggtgctcactgtgtcctgctgggtgctccagggaaggggtctgtttcctcttaagctcctctgcttgggtcaagccatcagggacaccttcactgcaccatgttacgatgcctcttggacacggctagatttctgcatcagaaagagggctgggtaccaattgaatctgcttacacactccagaaagagagtccttggggcaaatccagctgtggattgtgtgacaaggtgaaagttcagttagtgcttcctaattgtgtgattggttattagagaaactagtaatgatgacattagcataacaaataatttgatggagtttacaaagcacctttgtattcattcaattaattttgttttccaggcacacccaggtcaaggcacttgcctggcaggtgaataagatccctttcccaggaagcccaggcccatgcactgccacatctgccaacttttctcctggctctcacccactacttcctgctagtgtttcctcagcttctgacttcatctgcatctccatgcctgcactggccccttggatctgatacagatttagtttctcaggcttgaactgggcaccccacttcccctgatgccatctacccttgacttctggttctcacttctcccagttacaactatccaatatggcctcagattttaggactctacagcacagctaggtcaatgaccaaactcccttgcttggggtgtcaaccacaaaatgacaactcttgccacagcctgcaaatctctagcagtccagcccctcagagcctccctgtctctcctccccattgcttctgttggtcccactgggctggcttcccagctgttcttgaagtcagccaagcaactctgtctagatatctgcagagccccttcttctatgcccccttcaccctatctctcatccctctttattttgcttcctcgcacttatctgcacttgacctaagagatttggcttgtttgttgatgacccatctcctctcaccagaatgtgagcctcatgggggccgaggcttcattttctcccctgctattgcccagcacaggaacagagccaggcacataggaggggttcaggaacctctggtggaatgagtgaaggaatgcactttcaacctcccagaggctcttgaaggaggcgccacctccagcccagcccttgatctaagccccttctggcagagacctcagactcacgacaagagctgctagctgtgcttccaggcccctctgacatctctggtccctcctctttcctccagtctcccttggacatcacctcatttacttccttcttagtaatgttcacggtccatacctcccttgtgtattccttggttttctcctttcctgtctgttttgatcttcagaatataagtgtcacgaaggtataaacttgatatcttttggatgcttggggtccaggccagtgcttggcacacagcagttttttataaaaatggaggaaagaagacagggagggactgtgacctggcttcagcatcactgcccaggcccttcagggccatgcttccaccaggctactgcttggctcttcattatggcctcagtccagaatgccactttttgagtgctcttatctctctggtttccattccaccctctcatctaataaccaatgcaagcttcccaaattccagcttgttttacagataagggggctgaggtacatatcaatgaggttactgagctaaggcaacagtgaacaaactggtatgcgagactccaaagtccagacccctgaccactttccaatagcctctctgaagccaagtagtttgtttataagtggagctgcacagagaacactgcaagcaaagaacaactctgactcatctgatgcttctgcggcacgaacctcctcagctcctcactccaaggttcccttagcaaaactctccccaaatggccatacctggtagctccaaggcagagatcttttatgtctaatatgttctttaatgtactgaccttttaaaattaatagaatattttttatcatctgaatttctcagagtgaggacttgcggttcattgttgttttaattatgccttagaatagcaacctcgtttatcaggctgcaagattactgtttgtgagaaatcttcctcataaagtaaaaggatctaaacagaacactcaaaaagaaaagaaaaataaaatgaaaaggttaaaagaaatgtaaaagggtcaacattaatagcaaccaaagaaaagaaaatgaaataaacttcctttctcaccaatacatttagaaaaatcaaaagaacagaaaaacaaaattcttaattattgattaagaaagaatacaccagtaactgtattttggaaattaacccagataagatatttttaaacttttaaactgtccattcgtatgtgtaaaacaaagtcaaagatacacgccaagcaaagaccttcagtattatttataatgaagtaatagtggaagacaataacttctttaatctacatctaggggaaggaatagtataaagccattaacattatattaacacaattatgttagcaaatattaaataataatagtaaaacgcttatgttttgtgtaattgtgaaatgagtagtattttgaattataggatgtaacttgaacaaagaaaaaaacatgcacgagagctcctaaaatgttgatagtgctttatattctttctgcatgtggcttgacgattttctttagcaatcttttttctattatttattttacaatatttcaaaagagaccagtattaatttcatcctggaaaaaataaacatacttaaaaaaaactcaaaatgtaacctttctacatcttagtttagatctaccaaaagattccaaacatggggtgttatttttctaagaacgtcacatttttacctgtggaaattctttaattcttactatttatccaaattcaaagcgtgccttaaggacctgccaaagtgccacattgccaagtcatttgcaaacatctactgagcatcaaccttccagttccagcagaaactgagaggaacaaacaaaaaccatgcccttgagatgctcaggtcagttgggggcaattggtgcttaaataaacaaacaagaaatgaggacatgctgggacaaatgttctagcaggggtttttgtgcttgctcctgagtaagcctcagaaggctctgctgaacgggaagtcaccaaggtactttgacaccagaaagtggaacatttgagccaggctatgaaggggacacaggattaaccaggcaaacagggggaagcatgagaaagcagaaatcaaatgagtgcaaagacaagcaggcttgaagggcatgtggtctttagaggcaaggaagagtcacagctggtaggaaagcagatgtattgagctgggaaatgacaagaggccagaggatttgtgaccatgtgaataagtcttgtaatggggcgagggcggcgtgggttgtaaacagggagcaacacagtgtacatctgagttcctaaggctagagggaaggagaggcaagaggtagtaagtcccatcaggtgaagatttgaaaagtgaagggggagttaccaaaacctggacactgactggcacctagtagactcaatgggtactgccaacaagacagacaaagtcacgggaagggggctaagaaaccagatgcagaagcaagcagttcactcaatcaaaccagccacttgtaggagagctaacaagatgtgtagagcatctctgattactaagttcaaatttctgaagatgtaacactagttcagaaggatattggtagataaaacgcgaaaccacaaaaggcgtcatttggaaaatgagtttggaaaaggctagattatgatgctggacttctcagagcctctaataagttaaataaattatccaaaagatgggtaagtgtttcccaaattatctggtcattgaacacttaattccataatttattaaggtcccttggaatgcactttggaaaatgctaccgggccaggtggtgtggcaaaggaggaaggggagagggtgtcaaaagtgggccttatacagatttattaatatatttacataaagaagacccaagagcacctataaggaaacacatcaaggaaggctcatacatgcgggcagcccctgtgttcctcaagtctacatctccaaaggcactggctggcccaagcagtgatctgattctgtagtacgctgtcaccatccaccctggatgccatcttttagcccacagacttagcctagggcctgacagtggctcccacgtgctcacgtcattcactcttgtttttatgaaccaagttccattggaactcctgcatcccacagctcctgcttcttctgcttgtcttactttctttctctctttcatactttctgagtgtcagctttgtgacaggaactggtgaattttaagccaggctgttctagtttatccctaactgaccctgtatttaaaactacaatctctcaacaaatctaaactcagatgcaattatctgtctttttttaaaaagttcatgtgtaccattgaagtttgtgttctgttcttggctagcagcagcataatttacctcttcatttgtgaccctgatatttgcaggaaaatattgtccccaaatgccattagtttcaactcctcaatccatcttccttgacacttagtcaatgattctctggctctaattcatcacatcgaacataaggcaaaaattgaattaaaatatttcagggcagatagaatacaaattaggacacaggatgtggttagcattgtgaaaggcaacactggagaagatataatttgaaacatttaaactgtgaattactttcagtgggggtagggagtggagaccaccccaatctagggctccaagtgttggcaatacatctatataacaaatagcttactgctctgggctgatgatcccacattatgtggaattgcctaagccttttccattgaatctgcctttcactgagaaaatgttccccatcatgccacgggaggtcacttctgtttatcatctcaaggttcaggaaataaaatggtgagagcctaggctggagggagtgtcactgctgcttttttgtacattccagaaggcaagatacaggtgtgtctggctcacaattataggctcaggacctaggacagttcctaacatatagtaggcaattttatacagtatttaacaaatgggtttagtaaatggatggacggacggacggacggacggacggatggatggatggatggatggatggacgatgaatggatgggtggctggttggatggatggatgggtggatggatgggtgagtcagtatgtacttatgcaaatactattacttatactactaatcatataataacaaacattgataaatttcctaagtatttttggggtatatttaatttaaccctttcaacaacattgtgaagtaggtactattattacctttctccttttacagacaaactaagcagggagtgctaactttatcagaatcatagagctaggaagtggttgtctcctattgtccaccttcatctggtgacagagaatagcaacaggaaagtgaaaaaggccactcaagcagccaagatactggtttttgttttacattttgcctgtgctgttgtttgtttattcaatttcacaccatttaatattcattcctttttacacccagagtcaagcatgaagactagccacagtgagagcataaaaaatcacgttgaacaaattaatgaacaaaggttaaacctactttatgctgggctctgcactaggtgcaaaggagctaacatttattgggtgccaactttgtgttagatgcctccaacttgtgtgagaggcgtgttagacgccttatgtgggtgaactaatttaattcttacaataaacccaggcatttttcctacgttttacccataaggaaagacaatgagcgtgggtgcctgtgcatgttttttctatcataccatctgcttccagttgcttcacaatgtagcaccttggctggaggggatgaggactgaggatgtgaactcccagggttttcaggccggctctgacattttgcagctgtgtgcaaccttagataaattagttcacctctatgtgcctccatttcctcagctgtaaaatagatgtaatgataataattgtacatgcctcaaagtttgttgtcaagtaaatgaatgctgataataaatgtaaacagaatctggaacaaaataagtatttgatgtgtgactctacttgtatagtttaaaaagtaccactatggcttggcgcagtggctcacacctgtaatcctagcattttgggaggctgagacaggagaattgcttgaggccaggagtttgagactagcctaggcaacatggcgaaaccctgtctctaccaaaaatacaaaaattagctgggtgtggtagagcatgcctgtaagtcccagctactcaggaggctgaggagggaggatggtttgagcccatgaggtggaagttacagtgagccaaaatggcaccactgcactccagcccggttgacagagccagaccctataaagtaccactatatttatattgagataaactatatagagaaatacagacagaattgacacctttatcttgctgaggcttctgaagagcccgttagtctctcccatttatttagggctcttgttgtttaccttcagagtatttttaagttctcttcatatagctcttgcacatttttagttaagtttatccttaggtattttatattttctattacttggtaaacagttttaccattactatggtaaattgtatatcgatttctatatatgacttttttgccttgcccccttattgagtctctcttagtagtacattttacttgatttaagttttccaggcattaattatgttatctgaaaacagcaataattttacctcttctttcctaatcttcttgcttccaattcctttctcttgcataataacattggctagtacctctagttcaattttaaataatagtgttgatggtagatgctcttacctttcaacctccttttttacttgaattcccctcctttttactctgagttcttccattgttttctcagtaagcatgatgctggcttttgaattaaggtaaatatattttaccaagtaaaattaagtatcaattataatttaactaagaaaatcttcaagaaatctatattgaattttgccaaatgccttttcagcatctatcaagatgatcatatgcttgttctctttatatcaatttactgaggtgaaaaatgttatagatctcctaatgttacataccagaacaaattccatttgatcatggaatattcttttaatgtattgctggactctctttgctaatattttatttagaatgtttgcttaatttcataagtgagattggactgtaattttctattttggggacctcttcattgagttttgggatcaatattataattgcttcataaaaaaaactttggaagtttttcttatttttcgatctgagctgcacatcttaaaaaatgagcaagaattagccaggggaggaggctggaggataaaagaaggagacattacattaaaggcacaaatgaaagaaacaaacatcagtatatgcggagaactctgattctgaggctgcttcacacaaggcctgagcccttctcctctccttcaaccctgaatgtctgtttggaggtagctgtatacaattgttttctcttgcatattgatccacaatgatttcccacatcagtatttttttttccttcttcaataagattatgagaccttccttttgtttatcccagagaagttcttggattaaactccagaagcacatgggtgtataaagagctaggccatggtatctcactaataaattaagtcattcacactcattaagtcattcagtcacttattcttccaacattatttaaacctgagggcaagatctggaaaaataagaggcgggctttgctttcaagatcttagagtctagcataaaagaataatactagtactgttgtaatctgcatgacaaattaagctactaggtggactttgctttttcaatacaaaacagttttccctacaaatgtttcttcaacctctagtgcctgccaccaaaatctcaaagatacaatcagacaataagaagcacttctgggataagctagaggcagcagcacttgctgagtttcacaagtccttctggtagacctagtgctgtgggtcagcttgtactttctgctaatatctttctcccagatcaattctcagaattaaatatgctcatgagtcactgtgccccatccagaatgacatggctcccaggatctctacttctcccagacatatttgagaatcagaactcccagagagttagagaatatgaccgggcatgcagtaggtgattattgaaaacttactcactgacaaatattttgacattcagctacttaaatcctgcaccatcatgatgcaggaatatactgcaccatctagaaagggaaacaataccaccttcttcaaggaggtatcacaaggcaacagcacctcctttcacaggagatatgtgagaactaagtacagtaagttctcagttcttggaaaccaagactttatgtgaaatgatgtataacaaaatcgttttccttataaacatcacagtaaaacaacattattcattttttgagggccagctgtctattgtttttccaagaacctattgagggcgttaagtgaggacttgctataataggagtgacagaatcaacatgaattgaggacctcctctgtatacacactgtgtgtgctacgtaatgcctcatgtctttttcacaacaatcttatttgatattaataatataaatagtcgtgcacagtggctcactctgtaatcccagcacttgaggaagctaaggcaggaggatcacttgagtccaggagtttgagaccagcctgggaaacacaggaagaccctgtctcaacaaaaataaaaataaacagctgggtgtggtagcacatgcttgtagtcccagctactcaggaggctgaagtgggaggatcacttgggccccgggggtcaaggctgcactgagctgtgattgtgccactgcactccagcctgagagactgaaagagaccctgtctcaaaaaaaaagaaaagaaaagaaaaatattttaaaaagaaatgttaaaaaataaaaaatagcactacctactttttacaacaaatgttttagtttacactagttttagagtaactgaaaacttgagaagatagtacagggagcgctcatatatccacaaccagtttctgtattgttgacatcttacattaccatggtacatttgcacagctgtattaactacagtcacattttttcagatttcaccagtttttcctttactgacccttttttctgatccgggatcccattcaggattccatatgatgtttagttatgttcattaggcttctctagactatgacagttttttaactttgcttgtttttgatgaccctgacagttttgagagataccagtcaggtattttgtagaatgtctctcaatttgggtttgtacggtgttctttcttctcatgattagactgggttatgggttttggggaggtaaagtggcattctcattacatcatatcaagagtacatactattaatgtgactatcactgttgatgttgaccttgttcacctggctggggttgtgtttcttaggtttctgtatatgaagcgacttttatcattccttctttacatactgtactaattagcagaaagtcacaacgtgcagctcatacttaaggggtggggagttatgttctacttccttgagagggagtttctccataaattgtttgaaattcttctgcactggagatttgtctagaagcaacgatgccccagtagcaatgagcatacccaccccattgtccaataaaagaaaccagggttccttagagtaatgtctgattttagggctgggtcagggagtataaaaatgaggctggagtatctcatggtactacaaagtaaggaggtgctcaaacagcacacacacacacacacacacacacacacaatcacacaatggggggttgtcaaagagacacaggagccaacagaaatagttcccaatgatgaggaactgtggttcagcaagattcagttactcgtccatgagcactcagcaaatggtggtagagccaggacttaaacccaggttctgatcctggaaacaagttcttataagcatcgtccacacacctggcattataagctccttgagacaatgtgttcagggtgtttaggaagacatggagtatgacaaggcttgcaataaatatctgctattaaattatcaccagtactatccccactcacagtgaggctctttatccacactttcctcactctagggctttcccatgctgctgtattccctctgctgacaatgccctttcacctgttctctcttgctggaattccactcatcttttaagacttggtggaaacctctgctgtctcctagcctggctgactcttatattgtgactctcagcactctatctaggcacctcctctgcctattcatggtttccaacggctgttacctccagtttgtcaagttcaaagagggcagggacaatgaccacacacaggaagcactcaataaatatccactgaatgcctgcatgaataaacagcgaatttgtatttgtgatcctcttggtgcccagcacaatgtttgagcaacagcaggtgctcaatgcgtgcctgctaaatggatgaatgaatgaatgaatgaatgagcaattgaaacttcttgcagaaagctgaccccacagcaggaacaggtatgattaagggtatcctgtaatgtcctcctgatgctttgatgcttgcctgggggtgagtcctcagcaccactggccagctggtgtggggacagggcagcatctgtgagctgccagccttctccttctggcagtctctctgtttgtgccattagagccccgtattttcctcagggcctcaagcagctccaatcaattgggaagaggagagcaagacgttcaagccaaattcagctgcagagcaacgggggaactcgttacacagttggtgtaggtgggaatcacgcactccaggctgagaagaccctggggagcagagcccatagcgaggaatgcccttcttactggtactattggaaataataatttgttaataaaaatattaacaataattggttaataacaacaggcagtatttctcaaatgctaaataatgtgccaggctctgtgcaaagtgcttctcatgcctaacctggcttactacttaccagtacccagtgaagtcagtcatcatctccattttacagatcaggaaactgaggcatagaaagttacactgtccaagagcaagcaatttgcaaagcaggggccagaactcctaagtctaagcccttaaaccactactctagggttctgtgcactccccattttgcaaatccttgggaatatccctcgtttcctggacaccctctctgatcactcccagatacatttgttgttgactcccaggcacaaaaagattggtgtggacacaaatcttctcacaaccttgccttatctaccctctcctccatcttcaatctgactctccttgaggtcaggaactgaggttttcttatctccctcagtgggacatggggcacttagtaagcacgtttgttgattggttgtttcacagggctctggtagtttgttgggcctatgtcttcctctggagtctttagccaccatgtggcattacaatatgatctactggaaatgatactgaatcccaaagcaggtacttaataattgctctaaatgggactaagagtctgcctactgcggttgtcaactttatacatgggcagtcagggaagacttcccaaaggaggagatgtctgacctgcgtcatcaaggatgagatggaaatgactggcaagtaaataaatacaacctatggctaccttcaatatgctggtccccacagaagagcaggatttgtgaaagaaatccctctctgagatcttgctctgttataagtggggacaagtatcttcagaggcagggaaggcgatccttgtctttgtgccaagctaggacaaccaggcaaaaaccaacaccactctccacgaagaaggtaaagctttatccctttaagagtcaagaagcacaggctgcatcctcccaagcactagctccaacatgaacagtcaataattccaaagtaaatatcagcagaggaggcctcatgtttcttcaatctccctccgcacacagcaaacacattatgccactgccctttggtgagcacccttccttaggctgcggcctgtgattgaatcctgagagctactccaggggggatttgtagcaatttaagataccgttttgcacagggtaggtacaaagtctcggtcaagccttgcagtaaagagcagccagtctttcaggaccatttagccccaggccttacccatcactgccagctcctgtcccagccctatccttccccatgctgtctccttgcttcctcttgcagccccatctccagcaggggaacatccgcagccctttaatggcttcctctgggcttctaacatcatgattctatttcttagtggttctgtcccaaaattctgagttccctttcttgcttaccccactggtcagctccatagggcttttctttccaaggctgcagcttcatggatggctccaaaatcagactgtctctgacactgtcttatttctgatactcactagctaaatgttcttgggcaatttcctccacctcttcaaacctcaagcttctcatctgtaaaatggacaccttattaatactactcccccagggctgttctgagatgggagtgaagacagaaaagtgcttagaggccgggcgcagtggctcacacctgtaatcccagcactttgggaggccgaggctggtggatcacttgaggtcaggagttcaagaccagcctgaccaacatggtgaaaccccatctctactaaaaatacaaaaagttagccgggagtggtggcatgcacctgtaatcccaactgctcaggaggctgagacaggagaatcgcttgaacccaggaggcagtggtttcagtgagccaagatcgtgccattgcaccacttcagcctgggccacagagtgagactttgtctcaaaaaaaaaaaaacaaaaaaaaaaacgcttagagcagagcccagcagggtaggcctcaggtaagcatccatcactctccttagcatcagctcatgggccggttgtgaatattagaggcagaaaggcatgcaaactgcagagctcggctcctgggggaaagttccctgtgaacgttagcatggttagtaaggagcaggtagaggaaagaaagaaccaaagaaagaacagaaacacgaccagagataatagaatctcacagaattgccatggcctcagtccctggttgggattctgattcgctaatggaccaccaggctcatcccatctccctgcgctttcccagccccatgcaggcctttttcaccagggggctcctcttaaggcaagtcatactagcactcagggcctccagcgtcagacatgggcattgaaaagtcagccacctgcggtctgaacacacagagagggagaccagagagagagccctggagacctacacctggggtaagtcacttaacctctctgggactccatatatttattaatatcattgggcaataataaaacacctaacacgcagacccactgtgaaaattacatgaggagatgggccttaaaacgctaccagatattaatatgacatactaagaatcatataataagatgctgggaatgacgtttatcaagtgctttttctctgccaggacaagatctaagagcttgacatgtacttctttgtctaattctcacctacctacatttaactacttgcatatactatctccatttgactcagaggaaactgaggcacagagggattaagtcaatgccccaggtcacctagctaaccagcgtaagaacaaagtgaaagtccagagcccccaagacccaatgtcaattctgcccctgcttgctgactgcattgaaagaaattaacaggtctttgctcttctctcccactggacaaagcttagcacctgacagatacaggtgcccagtgagctctgccgaacgactacaaggagacagctgccctgaagatgagtcctccctgcccagctccccaggttggttccacccaaggataagctcatcactcctgcacactttttccagagcccctgcaggccacggcaaccccctcagtggagagtcatgttgccagatcagcatccaccaccctccccgaggtgggtaccagcgtctgagtctacccccacccccagggtgcatgttctcccgggtgccatcagagaccaccccacagggcagccagaggggaggacgaaagccccaggcagctctcatggaccagagcctcaaactccctcctgtcaaggaggccacttgtcaccggagaaaactgcaagggcccaggagtcagacagacctggattggaacactggccatgacccgacttgctccatgttgtcagggaagccacttgacctctctgaccctgtttcctcccttataaaatgcaaaacctaccagcccctttctcatggtgatgttgtaaagacaagataaaggacaaatgccagctccagcaccaggcacctcaggaaatggtgactcacttctagaacagagagcagagaaacttcatcgagggatgtcttgcctgaggctttcccttaggtcactatcctcctccactccccgacccacttctatcctacctcctactgcaggggccttggcccagtactgccccccgctggcacttgcggcacacacccctcccggaaagctaacgttttcaaaaaacttttatttcaggttcacaggtacacgtacaggtttgttacataagtaaactcgtgttacgggagtttgttgtacagattatttcatcacccaggtactaagtttagtacccaatactaatttttttctgctcttctctctcctcccagcctccaccctcaagtaggccccagcgtctgttattcccctctgtgcatccacgttttctcatcatttagctccaacttctaactcagaaaacgtggtatctggttttctgttcctgggttagtttgctgagtatgatggcctccagctccatccatcttcctgcaaaagacatgatctcattcttttttatggctgcatagtattccatcgtgtatatgtaccacattttctttatctaatctgtcattgatgggcatttaggttgatctcatgtctttgctattgtgaatagggctgcagtgaacattcatgtgtacatgtctttgtggtagaatgatttttattcctctgggtatatacccagtaatgggattgctgggtcaaatggtagttccatttttagctctttgagaagaaagctaaccttgaatctagtgtttttttcaaacttaaattttataagaaatcccaaagaactttcatttatatggatcatattcaccaacattcactgcattacaaactaaaactaagatattttaaacacatttttatttaaatataaaaatattaaacttattacctgtttgcataaataacatatatttatgaaaaaatcactgcattttccaaaacaggtaatcactactgaaaagaatggcactgtttttgccttttgggaaatctctcgagtgtctggtttaataggagacagctgagctcacatctctgcttcagcattcaatctgtcttgatctcactcattctgcagccgctgagaaactccactgtacacccctgtgagaatgcgactgaaaagggcacatgaaatcttagtatcatgaaaatagctttgacttctcagacttcctgaactgtctcagaggtcctggggtatggagaaagggcaaaccacactttaagaatagctgctttaagtggttctgttccctgctgatctgttccctagaacattgcctccctctcctaggctctccctgcaagaatgcacggaaacaattcccacccatgtgtctttgctgggttacactctctctccaatgccatctcggcctcttctcctggcccactccttttcacctctggaaattcacaacaggtatcactttccttagaaattctcccctgacccctatcacccccatgccaggtaaatgctcacctatgctcaccttacgctctcttcgcatgtcattcgtcatgctagcattcaccactctggagtcaattattcaggttagaaatctgggtctcatttactgactttcagctccttgagggcaggggggtgtcttattcactatgatgagttagcacgaagcccaggcgtatctcttgtcaggccctcatggaacatgcatgtggatgtttttctctaattgaacttgcctagcatgccagatttattttctctctacttaacttcatctgtccaaattcccatttccagtcattaccctaattcttcaattctaagaggcatgcttttttctccccacattattatatttctaaaatcagaatgtcttaagttgatgtgtacatttaacacttctgatatttctcttcttgaaaagttgtagttaaattgacggcctatcttccaatcagcagcattttacattataacgagtataattactctgttccaaatacggtgctggcagtccacaattgtgcatttaactaagtgcttccgtacagtcctcacattgttctcccaataactcacccagccctgggcacacagagcagtggtttcattaattgggagttttttggcagggagaaaggaatggaagaatttcaggcaacgaagcctacatgctgagaactgagagagaaaagtgccatccagcctcaagcgatttgagctagaaaatacattgcttttgtgcatatgactgtccagacacttaagaggttgatcccaatctctttgttttttgacacctgcaaattgtaactcacatttgcatatcttttgccacttggccagttgttttgatatctattagcatatttgatcttcccagccatcctatctgaaaatcagagcaggaattgttctcttcatttcattaagaaattagaggcctagagagataagtgacactgacttaaatcagaagaggaactggatccccaaagagcagagcactagtcctgtgctaatcccctccaccatgcaactgccatccactccaaataattggaaggagtgggtgaagggaaagaatgaggaagagactgagctatgaaaataaggacacaacgaaggagagagagaggaaaagggaattgcgatggttaatttcatgcgacagcttgattgggctaagggatgcccagacagctggtaaaacataatttctgggtgtgtctgtgaggatgtttccagaagagattaccacttgaatcagtagacttccctcaccaacatgggtaagcaccatccaatccactgagggcccaagtagaacaaaaaggcagaggaagggcaaatttgctctctctgctttggttagggcatccctcttctgcttttggctctggacactcctggttctcaggccttcatagctggactggagctaaaccactggcttccctgggtctccagcttgcagacagcagatcaagagacttctcagcctctaggatcacatgagccaatccctcataataaatgcctttctagatatctctgtatatcctcttggtcctgtctctccctgactactacagggactgatggttaaagagcaggaacaaaggaacacgagaggtaaggaaggggcaggaggatgaggctgggtcagggagtccgcaggcctcccaacccatcacagaacttgtcacactctactgaaattgctggcttgcttgtttctagctctggctgagactataaaacctacagggatggtttgtctgtcctgtttatgactctcatccatgtccaaggctaagcacagtgataggtaaatagtgggtgcttgataaatattagtggaattatcttcaaatatggtagtccttggagccagagtaatatcgggtcaataactttactctctctgattccgttttctcatccataaaataggtataacaaggtctttcttagaagattatgtcaataattaaatgaattcactccgtcaagagcatttggaactcaagaaacactggcattgttagactgcagcccataggataccaatgccattgccacaaatagggcccatccttggaaacacagtaatcaacatattctatggacttggaagaaggttctacaaggacagattattttatttgaaaaaatgacactttttttcaaaaaaaagtggaagatactgattatgaaagtcatacaaacacattttttaaaagtcagacaatatggccgggcatggtggctcacacctgaaatcccagcactttgggaggccacggtgggtggatcgttggaggtttggagtttgagatgagcctggccaacatggtgaaacctcgtccctactaaaaatacaaaaattagccaagcaaggtgggtttgcctgtagtccaagctactgggaggctgatgcacgagaaacacttgaacttgggaggcagaggttgcagtgagccaagatcatgccactgcactccagcctgggtgacagagcaagactctctctttaaaaaaaaaagaaagacaatactaaaaacattaaaaaggaggtaaatataatttatactccaatccgatggcaacaatcatattgctatagcaattatcatcttggtgttactttttccaggacacttttccatacattgccttaaataatggaactatattatatactaagacagatactcttgccttgccacttccaaaccattctcttacagtttgttcactaatagaatatttttgtttgttttccgaatggagggaccatgtgctttaaatgaggtcccttctgttcccaaaacatcgatcttgactaggccagaggaagtctcactcgttccattcttattgctgatgattgatttgtgagtggaagaataggcatgtggtcaattctgggcaataagacacaagggagtctggtggaagcagcaaaacacttctaggcttgcaaaagagacttccaataagaaatactttttcctgtttccaaacataattgcagaaggatgtgatattcagaagagtagcagccatcttttaaatatgagaggaactcacagttgacttcagaagaagcactgagaagccaatgggaaccataaagttattgaacgttgagttaaccaagccaggacttcttgttctataggatgataaacacccctcatacagaagtcccctgcattttggttggctgttacttgcagcccaaagcattctaaccatacagttactatttataaccgggtttatgtgttcactttttaccttctctacacatcaatgaacagaatcataataattaatgtctatgaaagacttcctatttgcaagtactttatcaggcacattacatggattgtctcatttttaatccttgtgacaaatttctgtagtcgaaatgattagctctatttttttttcagatgaggaagaaatcaaagatttggaaagagcaaatgatgtaacaacaaaatggaggggcaggaatgtgactcatgttgttcagttgagggtctaagtttcttctccacaatcacagcccttaggactcccctcatcatttacaatagctccaagcaccctattttctgtacataacatgcacagctaccctcccactgatgcccacatcccacaaggtgaggtctttttccctcttcccttcccaaacccttaccagttggccctacgtgtttggcatcttctaattatcctgattcctaaatatgcaaaataataatattccccaattgccttctgctgtaatttcatgtaacaggtcccctcaaatagaacgtccttaattttaattgcatattatgttccctttctcagggcagacagctatttattcatctagctaattgttcaggagacaaagacatggaatatatctcaccaaattaagtatttgctagttgctattactactggggctataatatatacccctgatttactttctagaagctcctgaggaaatcttggcatctttttttttttttttttacatttattttttggcttagcctacctatgattgtatgaaggccaacaaatcctggggtattattattagttgtgatgataatagtaattaccacaatgtatagatatacaaccatacgctttattcaatgaaagaagaaacacagtaaaaatggcttttattgaatacctattgtgtgctaagcattggcctgggtatgagaaaatattagcccattgcctcatttttgttcaagaacttcttaagagactaaaagcctcatgtttactgatgagaattgaagggtacagaagtgaaaccacttgtctgcacccggcacacagctgggcatcagaagagatgggttttgagcccaggccagtgaagaccaaagtgtaggttcttgtggggctaccacagagcacatagccccttccaacctcactaattacacgtgagaaagaggcaagcatctcaatggctactttatagatatggtcacctgcatagataggaagtgattttcccgaccttattggatcaaattattcatcagaagccggatgaagaagtgctaattatcatgcaaaacagcccccgagactaagaagcctgccttgcctgcttcccaagctgcccacttccagaaagggtggggctgttttgccacctgcacatgtctggatttccctctccatctcccatctcccacccttcacacccacacacccgtggctgcaggaatttcagccacattcaccagacttaccttctgttttttcccaccctccctccctccctcccagggagtcagtcccacagctgagtgagagaaaatgcccagagaaaaaccatcagttcatttaattaatattcacggaatagcctgcctgtattaggtgggtactgtgcatgcagagatgtgataaacacagctctgcagacctcccagccccagagaaagtcccagctcatcacgaccctatggccaagggagattgggcaggaactaataccttctttctacagaaaaagaaatgaaagcagaaaaaaaaaaaaaaaaaaaacaaccttggaagaggagaagggcatggtgcatctgaatagaggctccaggaacaaagaagggaggatagagcaaagggaaaatgggcagtcagcgctgccaagaaacctattaacctgaaagtaattactgagtgccactatgtgctaaggacattgcacacatatgctcgatccaccacaaggttggtccgttatccataattttagacagggaaattgaatctcagagaggttaggccactggctttagactgcacagctgaatagcggcagtgttaacattcaagcccagatcggtctgataccaaaactcattccctgtgtgagacagtccaacactagcccgaattggcattcatgaaagctccacagctgagatggcacactcagacaaatgtgccagacacttccatacactcaccaagtttcatggttttggattcattaatagcagagatttccactaagaaacgacagcccttcccaaacaagagctgtagagaattctaggtcttctaggattctagggaattctagggcttctgtagggaattctagaactgttatgctggaaacacaaggagaacactttccccaagagaaaaattccccattaaggtaactctcctaccagcctacaactagagctcgtttattcaacaaataacttattgagcacctactattgccaggcactgttctaggtgcttaagataatcagtgaacaaaagaagtgttgaccttgtggtgctgacattctagtgatggatgcagaaggagcaaaacacccccaaaccccagctacaaagcccagcagctcaaatgcacccagagagaccttccctttactttagcttagtcctctcccacaaccttgtacaccaggggatcagtaaggcctgggcagactttgtctcaggtcaaactgagcctcctggcagaaatgaacaggaaacatcttgaacatgtgggtagaccccttctgggctggtcagccaaatcccatctcaccaggagcctggaggcctgcctcgtcatggcatctgtccactgaactaaccaggcagagagcttgtccaaatcagattaaatggcaagcaatgcaggactgcctcccagggcaggctggccagaaggcatactgtaatacagttttaagtctttacagttcttttttctttaataacacctttattgaaaaataatttgcatgctgacatggtctggatatttgtcccttccaaatcttatgttgaaatgtgatccccggtgttggaagtggggtctggtagaaagtatttgggtcccggaggcagatccctcatgaatggcttggtgctgtcttcatagtaatgagtgagttcccactctatcagttcatgtgagagccggttgtttaaaataacgtgtatcctgctccttctctcttgctccatctctcaccatgcgacatgcctgttccccttcccacttccgcatgagtaaaagcttcctgaggcttcaccgaagccaagcagatgctggagccacacttgtacagcctgcagaatcatgagtcaaataaacctcttgtctctatcaattacccagtcttaggtattaatttatagcaacacaaaacagactaagacaaatactgtaaaattttacaggatttttaaccaactcactgggagggtcaggcattttgaagtgaagtgtggagacaggtggccagctcccaggagaaagagatgcctttagcctcaagagagtcagtgcaggcttaggctctgagctccttgtgaattggggaagggaataggagaagggtgtcacgggcataccatccttggtgggtaataatatggaagagccaggtaaggaatcaaaggagcctgcacccggatcctggttctgccacttctcagctctgttcatttggctaagttattgaccttctgtgaacctcagcatttccatctgtaaaatggagctaaggcaagtacctgcctcagggagctgttggaaggattcaatgaggcagtctctacattgtcagacctggctccagcctcacagcatcatcatgagaagtacctaattcttcctaaaactctctcattctaaatagaaaaaactccaaccccttgtcttccaagggtccacagctgaaaatgccccacacccaaaccatccctttgcctgctttccccatgaatccagcaactttgctttgaagaccaagtgctcacccaccccaatgaagatccaagataaggggaacagaaggcacaggttacagaggaatgcctccggctgctcctgaggccacacggcaggcagggcctgagaaatcagacattttctcatgaaaaatggcttgtgcagtgagtttttcttgcagattgtaggatcgccatggtgacagggccagggcagcgagggaggtagaggcactgccagcctgcagggatctggggctgggggaggctgggttggcagcagaggtcagagaaagcctcaggctgcaacaggggccatgaagcaaagagaagaaggcagtaactacaggctggtccattcacttgacactgagatggtttggggtccccaccttcatatacttaacagacctaggctgagccccagcgccatataacatcaactgggtctaaacactggagggacagacaggactgtgcttgttcatctctacagccctcagcagccgaagcagtgcctggtacatgcacacacagaggatgcagaccttgtggatgcggatcgggacagggtggtaggaaggttttcctgatggaaaaggaacctatctgtagatgacttttttcctgtttccaaagatcagatcggcctctgagcattgctgagcaaggaaccaaactgtgcctctacaacatcctggctgtgtaactttggcaaggcaggtaacctctctgggtctcagtttctcctctgaaaaataagggttaaatgagtgaatagaggaaaagcatccagagtttctgagttaagtcttagctacaatttgctgtaatgcactttgtcaggcctaactaaggagaatgggagtcagtgttagaacaccagtttgtttcctgacaaattcctatgcatccttcaaaccccaaatcagagttcaccttctacaagaaggcttctctgccctaagacctgccctctgactcagtgagtctttgctaggtctgtttccccagccatgctcaccccacaacactgcattttgtgttcttatctgttcccttgtcccggtgagctgctatagctcttatttttgtgtccacatctcctagtactgtgtctggtagggactgagcaccaacatatttcaaaagatgaataaacgaaaggatgaatgagcgatttcttcacaagaagagcagataggaaggaagtaaacatcttgaagtaatgtgatggcaatttttcaaattccaggacggagacaggctcctacaagcacacaaacagaagtaaagaggtcatgtacaatggaagataaaagaatcagagggccctaaagacttcttggtacagtgcattaaatctcagatggcagctgaacatccaagctagggcttaatgggaaaaggttgcaacccaagaattctatacctgtcagctggtcattcatgtgtaacggcaacttggagacaaaagcatatagtcatttgagaaacacgagttcaaggagttttttaatatttaaaagtaactgccaggaaaagtaagagcaggactcataactttctgatcgatagaagcagtatattaaagtcacacagtttaggtttcagtatcaattctgctactctatgtctagtgctaagtcatttaatctcttgctgccccactttcccatctgtaaaatggaaacactgatagtacctatttcacaggattttgggagaattaaacaggataatccacatgcaatgcttttcccagtgaccaggacatagcaggttgcagtagatgctgtgctgccaacagatcttccctctggaacaaaagatgaccctcatcagggtgccagagttctctacaagaacacctcagcaaccagctcttagaggactatgttggctgaaataaaactcctcacctaagatcatgcctctttccacatgtggtccacagccaatgactcaccaacacacaagtaaacaggcagcccccttgccccaacttgaaacaattctgagagatcatcccagcttcagagattgcgtgaagtcagttgaggcctttgcagggactgcaacgcagctcaactcttccctcagctcagtgctgcttcctttccttcccttctacagttgccaatgtcaaaggtacttccttagaaatagcatgcatgccattgtccaactcagagtgtgctttctggggagcctaacatgccagcaaagagactgcagtggtctgaatgtgttcctcagagttcacatgatggaaactcaatccccagtgcagcagtgtagagacgtgaggcccaatccaaggccattaggtcaggaaggttgtgtcctcaagaatggactaatgttatccagggagtgggttagtcatcttgagagtgggcttgttataaaagtgagttctaccccttctgctctctcacactctcctcccctgctcttccccttcctctcttccttccaccatggaatgatgcagtaagaagaccctcaccttgaagagcagaattctggctgctagaagttggtaggagagggggaaggggcaatggggacaaatttgtgtatgggtacaaagttacagttagaaggaggtgtggcagtgagcccctagcaaaaaacaaaaaatataagagcaaggaaaaagcaaggaagtgtaatgaacaggaagcataaaacacaacaagatggtaatgatgatggataatcaagcatataagttaaaatttgtttaaatatccttaaaacactctatccgggcaaaaaaaaaatcaagtctaacaattttgtatctactagaaacacatttaaaacaaagtaccaaagaaaaagtaaaaaataaattaaagggccctgggatggggggatggtgcagggaaagatcagactttcagggattgcttatgtggtctgaaaaatccttcaggagatcttgatacacgttcctaaaaaagcatactctcctgattggaaaagaaatgacatttcacatcagaatttaagggtggtatccaaagctgtccttagaggaaaatgtatagccttgcatgctgtcaatattaaaggaggctagattaaaataaataaagtaaggtttcaatggaatattttttactcttaaaaacagcttaataaaattaaaaatataaaaatataaattaagagtaaaattgaaatataaatcagagccagttaaacatagacaaataaaattttctcaggaagcatatataatcacagatatatatcagtttttaaatctcaaaaatattaattatagctagttctgtgtagttatccttgaagcagttggtgaaatagatggttttaaaggtaaatataaattaccaaagtgagttcaaaaaaaatcttgagggatatcatacaaaattaatcatccttttaaactatcaccattaagtaagatatttcctattggtttcaaataattcttaccatattaaagaggtttatttctatgctattgatataaaaaattggaaacagatatcaaactggatcaattgccaactggtcatctattaaggtgatcatataatttttttctttcacttattaatgtgatgactcctactagaaactcttctaatgttgcactatcttttattttcctggaatggtttatgaatcattcctctgactctaaggaaaaacagaaattctctaaatacattttggggagcattacatcatgttgaaaactaacaacaatatgatttaaaaaacaaagaatatcttcactacaaagaaatgaaaaatgtttgtggtgatggatatgctagttactctgatttgatcattacagaatgtgtatatgtattgaaacatcacactttcccccacaaaatacatacaattattatatctcaattaaaatttgagaaaccagaaagaacattctctcaaatactcacagaagccaaaatcctaagtaaaatcttggtaggagagtttagagcatattaaaagaaaaagaaaatcatcatgtccaaataggatagcataattcaagacgtctgaaaatagggctcatgccaagaacacaaaagatagcccaacattagaaaagtttccaatgggagtcattacatcagtaagtcaaaggaaaaatttataaaatcaccttaatagatatctaactggtaattaatcaagtttgatgtcgttcccaatttttaaaaatcagtagcatagaaagatactttcttaatatggcaagaattatttgaaaccaataggaaacatcttaattaatagtgaaaacttagagctagtttcactaagacaataaaggaaaggctttaagaagtagaaactctaagactcaaccacaaaatagaagactgatagaggagattttatacataaaaatgtgaattttttgaagaaggaaaaaacaatgaacttcaaagaaaagtgacaatttgggaaacccatctgcaatatatctgatatcaaaagattactgtcctcagtaaataaagacctcttagaagatcctgggagttataaaagaagaaataggctgggcccagtggatcaggcctgcaatctcagcattttgggaggccaaggcaggaggatctcttgagtccaggagtctgagaccagcctgggaagtatagtgagatcccatctctacaaaaaaaatttttttaattagccacttgaacctgggaggttgactctacagtgagccatgatcgcctcactatactccagcctgggcaacagagcaagaccctgtgaagaagaaggagaagaagaagaagaaggaggagaagaggaagaggaagaggaagaagaagaagaggaagaggaagaggaagaagaagaagaggaagaggaagaagaagaagaagagaagaagaagaagaaacataaagggttaaacaaggaacattttcaaattcactggaaataaatcaattaaaaattaaaattaaaatgtgatatgacttttttttccatcaaatgagcccagatttttaaaaatgatcatgcaaaagattctggaaaagaggcatagtcgttgtttgctcgttaaattttaaatggacttaaatttgttttaaagcaatttggtaataagtttaacaatatgtataaatattcacaccttgaccaactgactccacttctaggaatacattctgaagaattaaactaaagagtacgagaaaacttcagaacagacagtgccattattctagttaaaaagtaaaaataatgtacaagttcaacaatagggtaccgattaaaaactttctacctttgatggaatacaagaaaactattaaacatcatcaattagaaaaaaaataatattagaaaacagttgagggctatacttaagtgaataaatcatgttgccagtagcatgtatagcatgacttagatttgtatattacaaaaagaaaaaaaagatgagaggggttaaagtttcattcatttattaatttcttatttatttcaaaaatattgattaaacatctactaaatgtcaggcctggttccaggcattgccaaataaaatagacaagatgcttctcttcataaaatagattaatggagttatttctggaaggtaggattagtaatagttttcacatttttcattgagattttatacttctacccaagtgtcttacatgaatcgtgattttaatcatcatgcagtggttctgattccattctatgatttggagtatagtgggaaacttaaataccgacacatacatggataaagcagattcttaaaaaacattctggaaaaatttggtatcaagtggaaagaaaaaacaaacacatttggcctctacctcacaccatatacaaaaataaattccagtttgatagtagacttcagtatttaaaaaaatgaaatttcaagaaaaaaatggagaaatcttcatgaattctttcataaaggacataactgatcaatctcactgcattaaaagtaagaatctttgttcaggccaggtgcagtggttcacacctgtaatcccagcactttgggaggctgaggtggttggattatctgaggtcaggagtttgaggccagcctagccaacatggtgaaaccccgtctctaccaaaaatacaaaaattagctgggcgtggtggcacatgcctgtaatcccagatacttgggaggctgaggcaggagaattactcaagccagcagcgagctgagattgcgccattgcactccatcctggccaacagagcgagactctgactcaaaacaaacaaaaacctttgttcataagacaaaacaataacaacaaaaacatcaatagagagaaacaccacagagtgagaaaaggcatttgctgcacatataactaacaaagggcttgtatccagaatctgtaaggaaaacacaggcaacctaatggaaaagtgatgtagagactagagtactcacttcatgaaagaggatatccacatggcttttaaccaaatgaaaagtgagcaaccccagaagccactggggaaatgcagattaaagccacaacaagacactgccacatacccatcagaatggctaaaattaaaaccattaacaacatgaaagatggttgagatgtggagtagagagaactctcacatgctattgctacgagtataaaatgctacaccctctcaggaaaacacctagacattatgtaatgtatttgaagattaatgtaccctttaaccagcagttgtgtacctaggtacaaactttgcaagcacacacgcatgtatgttccaaaaagcacatacaaaaacactcctaacagcattatttgtaataataaaatataagaaattacctaaatatccatcgactgccattggtagtatggttatacaatggaattctacacagcaatgaaaaggagctagagctacatgcaacaacatggatacaactcacaaacgtaagacttagtggaaaaagctagacacaaagttaacaccttctatatgtggttccagttatataaaaccaaaaacagacaaagaaaaagtatagcatttaggaatgaatacttagggaatgaaagcatacaataaggtaggaaagtgatgagcataaaagtcagaatagagattgcttttgggggagagtctgggcttgagtagaaagggacagtgcagcacctcccaagtactggcaatggtcttgctcctggtggtgacacagttgttctctttacagtaattcagctgtacattgtgttacgctttctgagttataatacaccataaaaaatgatattaaagcaacccaacaaaaacattaatgcctgggccccactccagaccagtgaaatcaggatctttgagagcagggtgtggtatgggtgttttgtaaacattcccaaggagattctcatgcacctccagggctgagaagccttggaacactgtataagagcaagctcaggttctccagtctccagcactctccagcacttctctctgggcctccatttcctcttcagttaaatgagtgcaataacagtactgacctgatgaggttctaagaattgcaagtgccaacaccatgaagggtttggagtatttaccggcacattttaagtacccgatcagcattagactggtatctttataataataaagtagcaatacatttcgtttcttaaataagacaatggaaggggtcaacacaattccaaatatttagcaaacactttcaaaatgttattactattaaatatttggtttccacaatctcaggatgaactaggatgcccctcagctctggaaagcctgtctcctttgggcttgggaaagaaatgcagtttcagaaaacagtccagtgatattcagaatctgctggaataagtggatattaccacttggaatacccccactagtttttagttttgtaaattctccagagacaaggcccaggcccttcttttcttgattctgctgatagtgcctagagcataccttgtaagtgctaatgggcacaatgctattttgagccaagactcatgggttcggtaggtcagaaactacccatgaagatgatggtatcaaaagggcagagcttaaaacttcattccaccctttactggctgggtgactttggccaaggcatgatctctgaaagcctcaatgctcacatctgtaaaatggagaggatgacaaagacagctgtctcgcttcttcaagatgttttggtgaggcgacgccgagaagagatgcaaaaactcctaacacacaatatgtgcccagctaacaaatgctttacgcctctgcacccagcaggtggccgggtaatggcactgacaaaacaaattctgcaggctgaactggattttgggagcctgttgagctgaaacaaacatcaccaaactactctgcaaggatacacttgggtacacagtgctcaatgagggaaaagagagtaggaaaaatcctttcccactggtctacagctatcagaagagagaaaacatcttccccctcccaacagcaggacactcaaaaatagctaccccatttctcttctttctaaccccctcaggacatattctcaaccaaatgccagccagccttctgcaccacattccatgtggggagcagcatctgcttgcaatacagatgcccatggcacctgggcagccgctgtcttggaaactgcttcctgcactggcgagggtgggtatggacacggtgtgttttcctgagccacagaagggaagataataatccatcctaaagcctcaccgaatatacactctgacactgtgtgtctgcatgccacacctgtgggggtgtgtgctcatttttatcggcacgcatcctgctgtcataaaccatggtggactttaattgcccagcagtaccaagtctgtgccttttaatgcctacagcatccggcctcccccctggagaaacatatctggttacgacgtaattcctctgacctaaattgagtcactttcttgtcaaagaggacagtggttcttcagctctggggtcaattttattgacttttctccagcagaaaccctttggaactcatcttttgctcctgccttggattaaaagtttctagaggagaatttaccatttgtgttttttaaaacagaggtgctatgctggcccccgagccctgccccagcccagtactctctgatttcaaatactatcatgcgctctcctcttcctaaatcttccccggccacaccagcatccttgctgttcctcaaaaagatcaggcccatttccatcccagggtctttgcactggtccttacctagatgttcttattccccatgtctgcctggctcactcctctctctctctcaactctatattcaaacctcacctttccatgatgcctacactgattccccatttaaacctgcaacactcttaacctgagcaacacagcaagaccctgactctacaatttttttttaattagctgggcatggtggcacacatctgtaatcccagctactcaggaggctgaggcgggaggatcacttgggtagaggctgtcaagagccatgattgtgccactgcactccagccttgggaacaaagcagaacctgtctcaaaaaaaaaaaaaaaaaaaaaaaagacgacaaaaaactgcaacattctcccccttatcagctcttaccatatcatattttagtccacagcacttttcatcttatatcttaagataaagtttaaacctttttctttttttctggtagttctttattgtctgggtcttgcacagactctagaataaaaatcagagaatgctaacaattttccagttttgttcaacttctcggtccccagcatgaagcacacagtaggtactttaaaaatattcctgaattgttactgctctcctacccttccccaacagaagctttctgttgtgaagaagtcaatggcttcacaggcagagctctgtttaaatgccacctcaagggagaagccactctatctaaatagccatccactctacctccctactcccctgcatcactttattttcttcctagtccttatcaccacctgacattatataatgaaaacagagcacagctttgtctttgttggtgttcatggctctatcctaatcatctagaacagggattggcaaacttctcatagaaaaaggctagagagtaaatatttttggcttcacatgacctgatgtctttgtttcaactactcaactctgcccttggagcacaaaagctgccacggacaacatgtaaacaaagagtatggctgggttccaaaataaaactttatttacaaaaacaggtggtgagtcggggctggcctataggacacagtttgccaactcctgatctagaataatgcctggtgcatagtaggtgtttaataaatattagctgaatgaatgggctgaatgatttaacaactaaatttatcaatttaccgcatgtcaggcactccgtcaagtgatctttcaatcatgtcatttcatccatagaacacctattaaactaaggactgttattcctcatttagaggtgaaaggacagaggataaaggagtttctgactttcctgggctatcaccatgagagaggattaaaattcaggtctttcagcccctgctcattccactacagaacatgtaccctcccagtcttgttgcatgctctatgaaaatgcaaagtcatgctgaagatgataactagaattcttcaccacccactgtgtacagattccttcactcatctgccagccagggtttgccagctacctactgtgtgcccttgctgtgctcagtgtgataaagctctagtcagacccaggctccacaagacaacagagctacggaaacagccaaataccaaccacgtgccaggcaccaggctgccggcattagaaacctgactctgattcagggcttgccttcaggaggaagggcaagaagcgcaagtactctcagggccttctatgtgccaagcactatgcaacatatttccttgccatgcaagtaaatgacaggtgtccacagaaatgcaaaggttgggggagttctgaggagagagcatgggatttcctccttgggactcaggaaagctgagttaaagggcatggttaagccatgtgtttttgggaatcagaactgggaccacttcccattccaagaacttactagctatgcagaccctgggaacatgactcagtttccctatgtcatagaaaacagtgataagagtgtgacctcccaccccccaaattttttttgaaaaactaaatggactaatatatatgtaataagtgattgcttgatccagtgtagggacttaaagtgtggccataattatcatcctgatcacccttgaaggacctgagagaacttcagagtgaaacctttggtattcagagatctacgagtgaaagtggtaccctaccctacttaacacactggttattatcaatgatgacaatggtgtgatggggtggggagtgatgggagaagttcaaggacttcccagatcatgaggctcctgtttgttaagtagcctgaagtcagacaggaccccagattggatcatttgcttatttaaccagcacccagcactgagtaggaattctgtctgctgccaaaagccagtgagtgcgccttggccagagctgcctggatatcctgagaagatgaactgtgcttctgccaaagacacaacttaagaccaatggtcaactccagctgcgtcggatgtggaaactgagctgggcctgctggtaatggggcctgcctccctgacaaccaaccctgcctccatcagtcagcgcaagcctcatgcatttagagcacccagatgggagtgtagtgctaataggtgcttcattactgttcccttccaaccctgctggtttttaaaaatttaatgtaatttaatttttaacttaatttgtttaattgacaaataataattgtatgtattcatgaagtacatagtgatgcttcgatatgtataatgtacagtaatcaaatcagggtaatgagcatatccatcatctcaaacatttatgatttctttgtgttgagaacattcaatatcccccttctagcaatttgaaattacatcatattgttaactatggtcatcctacaggagtatagaacactagaacttattcctcctgtttttcccagaaatctccaatcacaccatgctctaatagaagtatcaggagttctaacacgacagcaaagagatgccttagagtctggtttgacataatgcagttcaagtgaaagtggtaccctacctgacttaatacaccggttactaccaacaatcacctttttaaaactcaaccaaacatcttagctcagagcaattcaaaacacaaacaaaccctaggaaagtgatgagactttcagtcacagagtaacaacatgttgtgtttatccagcatctcctactaaagagatttctcattcaccaggcattctggagcttctacaatgatatgcctggagctttctaagttttcccttatttattccttttgaaacctccttacaggcattgggctcatccccatttggctgaagccgaagctcagtgagctcaagggacttacccaatggcatctcctaggggaatgtcgcatctcagcaaagaggaggaatggacagaacccatgggaacacgaactggcccaaagcatttagcagagactcttgttttccagatgctaggccctggctgtggtcacagccgggcctgagttcagtcccagctctgccaatctgatctggaaggatttgggttactattacctagtactctaatttcggtttcctcatccactaggtggagctaacgccagtctctctgcctagcactgcagtgaggattagaggagatggcgcaggagaggccctcaacgcactgttgtcaaaaccttcatttccgacccaggcagtggcctgggtgctccacatatgtttctcatttagtctccgaaatggcccactctttgccccattgaacagagcagataacaaacacttgggtctcccatccagtttccatggagcatcaattctctaaccactacgtacactgacactctgcaacttagaactgatctttgcagaagcagaatttacatgagagaaaaacttccacttatactgcaaagaaaaaaacaaaacaaaacccgacctcctcagagccatgaacttggtaacaggaaaaaaaaaaaaaacccagagcctgacacagcagtaacttcatgtattatattatcaaaaggaagacatttcttccttttactttcaaatttggtctcagatttatccagccttgccgcctggtagttcaaaaatctagataccaaaactttggaaaaataccagaattgaggacagttgtagcgggtaagattataagacagtccccaagatttctgcctctgcgtacccacaccattttctcctagtgagttaatcaaacacaaacccagggactgctctgaagggattctgctatcgtcattaagatcccaaatcagctgacaacagatacagagagtatctgggtgggcctgagctaatcacctgaattcattaagagcagagaacgttcttagaagaagtcagagagaaatgtgttagctagtgaagtggtcctaagtagttggagggatcttgccagaggtgaccagaattgaaagaaaagcaatcaaaggggatgggataaaaattgtgtgtgtgcgtgtgtgtgtgtgtgtatgtgtgtgtgggagaaagagagagagagagagagatttaagagagtcagggagagcctctctagttggtgccatttattccccaagtgttttattattattactgcatttttcattaaaaaacacccattgcttaagacacacatcaaatccagttggtctagttgggtggtggcttaatgggctaatatcgcattttaccaaatcactactggaaacttcattactcagtctgagtggctgagtcaggccctctgtgcccagaggactgctgcctgacatgacctacatccccatctgaccgtgcctcagtgccctctttcaaggtacaatgagctgggaaacgtggttatagtcacctttccagttgggtggaaagatggcagggcatgagagcaaagtactagattccattaaggccttcctctccctgccagggtaacctagacaagttgttcagcctccctggaacctcttctgtgatgagggaatcagaataagactatctacgctgcatagggttgggatgggaatcatgtaaaatcttatataggtaagtgctgggtacaaagtagatgctcatgaattgctgttcttctgatcatgatccttagggaagtaggatatgttttacagagaccatcagctatgaatttaaacaggttttcagtacactgtgctcagtaagggattaccagaggagatttaaggaatttccacggcttacagactatctggtttgaaaagtcttttcccttcattaataataataataaagactaaaaaaagttcccagagaagctaaggaacttgttcaagggtgcacagaaagattaaaggttgttatctcacttcaagttcaggaatttgttttcttcatcttcagctaaatgccaactatctatccatctatctatctgtctttctgtatatccatctatacaaaatcctaggaaattttttaaggaaaagcatatctactataaaatatctaatttataaaatattaactccctgtgcaaaagcatacacctttctttgtgtcttcgggcggttacacaagggtagcaagggaaaccagtggtttcagaggcaaacactgagaattacaactccagagaaacaacaaattccatccacagcaatttttttaaatcatcaagtttttagagaaaccttttggtttttttgtcaaaagtaaacacaactcccaaaatttaatcttcactctctacaggcagtttctgctgaaccaaaagattagcaatgggcttcaaatctctccccctcttcaactagttggttaattgttactttccttttttctgtgtgatatagtctcttcaaatttttgagtctcttgattttgcttttgcattttatatattttatatattttttgaaaatgctttatagaatcagatagaatagaaagaaaccaacttacactggcttttatttttaagtggcattttcttaaatgatttgcttattattcacattttctaaattctctatgtactttgttctgccaaataaaaatctcaaatgtcaaaatctcatttggggtgcccgaagaactcaacatttcattaaaggttacttgcataagcctttgcccctattaaaatgctattttaattttgctacagatatataaatagtataaaccagtacaggttaagtggctggaaacagaaagacaggcagaaattggatgatgcttacagtgcaaaggtgggggatacaggagaggggagcatgagagtaaggaacagagggaatgggcagatgagcctgaagcaaagggagagaaggaagccagcaaataaattaggcagcatgttgatggattgaatgggctttcccagaaccccaggtgatactggcatctgcccaaagggaagggaagctgaacttcagaccacctccctgagacagcctaagttaaaggctgcagtggtgagcccaggcatcacatgacttctgaccacagagtgaaggcaccatgcttgcatgtacctaagacaaattcagtgcagccatatgtcaagtgcctttgagacaggcatcgtaaaagtccaccaacaaaataggaaggcttctccagaccttcatcccagaatgcacatcaatgacagaatatgcagatacgatttacaaatttattcagaagacaggtctctataggcagtagaatcaacttgttttgcaggttcttattaacaatgttgcaataaaaaactcttttaatgagttacagaaatagctctcacctctgtgagcccagagagcagcctgagcctcagcttttggggagtattttttgctttcaatgcattcgatcatttttgcagctctcacagaaagcagttatattagatgacaagcaaccagctgcctgtccctcaggaagacacttctcccatgcacccagtgtttcttggtttccagtcttctacttcctcttatcccatccctggccatcctggcctttgggagtcattttgtatgtgaggcccacagagataggagaagagggaaaaagcgccctccacccctggaagaaaataaaccctatgatttgtataactccttacattgtccaaaattccaagcatcaagaactcactcctcctggattggaagaacttaatggacacctcccattagaatgaatcattctaacaattccactagaattagtatcgcaatacacgctcacacatatgtctgtgttcttactcgatggtgagatacttgaagagaagggaaatcctacacatctcaaagtcccaggtggccacatctttaccacctgtttggcttcagataaacactctctgggcctcagtttctccttctgtaaaacggaaacactaggcacttgtaccttggaggactgttgtgaccaggcatcagaggagttaagaaaggcttctgagtctactcagctctgtgaatgagctaattggatgatctttacatcaggttttctggctcccaatcctggacaacaacctaccgggtgcccatgacaaggaaggggtaggtgcaggagaacgtaacctggccaccagggaaggcttcctggaagcaaagacacctaggcagacctgagcagtgagctggaattgtcaggtgaagagctgcatgttcagggcaacagacaaacacgtatgtactattcaccaattccattcatgtgttaaatgtattcagcgtctgtgtgcctcttgtctggaatctgcctcttttgtgaaatgagaagactgagctaaatatgagctcaaatcctatccacttctaacattacctgacacagagctgggttcatggctcatttaaccacgatccgatttgcaggcattgagtggaaccccagggccggcttttcacacatccttgtgctcaagtctgcgtttcatacaataacctaaatgaaagtgacatttgatcaactttgaacaactgactctcaacagcacggctgtgagtggtccaagctgtgggatgaggcttgtctatggctgatcagcaagagcacggagccagctcctaggccctggtgatgccaccgcgggtccctgcacacacaacgacccccacaccagatccagccaggcttctgaggaagcccacgtgggtacaggaaagcagcagatgtgctctctgaaaggatcttctctcctgtgtcatttctgtaaggaaaacaatcacagaggcttatcccagtggggagggaatagtgggaaacagcgtataaagaatcagagtggtgaggccctccagcagtctggggctctgcctttatattcagggccttgctatgcctgggcacaatggcattaatcctaatccctttccctcccctcctgcttacgctgtctcacttgccctttcaccacaaccttgaaaggcaggtagcattgttggtcgcacatctgcccagagggagcctgtccaggcccctgacatgatctcagcattctgttaattcccctagcctttgcagaggccacagccagcattcaggataagcaagctgggaaacctggacttagtttcacaaaacaagagcaggaaggtccaggctagggtgaaatccaggagagtagctgggcaatccccaccaggcggcaaatctacatatgaacccctgcctcagaaactactagaaaccagggctgcatccatgaacaaggagccagccacatagagactgagcggagaatcctcccagctgataggctttcaaggaaaacacaaagagaaggcttgatttcataagatggttgaatcttatgaaatccactccactgtggatttcacccacttccactccactgtcccatgtcccttttatccccatgaatacggatcctacaaatgccaatatttgggaggaatagaacacttttttttttttgagatgcagtcttgctctgtcgccaggctggagtgcagtggcacgatcttggctcactgcagcctccaaatccctggctcaagcaattctcctgcctcagacccccaagtagctgggatgacagtcatgtgccaccacgcccagctaatttttgtatttttagtagagatgggttttcaccatgttggccaggatggtcttgatttcctgacctcgtgatccgcctgcctcggcctcccaaagttctgggattacaggcgtgagccaccacgctaggccaaatagaacacttttaactgcaacaatatatctgcaactgaaaagctatgaaagaatctcctttgacataccaccctctgccctgcccaccctcctcctagtcagtgctgatgcaaaacgaaatacttgaggtttacaccaaatgctgcagtgattctaccagcactgccctgacgtggggagggacaggggctaactagatttggccagtagaacctgaggaaggggctgggagaacctagagtctggaactattgggacactgaacacttctgagtgagagggctggtgcagtcgggcagttgctagagccaaagcaaatccctggggaaggaggctgtgcctgggttttctccttgcagacctgggggcaagggtgtgcatgtgcctgtggtgtcataattagtctggggccacaggctttggaatgttaattctggagatatcatgctacctagagctgtctcttgcccagagagcgtgtccgtgtgtgggcactgtgaggggccacagcacttccatcttgctcttatttctatctatggcctgttgagtgccaacttccttcttggcttaatgagaaggagcccagcttgagaactggcccccactgcccagccccttcccacactgcattgtgtctggtggcaaagaacttgctcggcttctggtgagcaattagtctaattcattctcccagttctctttcctatacaaggcaaatctcagctaaatagaaaccttcagcacattttaattcagaatagcactaaaaactgttattatgggtaaggtctttcgtgtatctcctgacagagcctaaaatgaccaaataggttgtttataaatgcattccttggcacatttaaaggccttgcatgctgtttctaaatggtttctaataggtggagcacagggaacttgtaaggcagtaaaactattctgtaggatactataatggtggacatatgacattattcatttggcaaaacccagagaattgtacaatgcaaagacagaattataatataaattatgaactttagtaaataataatgtattggcccggcgcggtggctcatgcctgtaatcccggcactttgagaggccgaggtgggcagatcacgaggtcaggagatcgagactgtcctggccaacatggtgaaaccccatttctactaaaatacaaaaaattagccggggcgtggcagtgcatgcctgtagtcccagcttactcaggaggctgaggcaggggaatcgcttgaacccaggaggcagagattgcagtgagccaagatcgtgccactgtactccagcctggcaacagggcgaggcaccatcacaaaaaaaaaaaaaaaagtgtcaatattggtatgtcaattgtaacaaatgtaccatactaatgcagaaagttaataataagggaaattgtttgtggagggggcaaggaggcatacagaaaccctctgtactctctgctcaatttttctgggaaacctaaagatgcaccaaaaaaatctattattaatttttttaaatcccaaggcaagaaaacgcccaaatgaggacttgtctcaaaagagccatgaccttgggccctcctgtggatctctgggtgcccctaagaccccagctggtcccatggctgctctgggccatccccagcccctgtggatggagacactccctcagcagcccaagccttctgtgcacagacctgcagcccattcccgctcttcagggagcagcctactccaaatgccatgtctggcacattcgaggagcaacagcagctttgggttcattaattagcaattggggtgttaagcagcatgcagacccaggctgcaggtgtgaaccttgcttcctctccatgccttaaaaatgccaccctataaggaacaccaatgatccatcccaaactgggaagatggtgaactgtgaaaacttcaaggaaggaaagtatcatagttccatttccaccagccatcatctatgaatccttcaaatctcggttcagtgtcatctccccaggagcttcctctggcacacacagccccgggcctctggtttgctcctgctgggttcccagggcacccaggctcaccctgctggagataactctttccccatctgtctttgtcactagtctggtggatcctgcagagaacagggaccttattttattcatccttgggaaacctcatgaaacatcctgtagcctgtcttgtttttagctactaaggcctcaccaaggggccagagccatggggtgggtacaacaccgccctctctccagggctcatcctgaatcacttctccctgaatctcttctgaggaccaagccccattctgtacatctccacagaccagcccttgcctggaataacatgtctcagacttgcccacttccccttctcttgcataaaactcagagcaaatgggaggaagaaatggggcagaaacgtttttactatgaaagaagaaagtgggacaaagggatgctctgtagagtcgtggttctagatggaaagaagcatcacctaccaaaagctccagggaaggtgggcttctgtcatttgagtaagttgcctgatgtctcagacacccctctctcttttccctgggttaagaagttggttggtcttctgatatgtcagttaggctaggtacagcccccagacacttaatcaaacattaagtatttcctgtaaggctaatttatagaggtgattaaagtccctaatcagatgattttaagaatgggaaagtatccaagataatcagggtgggtctaattcaatcaattgaaaggccctaaaagcaaagctaaagcttccctgataaaggcgaaatcctgactttgggcagtggcttcagtccatgaagacaattccagtcttcccttcctgaaagcttcccctaagggtttcagatttgcctagccagcccccgacaatgacatagccaactccttgcaataaatccctttatatgtctccttctggctctgcttctctggtggtaccttggctcatacaagaactaaaactcttaagtagtcactgcagttctaccagcagacactcaggaggatgagacaaggaggggacttaatagtcatcaagcactttatataaagatcaggcaattcacatgtgataagcatttatcttaatcttcgcaataaccccattgtcctgatttttcagtgtgggtgacttaaattttctgtgcgacagcttcattttttgtgagggaaagggaagactcccaagtgacccacctatgaccacaattcagagacaaagcaagggtcttgctgtgttgagtacacatctgcaggaataggaagcctatatttcccataacaaacaatctgggcaaagtgtgagggggtgcaggaaggggtgaaagcacaacagcagcagatgacacatgctctttgttcatattctcctcctcaaaagtttgccactgagctgccactgggcaagatttcaagaagcagccagagaggagagattggctcctcttgacgatgcatgagaatgcctaagaaggtgacaggtcccactataagccatcagccctcatgctgcaagggcgggtatgggggaagcagagttttcccacggcataaaattgcctgttccacaaaaaacagctcctggatttcccaagtcaaggtcctcagctcacctttcctccagcacccagccaccagtggcagtgaacaatagagactgatccaaaaagactccttcttgtatttggtttcagggtgagagaagtgctagggctcatttccctaatatctcaaaggtgaacttcaagactgggttgacgggtgagtactccactccaccaaagaaccctgataaagcacccactcagtgtcaggggctgtgctggggaacaaggtgggatgcaaaggtgaatgatggggggaccctgccttccaggagtttataacttagcatgtcgaccaactctacgttcaaaaacaactctgggtcaaagcaagatcagtgctatagtagagtacaatcatttcttcattccatcaacatttcttcatgcagtaaacactgcagatttggtgttggagtgctttcaccaggcagcaggaatgtgacattgcaacaaagatggaggaggagaataaaaaggcaccctagaggaggtggtcctgaaatgaacagctcctgtcctgaactgtaactgcttctcctgaaaactctacttcctaacagcctccaggtcttaggcaacactgaactaagcagtcagccacagagaacaacaaaccaggcagaaatgtcccaggtgggtggcagtgtgtgtgtgggtgacttaaattttctgtgcgacagcttcattttttgcgaactgggagtaattatgccaacttttccaatttcataaggccatggtgaggattaaacctgatagaaagaagggtgtcgtttcaaaatatttaaccccaggtattgcctggatactgataagtcagaacagatgttgggcttgggtggggtggtggtgagggtgccaattgttaacactttctttgctggatcaagcggggcttggaatagcagtattagaaggtgggaaggaggggactcgggaggatgggctgaagctacgtccaatctcacagtggagaacttcgacatttcaaccactgaacttgtccactgaatatcaaccttgctcctaatgctcccacagggagaaaagtgctgatcagtttcatatggacatgaagatttggttctgccttctttgtcagtctcaccccctccacaattccattcctgagccccactcactcagcacaaatgcacacacacagacacacacacaggcatacactcacacacagacacacacacacagaaagacacaggcacacactcagcacacagacaaatatgcacaaacatggacacgaacacacacacacaccagcaatactaaactaccatgctttctcagttctatgacaccattaagcatatgaaacaccatcagtttaataataagtcaggggatgtggtatggagagagcagaaactacattaaatatacaattcaattgactccgacttcaagagtcaaccctgttttaggtattaggccaagggttctcaactttagtgggcttaaagttctaaaaaggtgtgtacaatggaagaaaggggccaagacaatttcatgtggtggaaagatcacttactgacatatgaaggacaaagggaaagtgaagaagtggagggggaagtgacatttctttccccttttatgaacagagccagcccctgtgccccaaggctgggcacctttgcttcctacactggctgtcaaaacaaactgaaaatgcctctgcgcccatgcctttaccagaagacattcctgcacagacaggcaggtcctgccgaggaagagagagaaaaagagggagtggtgagttaggcaggagggcagggacccttcccagtttcaccccaggagcagccacagcaaagagccctccttcaggccagcggagacagcacagccacctgcctcgaatggacccacgatcttatttatttggtctttcagtggcagcggacaccctttccaaaaactcaggctctgagaccatctctagtggacaaggagagcagaatcccaaaacaaagtgattgatttttctctccaccctggaaagtaggggcttacagccagattaaactggatttagccaggttacactggcctctgaacaaatcaatgtgtgtagcacttcctgcatctaagaaccgggtggtacttcagacgggcttcacttcagtcccaggatcaaagcagcacactattttcagtcccaagccatccccattttctgtcctccctgaccgtctttggtgctgttccttctccctgggacacccttcctccctcacagggatcaaggcacagagtgaagctgttggcttgttcccttgtctttcaccatcactacagtatttggctggaggtgctcaaatgtacactgtcctcaactacctgaacagtttgtaaaaaatacatcttcctggtgctgcctccccagagtctagctcggcaggtctgcagcagagtgcaaaagactgtatctttcacatgctctccaggtcactgatgctcaagcagcccccagaccacatcctggggaactccgagctaacgaagagggcagggactctgtctcgttcatccattacatgatggggtggaggcttcaataagtagatgctggctgactggcaaaggtaagtcgctgggtgggtagctcaggaaatgagtgagtgcataaattaatctagtttattactagtttattactgactcattcagtcagccacccacccacccaccacctaattaattatgctacgtatccaatgactgtaaaaagtattgtgcagaatataaatgtcgggtaacatcgtttttttaaaaaaatcattgtggactatatgtacaacatggaatcgttttcccaaaattctccctggaaagctttggcatatattccatggagatactttcaagaatccagcaatgtcttaagtctatttgccaagtactgctgtggagagtttgaagcttctgcctctactacatggagtgagcttctgggtgtactggagtcccttctgtgcacgctctggtcttctaggcatccccctgaccttcaatccagctggtgttcgactctctacctgatgctgtgccctggggatacagggatgaattaaacatgggccctgctacgctaggcagacttgtaagatctcctcccctggtgtgcataccctgtacagcgccctcccatgaaagtgagcaagacttgtgaatatgatgggatgaaattccagcaactatttttaccttatgtggccaaaaggattttgcaaatgtagttaagttcccagtcaactttgagttaatcaaggagattatcctgggtgggcctgaccaaatctagtgagccataaaagaggtcagagagtcaaaacgttggggagcatgttgccatgtaaccacaagggctgtatagtagcaaagggcactaggcagctaggagctgggagccaaccctggatgacagccagcaagataacaaggcctcggtcctacaactgcaaggaactgaactctgccaacaaccagtgagcttggaagaggaccctgagcctcagatgagatgcagccctgcctgacaccttgacttctgccacatgagactcttgagtggagaacacagctataccatgtgaggacttctgacctacagaacttgtaagataataaatgcatgttttactttgctaaatttgtggtaatttgttatatagcagtaaaatctcaaagagtcctttcccccaagaagctcatagacacctaggggtgacaaatacaagctagtaattactatgctatattgttttcaagactacagcaattcatagctttagtagttttcaaaattgagccaaatttgccttcggaaagtttttcctattcatctctaatccatctatgcattcacttaatttacaaacttaagttgagctcctattctgtgcctgatactgtgctagacactgaggacccaaggtgacacagatacagttcctgatcccctgaccttggaccaaaatgtcactcccaaaccttgcacaacccactcatgccccaagacccagcttaagtgccacctccttgacctaacttcctagatctcctgtaaaaaaaaaaaaaatagctcatcctcagaattcccacagcaaatttcatggtactctgcttaacacgtcttggttccactgtgtgtttcaactgtctgtgcatctcactggactgtgacctccttggggacaggggacatccctcatcatctaccttcacaacctaatgtctgacaggtagaagatgctctgaaaatactggttgaatactgaacacctcaccatttatccctggatttccttgccagctagccctgggttctccctatttccctattttagagtcagtgcatattgaaatggaggagaaatacatcaggggagacactgggacagttggatatgcagccccattctaagactggccttgcctgatgcccagaattaaccaaattttttggaataataatccttggtcatgattataagggttctttgcttatgcagaaggtcaatttcagaagcattactcatacactgaatttttgcctgtcaaatcttattttttaaatgatagatttagtctggcatggtggtgtgcacctgtagtcccagctactcaggaggatgaggcaagaggatcgcttgagcccaggaggtcatggctgcagtgagctgtgatggcaccacttcagtgtagctgggtgacagaatgagaccctgtctcaaaaaatataataaaagataaaataattaagtgatggggttgctgtttacttaaggaatgactataatgagccttattttaaagtgaggactcaaaaattgttttttgataacaccactaattcgaaaagcatgtaaggtaggctaaaataactggagaacatgagagtaaaactgaagattttcctaaagggagaggtaaattaatgggaagaggaagaagataggccaagaatttggaacaagataatccctgagcttcttgaaaaccaggacaaagagggaaagatgttggactttttagctgttgctgccagatggaaagaagcaaacccgtccacctgggagagaaccctgtccttggagctcaattatgagtggctcatgagcgagatccatgagaaaatgcattgcatgcaaagtatgtgtcccagtgcctggcacacagtaagtgcccagtgacagccagttagcagcagctgcagcagcagccacctttccctttcccagaggtctttaaaaggagaactggatggcatctaacgttacctttcactacagctctgcagaaactaacagtcacatttttctaacaggaatttgggatatgctgagttcccccaatgtcaaggtcatgtcattacacgataactctggagagagatttttggagaccgcttagctaatttatagatagcgtgacagatggcaaagtgggactctgttgcttctgtttcagccagggcttcatacagcagccatcacctgtcccctgaggccagaccagcacatctctgacctcaccgtacacacaaagcacctgggcatcttgctccggtattttctgaccgttaagtctgggatgggccttaaaagccccctttctataaagctgcaggtgatgctgttgccacagaggggcagccagttcctggagcaggtgctgggacaactaaccaggtctgcatggagcaggctgctgaaaacccattgtgagaaccaaactgtaaacatgcataaaggttccacctgaggaggtgagaatgagagtgttattttaagagaaattgtttggcaccttgtgaattcaatgttgcctgagggaggtgagaggggcttcacacattgttgcctaagatctagcaagaagagctctaagagaccacctggagagtgtactgtccccagaccttggggacgcagcagtgagtagtgcagtgggcttgattacaggggacaccgacatgggattggggacgtaggaatgtcagactagagatgcaatatggcttctgccagctggaaagcctctctcagcagaaagagaaggaggtggccctgggtgtggcagcttaagaagcagctctggagaggcactgcaggggtagaaacacagaagcaggacaggtgttggtgagggaggctaccaggaacccaggaaagacccatgagaggaatcagcagctctgaatatctgctagggctagagaaaaacaaactggcatatcagggacacacacacacaacacacacaaggcctcctcctgtctccacctccctccttttccatgctcccacccaaaggaactagaagctggtctgggactgttgctgcctacaatgagatcattcctggactcaaacaactgagggacttttcattctctaagcacaaggacaagctgtgggctgccgaggtttctttgaccatggagggatggagcccagctcccaagcgtgggttgaaatgggcaacatggagatgatatggagctgctgctggtgccccaggcatccttcctgttcaatgaatggcttccaccactcttttgagcaccaattcagtgccagctggccttttctggggttaggcaactaggatttgaaccttagctctatcccttacttatggtgctatgcagaaggttcaccccatggcaccccagagaggactgagaatgaagcaagcccactgcctggcgcttggcaccccacagggcatgcagcagggctcagccaatagtcccttgccgattccagaaaaacaatttgatttgtgctttaaaatggtgcttctccaacatgaaagagcttgagaatcaccaagggcagtgattaaaaatgcaaactttgtagctccccccacccgccggccccaataaattctaattcataagtctctatggggcttaggaacctgctttgcatctccccttctgggtgattctgactcaggtgatccataaagcacacattttgaggaaaagatgttcaggatggctcttaggtgaggaagcagagtgtactgttctaccccaaaggaatattgcgacacctccaaccaggattcagccaggacctcagggctctgggtgaaatgttttaaacaggaaacagaaattcttttgcaaatgccactggccacctgagctgtctgaaggtaaaagaagtgtgcatatttgtgcatcccacatatgagccctcagcctaagcaatggtagtgtcacagggaagaaacccggcaaactcccagagacaggagagaggcatgatgatggccctgtggggattctggcaatgaaggcaggatagcatggccacctcccctgccctgggacagcaggaagaggacagtccatcactgtccagcagcacttctggggccacatacaatgtctatgaatggaccccaactgcaaaacaggatcttgctgtggtcccaaacaaacctagcacttagttctgttgacccggatcacttaaagcaaagtgataaagcaagtgaccggggaggcagcctcgatgtcccatccaccacctcccaacacatcagctttgtgacctcctctctccactgacacctcacagtggagtacacactaggctctcaagaagtatttcttgcataagtcaaaggacgaatggatgaattagtgaaaaaatgagtgaatgaatgaatgcacaaacttatctcccatttccaagtttcctttcttcaacccaatctatatcatgcaaccaaagtgttctcgccacgctacaaatgatcttaagttccccttctacccaacaccttcagtggctcccactggatcatgtgcaaacctccagctatgtcaggacttccacaacccagctcctacctatacatccttcatctatttgcagctttctgaaaacactgtttctttgtgtttctcaaaatccttcttcacctacgttatttcaagtcacccttcagacttcagtttaagcatcacctcctccaggaagccttccctgactgcctcaggctgggttaagtcgcctttcagtcagcagttaattattaattcagcagaaggttattaaatacctactctatgtagcacacacaccactttgagtttccataccattctatacagatctccatttatctccattccccatgcctatacaggccagaatccgagaggaatgaaatgattgtgaaatgaataaataaatggatcctataattcccaggagacttgtgtgctgcaatggttaaaaaacgttgtcagacgttgtcaaggacaccgcaagatggaaaataccccctttcttccagaacagagcaaagcttttgtcacatatggcacaggcagcagattcaccgcatccattttcagaagctgaggttcctgcctgtcttctccaagaagcagggcttcctcagcatgtctgtgtcacgctgtctgcaggagcacgtgactggggaatcccatctgttctcacccgcacgcccacccatgacaaatgaattcatctgcagtgtttgccaggagctgctttgggagcaaaggagactggtgatgaattattcatcctgaggagacgcccgccccaggtttccagagcgggtgcagcctggagaagcacccctctcaattgcagggctcaggccccaggacacaaaataatagttaataatagtgagcacatctaccatgtattgacttcctacaatacacgctgttgggagcttaacctatattatttaatttcattttcacaatagcccctcgaggtttcatcatccccattttcagcatcacagagggggaaggtcaccagcccctggtcccacaggttcctctgatctccagaagatgtgcttgcccctgcagaagggcagagagtcaggagggagcagggaagcccagggaagcccagggagggatcccctctagcagagctgagagatgcctggcctcccctcaaccccactagatttaacattcagagcagcacgtggatcaggcaggatgacttgctcggagccctcgccagcccaaggcatgtcaaatgcagctgctatttccagcagctattctaccagagaagccctgtcacatctgatatggctgaatccatgattttggaggatggatgtgcagtcagcaatggctatacaacagatactagaacagttgctgggagctggggaggaaggggaatgaggggaccccttaggagcttaacacttgatacaattatccatcttcaaccccatggactgacggtgggaaaggtggagagacaggtggcatgatccttttaggagaaacaaaagcggaagaatagcaaaataatagacagagggagcaaacaaacactgtgcgtgatagaaacgcatgacccacctcatttcacaaggaatttggagccacctacaagatcaaatacatgacagcaaaaccgaaatatgtaaataagaaatcagaggagtgataaataatgaacgaggaacagtgtgtccggcagaagtagggatggaaagtgggctgagttagggctccgagccccagaccacctcccggagaggggctgctgtcagctgtgttagagacacttggcagttatcgtgtcactcaggtgttctctggcctgattgcatcagctcaccaaagcaagggcaattaaaggccatcaaagctagcacaggtgtggaaaactatggctggcagcctcctgggctgactctcagcctccctctgaaccatctactgcctcaacctccactccagggtacttctctcttgtctttcatttctactcctgcagcctgcgtttcctcccaggtctgctcctcagcctgatgtctttgtcagcctgagtgtagcaggcataaggattcaggaacctctactccagtcagctccagtgcaatgacaccagaggagtgtcaccaccaggctggacagtgaggtgtctcttgccctggcatttcctctaacatggtgcttagaatgtcaccaccatcaccaccccatgagtctgggagctctctgactgcaggagccaagtcacccacctctggatttccagggcccagcccccggctgcagaaccaaacagcagtgacattcagccttgtgactcatgtctctgcagtgacagtgctcagggacccaggctgctctgcagaccacaggctcagatgggattccatttctgtctgttctctgactacgaatgtctcaagagcagccccaggctctggtgctgtacatggtatggacagttcttttgatggccatcagagagagataaatcatcgtaactgtgcattctcaacgggacaaaaaaaaaagctacttagggagcaaaagctatcttagctattgccatgattttcggcccttcaacaggccacagtacatatgcagcatatctctggcatgaaaatttcatgggattaggaagaaaagatctaaaaacactcaccatggcaataatcatgaacaaaatattgagaaacactggtccatactactcatgtgcatgagagcccatctggtaggcacctcccagctatgccttcatttctccaaagaataaccagaggttcagggaggtcatatgactcaccccaggccacagtggtggcactgaaataggactcagacctggtgctagaggttctaccagaagacaccttccatgtgtgggaatgagatgtttgtcttgaggcactcacgtttgttgttgttgtttttattttgatgtttgtaagcaaaaaataattttccttttctaaaaatgaaattaagttctataaatattcattgctaagacggtatttctctaaacaccatctctcaaaatcccaaactggacttcctctttggaattcaaaaacttatcttctctttggaattccaaacttaacagagcttaataattcataaattcagtcgggcacagtggctcatacctgtaatcctagcactttgggaggctgaggcgggtggatcacctgaggtcaggagtttgagaccagcttggccaacatggtgaaacctcatctctactaaaaattagccagacatgatggcgggcacccataatcccagctactcaggaggctgaggcaggagaatcatttgaacctgggaggcagaggctgcagtgtgagctgagatcgtacaactgcactccagcctgcgtgacagagtgagactccatctcaaacaaacaaacaaaaaaaattcatacatttactcaagaggtgtatacatccagtaggtactaggcatggggcactgaagccacagagaggaattagatacaaaacctgccctcaaggggtttccagtctagaggaaggtggtgcccagggctgggtccacgctgagaaaaggtgagctcggaaggctgcagggtacagacaggaaggctgggtaagtgcagtaggctttgtgaagggtgtccctctgaactgaaactgaaaggcagggcaggtgttccctgttggagcagacagataaacaatctaaacagaagggctaacatgtgcaaggggacacgtctgagggagcctgaagggtggaggagctaacagttctctagggagaaacttatgtccaagtagccaccaccattattgggtcctccttctgcccaaagaaactgagcagaacaaatctaatcctcgtttaacctgacaggctgtacgagtctgatcccatacactcatgcaaacattgaaagtgggcaacatcccgtatagagactatttacaaaggtgaggaaaggtttagggaaatcaacaagggagggtgaagcccctgggattggcaataacagagaactgttaccatctctccaactaaggaggccaggggagagaccagttgtcagaaaccaggaaaactgatggctcttggaggagctgtggcccacaacagcccaacagggaggggaccaagtcctctcctcctgactctgacccacccatctcctgctggttcctcctattgtctgcatccaatcagaactcagaggcaaggaggccactggtgaaatttacaagttgagtctcccatgggtggagaatagatttggagaggcaaacagaaaacacccagcacatggacagccatgggagacatctacttttttttggaaacctaatctgcatatagtctctgttctggtgacatacacactctatgtgctggggacttgaccacttagcagcaagacggtcatctgactcagacctgggcaaacccatccatccatccattcattcattcattcattcattcattccaggcatggcactgaataagaaggatatgtccgtgaacaaaacagacttccttcctaatggagtccaagcaatgggagtgctcccggaatatatgccaagccagtcattcagaacctagttctggaacctctgtggacaacaacaggaaaaaaacgttctctttctgttggccttggagttgagaggatgcaggtctggagacccatgagccagccagagggcagcagagtgaggatggcttcaatccacccaaggtccgtggtgcaaaagcactcctggctctagttgaggggaatatttctccaccgagccacacccccctcatcttccagaggagatttcctgagatcaccgggattggatcagggacacgacacttagcatgctggcactgtgattgggaataagaaggcagtgctggtttttttctgagtaagaacatattacccacactgagtgacccacacgaaaacaaggaggaaacaattactttgaagcctgaggacaagataaatataaactcagaccataggctttctgaagaggagcggcattaactggcagccagcaagaagggatctgtgacagagctgaggaaacatccgtggctctgtgtgggtttaatgaggcgtgctcacggaaggctggcaggagagcagaaaaaagcaggcgagggagggaggagctctgggtctcaacccgactctgcctctaatggtctctgtgactaggggtaagtcatttattccccactcgggtttcaccctcctcaattgctaaatttaaaaagtagatggccgggcacagtggttcacacctgtaatcccagcattttgggaggccaaggcaggtggatcacttgagaccaggagttcaagacctgcctggacaacatgatgaaacatcgtctctactaaaaatataaaaattagccaggtgtggtggtgcgtgcctgtagtcccagctacttgggaggctgaggccagagaatcacttgagctggagaagcagagggtgcagtgagccgagaccatgccaccggactccagtttgggcaacagaatgagactatgtctcaaaaaataaattaaataaatttaaaaacataaaaataaaaagtagatagttgtaagtggtgttggggtgtgggcacagagaaaggtgaggaaaagcagcaggcagcattgtggtcagacttctgtgcaagtcctaattcaatctttaaaagccatgtgacgtaaagcaagtcaatttctaagcctcagttttctcatctgtaaaatgggcattacagcacctaccataagatacacaaagatgtaagtgatctccagcacatgggagaagcatgagaaaagcgaggcaacatcactgagaagagatccatctaaaaaggtcaatacaatgccttccagtccaaaagcctaatacttcgggaactaaggcatggcagcactctttgaagaaggcagaaattccttccactgccaggctccctgctgaacaacagtgttctctcaccttggcttaaccacagcacctttcatcctctcaattttcagctgtaaaaactttgaaaactattagaccatttgtctggatttctggatttgcaggtcacgggcatcattgaagatagagccaccctagtaaaatgcaataataaatgcaacatttttcatttaatactgcttttaagagcattatctccctctgtcttaccattgttaatacactgatggaaaattcacacaaaaaacatccagtgcctgagtcctgattcctgagaccagacagagcctttcttagccttaaaatgctttccaagaaaaccatcatgggggccagagaaaagacaatggacagccacacccatctccaccactgaagctgcctgtgccctcagagagtgctctctgtagatcagaaaaatgagaactgttttctatgtagccagtagatacaggtgctattctaggtgttgtacctcaaaacaaatctttgatggtcaatattattagcccattttagagatgagaaaactgaggctcaaaaatcccgaagtgactttccaaggcaacacagatattcagcatcagtaacaacaactgataagtcctgtaattgagcccagtactgtcttaccggaaaacctgcattcttccaaacatgtcaacatgtcatctcttaaaggataaaacgaacagcactcccacgttggaatacatgggtactgtctcagtcagtttgggctgttataacaaagtaccagagagtgagtagcttataaacaacagaaatttatttctcacagttctggatactggaagtccaagatcagggtgccagcattgctgggttctggtcagggccctcttctggggtgcagactgacaccttctccttgtggtctcatatggtgggcagaacatgggagatgggagagggctctggggtctcttgtacaagagagcactaatcccattcttgaagattctcccccatgacctaatcacctccccaaagccccacctcctaatactatcacacttggagttaggacttcaacatatgaattttggagggatgcattcagtccataacaggttgcccacccaagggaaggaatatttgggtgggtaagtcccgatgtgccttagaaaacatcactgcagtgtcatggacacagatgatgagggtccatccaccttctgggctgccacagccagaagaccagcctggcgacatctgcagcttgcccttttcccttgagccagaaacatccagcaccaggacttgatgtctcctcggctgacacccggaagtgtgtgggctgtgttcttttttcaaaagatagagatgaaccagatcccaggcagcacataagggaggtcactgtggtcagaacagactcacggagggaagaatcctctgggaaagcataagtgtttatttaactgctttatgcttcagtgaataactaatttgtaccaaaccccagtgttcttgaggtgtgttgataaatgatgttgcaaaagcaacatgtttctgtgctttccatgtgttttgcatttctggagtctgtgaggagagtctctaactggggctctagatgctgggaagcactgcacagagggcccctgggtttctctcctcgacctcggtcactccagctcccagaccagcacatagtagagatgtagagatgcaaaaaaaacaaaaaacaaaaaagttgcttgggttgacatgaaatacatttaaattttaatccccaaagtaaacatgatttgaaaggtaaacaggaaggcatgactggcatataccaggaggagtttgagcaggggatgtggggcagaggtgaatgtttaaacggctcatacagtcaacctgcaaacattaggagggctttgctgtgttttctaaattttctggttagataagtgtatgtgcagatgtaatacacagtcttgtgttccaattatctctacatatgaataatatattttaagatatttgatgactaaatatattcatgcatgtgaggggcacatagaagctctcaggaaaatggtagcagtctgggcccaagaaaacgtctaggaaaatgaaattccaacagttcatcacaaattgttcgataagaagtctcaagaagccaacgctgggaaatgggaaaggctccctcataactaccagctgacaaaaggaacaggtgacacgaggtgctggcaggaaacatttttaaaagttcttcaaatctacaggaagagcgcagaggagcagaggaaagacaggttatgaccaaaatgatgctgcctgacctagggtgtgtgggtcgggggaacaaaatgggggattcacttggggcagatggtgggtggtaacagatgacaggtgaaagtggtcttttttttcttttttaacctcaactggtggttctgtcttcagcaaaaacaatgtgttccaaaggaaaatgaggaacaaacctccgtaagcaggaacaagttggaacatatgaaagatcctaccaggatgcgagacactggcctagatgaaccctccacctggatctggctgcagaatcacacaggaaatttatcttttaaaacagataatgggagagacatctggatcctgaagatgggcaaatacagacttcattttccaaagggagccaggatgggccaggttcaaatcctcatactagttccaatgttacaggctttccgtctcccttcctcttctggtgggatcatgaggcctctagtgctggggctgcaccatcagtgttagaaagagagaaaggttataccctccactcagagctacagcctttctacccagatgcctggtcagtgcctccaccagctcacaccaacaatgactgcctgccaacaccagatttcacctccaaactggcttggatttcacctccaaactggctcattccaggatcgcccttctcaggaaatgacaccaccatctaatgcagttgctccaatcagagacccacgagccatccctgactcttcccttctctttatctgcacattgacttccagaataaatccagaaacctctgtcttcccttcacttccatgcctagaactcaagtccagccaccaccaccactcactgcatcactccagtgatcgctgacctggttccccactaagactcagcagagccgccagagggacagcctcaaagcatatgttttgcattatatttaaatctaaactctctatcctgcaggtgtgcctctgcaaacccacctgctactgttctcccaactgtgcatcacattctagctcctctggccttcctttagttcctggaacacgcaagctttcctacctcaggatctcaggacacgtgatcctccaagaaataagttctcttccaccagctgcacccgcccccagcactgggcctgactggctccccttccggcagctgtatctcagggacgcctcctcccacgactctccatataactaagcacttcatttccaaatcctaatgcataattatttgtccatttacttgtctgtctgcctccgcaactagagagtcagctgcctgagagaggaaatgatggatccgtaagcttaggcctgaagaaatcctcaggatagacagtggagggagagaggggtgagaatttcaggtacaagaaattgcatgagtgaaaacacagccatggaacagcacacatagagaggagcaggtcctgaaggagggcaggaaggagatggggttggagatgtaggcagaggaaccaggagacctcagatggctagactgggagccccttcccaagacgcacacattgctaaaatgccaatcccctctccagggagaagccagaattaattgtttgatgtggtctgacaaaatgggtattaccatgtaattaccaccccccaccaccacccctctgccacctgttgagaggagggtaacacagtgtgtatgtcatctggaggacacagcagcttccaagacaggtcactattgccagtaaggaccaaagggaaccatttctgagaaacgacaataaagttgcccaccaaacaggcattggtgttccaggactttaacccaatcccattgagttgggtccacattctagcaacctctcttccttctggacctccagagtgggcagaattgagcttcctccacaaaatggcaatacttactcctgaacatccatcatctgaaaaggcaggaaggggcaatggttaagagcatagactcaagccagactcctggtttggccactctatggccataagagatggccacaaaacagttacttaacctccccatggggccaagtcttcgtctgtagcatgaggtcactaacagcttctatctcacagggctgttgtgaagatccgatgagcaaatcactaacaaaggaaggcacatttatccccactgtgcagttgggtaaactggagttcagagaggtgagtaattcagccgaggtcacacagctgtaagtggcagaaccagtgcagtcccatcccagagccccacgctcctgacctttgagccacagcgacctgctcagctttcaggtggagcctcacccggatggccccaccatacagtatggagaccccagcacacaagcagctctgaaggtggttccaggacactgagtaaagcgtattgtacttcagcttagttaagtataaccacatcctgagcacaaccaaaactaggtcaaaaaaattatttctgggatttaaaaaaaaacaaaagctgcaggactatcttaaatgtttctcccataacttgcagctgaatgatctcaaatgaaaagcttgatactcttcttccccaccatgtcacatcgctgtggctgactggaatgaactggttccgaatggcagcccacacccctccccccgccacatagctggctttctccaagcacagcaaagtgaactagggacagaatcagctgctgcctctgaggggcgcagacacacagctgttctgcctctgtcgcccattccacagaatagacgtgccccccaccccagcccttcctccctctgtctcactgccctccagcccctacccctctccaaatgtcacaagtacttgacaattccagtctcaacagctccaaaaactctaacctctacctttggtcactctgtgttcccattttgtactcccatccccccaaccctctcattcgacagatgaggaaactgaggcccagagagcaaaatagattgtttcaagtcccacagtccattggtggcagaccagggcaagagcccagggctctggggactcaggctggtcatcttcctgcctcatcaggagaaggtcctagggacacttgggtggtattcatttcctggggttctcttaacaaattaacacagacttggtggcttcaaaccacaggaacttatgtgtcacagttctggaggcagaggtctgccatcaagaactcatcagggtatcactccctccgaaggctctagaggagagccctttcttgcttcttccaatttctggtgacatcaggtgttcctgggcttctgactacataactctaatttctggcccagtcttcacatcatcttatccccctctctccatgtgtctcaccaaggacaaggacactgtcactgtatttagagcccaccaggtacatccaagatgatcttatcttccgatccttaattatatccacaaggagtcttttaccaaataagttcatatccacgggttccagtgtttaggacaccaatatgttttggttggtcatcagtcaacccactataatgagtttcttcctggggattcccagtgcctttggaaatcaggaatttgtgaggaccatggtgggtaaagaccctcagatcaaagttacatcagcccacacagtggagctcgtggatctgcagcaatgacctgcccccaactggacgccaatacatatagtctcaggagagtcactgggatacagaatgccagaataaagagaagagctgctatttatagaaggcgtagtgggtaacaggcaagctgctaaatcattatacacattgcctttcatccttacagacactctgtaatgtaagtagcaagtaaactccatcaattactagctgggtgccttgagacaagtaaattctctaagcctcaatttcctcaccttaaatgagtaataatacgatctgcattgagaggatcttgtgagataaagaagaaaatgtatgggaaatgcctagcacagtggcacacacagacacacacagtacgaactgatatcatcattactcctctctttattcagtgtcaccatctggtttttttaaatcaatgttatatctcctttcataacagccacgaccccaaaaccttcttagggtgtaaacagtagttatatacttggagtgttattttacatgcacgcaggaagtttgttgtttcaaggaactcctgggggactcctgctgtaaggcttaagatcctccggcagcatgaatatgaatctgccaaggtatctactctactcatttgtattcatggtcatgttctaattatttaagtggggcacaactcctgtcagctcaatgaatctgtgttttccagtcttggctgtattcagtcccactgactggttacctcattctctgattctaagacaaagcctgaattaatttagaatattttggttctcccaaccactacaacatgacagtacaaagggaaattttgaagaaaatgaattctattgttaaaacaatgcccaggtgcaaagaaatacaaatgcagacacacacagagaagaaccacttgctcacatttgattccacaagcttttggcaggagcctgcatggcgggtgggaaagatcacaggctttggagtccaacagccctgggttcaaatttcagctctccctatcccagttgtggtgtaaggcctaaagttaaggatcaatatggcgggctgccttgacatctggtgaaactggcagggcctcagatggcctaactgaaagttcccctccctcctccgctcccatggataaggccctcaggcaaacaaccctacccatcatgacaccggccatggttcctgcttatccctaaatagcgtgtttcagttcctcctccaacagagttgttaaaataagccaatcagatcctcttgtgggaaccagggggcacccccgccagtaagcctgcctctcacagcctggtttgctctctgtgatcccacatgcaatcctcatggggaccagcatggcacgcagtgtcctccccctggggctgtgagtatatgtgaagaataaactatcgatctcccccgtccagtgtcaggtgtcataggtgcagccaatcccatatcccataactgtagggtgaaatccttccctcaccaatggaatgattaaagcaccagctggtgtgctgtatgaactttgccattctctgaccaatggttttatcaatttctccaaggaagaataaaaacttccttgccaagaaccaaaacacaaattcctatggtctcactacaggtactccctggtcccccaaaataaacattaaagcaaggagataaataacccttgagagtggatggttgatacattccctcccacttcctcactttcatgcataaacaacacttacattcctaagaagggataggctagatcccttctaactctaacatcatatgtgttattattattaactagtggacagtagggcacagattaagactataaagctatctacctaagaaacacaaacacatcagcaagagagtggtgcttggtgcaggaagggcaaaaagaatgcatctcacatgcagaatctaaatgattgttgctgccccctaagcactgtcttgagaagggtccagcagcagcaggtcatgattgattagtgatgcctatgattgatttgtgatgtctgccatgggcaggggtgtgggccaagacagcaggctgatggcagaggtgcccttctgcctctatgggagtaagagtaccagtcttgcacacagagccctagggttctagtcagtctccttctaggcacctcagtctctgtctgtaaaatgagataaatgaactgatgatttctgggatcttaccctcctctttcttgcctggtctcctttaacgtaaggagtcacatcatggtgagggacctggatggggtcactgacctcaggaaggagagagaagaaaccatacagaccatgcttttggctaattgtgttttttttctgcctgataaacacttaaaatcctaccaggtctgaccccaaagaaagtctcagaagcaaaggaattgctgtaagctgcatttgctctctgcaacagcaatttaagatgtaaaaataacagcctttgttccagcaaggcctgggatctgggcagcaaaaccaaagaaaccaaggcctatgctggggctctgtgtgggctctctccagaattacctggtgaggtggagctccccagctctgccacaggcaagaggaagaccgggggtaaagccacaacctcttctccaaggagcaactgaaacagcagctgagcacaggaagagaaaatggcccaggttggtgtctcttcataaagaggcccagatctcttttcaataaaatggcctttaatcgctttaaagattttattaaaaattccccatggaccaattatcggcaatgcttcctagccacgatctatctagcaatcatacccaagtcttatgccttcgcctgcagatgctcccgggcctgctgtctctccttgtcttcccagtgcatcagtctctgtgtctctgattctgattatgtatatgtccccatctcaggccttgtccctctgcaactctgtctctctcgacttctcggtctctgcacctctgctctctcaatacccatcttcctctttgaaactgtcctcctctttttctccatctggcacactctgtctctctctttgtgccattctcctaccaccccctcctctttaagatcttctttttctctagacagagtcctgctgtctaggcattcttctcattctttctgccttaaagaagtcttttccaacatgaaatgcctgctgtagccagacaaaaattctttggaagcacaccctctcaaccacttttgcataaggcaacttatttgaaaaacattattaccataattaacaaaaacaatttaaaaccgcaatcagcctctggtccctggaagcagcacatagaaattagtcagtattctctccttgcccccagaacccaggagaaaattcacacacacacacatacacacacatacagagagagagagagcactttctacccttcactacaaatggtgaacacttaaaatgctaccaaatttgtgatttcttttattaggttttaaaaatataagatacagcaagctatacctctgttaaactgtatacgtacatttacattttataaagctatttagcatatgcctcgccaggagcactacttaagtgcagtttagagcacctttccagttggcacaaaattatattcactcagttacataagcagatattaagcatccattgtttgggggaggttgacatatgaaaaagagaccacccttcctctaaaggtatgtaaacactgagggaggaaacaaacctccaaacagaacatcacgtataataaaggtagtaatagaagggacacctgagggaggtaagcaggaacgacacaatgacttccaagcttagttcccgaaggacgggatagccattccagaccatcaactaggggaagacattggcacttggagcaatgccaaactttgaattaatgctttatatttttcaaaacattatttgtaagtaccttccccagcattcatgttttgatcctcataagcacagtgccaggcaggttgcaggcacctgatgcaagtttgtcaaccactaatgaagaagtggatgacctggtcatctctgggtgagcaggcatcttgtctccttatcattgcaggcttaacagctagcccatgggtggttccaagtgcacctcccatacatgtgcactgaatgaatctctgggcgaggcagaactggaggcatcagtagctctatatgacagatgataaaactaagacagaaaagataatcccatggccacggtcatctagctaattaggaaagctgttgaaaacagatgagcattcatccactctgagattccatccatccaatacatgcattattctcttaagcatttctagatccatctcaaagttttacctcgctgtcatgtcaataaatagaagaagaaagagccctaagaatgagagttttctggctgggtgcagtggctcacgcctgtaatcccagcacgttgggaagccaaggtgggtggatcacctgaggtcaggagttcgagaccagcctgcccaacatggtgaaaccctgtctctactaaaaatacaaaaaattagccaggcgtggtggcagatgctgtaatcccagctattcgggaggctgaggcaggagaatcccttgaacccaggaggcggaggttgcagtgagctgagatcgtaccactgcactccagcctgggcaacaagagggaaactccttctcaaaaaaaaaaaaaaaaaaaaaatagaatgacagttttccaatcaccttacctagagttcctctgctcacaccttagaccacttccagaatcaagtgctctagttcattcattcattcattcattcaaaaagtgttttctcattctcagacacctactccctcccacaccctgggccaagaactgggcagagaggcctaggggttgccctttccctgagcagctgactgtcattccaaaaccatctcaaccagagaggcctcttggttctttcatgagctgtctcttcacagaaattatgtttagtgtgtgctttagatttagatttccacccagatcttcctgtataagcatcaggtgaagttagtttaacaaagactagcctcgttcccatccaagaccaagtaaataataatctctaaggtgaagaaaagataaaaatggggcattagaaattttaaaagggcccaagcaattctcacaagcaggcccttcagaagtgaccaaaccaagcatttctgtcatgtctcaataatgtggtagagaaaggtcactgagtcaggagccaaaggtccctgtgccagccctgggtgtcccaccaacctgctacatgatgttgagcaggtcatttccttccactgggctccattttctcctgtgtgaaatcaagtgttgagctaatggatccatcagcggggccttcccgactgtcctagaccatgagtctgtgcaagttctgatccatttttaaaccactcctttcctgaagatgaacttctgcagcagtgaggaaaccggtcatcaccaggagttcctccccatgcagacagacaatgagtctatttcggaaacagaaagtgtttgctcagcgcagtgtacctctccctacaggacagaaacgggaagatatttccatttaaactctcaaaagctaataaatgaacacatctcagtggtggttccgtttgtccttcctcaacaccttccaacatgtttgtccctgatgtctccttgggacgtcagtaaaagggggctcaatagagtcgctcagcattgctgttcctcaagcgtccatatccttctctggctgccaacctgggggttcactcactccttccaattttcccttctcccaagaatacgctggacaatggcaatctcctggtaatcccctggctttgtgtctaagagcagctcctcatcttccatctttgattgcactaggctccagaaccttctccaagcagtgccaagcccagctcatcccagcctcctggaggcctcttgcatcccttgaaaccccaaacaccacgcatcaggacccatgttccctactctccctaggagagtttgtccttgtcactgatgtggaccattcccatgtgtcactagccggggagaggatctcccttccctcctgctgctactcacatcagaatcacattgtcccacaggatctggaggcactggggatggtaagagtgagtctttcagttccaggcttgattcttgctgcaacgttccagtaaaacgcccatccagattccacctcctcagagtcgggggacacacaggcatccctggccagctctggtctctagggaggtcggtgatgtccctgaatctcctgggccagctcatctcagagctcagagctcagcgggagtctatccagaaaggcagggccagcctcttctgtctcactgacccatggagcctggctcacctgagagaggaccccatgtaaacatatgtatgggttgaactgtggttggtgtggtccttaccaggtggctccagggcccccagactgggagaagacagaaataaacagtgctttcagggtgaaacatctggggattcttcactgtcatggccttattgaaattgttttcatcgttcccgctgggacctagacaagcaactccctgggtagtatcccaacttacatggagatcccagacagaagcacacttaggccagccctgtgctccccacccaggcacctgctgaactctttccatccatgatctttgactgggtccgggggcctggattcacacctcattgataaaacctaactgcagccttgtgcaagtgtcctcacagctctaggcatcaatattggcttctccaaagcaatttaaagcacatctccctcagagggttgttatgagggatcattgagaaccacaggtacaacactttctgatttcatttaactgaatttgatgcttgtccaaagttttcctattaattctccattgactcaaaacagaaattcacatccccaggggaccacttcatacttgtcatcatatcagcagcacctgggatgggatggggcacttggcaggccctcagtgcacgtttgcagagtgaattggaatcgaattaatcaggatcagggaaataatccatggcaactcaactctccagtggtagagatatgaccagctaagacatctagggcctaaggcagaaaatgaatacggcaaaaaaaaaaaaacctggaacataacaaaactatgatctgtcaaaatattggacaatgttttcaagtccagaggttaaaacaatcaaaaatgtattaaatacattaaagttgaacactctgggttgaaggacatgcacaattttctgtattccagtataatagcttcactagcctcctaactcatctccttccttaagaggcttgcctttccattccaaactcgactccacaggcagagtgacccttccagtgcaaagccagttctctcactccactgctgaaacattccctactctgttaacaggacttaggactccattcatcatctaggtcctgcctacttcttccccctcacatcctatctctgtcatccactctggacacactgcacttaccaagcaatattgcaatccttcacttaaaaatgcctgtcttcttcaccagaataaaagcttcacgagggcaagaattagtaggatgtctttcttgtgccaggcacaggtcaagagctgaaggagaatttggtgaacaaatgccccagttcatgctaagaataatttgccaccacttgaagagaacacacaaacagacacggcaactacataacgccaaattctgggttagataaaatcacaaatggtctttcctacatggtagatgaagaatccccattctgagttatcataattattgacctgttcaatgcccaaagaaattggggtcaaagaatcttaccatattggaggagaattggaggaggcctcctgaattctccaggaggttacctcatactttattgtgagtgtctggattggaaatgtcctagaagtcaaccccactcatctggatcattagcccctcccagttcaaccccactcatctggatcattagcccctcccagttcctaattcaatgaaaagccaccactagccattctgttcatgaaaaacaacccctgactgatgtgtgagtttgttctgacccagcagagaaggactgggaacagccagatgtttctccatttccttttgtatctgctttcttttcaatccaacaagaatgtgttcatctctgaccgcagacacagcattctcatttctgaaaatcgccatggagaaatcaacgccaaggccaacacatgtgggagtgctggatgctgcctttgtggaagtttagggcagcttatgagagtgtactgaattgatgattgctttatttttgttagtatagattttaaaaataaactaataggctgggcacggtggctcacacctgtaatcccagcattttgggaggccaaggcaggcggatcacctgagatcaggagttcgagaccagcctggccaacatggcaaaaacccatctctactaaaagtaccaaaattagctggatgtggtggtgcacacctgtaattccagctactcaggagactgagacaggagaatcacttgaacccaggaggcggaggttgcagtgagccaaggtcgtgccacggcactccagcctgagtgacagagcaagactccatctcaaaactaagtaaataaataaaataaaaaataataataggccgggcacggtggctcatgcctgtaatcccagcactttgggaggccgaggcgggcggatcacgaggtcaggagatcgagaccatcccggctaaaacggtgaaaccccgtctctactaaaaatataaaaaattagccgggcgtagtggcgggcgcctgtattcccagctacttgggaggctgaggcaggagaatggcgtgaacccgggaggcggagcttgcagtgagccgagatcccgccactgcactccagcctgggtgacagagcgagactccgtctcaaaaaaaaaaaaaaaaaaaataataataataataaacaaatatttccatgatatttttaagttgctaatttgtggtcagtctttccatcatccatctcctcctctgtttttctttgtaggttttatcactgcttaacatactattaacattgtcttacattttatatctatcacctgcctgccactcctaactagaatgtacaatctatgaatgaagaggttattttttctcttttgctcacagatctattccaagcacctagaacagtggctggcacatagtagatgctggaaaaatactttctgaatgaatgagtcagttcatgtggtcaaccttctcaggtaggtatttttatctccattagataaaagagaaaatggaggcttagagaagttaaactatttttggagatgatgccccaaactcaaaagttttaagcaaaatcaaaatgatgtgaatgcaggaaacttcagagccatctcagataagaccaggagcttataggacgctagagagcaatagttagacatgggcaaggaagaatcatgtagtcagtatcaaatgttgtccactatggatggcaaatgctgaaagaggtaagagaaaaatggatttgtgagccatggaaacaacagaaggcctcagaaaggaggtgaccccggaagaggctgcctcttaaaggctaggtaggggtcattattacttgaaaccaatttgaacctctgtaaatttttctgagtctgaacagtttctggccccaaagttcagctctcttaaacctatagcctaccatagaaaggattcattcattctatacacatttactgagcagccacctaggtcaggggtgctgcctcagtgctgagcatgcaaggatgaaccagcacaggcctgggtgggggacagcagcccctacactgacagttaagaaaggatgtgagaagttcaaggagcatgcccaggacctcctggacccaatgctcccactgatcattattctctttgttttattttatttgattatttatcagtgtattcacatttacagacacaatagatgctcaaatattctgtggaatgtaacagtgagtaaaggaatggtacaagaacaggtctggggtcgttccagcaactgaagctcagagccaccgtgccagccgaccagttcagccagaggcggggagggatctccttacagctaaggaactaaagctcagagagcttaaggaatcactcagcaagcatgggaatcaggatattaacctccatctgtgcgaccttaacctctgtagactgcctttggtgcattcaattttcaaagcacctttgggggatatgtttgttatctctgctttattgataaaggagctgagagtaattagcccaaaatcaaactgcgttcaaacctagaccgagtagtgaacaggagctcaaggaagtagcgagagagcaagcatagaccatttttttcttcttttccaaattaaaatttatatttatatttatttaattattttattttattaagatgaggtctcactatgttgccctggctggtctcaaactcctgatctcaaacaatcctcccaaagtgctgggattacaggtgtgggccaccattttttcaagaacctcagggcggaaggctctcagcctttattcaccccctagtcttgtcctcatcatttggcattgcagccaagtttgtattgccccgccagcttccacggtcctgccccagctctgccagggttggcccctgaaagagaggcagtgtccaaatttattaaatggcttttttaaaacatctcataatgattacatatttttccatgaaatttccttgccttcattccaggaaaagcattaattatggtctataatcaagattttcatgtaatatgcactctgttgacataatgccagataaaacaaccttttttgaattaatgtagtttcttagtttttaattaactttaatgtggagaagggaattatcaataaaattgtaaatacttaggtttggaaatgaaccttaattttcacattaccacattgaaacactataagagagttacatatataacctgttatatcacagaagatgctataaaattttttaacttgatcatataaatcaccatcactaataaacattttcaatatttcttaagcagtatctagatccatgcggaatacctcaagttcttttgaattttaatgcttgtacattatgagaaaaactgaaaacagcagtatgttgttcgactttgtttaaatctctcttcaaatgataccataccttaccttacaatgaatgaaaaaacagcttctgtagacactaactagtttggggattatttgtacaggaatcttcttcaggccaggcaaatatttttgtttctttgtttcaatgtccttatattttactggaatttcatttttgctatgtatttcacgtgaggttttgagtttgtttctccacacttttaaagactcatttttaatccttaagaagtaaaatagccaaactgaaataattttttaaattgtgccaggaggatcaggaagccacagagaaaacccttggtagtggttagttctccctgggctcagtcctcagaaggtagggctggaagggaccttttgggggtcgcagatttccacccacttactccaggcagcccaggtgtctcttgtctttggcctgctgtcattttcaggacaggcttttcaggagttggactaatcttttgtgccagtttgcttctcttaagagtggggtggtgggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgaggtggcgggcacctgtagtcccagctactcgggaggctgaggcaggagaatggcgtgaaccccagggggcggagcctgcagtgagccgagattgcgccactgcactccagcctgggcgacagcgagactccgtctcaaaaaaaaaataaaaataaaaaataaaaaaaataaaaaaaaagagtggggtggtgtaggggacaggcctaaggttttagttctggcatccactcatggctggtctctgtgcctcagtttccaaaaatatgagatggggacagtggttcctctcctgcctcccttgcaggtgaacatggaatactgaatgtgcctgttctttgaaaagctaagtgtctatttctaacatttaatgtattaatttagtttacacatagaaatttaatactctgcatatgtgttcatttagaaatatttctaatttgccatactcgtttggtgaaaccatttattctttgatatcactgtttctgatgcctctatacactctaattcctcaactcagttttccagagcatcctgtattctcatgcaccgtattttcatacatctaggttgcttgggagagatacatcttttgaagccatccgtgatgctgtcactgaaaactctgtcccaggccccaaatacccatccatctttgaggatccttacactataaccactaactctactgaggtttttctttcatagatctttaaaataattgttcacagcagtttaacttgcatttgtaagaacattgccccaggaaaaatgatttaatccattaagcagctttgtgtgtgtgcatgaagccaattccattgagtgactgctacaagtgtctaaaactagcactactatgtttatatcacaggcaaatgtgccccttcctcaaagaacctgttgtaacctgttgtcacaaaataaaacaactgagatggtgccacataataaaaattttgtaaggactcaattattaggtgattcttcttatgagaaatacatttaggaaaaactgctttctattataaggtgattcttcttatgagaaatacattcaggaaaatttgctttctattcaagtctgtattatataagactagaattcctgcctctttcaggtcctcagatacactgaagtctccttctgacatttggaccaagctgtctcaatccttggagctcctgggctgcttccaaatgcagtagaaaacagtggcaggatttaaggtctctgccccgatcccaccctgcccattcctccttctatctgaggaacttagggactccactgggccctctattattttcctgaggaagagttctgcctgatttctctgtggtctgtgatgaccaacaggagatgtgctcagaacccagaggccctggccaggctccttacccaagggaacccctgcaacgtgtgggtgcggctgcttggtgatggaccagtaacatctctgagagtgatgatggatggtggtcagctgctaggagagccctggcttgggtggtctggtcattctctgtggcgctcatggagaaaaactaatactgtggaaagagacagcgagactgagctcaggtcaatgaaaggaaatgtggtcagcgtaagactctctaaggatagctgccgtgtgttgttccctatcactaagaaggttcatgaacagtctaggctacaactctcagggctcttgaagaagagattcaagcattgcatgtggacttctatgaagtgaccttaaggtccattccagccagatgtaacattttgtttaaccatttccagcctggctgaacattctgctatctagtggcagtcttcagccgcagtgtgacggaggccaaaagcagtttctctttgtaccttattttctgaagaaatcctataaccctggatgacccaaatacccatcccacttacgccagctcccaatacccccatacttcctaccaagctttatgccgaaccaattttttcatctgggtttagatttcccacatccctaacccattctggcaaagtcaagatttacccctgctgagtaacatgacttctggactgagcggcaaactcatcaggataaaccagactccggaaattcaattcatcccagcaaacctttttgaagtctactgcacaattataaatctaagtttcagtgcccattttaaagatggaaggctcagagagatgaagagccacagtctcccagcagttaggcagcaggaccgcggcttcatggcaagccccctgactccccagcaggacttctcactgagaaagaaggtgcttccattaaagcctccattaaagatattcatgttttaacccccagaatctgtgctatgctaggttacatggcaaaggggagtcaaggttgctgaaggaacaaagtatggtaatcagctgaccctaaagagatgattccagattacctaggtgggcccaacacagccacaaggttcctttccagcagaggagggggcagagaggagtcagagtgagagagagattgtgctgctggctttcatgatggtagaaggagccatgagctgaggaatgcaggtagcctcaaaagctggaaaaagcagggtaacaggttctctccagagtccagaagggacacagccctgctgacaccttgttttaccccagtgagacctattttggacttctgaccttcagacatgtaaaataacaaatttacactgctttaagctgctaaatttgtggtaatttgttacacagaagtaggaaactacagtcatccttcagtgtccatagaggattggttctgggacctcctgaggatatcacaatcccagatgctcaatccttaatacaaagtggtgtactatttgcatagaacctgcaaacatccttccgtatactttaaatcatctctagattactgtaatacctgatacaatgcaaatgctatgtcagtagtcattatactgaattgtttctttttaatttgtgtcatgttttattgctgtattgttcatttttatttttctgaatgttttccatttgccattggtttgaactcttggatgccaacagtcatctctgtggctctctcctttactcattccagtctctgctcaaagtcatcttaacagagagaagctgccagcagcacagatgaagtgctgccctgcacttgcacagtctttccaccgttttccctgctgtccttcatggcgctgaccaggtcctgcagtatcaggcgtcccctcatttcctggcatgtgtttgtcttcccacactagaacgcagccctgaggggtggccttgagtccttccttccctctgtgtccaagcccagagcctggccccgcacagtgtaatgtgccctgaagagtgaacacatgaatgtgttggtcaccgccccattgtgcagcgtagttctccatgtcggtcgtgggaaactgaggcttggaggggttacgtgaccctttccccacgccacgaagacaaggaagcaggaggagaaaatacactaactttggacagcctcaatctggattcgaactccacctctgccccttaccaactgtgcacacacaggaggggggttactaaccccttgaagtcagcttcttcctgcaacacagacagatgccactctgccaccctcacagaatgttaaagtgggccaaaccctgcaaaatacatagcccagccctatcaagtcctattcaaaggtgacactgtatttatcagggaccatgagtgaccttctgctcaaaggggtatggcattgccatcaagaaatcagaactctgcttctcacctgttaaggtttagggcctgccagatatccgacctgcacttaactctctattcttatcacagaaactgtggagtggatttgattagttctattttacagaccagacaactggggctccaagaggagaagtaacttacctgccccacatgccacagtcaagactggcagcaatggtggggcccagtacactctttcccacccccaacacttgaagacctcaaaagtaggttgatgccatgtctttactactgtgaatagtgttgcaatgaacatatgtgtgcatgtgtctttacaatagaatgattaatattcctttgggtaggccaggtgcagtggctcacgcctgtaatcccagcactttgggaggccaaggcaggtggatcacaaggtcgggagatcaagaccatcctggctaacacagtgaaaccccatctctattaaaaataaaaataaaaaattagccaggcgcggtggcaggcacctgtagtcccagctacccgggaggctgaggcaggagaatggcgtgaacccggaaggcagagcttgccgtgagccgagatcgcaccactgcactccagcctgggcgatagagcgagactctggctccaaaaaaacaaaaatcaaaaaacaaaacaaaacaaacaaacaaacaaacaaatatatatatatatattcctttgggtatatacccagtaatgggattgctgggttgaatggctttctgtctttaggtctttgaggaattgccatactgccttccacaatggctaaactaacttaaactcacaccagcagtgtataagcattcctttttctctgaaatctcatcagcatgtcattttttgacgttttaatagcagccattctggaatcaacctaaatatccgtcaatgatagactgaataaagaaaatgtggtacatatacaccacggaatactatggagccataaaaaaaatgagatcatgtccttttcagggacatggatggagctggaggccattatcctcagcaaactaacacaggaacagaaaaacaaatactgcatgttctcacttataagtgagagctaaatgattgagaatacatggacacacagaggcgaacagcatacactgcggtctttcagagggtggaggtggggaggagggagagaatcaggaaaaataactaatgggtactaggcttaatacctgcttgatgaaataatctgtacaacaaacccccatgacacaagtttacctatgtaacaaacttgtgcttgtacccctgaacttaaaatgaaagtttaaaaaaagaaagtaaaccggattcttcctccatatgtatctcagtttcagcatctgtgaagcccactctgagctgtcccaggagtataaatccctttatgcattaggcacctctcctctggagagcttagcaagggggtaatttgacatttatttttgtaattccttgatcaatgtctgtctcccccaccaggctacaaactccatggagctgcagaaccatttggtgtgggttccccactggtttccctggtgcctagcacagagtctggcaacgagtaagggctaaaatgtattttcagaatgcaccaacggctgcaaagatggactctaaatgccaatggcaaacttctcaaactctagcttccaacaaggtgggccagattttctcctagttccccatacaagctaccccggggagccgcatgagctcctacccgggccttggtcttcaactgacctatgaggtcaaggcattctctctcccagaagaaatatccagggcagaaaaaccactcccacagtgctcaaagcatttccctttgaacattttcctgcttcttattttattcataaccaggcaacctgaagctttaacaggatacatggttttttctctcttctagatttagagaaaaaaatgtttgagatatttttaactgacaacacccagccccttctccctatgcaacaccctctccccgaatctgtgctcagacaccctatctcagccccagagacaggctcctgaggccgaagcagctcaggccataaatcctcatctgcggccggtaatccacatggagagaaatgacaaatgctctatttagccctatttccaaggaaattagaattacaaattggagctattgctaaggccccgcttgagcataacctttggaaataaaggtagggagggggtcggggaggactggcatttggctagccaatttctttgtcaggttagcttttgtagaaatccacccccacctatgggaatcacacccctccccatcccttccacccccagcactaccaattagagccggtggcccaggagtattcgctctggctttgttcaccgctaaaggcaccaccaaaccaggtaagcaatcacttccagggaaagtacagaaaattgcttctttaattgggtagtttctagggctaagaataatttaattgaaaaccagcattaatgagaatcaaaaggttttaaaaaggggcctggatggagatgtccacgtgtcagagacaagtgacagccagagttctcaaaagatacatggagaaaagggctttgttaaaaatccatcatcccccactacccacactcctacctcactcctccaaacacacccctccccagcacagatacacaaggctgatttggatgcttatttcgaaaagcttcatctcatctccaaaccactgagaactactggggtagatgttttttgttgcattttgtttcattttcttaattttgcagccagtagtgaaatataaggcaaacagtgtagatttctacggggtgagttccaggagccctgagttcaggtcccgttccagtacccactatgaccctggtcaggtcacccagccctctgacactctttacccccccatttgtcttatttactcaacaactactttcacacagctaagggccaagcattgttctcagcactttataaataataagtgatttaatcctcttaccaagccccttggacaagcacagttcattgcccccttttcttcttatcagaaacccaagaggtaccgagaggttcagaaacttgcccacagtgacacaactggcaagttatggagctgggagttgaacccagcagtctggttccagggtccacctctggcaatacacagaaaggatgctaccactccatcaacccctatttcccaaggggtgtgagtgctcagaggacgtgggtgggttgctgagcccatttatctcatgtgatttagctcatgcccctctaatccttttgcacctgtctcctgtgtgctccctcctgccctcctcacccagccaccaactggttcctggcattaagctctgtttctgcactttgagttctgattctgtgccttcgtgctacaactggggtcatctgtcctgccccgtgtgaagggtctacactgaatatcgttgaatcaaggattttattccaaataatgaggcacaaactcataataaaaagacagcatggtgtggaggacaggagctggtgctttgcagcccagcagctgggactgaacttaggaaaatcatgtaacttttctgtgcctccacttccttcttgtaaaaataaaggaataaataatcaagcctacttcatagagttgtgcaaactgagcatgttaaaataagcatagctctacagaatgctacggggaatgtggtatgagacagatgcacagtgtgacacagctgaccacaggtcagtgctgctccgccaactgagctgcaggatggcagattgcagagagcgtgggatttggtggcattggactttgactgaatcaggcatgggactctgtcacatcaatcagcatctccaagcctgttccttttatataaaataaagagaataataattcctgctcacttcacctgattatgaggattcgacaaagcagagtgctttgtaaattacaaaggaacctagaaacattgagctttattcttagaacaaaattagttggtgagaaagtataaggagacttggatttcagggtagtaatatgggctaaagccttcagggtgtttccaggtaacatgctctgcacccgatcaatatcaccttgtaattctcacaattctatgcctgttttatagacacaaaaacctaaggctcagaaaggcaaaaggaccaactggtgtcatgaaaaggggtggactgacatttggagccaagtcccaaatgctcttaattgtggcattacattgtcacattcctgtcactgtcacttcatggggaagcaaagagctggcctttcagaaaggaagagggtcaggccccagatcaggagaaggagaggcagctgtgagcaaaggtgaggaggaggaaaggcaaggcaggtctccatggcccagggcagcacagccacgtggctttcccgcctctctcctccagactctgcgttcactgtgggcagggacacattagcttgcatttatttcccccatgcacctgccccatccccaacataaatatttttaagggaaatcaagtgaatgaatgaatgatattactcaactgaggcactaagatcaagttaagaagaaataaaaaaacgactctaataagcagtaaagccatgaagacatcttccccaatcctccaaatcaaattaattttccctcctttgggcacccttagttcttctgtgattagccccacccacccaccccaaaaaaacacacaaaaaatcattattattaatatcatttactgagctcttactatgtgtcaggcatttcccatacagaatcctattcaagtctcacaactaccccatttcacagatgagaaaatcaagactcaccaaggttacagaactgggctaggtcatacggttagtaaacccttggtttcatctcaagtctctgtttctgaggctagaactcagctgtttctctacatccctatctgtctgcctattaaacactttataattttccaagcaagaagtccatcttacatgcccatatgcccgggataccttgcacaatgctgggcacagagtagtgtgctccatgggcatttgcagagctgaactgtatttaatcatctgtgtctattcaaaggctgaggtctttatcagatgggagcccagaagggataagctgggttactgtttgcctctccaagcagaaaaagaaccagatgacctcctggtacatgtttattgctaacggccctgcagtggcttccacaacatgcctcagcctggatccaccaaccttaagctactctaaagacaatgccttcgtggtggccaacaagattagcacccaattaataactaattttggaaagactgtgcaaaacagcttggcaaactgcatcatgccacacattagcttgacctgagagcaccctgcctggaaatgaacagatcaaaggggctgaaaacatgagcttgggagacccggactctgctctccttggctgctgggaatctgtgtgtccttaagcaaactccctccttccctgggttcaggttcttcgtcctttaagctgaaaagcagagctagactgggaagacctcttccaggtccaaaattctagagctccgggatacaattaaagccaagcaaggcaaccaggatgagtctatgtgtcagcaaaaattcccaggcaatgctaattactggcacttaaccaactcatagttaactccaggtaatttccagaggatttattacaactctgaatgctattttgtgagggaagcaatgtatatttaatattgacagtacaggaggcaagttccctttctccaagcccataaattatcccaggcgacgggaggacaaaactaccaaaggcttgttacaagcaggctgcctcccaaacagaaatcaggcttaatagaaaacaataaaaaagatgtaggaggagacgaatttaaagccagaagcacgtgagtggtgtgtgagacagcatgtatgtgtgtatgtgaatggcctcagaaaatcaaacgatagaagtcagagaggaatgtccaattcatctcattaccattagggtagcttcctgttcaatcccacaacaaacatttcctgagccccttctgggaacccagggtgagtcacatcagacttggtccccaaataagctatgcatgtggctctttccctctccccctgggactctgagtccatcttaggatgctctgttttcatatccccaaaatgagaatccaaagacttacatcgtggcgtgttctgcaggcttcataaggtaatgaatgcaagaacaagatttttaacaagtggtggtaacacagctagaaataaccctaaagcagggcagaatgggcagacaagggtcaagataaaggtaccagagaaaaccacaggcagacagtaaaggtttctaggacacagatgtggagaagacactgcaagaaagaagaaaaggaagaaggtggggaagaggtggtggaggggtacctgtgcgtgggccacacagtgattggctgtgtgtgaggaaagagactgaaaaaaaacaggagaaagaaaagtaatggagattaaagaaaaagaaggagcagagtagcgggtgagctgggttatgggagagagagaagaagaaaagaggacgtaaaccataagggagataagataaaacagaaaatcaatagaagtgcctaaagttgattcatcaagagacagcgccacgagcgcatgatttagagatgcaggagaaccaccaggagttctaaggacagaaagaacgagactgcctctggggaaggggctgtggcttttcatttttaacctgccttcctatatgactttgggcttgaatatatgtatgtattcctctgatagaaataaaaataattgaaagcatttttaagagatggaaacagaagttaccacggcgtaagtactacctgctgggcatcacccccagcgacttataaataccatctcatatttgtcatcccaaccacactacagcgtaaccaaatccccttttcattttcctttagctgcacttactttgtgggggctgttgtgaggattgaaagagcacaggtaagcagctacaaatgtgcctggaaaatgcaaatgctcaataaacgttccttcctattgtcaaagaatttgacaagcactcctcctttttcctgggagctattctgccccctgttttgtagcagctgtcatgccatcttatgatcctttcacccaaagacacaggtggggaataagggttctgcagtaggcacctgaacccagctaagtcagtgggtccctaacttaggaaggcaattacttttcttcactgtaaagccacacccctcactcaggccttcttgatctcctttggggtccatgagcaagcaagatccctgggacgcctcagctggtttaagatgggtttctgctaccagcctctgaaagtgtcaagggagttcaaaagcaaacgtcatgtaggcgtaggtctctccagggctttcccagaacaagaagctgagctctaagggggaagaaggagaaatatgaaaaaaaaaaaaaaaaaaaaagaggaagaagaagaaaacaaaagaaaaagtaaaaaggaggaggaggaaggatgtgcttgcagtgaattctgcgttttgattgttgctattttatagctaggaaaaactaagcaaaagcaatctctggtcacagctagaaaactattatttgcgtttctatctttccaacccacgatctgtgccatttggatttcatggtctaaaaatatctcccaaacttccaagtatgttggcatgggttgatggacttaaatttttaatccatttgtaaatgttttcacacagctcttttttccctctcaaattctatttcttgctgaatcacttccatcatttagtttggcatttatttgagcaggaaggaaatcatcttgaacacaaatgtcagccctggccccgaaggctcctgagagagaaaaaaggccaacacgggttagagactggtgaggttgccacggtacggcaggaagagcaccggcttcggtgtagactattctggttgcatctggctgtgtgaccctgagcaaaatattggacctctctgagcatgttcttcagctacgatgtggggataatgaaattcagtttaacagtgtttgtagccattcagtgggttagagccccatgtgtacctcctagcacaaggcaggagcttagtaaatggcctcattgtgaattttaggtgtcatcttgaaatgtgtcttgtaaagaccatgtgtatgcctgcctccgcatcccttctccagccaatggcagagtcactcttgcaaaacacaaatctgatcatgccactcccgaccgtgaaacccagtggccacaggatcggattacagtgttcactgtagaccactgtggtctacagtgaacatagtacagtggtcaatttcccatcccccctccacaccagcctctccaactgcctgccctccaacctcccagcaggatgctcctactctgcctctcatggttctgatttccagttcaggtcctccagctcagccactcccttttcccacctctctagacccccacaggaaaacacatctctgatggtattccctctggagccctcagattcaccccccacaccattggccctgcctgggtcatgtgcttactcctgtacctgtcatcattgtcaggtgaggccagctgatcccaaacaatgaagtgaagaataggaaggaattaagtccccaaagtaaaatcatggactgcttggtgggagagctgggggctacatgtgggaggcaaggatcaagggtcaaggctagtttccacggatgagcaagggacagttaagaaccactgaggccttaaagttttacaaactgagaaattaaggtacaagatgggcccatagcaagcccaagttcatgccaggccatcacccagggtccagcccccaactctagccctcttcccactgtgtccagagttggtgccttccactgggttcacagtcttgctgactttaagaatgaagccgtggacctccgtggtgagtgttacagctcttaaaggtggcatggacccaaagagtgagcaacaggaagatttattgtgaagagcaaaagcacaaagcttccacagcatggaaggggacccgagtgggttgccactgctggctggggtggccactgctggctggggtggccagcttttattcccttatttgtccccacccatgtcctgctgattggtccattttacagagtgctgattggtccattttacagagtgttgattggcctattttacaaacctctagctagccacagagtgctgattgtgcatttttccagggcactgattggcacattttacaaacctctagctagccacagagcgctgattggtgcattttacaatcctaactacagagtgctgattggtgcattttataatcctcttgtaagacagaaaagttctccaagtccccactcgacccaggaagtccagctggcttcacctctcacctccgtactgccaacaccttccccctcccccaggaaaacagcttcatgattaaatcaattttgcaatcagcagttccagccacaggttccatagcaggatgtgaggaatggtttctgaagatgaagtggaagggctacagggcactgaggggcaggcatcacaccatctcccacaggcaggcccaccagagtcaggcccaatgcaggtgaatgccctttgagccgaagaggaagaaggagctcacatgcttgttctccttctagggaagcctcagcactccagctttccctcctttcctcatcatcaccgtcctcccttctctgcccttcaatggacgactggggcaagggaagggggtgagagaaaagaaaagcagaactgaaaaaggacatcaaaggatgggttgattagaaagctctttccagagaaggatggaatgtctagaaataacgtctgtgatgccctttgttctgagcctttctgaagagcttttactcatcgctcaagatcagtaatatggtttggctctgtgtccccacccaaatctcaccttgaatggtaataatccccatgttccatgggagggacccggtgggaggtaactgaatcatgggggtgggtttatcttgtgctttctcatgatagtgaatcagcctcatgagatgtgacaattttataaaggggagttcccctgcacatgctgtcttgcctgctgccatgtaaaacgtgactttgcttctcctttgccttctgccatgattgtgaggcctccccagccacgtggaactgtgagtccattaaacctttttcctttataaattacccagtctcagctatgtctttattagcaatgtgagaacaaactaacacaatcgggctcaagaagcatctcctttgggaagactttcttaatgcaccatccaccacgggcagacgttatcctgtccctcctctatgcccactggcctgagacacactgcacattctatttgtgtgactgtccccacagcttgcattttccagaataggaaatcagtaggtgctcactaaatattcaatgaattggctaaggatgcagatgtacaaagtgcagggaagaagaagatttagtcatgcaataaacagaaatgtaggtgcaggaaggaagaaaggcgtctattcacacaggactccaacgctggagcagcagaggatgagttttgcagagagtcacagcacacatctatcaatggataactgttgcaaatattcacccacgacattttctccatttaactgatgagatgagatctgatgtctccaggccttaaaataggacaggagcagcgggaaggtggggagagaggacaagcttggatgtttggatgttcggtcagcctgtagtttcaagcggcctcaactgggacaggtcaagagaaaaatgggacattttcaaatgaagttcttttttttgcagaactaaacaccctggagttggagggcacctttaaagtcactgggcccaaacactcacagtggtatgtggtttctcacaccgcagccaagggttgggtttttcgtttgtttgtcactgtgctaaaatatacataacataaatttttatcattttaaccatttttaagtatgcaattcagtggcattaaccacattcgcatcgttgtgcaaccatcacctctatccacctcccaaacattttcatccccccaaaatgaaacactgtttccatcgagcaatagctccaccttctctgcttcctccagcccctggcaaccaccgccccactctctatttctatgaattaaactcctctaggtgcctcttttaagtggagccatgcagtatttttccttttgtgtctgccttatttcacttagcctcatgtcctcagggttcatccatgttgtaacaagtgtcagaaatgctttcattttaaggctgaacagtattccatgcacatgctgctttatcacctccactggaaccttctggcaaagatgagacactacccgctttccaagactgctgtccccctgcagtcaagtatgactggcaccaaatccttgggttcagcagctgagcatcttgctattactccttccactggccaagggaaccacagaagagtccattcctcacccacaagacaggactagggatatctgagggtaaggctcacagtcctcttcactctctgcctccaagctgggcatgctccaatctgtcaagcatgatagaggattcaggtcattttcgggccaccttgctaaccctcctccagacacatcccagtgagtctgtctgggttattcagtcagtcaacaaacacacactacagtctggccccaagctgggagctggggacaccacagtgaacttgacatgatgtttgcctgcctagagctctcagtttaatgaagggagacaaatcagtaaataaacatgtaacttacagattatgataattacagctcatgacagggtgcttggaaggatataaaaatgaagactggctgagagtaaggtggtcagggaagcccctcctggatccgaaagaagccacttcatgggagttaagggtgcggtctctcgggtaaagcaaaggcccccaggggaggcaggaccttggatggtctaggaagggccatggtgatctggagagcagaatgctggggaatggctggtccacccaagggaagagaggtgggcaagggctggatcatctggggcctgttggcctcggtgaagacggcgggtactagtctaaggtcaacagacagcccctgaagggctttgaaccatggaaggcatcatctagctcactctggaaaaagacaactctggctgccggttggagaatggatgggtaagagcagaagacagaagagaagtcaggacattgcaggagatgaggcctgagaggtcgggctgcctggaattattatgtggggaccctcaaggccagtggcacagactctgtgtatcttcggctgttcttacagtagagcctggggcctaaaaccgaggggcctgcactggcctagaggctccttctaggcgggtggtgtccctgagtgcaggctcagaggccagctggctccctcctcttcccctccaaaccctccaactctcttcctgtgagctggatcatgatcagtcaattctcattgatctgttttttttttatcatctctgagcacaaacattgaccgacttaaaacctggagtagaatccacacacaacagactcctgataaattatcctaaatggcaaggatggttgtgggggaacagcagtggggctgacctggcccaactagaagacgaaagcttctccccaggctgccaagcaagcgcctgccagaagcgtgtctgtgcagtccccacggaagccagtggcattcaggcttccgagagcagcttccaatgccagtgttcgcatctggggtttggtttggaactcagctcggacactgcaaggccaaagggattagtcaacagtgcttgttaaacacagacctcatgcctaacaccatgcctggcaccgtcgattaaaagaaaacatttcacgcgtggctcttacttgcaagagaaccatgatctggttaggatgctcatggcagaccctgccaaggcagagactaatgagagatgaatctgagtggtagctccactggagctcagggagaagggcccagaaaaggactgggagagaaagcatgggcctgtcctcgggttcatgtaggttccatcccagcttcacagcctaacagctgtgtgtccctgggcaagtcacccagcctctctgttcctccactctctcacgtccacaacaggtcacagtcattaaaattatgtacagtgtgcccggagtgttatattaataacagtagcttcatttctacaaggtgtgctctgtgtcaatgctgtaccaagtgctttacataaataaactcattttattctcctacattcttttttattttattctcctttattctctataaggaaggaaatctgtaaaacgagtacctccgttttacagacgaagaaatggagacccagaggaatgaaagaactgcccaaagtaatccagctaggaaggggtcatctcagtgcctgatacccacactgtcctttcccccgactgaccaccatggagcagcagatgaaatgggcttgaggagccagagaaactcagacataattagatgcattgatatggtgatgtacacccatggtcctggaagtattactcataatagccaaaaggtggaaatgaagaaacccaagtgtcccttcatgaacaaatgacatatacacatacaatgtaatattatacagccactaaaaggaaggaaattttgacacatgatgcaatagggatgaagcttaaggacattatgctaagccagtcacaaaacaacaaatattctatgattccattcataagaggtatttagaggtgttaaaatcacagacagaaagtagaatgtggtcgccagaggctggggagagggggaagtagtgagttactgtgtagtgagtacaaagttttagtttacaatatgaaaaaaacattctgtgccactgaactatacacataaaaatagttaaaatggtaaattttttttaagacagagtctcactctgttgcccaggctggaatacagtggcgtgactgcaacctccacccctcgggttcaagtgattctcctgcctcagcctcccgagtagatgggattacaggtgtgtgccaccacgtctgtctaatatttgtatacttagtagagacagggtttcaccatgttggccaggctagtctcaaactcctgacctcacatgactctcctatctcagcctcccaaagtgctgggattataggcgtaagccactgcacctggccattaaaatggtaaagtttatgttatacatatattttactacaattaaaaaatattgatatggacataagaggcctatgcttcttcttgggtgccccagtattactccctgtacctccatttagatttattcgtttcttcagttggcattttttgagcttggtacctattctatttagagctagaatccaagaggcctggattcatcaccactgtcctcgtcattgctgtcctagcagatacttacactctcattgtgcccagcacttgtccatgtgttccatctgcactaactaactcatgtaatcctcaccggcacccttagaggtggatacttttttttgtttgtttgtttgagacagagtctcgctctgtcgcccaggctggagtatagtggcgtgatctcggatcactgcaagctccgcctcccgagttcacgccattctcctgcctcagcctcccaagtagctgggactacaggcacccaccaccaagcctggctaattttttttttgtatttttagtagagacggggtttcaccgtgttagccaggatggtctcgatctcctgacctcgtgatccgcccacctccgcctcccaaagtgctgggattacaggcatgagccaccgtgcccggccgatggatactattaatattctcattttatagatgaggaaactgagacccagagaagtgaagtcccttgcctgaggtcacaccactgaaattcagactccccatagtctggctaaaaactctcagctccaaaaagcctctgtcacctgaaacacaagccacattttcatccacattctctctgcccacccccaatccaaccaaagtggccaggcttctgtagcgacaagttgtgcagcctggggaggggtccgagtcaccgcaatattcctggaacaagcccactgcttctgctatttccaaagtcagcatttcccactgctatttttttttccagatgaaagcaacaaggtccccagcttaatatgtttcatacagtgacactaattaatgttccaaacaccagggctcggggattgagggtgtgtgtgtgtgtgtgtgtgcagtttgtgtttaggtaatttattttcccccacttttgaaatgatgtatgtgggtattttacctgagccaaactgttcataattcaaaaagcaatgttagctttctattttttttttattttatttatttatttttttttgagatggagtctcactctgtcgcccaggctgcagtgcagtggtgcgatcttggcagctcactgcaaaactccacctgccaggttcacgccattctcctgcctcagcctcctcagtagctgggactacaggcgcccgccacaatgcccggctaattttcttgtatttttaatagagacggggtttcatcacattgctcaagctggtctcaaactcctgacctcagtgatctgcccgcctcagcctcccaaagtgctgggattataggcataagccaccgcgcctggctgactttcttaaagtcataaaaaactactgtgaactacacactaaccccacaagtgcaatccattctgcagcctcctacatatgtatttttgcttcaaccaacagtgtgaatgctggaaatctgaagccaggagccttaggcagtcacggcctttagaacaagaagtttacagatcacagaagggagtggtcctcaaacaagggtgccaacaccccaaacagaccctttggaaagagtagatctgatttattacaataggggaactctaacagtgctgtgggcaggagcccaggatgctgaacatcctgcagaatgtgtgacgttcctgccccatgaggaacagtcctatccataatagcttctccactgagaaatcccaaaatgtgtccatatagtttttcagggaagaaagatacagagacaccgaggtttcttgcctaaggtaacacagtaactcctcggttaagaaaggtcacacgcctccagcttcatctcttgtttcatcctctcaccactcagctaccccaaccctcccacttccatataaagggtccctggatgcagccactgggaaatgatcacactttaggactttaggaacctgctgcattttctcaacaccaagcctttgtcagtgactcccttctgccttcacccctaacccagactctgacaccgttccatctttccacgcctggatcaaaggctgcctccagaaaaccagccagagctctatcccggaacccctcccagcagtccatctgtattttgactttcagcctgtgtaccaatctgcctttgtactggagttggttattagcattacccttaccatcaacagtactatgccagctgctgaacaaccattctgctatcctatggtcacagtgggctggtggggcaagacatattatcctattttaccaacgaagaatttgaggcttaggaaggttagcaactttctggaggtggcacaggtggcaaaacagggatttgctcccacatctgacagcagagcccacactgttgtgagagggccaccctgcctccctccatggtcatgagcatgtccacctcctgtccagacgatgaggtctttgaaggtagaagccctgccacttccatccacatcctccttatggcaaaccctccataaatcatgtcagagtccaggcatcctgactcctggtccaggacttgccacctggtctaggtccttttcaccccataagccacccttggcccaacatgacctccctcaaggtacatggtgagtcaggagtagggccacacagtgagaagttccctaaggttcctacctcccagcaaccaacaacagttcactacagtagcttcagttagacaaaataaacaccttcccatcatatctttctggctcagcagtctgctttagcattgcagtgggccaggcccatgaaggcatccaagcttccagaaccagaggacttctacatgtgtcatgcctacatgcacaaaacccgccaatcaactttttaaaatagactttattttttagaatagttttctttttttctttttgagacagagtctcactctgatgcccaggcgagagtgcagtggcacaatcacggctcatcgcaacctccgcctcccgggttcaagtgattttcctgcctcagcctcccaagtagctgggactacagacatgcaccaaaacacccagctaatttctgtatttttagtagagacggggtttcgccatgttggccaagctggtctcgaactcctgatctcaagtgatcctcccgccttagcctcccaaagtgctgggattataggcatgagccactgcacctggccatgaatagttttcaatttagagaaatactgatactgtagtacagaattcccatataacctgcagccagtttcccctattattaacatcttacattagtatgatacatttgttacaactaataaatcaatcctaatgcatttttattaactaaagtccatactttattcagattcccttagtttttacttaataccctcttttctcccaggatcccatctaggatcccatatttcatttagtttcacatttctgtaggcatctcttggctgtgatagtttctcagtctttccttgttttagatgaccttgatagttttgaagaatactggtcaggtattttgtggaatgttccttggttgcaattcatatgatttttcacccataattaaacgggggttatgggatttgtgggggaagaccacagcaaaatgccattttcatcacatcatgtcaagggtacatactatcaacatgattaatcatggttgatgttaacctggatcacctggtgtcttagtccatttagtgtgctacaaaggaatacctaaggctgggtaacttataaagacaagagatttatttggctcatggttctgcaggcatggtgtcagcatctgcatccagtgaggacctccagttgcttccactcatggtagaaaggcaaggggagctggtatgtgcagagatcacaggataagagaggaagcaaggcagaggaaaggtgtcaggctctttttaacaaccagctctcacgggaactaataagaatgagaactcacttatgccccagcacacagggaggggattaattattcatgagggatctaaccccatgacccaaacacttcccattaggcccctactttcaacactgggcatcaaatttcaacatgtagtttgagggtacaaatatccaaactataacacctggcttgaggtcgtgttttacaggttcctccattataaagttttctttttcttgtttttttttttttttaattcccttttcataccctttggaaggaagtcacttatgcagccctcactaggctccctctccttgagggtagaatatctacataaattagttggaattattcttacaggagatttctctcttcttctcatatttattcactcatttatttgttatatatcagtatgggcttgtggatatttattttatacttttgttatactccagtattactttattttttgtctcaaattgtcccacattggccagggggtgctctttcagttggcttatgtgcccctttgacatactcccatcaatatgggattttttaaaacacctttttactttctggcactataagatgcccttggctcaccttgtatatatttcctgccccatcctagaatcagccatttattcagggatccctgtatcctttttgtgcagaatggttttagaaaccaaggtctggatgccaggtgtgctcactgcaatcaggatttgactttctaggctcaatgagctgacagcgcaaggatatatatgtgtgtatatagcccatgcatatacacatatttataaatatttctattcataaccatctatgtttgtattaagttaaacatgagtttacatgcatatttccaactcaaatccatactacatggatactctagactccatttacttttctgtaaactctcacttcagtggtgagaaacctagctcccaccactgccatattttgcttaaccgttaaactccagtacacaagtataacagtatgagaatcattgacccacaccccatgggaaacaacagcacttacgtactaggccttttgtttttagtcttagaaactccattcattgccaaagttgcttaggtcagcaccttctccctccatcaccttcaatgaggttgtttcatttatttgtaatatagctagattctcttgtcaccatctgcatcccttcctggggttccgtgacctcctaaataattttttaaatttacaaacacaaggctcaatttttgtgccatgaagttctgtgggttttgacgaatgcactaagtcatgtatccaccattacggaatcacacagaataatttcaccaccctaaaatatcacctgcgcttcatctattccaccctccttccctatcttctagcccctgacaactactgatctttttcctgtcactaaagttttgctttctctacaatgtcatataattgaaatcatacagtatgtagtgttttcacttagcactgtagcttttgtcatttagcaatatgcatttaagattcattcctgtatttcatgacttaatatctcatttctttttaccaccaaatacaatagtttattgtatggatgtacaatggtttgtttatccattcatcttggttgcattgttcttggcaattatgagtaaagctattagaaacatttccatgaaggtttttgtgtggatataagatttcaaatgagttgggtaaataagagcacaactgctggacactattggtaacactatatttaggtttgtaaccagtggctcacacctgtaatcccagcactttgggaggccgaggcgggtagatcacatgaggtcaggagttcgagaccggcctggccaacgtggtgaaaccccgtctccactaaaaaatacaaaaactagccgggcgtggtggtgggcacctgtaatcctagctactcaggaggctgaggcaggagaattgcttgaacccaggaggtgaaggttgcagtgagccaagattgtgccattgcactccagcctgggcgacaagagcaggactccgtctcaaaaaaaagaaaagaaaagaaaaggcacccactgccttccagggtgcctgtaccatttttccattcccatcagcaatgaatcacagttcctgttgccctacatccttgtcaacatttgacattgtcctcgctttggattttagccacccccattcaaataggtgtgtagtgatatctcattgttgttttaatttacaattccctaacgaaattatattcagcatcatttcatctgcttatctgccatctatatatcttctttaatgagatgtctgtccaaatattttgtctatttttaattgaattgttttccttattgttgaattttgagagttctttgtatgtttgctacaagctctttatcagataggtgtgtggcaaatatttctccaagtcagtggcttgtcttttcattgtcttaaccatgtcttttacccattaaactttcacaactactcttttgcagatgagaaaaatcgaggatcaaaatgacagctttttatagcaccattttagtcttgactaacactagcaagtgtaaaccataaaaatcaacgagttattacaggaccttgtaaatatactaaaaccactgaattgtacatttatttatgtatttatttatttatttatttatttattttattttttgagacggagtcttgctttgttgcccaggttggagtgcagtggcgctatctcggctcactgcaatctccgcctcctggattctcatgcctcagccactcgagtagctgggattacaggtgcccaccaccacgccaggctaatttttgtattttcagtagagaaggtttcaccatgttggccaagctggtcttgaactcttgacctcaagtgatctacccacctcggcctccaaaagtgctgaaattacaggcgtgagccactgcacccagctgtgaattgtacacttttaaatggtaagtttttaagttatatgaattatatttcaattttttacaaatgacaagaaaatcaacagtcagtgagtggcagtgctggaatatgaacttttgaccgcatcacagctactgcattcagaatatctgaagccaatcccagccctgcccctgaaaaactctgagaaagtcagatacttttactgaggctctttgtccccattgataaaacaggggccaattatacacatctggcagtgttatcacaaggatcaaatgcaacaatgtttttaatgattaacctgataccctggacacaggtcaataaggaatagaaagaatgattaccattgttattactactgttgatatcccaaaatggaagagtattctccctctccccatctaccattttccctttccctccttccctctttctgtctctttttctacacacacacacacgtatacatatacagtatatgcctcattcaagacacacatacacagacaatgttagcattccgggaagacacaaaacataacaacctttagcttttatcagatttttacttcataaatagccctgttttggaagacgacaatttggcccacaacatgttcacatctatgaaagaacccagactaatttagtgggattaaaaagaccagactgtgtggcttcagttggcaacttcatttacaatatttattgagctcttttgtgacagaaaaataaaatcaagtaacaagaagtatttggaataagaagcgtagttcaagagcaccagaggagaggaaaaacctagaggaagaaagaacccaggcataaagtctataaggctgtcactctagttgagccacaaatggtattcacagccctgacagccaaagcaaacaaaacagaagcatcctgcagaattcattttctacaagatgaaaacataatttactcaggaaagaaatccgctagtttctcactcaagtgaaccgaaaactctctagatctctttttccctactcttcaagtaagccgagtgtgtggttgggtttagacctctaatatgattgcaaaaactcagcactggcctccctggaggtcgtgtggtctaggcaagtgtttttcatggcatacctgcttcctcaggtgactcgggccaggcagcaaatcttttcttctacacttctattttcaatgtaatttcctcccttttatcaaaacggcacatcagaactcacactttatcatgctctaggacataaccttagggtaggaattatgataccatattgacttcagcattacttttattcagtgacatcattttttcttttattctttgagatggagttttcctctgacgcccaggccggagtgcaatggcacagtctcggctcaccgcaacctccgcctcccgggttcaagcgattcatgcctgtaatcccagcactttgggaggctgaggcgggtggaccacctgaggtcaggagttcaagactagcctggccaacatggtgaaaccctgtttctactaaaagtacaaaaattagctgggtgtggtggcgggcgcctgtaatcccagctactcaggaggctgaggcaggagaatcgcttgacatcatttttatattagcagaattggaaaagaataaggatggaatggccatttatctgatacctactatacgcccataagactgtatcacagccacttcatttaatccttaccatgtgcacagacatgttcacgttacagacgagcaaacaggtctaggctggttaagtggtttactcaaggttactggttaaacaatgatacagccagacttcaaagccagtacttatgtccatgtctgcttatcagtgaagccacacactccttggaggctggcattgtgtccattcatgcaaaaagactttgtatccagagtgtcatgtgttgtatcaggctctggaatgcagagaatggatgcttgcatgtatgtaatgtgccactaaaaagcaagtcctcattcgctttacacagctcaaggttcacccagctcaggttttatgattccactttcctttgtatcactttcagcaactttccttcagtgtgataaaaagtcggaccttggattcttcatggtcaaggctgtacattagaaaacgaaggaggaagtggtctccatcctgtacacatgatgaaatgtacttgatggaaatcttccctgatccatttaaacagtgttgacaacttttcgtgatccccaagtggactcagtatctgcatatattagagtgagtgtcacaccattatgcaattatttattcacctatttgaccactagtccctcaagagcagaaactgatttattcacccctctttctcctattcctagcccaaggtctggcatctgctgggcctgaaatcaatggcctctgagtgatcagtcagcatgtcttgagtttgatcagtgtatgtatacaaaacctaaaggattacattttttccaagaaatgttttcccagtcaaacaagatatgttcaagtcatggcacaaaaagaggaatttaaaaaagaaatctcaacgtttcctttcgtgttccagaactgtgaaggggaatgttaatgacttccccaggaaaccagaactccagcacagggcggaagtcacagcacctagtttacaaccaagatcctgccattttctccacaaaagatgtgtttgtaagagcaactggaatatctaatttaatcttgttttgaaaatgctaatgtttctcacattataaattagttaagattcttcattctttgaaagtatcacaaaagccactcagaattatgtcaccaggattgaagttgtccatgcttatttaaaataactctcttgggagttctagatcagcttgggcaacatagtgagaccccgcctctacaaaaaacaattttaaaaaaatagcgggcataggctgggtatggcggctcatgcctgtaatcccagcactttcggaggcggaggcgggaggatcataaggtcaggaggagatcaagaccatcctggctaacatggtgaaactccatctctactaaaaatacaaaaaataataataataataattagccaggtgtggtggcaggcgcctgtggtcccagctactcggcaggctgaggcaggagaatcacatgaacccaggaggtggagcttgcagtgagccgagattgcaccactgcactccagcctgggcaacagagcgaaactccgtctcaaaaacaaacaaacaaaaaatagtgggcatagtggcacctgcctgtggtctcagctacttgggaggctgaggcgggaggactgctttgagcccaggaggtcgaggctgtggtgggccatgattgcaccactggactccagcctgggagacagagggagaccctgtctaaaaaataaataaatacatgaaataaaacaaacgacaggaataaataaaaggaaagaactcgcctgttttatttggtgaaaattacaaatatgtgaccataccataatattaacaaggagaaagctcctttccccaaaggataatgcaggcagtgaccaggttttgcggtgcttctgagaacactataattccagcatatagtaacagcttagaaagtttagctatcattagataccaggcattgaatttacatatttacaaatacttttaattcatttttttccggaatgacaaccctaactgaaaaattaatgagggggaaaaaacactagagaatcatcacaacccttaggcttcaagacttcctttacttaagaatcaccgagaaaacagagccacatattgtgagctttaagtcacagtattttctttacttacgaaccagaggacgactaccagttgtcattggcgcccttcccatacctcctcaggactcactcccagcccctgccaaaagtagcctaaatgtggcatgtttgtctaagaaatttctctgcccccaggagcaccgcttaggcagtcatggctggagttcaaggaccattcctggctgttctagatggcattccagggtccctaggaagactaagcctcagttgccctcatggtaacctgcttaatgacacatccttataatctacttctttcctgtcttatttcacttaccttccccgattttacaaaattctccaatttgtcttggaatcacctcctaaatcatgcattctcactagggatgctattgcattccagggggcaaaattcgttctgggaggatagagtgaaaaaatcttactctttttatactttatatagcaagcccatataaacacacagtttacaaagacgtacacagtgtatcagtactattaacatttcatgggtgagggcaattggggaaaaacgtctctaaagactcctcaaaggggagatattgaaaacaagagagagcaactgtgtggtaaataaaccacgtgccctgagccccctggctcagtgtcggcttctggtgaaacacaaaccaagagagaatgccagcactcatccccacactctgttggccacagtccagtcctccgttgcttcacttaaccctttaaataacctggaaggcagggaaaattaggtccatatttcagatgagcaaactgtggccactagaagtcaaatgagttacagaaactagagagtaaatcagcagaagactcaaggtgagagcctgggtttcctggtggctgggcgggtgttccctgggctctcagccacaccttagaactatctagagtgcctagcccatggcacagagaaataacactacaaataaatgtaagtggctgttaataataaaaataaatcaggtctccatgaatggaacaaacaatggtgctaccccacagatgactttataaaccacacagtggatacaaacattagttgtcattaacatggaggttttgaatgccccaactcaaggctgagctcagtggagtccagagactattttataggactcttggtagagaactagatgggaggcaggggaaggacagaaaaagggggatcttagctctgggatttcagcagaaagagaagccttccttggcaagagtccatgtagggatgcctgatccctcagcccgacctgcgtttactcctcctactgttcatttataaacaaacacctcacctcacatggatcaataagaaaatgtggccctgagtggtaactcatcttgcccttgaaagaaaacattctgaacaaatatccctagcttttctgagataaagtgaaagaccccatcaagcaaaactatacttcggacaggattggacttagatcctaaggcagctgtgaagccagaggaaacaagactcttctgatggcacactgctgttggaggcacgtggggaggccagtgtggctgaatggatgcccgccttgcacgaagacttggtttgggtcctccccttagcagcaccaccttaggcaagtttcaaatggccctgggcctctgtttcctcttctcaaagttccgttccagctccaacagcctctgatttttctaagctgtcaagcgcctctctctctgtgctgggaccataaatcccacaaattgctgcaaagtaactgctgattagtgtgattagacttgctgattattaagtactgctggtttcatttttcagattggcattaccagggcttcggagaagcatccatagctgatacatgagacagctgatatggagtgagacgaagcagaagctgggattggagggacaggcagaaggagagagatgaatagtccaattgggctaagcgattttccattaggacacatggcctgggctagatgagtatattctcctagcccaggactttcaagtcttgttggataagatttcactccatctctccaagaagacaccatcaaggcagaaataaacttaaactgctgtaatgagtttacaaagaacctaaggggtcaaagacaaagaagttctatgcatattctgcctgcctgcctttgggtaggacaccagcttaggagaaccgggtttggtgatttgcaagcaggcacctgctgtagctgtttaagcaggttaccctgacattgaacagatggcagtgttgcccaagaggtgatattataaactgtgaaatgatgtaaaagcattagttattactaatatgctggttttaaagggctcaacccaactgttacactcttgcagagtgcagataattttacatgactcttaaaaaggagcgcatggagaggagaggaggttcccagccctgaggtctgaattcagtatagagagaagcccacctcggggggaagccagccagagcccacgtatgggataccccatgagccagctgcttaagttctcactcttacttagtcatacactacatgcaggaagcagtgttgctcttgagctgtaacccaatttgttccctagagaaaacgcttcaaacaactcttctatcacttctgtggcagaatgaaaagtcccatccaacaaggctacaatgtggatgggatttgatgtgcatcctgtgacatctgcaaaatcagagggggcctcaaaacttcacattgacttcaattttcttatctccaaacctatgactctcttcgtctttccaaagggtgatcaccttggggaagctaacatccctatagacacaggtgctgaggtatacggttaatagatgcccaataaaagtaagcactgaggatcaggtgatgcaacataaacagaaatgtggcaaatctgtgcatgggcagatgcttgaacccaatatagacaaggtcctaattgtggggcccacctgatccttctacagctttaggaaaatactgcctcaagatgctgccactcacgctctctcacacattgtgactgttacaccaattatatgcagcaggtattgcgatgcccattttgcagactgtatggtaagtgaagtgtccaaatcacatagttaatgctgctctactgaaagctcactcaacgtactctgacaaacccagagctatcttgggctacccaaaaccagaatgacagggcatggcatctaaaaagggtttttattcagtccacatttgaagatcttttccttgcttcccttgacactgtcccgcttgttaaaagatgaaacaaatcttgtttgcattctgtgaagacctccagacccaaaccaaagagaaaaaaggagcagggaccctgatcggtgggaaaaaccataggatcacaaccaagtttcatttgatttatgaaacttttaaacatttatgaaatgttaagaaatgaataaacttgaaagcgggagcttccaaataatgcaagaataatttagcattaaattgaagaaaatttgcattctgttctccaagcagaagaaggataccaggcatcacttggacaaacagtagtaccttcgtctcatccctggctgcttaaacctcccttgaagaagtctccattgatacaacacaggaaggtcagcgatgtttcctttccctgctctcctgaggttcctcatttgcattctattattgcatttgctagactctttaaatttagtcatttccagcctctctctccatacagaaagaactatgctcatctccctcaacagcctggcacaaggtttggcaacagggtctatacgcagaatccatgaatccaagaaaacccattcagctcagccaacagttctcagcctggcttcatgtcggaatcacctaagggcaatgcctgtcatctggactcacgtgtatgctttgtgtgtttgtgtgtgttgagtcttcttctttttaaactatttgcaactttttatttctaaaagttgtttttaagttaaaggaataatataacatacatatatattctctttacctggcttcaccaattgttaatatgtttgccacattttctttttctctttctcattttgggtgtgtgtattgtgtgtgtgcatacacacatgcacttatattgtctgtatatgtgccgaaccatttgaaagtaagttgtaaacatcatgacttgtcaccactaactattgcaccatgcatctcttcaaaatatagaacatccacttacataaccacaatgaaatgatcaggctcaataaattaaattttgatacaataatgttatctactatggaggccatgttcaacttttcttaattatccttatataatgtcccctgttatttatttttttcagctggaaccaatcaagagtcaagcactgaatttggctgttatgtctctttagcctccattaactgaaacagttcccctcatcttcttttgtttatttgtttgtttgtccatgacatgaagc

SEQ ID NO: 5 - DNA, 92735 bp, Homo sapiens
  misc_feature: 3′ region
cctgatcgtggcctgcttctgttccccaaagaagagagacagcatgagcatccaccccagcctgtcccacctgggtttccagaggctcttcccacccactgcacacaggcagccatgtgctcttcactctgcccctggcccacttcccaccatgagatttcctaagagagctggtgagaccagggctgctgctgagccaaagggaaccccccagaaaaacgcttgctggcaaatgcagccacagcttgtcaggctggaagcaagtgagttagtgcaaatactttgggaatgacagcttctttagaattcggagaaaaactaaaagaaaagtacattttggaacagaggagcattgcagcacagagacagcagaattgcgtggttgaagttgtttgctatgtgactccaggaaaaccactgagcctcagtttctctgtaaactgaatttgttgttattgttatttgacagttattaagctcctactacatgctaggtatggcagcaagggcttcttatattaacttaccacagccccttgagttggttctatgctcaccctcattcagagatgagaaaggagacacacagaaaagttaggtcccttttccaaggtcacagagctaggaggcggctgcaaacccaagaagcctggttgaagttggtatgcttaaccactaggcagactatttcacttctggctgtgccacacgaaaaaatgttagagatggtgactactaagttctttggaaagacagaaactaccttaagtttaatgtattcaaaaatcaagactatgctagtgttctatgaaaaccaggctttgaaattgactgatatggacacaaatgctccatgaaagagggagattctggaccattctaaactggggcagattgttctattcaaaagcaaaacctatagactcagaacgaaaggactatcaagctgaactaccctgggcaaatgactctacttctcagagcttcttgtttgtctgtaaaatgaggataggcctccaaggaccagggaagactaattcagaggatgtttgtaaggggcttgggaatgatatgctggcacataggaggccctcaacaagatcactgctcgttaaccaaagaacccatttacttacttattcattctaagtaattggcaaataattatcattgatttatgcttattaataaaggatccatagttatttacaaaagcagagtaaatggttaggttaactctgctgatgaaattttggcctagatatttttagggaagacagtgttctctatgagccactattttataaaggaaagaaatagtcttacagcctaaagtctgggcttggagaacagcccaagttacttgctgagtgacttgcaacttttattcttcctgagcctcagtttcctacatattctgtaaaatgggtataataatatttatttcaggttccattaagaagtggtcacaggactgagcacagatttttagatatttgaaatatttctccactggattccaggctgtccttttccaaattcccctctgcttacttttcttggagagagtgatcagtctgtcagcctcagtctctgcagccactgtgaacagaaatcacccgattttaatgatcagttcagctaggtgccaaagccaaacaggggaaacagatttgggggctgaggatgtaagagaggaggcttagggtaatccgatcagggcatatggctgcacaatggtgttcaccaaggtctcaattcagtaggaaaggggccagactagagccaggcctgtgtttaggcacaaagaccggcaaatgccatcttcaggggtgaagggccattatttccctgaggtttggaacaggtccctgtgggaaggaagcttaagagagagtttagaaggaaaaagtctctaatcgcagctttgccactcaggtctgtgtcttagacaattcatctctgaatctcgatcacctgtcttaaaatggaggctgtagcaattattccataggcgtgtataagcattaaattagattatgtattttaagcaagctgcccagtatccagcctatgatttgatagtgtgtgctgttttttatagccacacctgggagaagatactgatctagaatctccccatgcttgctctcatttaatccttatgccactctaaaggataggtgtaatcatcccctttcagggattagtgaatgatagactagctctcagcctgtttcataaataaagttttattggaacacagtcacttgcattaatttaggggttgtctgtgctgcttttatgctacagtagcagagctgagcaactgtgacagagaccctgtggctcacaaagcctaaaatattaactgagatagtttggatatgtgtccccacccaaatctcatgttgaaatgtaatccccagtgttggagatggggcctggtgggaggtgattggatcatggggatggatttctcatgaatagtttcacaccatcccttcggtgctgttcttgtgatagtgagttcttgtgagatctggttgtttaaacgtgtatagtccctccccactctcattgtgtgtctttgggttccacactagccccttctggctcaccttctcctgaaagggctgtcaggctggtgtctgtgggctccacaggaaccactggaaaaagagagttcccaatagcagtgactcatgcaacacagtgtcaaaaccaagcacagggtctcaaaggaaaacaggctggttgctggcccaagggaggaaaggggctgagaaagggcttactaagaagagctgttcatcctgtcttaggagtcacttgcccctgttactcctcccagagcacggagtgccctgcaagatcccagctcaggagggagcacagccacagctgggttcaggttcagatcctggctgaaggcaacgaaagaactgaaaaccccaaagatatcccagcttgaggagacctctgttccaaagggaggcagggggtgttgaggataaaaacagtgctggactcagaagatacatagagatatgacatgcacataagcacacacccatacacatgcactcacgctcacgctcatgtacacacatgcacacccccacacatgcccatgcacacatccatacacactcatgcatgcacacatacacctgctttctctttaacaaacaccaaacactgcatgactagttctgggagagagactccagctccaggagaaggatgccctgccaaggtgtaccttggacttctcatccccagatgcctaaaggtgttgggactgagagatcatggaaactggtgaacaaggagtggcaggcataaacctgactcccatttttggcaagatctatattttagattaggaatcatcacttgcttagttcagtgaacagttattaagcacctaccctgttctgaaaataaacatgcgaaaagcagcctccccatcaaaaagcctctgttggacccaggctgccccttttgctgcaggcgctggagagtgcgtggagaaaggggaggaagggagaggaggaggtagtgggtctgctcctgggaggctgtggcagccaggagcttacttagttaggagagacttcactccacctgagcactacgtcttgtcccagtcacactacataccagggctctgactacagagtgagaaaggggagagaatgaacttgggttctacatccacactggattaaattcacacttgtccacacagcatggggctgagtttgcactggggccatttgatgttgggcatttgaattgggtttatcagaagtaacacatctcctagccagagaaggcaggaccacttccagtgtcacagtcactccagctccagtgaggcaggcctggtggaggaggtgtatccaccggctcccatcagcatctgggcccttgtggttcaagagccaccaaccctgggaggccttacctctgggcagaagtgttgtcaagtcttccttgatggttgtctctgaaaagaaaacactttggaaagtctgttgagagcagtgggtttgagggagggtttgaagatgctggacttggcatctagagacatgaagcaataattgatcccaaaccagctgctgtccagatcctcagccccacggctgatgcagccaggttcttcctacctggagtctgagccaggcagaaggaggtgtccagaaggttcagggaagagttgaggctggatcgagccaagcactctatggcagccttatcacaggtacacagcaggtgctcacagttgtccttggactctgcacacagcaagagcatagccaattggaaagatctcagctggaatcccaagaggtaatgcaagctccaagactgagttcctcatctcccctgccaaaaatcccactccccttgcaatctccatctcattcatgtcagctcttatctctttaggggtgtagacccaaacgaggtccatacccactcctcttctctcacacttgcacccagggcagcactattggctccactttcaacatggagtctgaccacatctccacacctccactgtaccacactgggctgaggccctcttgtctcgtgcctggtcatggtagtggcctcctaactatgcccccagcttctaccctgctgctcaatctactcttatccaggagtcagaacaatcctttagaaattcaggtccaaccatgcctttattctgttcagaacgtcccagcgtcttcccacctcaacctacaagtctcactgtcacctaggaaacgtcacatgattgctcccctgacccacgacccctattccttctcaggccacatcctcttccactccctcctcatcaacccatttgtgtctcactgctcccttatttctcaaccacaacaggcctctcaggcctcagggccagtccctgagctgttcctctgtctgctttgctcttctcccatatatctgcaaggatggcttcctgcccattctttaagtcatcctcaaccctcatcatctccaggaggcttttcttgaccttcccatttaaagagaaccccccctttccctccacaggcctttcccctgattcattttcctccacagctcttgtccctctctaacatactatacaatttgttttggatagcttcagtgtctgacaatggtgaggacattccagaaggtcaggctatccccagcacagaagattgtcttgcattggtgtaggtacacaatacatatacgttgggtggatgaatgaatcatgttttctgggttcactgtgcctcccacctagtggttcacacccacctcctcccctcaattcctccttgatgctccatactccagccagtccatgttatatagacttgagtgttaagtcagatcagggttcaaaccctggctttgcccctttgcagctgtgtggctttgggcaagttatttaagctgcaaacaatggcccctttctaaatagggttgtaaggaagatcagagtttctgtgtataagcctacagcacataataaacacacataaagtatggctgtaatataggaacccctacatgctgtgttctgtcttcccttcatgcctttgcgtattctacctctgccagggttgcctagagacatgaaataaaattcctggagtcacatagcaaataaggcgggaactgagatttgaacctatgtgtttctgattcttactggcaaccaccaccgcacgccttattcacgtatcactacttccacctccacgtccatctctgagttcttaacatatgtcgctccctgtgctaaccatttcacaagcattgtgtaatttgattttcacaactaatctctgaagtaggtttctcctcccctttttgagggtcagaggagttaaataattctgcaatgctacacaatgtgtaagtagcaaacaatttgcccttcccaaggtctcagggttgtacaagtataaaattgtaaatcagataacttgctgatcagctaatatttcaactcaaaaacatttattgtgcctctactctgtgaaagatacagatgccattgatccagccctcgtggggctctaagtgctgccaacgatgctgccagtggtgacgatgatgtgagaggatcacaatgccctcagcctggtccaggcttgggatccctggactgtcatttctgggcccaggcccctgggacacctggagatgcttaccacatatgatcttcttgctgacacaattgacctctgtgctaagtttggcggggtcttggagacagtccatctcagcggcctcctcatagcacctgcggtgctggaagcagcagctgtaggaaggccgggaggaggcagggtgagagtgtgggttagagtgagggagggcaggcagctcatgatctttctgtgtctctggatggatcctggcagtggtctattttaaatattcagtgcactaaaatgaagttttgcataacagacataacataggatcttaataactttttagagtgagagctgatcacagatgacattgccatgtaaagatgagtcacccaactagtaggtagtggaattaaaccctctcctgagtcccaagtccaggggcttttctgtatttgtgctcttgccaaggtatcagccaggcctcccatgagctcactttttttggtcttgctcacctgtggaactcacttacctgtcagattcatccacaggcaacccttccatctcaaacctgcaggtgcaaccatagtcttcaaagtctcgggggcagagaccagccacacacttcataccattgacaaactggatcagcacagggaaattggtgaagacagcctgcagccaggtgaagtgggggcccaggcaatctgtgggggtggggggcagggcctgataagcactgggaactaggactaggagggcgtccctgtcccctggacttcctatacctcaggctgatggtgccttatcaacctagtaagcacctactctgtaataaaactgttttagactaagccatcatccctgctggttttctacctaacaaccatttaaaccttcttccttactgacaaaactagaaagttcttaaggaaggtgagccttctacctaagttccttagggtatttcataatcactctcagctggtcatggtaatcccatgttaccaactgattggtctatggctgtgcatgtgacccagatttggccaaaaaaccttaagggaaagtctttaagtgtcttctgagaaatatttcttttcttttctagcaacgaataaaggcaagtaggaagatccttttgatctacaccttttttctaccttgaatgtggatgaggatgtgctagctggagctgcagcaaccacattataatcaggaggccccaagtctaaagttgaaaaaacaatatgttcagggtagcagtggagcaagatagacagaggctgctgggtctttgatacctgtgttgagctaccacacgcatattggtgccttcacaccacttgagataatgaaatgtctttattcttgttgttatttttttaaaaaaatcaacttctattatagattaaagggtacacaggcaggtttgtcacatgggtaaattgatactgaggctcggggtcccaacaatcccctcacccaggcagcaagcgcagtacccaaccggggttttttcagcctatgaccgcctccctccctccctgactagtggtcccagcgtttattgttcccgtctttatgttcacgtgtatttaatgcttagctcccatttataagtgaaaataggtggtatttgtatttctgttcttgcattaagtcacttaggataatggcctcaaactccctccatgctaaatgcaaatccaaaccacaacgagacaccatctcacaccagtcaaaatggtgattattaaaaaatcaaaaaacaacagatggcgaggctgcaaagaaaagggattgcttagacactggctggtgggaatgcaaactagttcagctactgtgaaaagcaatttggagatttctcaaataacctaaaatagaactgctatttgacccaggaattccgctactggaattttcccaaaggaaaataattcattctgtcaaaaagacacatgcatctgtatgttcattgcagcaccattcacaatagcaaagacatggaatcaacctaggttctcatcaacagcggattggataaagaaaatgtggtacaaatatctttattatttgagtcactattagttcactgaatggatcccaaatgatacggtgatgacagtataaaactataagccagtatgggaagaaatgtggtacagatattcacaacttttgagcatcctttaggtgtcagaagctgcactaggtgctttcactggcacagtaattcagcctggcggagtgtgtaatgcattggaaagcatttgttttgtctgttgtcccataggcctcctgttaaccctgtaaggcagatgttttctgtcactgccaattcatagtaagtaaacttgctttacatttcacagtagtaaaccgaaggccaaacacatgcctaaggcatcaaccagggataatgatttgccctaaagtatacagcaaacaaaattggcaagaaaaccatatcaacagtttccaactctaaatctcgcattctcctccccattctctacctttctgactagaggatccttgagtgctgcatgggttacccctcactactgatttaacatgcacagggtcacaagacggagcttaagggcatcaggagtagtttgtgaggaccagttccacctcatactattcacctcattcaaacttcctttcccatgttttgtacccacaagacctaatcagaaaaataaagacagaattctatgctgattgaaaaagcaacactaagctttatgcatgtgtgtatttatgtaaatatgcattttcattcagaaacttacaaggcacctgcacttagttatacaaacagtttaagcacagccttatgcccagcatggcccgacccaaaagtaattctgagtgtctttgttcatgtcaatcatattatagtctctttttttggtacttctagtagttgatgataggaaccatcatagtgtcctgatagtcacataatagattttcagttgataaatgggatctttgacacactttgacgttcttaaactttatatttactagtgagggaagaaaaataaaactactttgatgtacaggactaagctctagattccaaggagagctcctcccttctagctgggtggtcttgggcaagtcactcaactcccttcggttgagtttccttttctataaaatggggataccattcccttcctggagagttgtgagtcaggactaatgaagtaaggaataggaaggcacatggtagctcagtggctcaccctgcaataaaagacaccactactctcgtcggaacttgggtctccttgttgggaggtagctgtggaggccatgggagctccgagggttgagaccaaggcccttgcaatttccttagatttgtccacacatgctctgacctctggtggataaaagcaattttagacttaacttaccaaaaatttcagccacactttccacatttttaaacatcccactgaagaaagtgatatctaagtgtaagaaagaaaacaaatgagagtcttggtttttcctttttttcacttccctgtccaccctctaggtaaagtgtacataaaacctttcttcattctccaaagaaaaaattgaccttgggctaagctagctttctactaacacaggttaccatggaaacctgatagagatgaaatgtgaattccattactgggtagcattgcacaatggtacaaatgacccctgggtggtaaggtatatctggactgacaggaattatgggtgaactataaatacctggtaggaaatcatcatcttgggtctcattcagtgtgatgactcttataaacggtcatcctactgggtgtcctagaaattatacccacgaggtcattactagagatactgattcctatggagaatgaagcttctaagccggggctggtcatacagctgctccacataagtcccattctcttgtacctgagttgtttgttatctgggatccccagaactgctgtgtagattgccgtgagggctactcttgctgaagaggagtggctgccatggcctgctggcatgctgtgttccttagctttgattcttctaaatgccaagtgtggcctgtgagtcttaatgtctaattaaagaaggcctcctggtgggcattgcacttcctctagaagttatgccatttggagggaggccacttttcatttgcatctgaatgggactgattactttgcaagtatggactcacgcatgaataatttggttgacccaactcactcctctgaaaggttggaaatgaatgcttgattcagggctgacatcaccagcgaattcactatgcattatggtcttcagccatgcccaccttcagggacccacctgagaagtgtttataaactatcgctgagaaaattttcgcttccccgggaaacaaatatggatcctccttttttacaaccagagcaagcaatatatcatttatccctcttggtcttgccttcttggaagctcagagctcattctgaagctaagatacaattaatatataaccttcacgtcagcccatttcctgccttctcctggtttcccttttcagctgacctttgtctttgacagaaatttttagactttgtctcagttgtgagaaaaaggttgggccagacaccaggggagattgttggggagatgcttttgagtcccacttcaatccctttacttacaaatgccacagcagtgagtggctctagggtgtattcagaagggatcattttctctgccaatatcctagacaactttctactctgctcctaagaaaagttctaagaatccttactgatattgtttgggagtcctggaggcagctcctgtggaagatgtggagtgtccagaggatggcctcctataaataaggaagagaaagtagggtaagcacaaacactgatacaattcacttgctggactaaggccagaaggcgacagccagacaaacaatgggaattcacataaattcaattccatgcatatttcctagagcctgctatgcactagatttaggaggtagttcctgaaggaaataaggggatggctgaggtaaccctgatgtcttatactggtaagccttaacagtggcattctggttggggatggagaggttgggagacttatcttgaagccgtctttgtgtatgaagcatcctgttttatttagattgcaggtttgttttggactttcttgacataaagcattggtggaaagaggtaaataccccaacccccaaggagtttatataataatagagaataagggttttccccagattatcagcatcagaatcacctgggatgcagactccgtaaacattctgatttctgtgctctcactcaagtttgattcagccagtctggactgggactcagaaagaagcaattttagcaagctccccaactagttttgaagctgatggtctcgaaacctcgccttgagaaatgttacttaatggtgtaataacaatagtgtcctagtactaccagtacccaaatacaaccaccaccttcagccaagacttcatcatgctgggggtgtgaagatgcagcctgggctaatgggacaaatatatgggagttggaatcccaggttcaacacagatttgctctctaaaagttcagagagcttatagacttgttcctgctcatatttttctttccccagcaatataaagcttgtaagtagttggaaatgatcaaaagggttttgtctttcttcgtcaagtggagagttatggagtcagcactgtccatctgacactgtgtagaagccccatcaatctgaaagtcacaaagggccatccttcacagaagcatgttagactaaatctgttcaggaagatgcccccaggtggtgtctgagttggccttgcttggcttagagcaggtttctcagcttcacattattgacattttgggttgtgaaattttctgttgctagggattgtcctgtgcatagatatttagcaagattgttctgtgttgtatatttagcaagatccctggccacagataccagcaggaaacacttccatacacactttcttctgataaaactcattcccttggagtttccacaacgcataactgagagagtcactgaggctcgccaagtccttcctagtggagacaaaaccaccactggtgagaggaataacaacagtgggtgcttgtccttgctaccagtaccaggactggcactaccaatataccatcccacaaggttcctcaaacttcagtcactagcatattgcctctactattacttattaatgcgtatttttaagtcagctcacttttttttttattaaaggagaatttgatttacttttcaattataaaaaagtttgtttcatctgggagttacagtgagtctaaatttgtatgcacataataacatttcttaatgcaacattttaagccaactcacttttttgttgttttattacagggtagttttatttactgtccaattgtaaaaagtttgtttcatctgggagttccagcgactctaagtttgtatgcacataatagcatgatccaaaatatataaagctaaatttgacataattgaaaagactattttaaatctgtcagtgggagattttaaaaaaataagccacattgcttctaaaaaggtaagtatttttacttttaaaggaaacttttatttgtaaatgggaaactatggtaaagtgctgactagaaagaaaaaaataagttataaaaattgtaaaacagtaaacatttaaaaattagaattataaaatggtcatctatgaattacccactatcatcaatgtaccctgctatggtttgaatgtatgtgttcctccaaaatttatatcttcgtacttaaacctcaaggtgatggtattagaaatcagggcctttgggaggtgattaggtcttgaggctttgcattcatacatgagattagcatccttctaaaaaggctggagggaaatagctaggtccatttgcccttccgcacttctttcatttgaggacatagcactcatcccttctgccatgtgaagataaaacgagaaggccctcaccagacactgaatcatctggtgcctcgatctgagactttcctgactccaaaactgttagaagtgaaattctgttctttataaattacccagtctgtgggattttgttataacagcatggacagactaagctaaatttgacagaattgaaaagaaaaactttcaaatatgtcagtgggaaaaattgttttaaataagccacctcgctttaaaaagggtaaatatatttttcttttaaagcaagcttttattcataaatggaaagccattgtaaaggcccaggacccaggatccacagaaggaactgagagcaatttgggatggttggagtatggagactaaggcagtcatggtcccagctgcactaacccaccgtggtcccccaaatatgccgcacattttcacagtattaagtcctgcttttgcctctttcccatttttcctgactgaaaaacccttgtcctctaatgactctatgcaacttttaccaaaaccccctacacccttagccacactctgcaccttagctgtttttctatcagcgtcaataactatcatcaagggaatggacagaatgactggggtcctcgtccttttatctggaaagtcttaaggtgactcttttgtccccactcccccctgttttacaggtaaaatatcagaggtttagagaggtggcacctgccacggccacagagcacagcaggaaagggaataaggatcagtgtttggccaacttcgtcacaagggatggctgaggcacagtctacaagagggcagctgcaaccccctatgacccatctcaccaaagggcctgcctgcagggggcctgaggctgaggcacccacagggtgtgtgggttgggagcctgctcttccacctgctaaaggtgtctgtgtccccagaggtaattccatgatgaatttgctccccaagataattccatggtgaaacaatgaggttgacgatgaaggtaatgtccaacaatgcaacccatttgttaatgaagaggggaaagggaacagtgaacataagaagctgttaatgataatagtaacaaataactggcttaacctttaaacacgtgtagcataccttctgatgttatttccttctacctttacaacacttttttgtgtcaatgttatcaagtcttgcagacctatgtttgtctcctaccactccagtactccaaaccttctgtgattctctaagatctccagcttcttaatgagtaatcaaggctcttggggaagcagcccagcctagtgatcatggctggaaaaagatcctgtccaactagctacctgcccgaggatctcacgcaattcaaaacttgcgtcctttatgttttcacagtggataagtgcagagtaaagggctccaggtcagggtgtatgaatgcataacagttattgtttagaaggatattagggaacaggaaggagtgttgatcttatagccatgatatctcccaccctttcattctctgaagatgtaaaagtgtgtctgctttaaagcagctttttgttctctcagaacatggatagtattttgtaaaatgctcgcaattaatagcctgacatacctgaggactgacagccctttccatataatttttagctatataaccttcttcacaccttccattcccaagttattggtttgaggttgcatttctcagcttgagctcaagccaaaccacttaatgaacaattacctgttgccatggccacgtgacatggctgtaatgtatcacagatgataacgtcttcagctgccatctttggactgctaagacgtgtgccaattccattattttgaactatttgtataaatggatgtgtcattagcattttctgttgcaccatctcacctctacgtgattcaaattcacataaaatatgactgcattataagggtttttttgtccaacaggcctgacttaaaaccttacctctaccattgtgtcaccctaacaagtcacttaactctcaaagcctcatttttctgttggatagaatggaagtaataagccttacctcactggatcattacagaagctttctaggtaaaggggtcaacaggagcaaaagctgaagggtctggaagcagttggctgtggggagggcagactggcaggaaagaagactgcaaagggaggaggatatcaatgatgtggaccctgaaggcctcagaggggtaagttgtggaaggttctaagtaggcacagtgacggtggagtcataggaaggcacgatccatgaagacttgttcactaggggtgggtaactttggaatctgtcactcatgttctcagtaacataagcctcttcctgggacatgggacatgtcctacctctaagtaagatcccagggatgttcatgaaactcactgaaaccacctctgccttaaggattctgagaggctgcaggggatggaaatagacactgaacatatcaggagaccaggttagaggcttcactttcctacatgccggctgtgagacttcgggcacgtgttctttcctcgttgagcctcactttgatcctctgtaaaatgagacaattgtatctgcctggcaagatgcttccaaacacaaaatgactcaatgtatacaatataactgcataggccgggcgcggtgcccacaactgtaatcccagcactttgggaggccgaggcaggcagatcacctgaggttgggagttcaagaccagcctgaccaacatggaaaaaccccatctctactaaaaatacgaaataaactgggagtggtggcatgtaatcccagctactcaggaggctgaggcaggagaattgcttgagcccaggaggcagaggtcgcagtgagctgagatcacgccattgcactccaacctgggcaacaagaatgaaactctgtctcaaataataataataatgataatataactgcataattccaggctcatggaagatctcaacagtctgcatttgtttttcacactgaagaagggaaaaacaacagggtttaaagagatctaaagatgatcctattcagcccccactctagagatgagaaaactgttggatagaacttgaaggcagagtaggaactgctatctgggattcctgaaccccagccaagtactctcactgttatccctcttctggctatgctgttcactgcagccctcacacttgtccctacatccaactttattttctctgttctccttttttgaagcatccttcactcacctatctaactcctatccaattcctgccttctctttagccaagctcaaggacaccttctacaattattcctaggctaatgagaccttccttttttgaagctttggcatttatgtgtatcatggcacagaagaaaaagcatgggctttagagcacaacaaatgtgaatccacattccagctcagtaacttcttggttttgttaccaggagcaaagttctatatctctctgactccagtcttctcatctgtagaatggggctatttgtgagtttatatcacaggacattgtgagattacatgaaatcttaccaagagataacatgaaatgacacatggactcctggcggtaacatgtgggaaatggggtccaacttaagaccgaggactgtagacccaaatctttgcagggatgggcagataatgtaaacaagtagcagtccaatgctgggaagcaatagggaatggtggggactgtggagaaccagagagacccaagttacagcaagacaggcagttgtgactcagcaccagggcttgagaaaggagtctcaccatttttatttttcaaaaattttttttgaaatgttaattttttgataaaatcttccaattttaaactcttggctcaaatttaaaataaaatgttttgtggcctaaacaaaacaggctgattgtggtttgaaggtgaaaggagggtgctttgctttgggtgtttgtgaggctgcgtgggacggctggtgctccatctaggggcagggatgggagctgaccacgcatttctcaggttccagtgccagccatgtattccctttgccaggtttgcggaatggttcccatgaacagggaattccctcctgcctgagaactacaggctttgaggggactgtgacctgtaaaatgtagactttcaggcagggaggtagacttgtgacttcagttccatttgagcctttgtcacaaagctcatgtacttgctctggcacatttttcactgggcataattgccttggaaattaatcagcgtaataaccagcagctaagaatatctgcggcccacttcagcagcaatagtaaaatttgcctgttccttggcaattactattactgcactctattcctttgtgcaactgataattaagttcctcttctggagggtgggagataatcttattacctatagatgttaataaggtgtgctttctcacccacactcacacttactttcaggatatagactcccacacgttattattagcaattaaaatagtcacccaggatgtgaaactctttatccccgaggactaagttagttcactcacaaagtccaacagtgcagcttagggagatgcatggtcagagggaatgtaaggacaaaactcaccagaatgacattgattctgattcagtaagcttgttacacacagaaggtgatgagcacctgtcctgttccaggagcaggacactgcactttgcaggcatcaccttgtctgatcttctcctcagtattgcaaggcacattctgtcattaccatcttatagacaatgaaactgagactcttcgatgaagctataaagctttttctcacccggtacgtacggctttccttgatctggcccctgctgcctctgctgaatcatctcctaccttgaacttcatgctccggagatactgaaaagcttagattttctggacagcaaactacttcttgccatcctgcctttgcttgtactgcaccctggggcctgggatgccttccccttgtctgttaccctcgcattactgactctcactgacattttatggttccattcaaagttcaaggatccctgcctgacccccaatctggcctgggtcagggatgctgcccctgtgctagcataatgttgcagacatccttctctcattgcacctgtcaccccatgttgcaatgatctgtttatgtaaccatttcccccactaaactagggacttcaaggtaagcactgtgatgtatgtaatatattgatatatgtatatacttacattatacatatataacagaaaccacaagttcaattgataactgtttaatgaaggacagaacatccaatagttgaaccacaccatcaaatggccagtgagtggtggcctgctagaatcagagaatcttagtctcacaaacttgaagcaaacttagaatataacatctttagatagtggaatgataatgtctattggtgctttagaatgggatttcatggtattatgtattctaccgaaaagatcaaccaggattgcacaaaacccacctttttcctcaggtcccttctcagaggatctggaaggaaacaaacatgcagaggacaagggggaaatgggagccaacacatggtggtgtgatgtgggggctgcagcatgagcttcagtgcagagctcccagacaaagagagcatgtgcttaggtgggagggccaagggatctccctctcctaatgcaaatgtggccaccatcccactggctgacgcctgcccttggaagtcacaaggcagctccgagtgagagtagaggaagcacagcagtcaccctggctgtgttgagggagccaacatggaagctgctccccgtcctgctggagggatagagacccctggagagacctcaccatgcacagagaccctcctcatggtcaacactcacaaagcattgtcttgtatctgactcagattgctactggggcagccatgagaagggaccttgcggagctccaactacaggaagcataactgaccaagggcctacggctgtgcttggaagtccaaggcagggtttgcacagaggcctcgtccccttcatgcctgctcctagccaatgtcccagggcatgtaggtataccacagtgggcaaggctcctggggcacacaggactcctccggctcctcttggatgaccttgccagcttacatccaactgaagtctagggcacattcacctcccctctgcccatctccttcacatgcagaactttctgaaggtctctacctttcacagttgccactccatgtttttcctcacaggtgtttcctctactgcaatctgcacacttgatctagtttgggcttctgtttctcagaagacccagattcagacaccactcccacatctttgagtcagaagcctcaataacaccttccctgctacagtgtctggccccagaattggcgaatatgttgggcaagcagtcttatctcagtggtggtcacacacttacccttgaaaaaattggggctgcaggtcaagcatgttcaattcacatgtaaatggatacgattgcacgtaaactttctatgactgtaattttttgaaaggtaggtgttaacattggagaataaaatctcttgggatgtaccaaaaatgtttgtaaacacttataatcgtagagcatataattgtcatttttgtcaccacaaggtagttgcaaactaataaaagagggaagaaataaagcaagaagacagagaggaatatgtttaaccatctcaattttatagaaagataaggacatttaaaagacacgtgggaaagatggtgggggtgggcctgttgaagggacagtattcaaatgaagtaattacatgctaagctggaactatggtgtaagatatcattactcaccggcatgggggatcatcagcacactggtgaggagaaacgcaatcatagcaggagaacaaaggatggggcttaggcagcaactggaacggactcctgggagagaagccagcagaataggatggaagcattttcagaattcccatgaaagaacccatgggacccccatgtcctttcttggtcccacaaggcatgtggtaaaaggagaaagccctgaaagggtgtaggtcccagttttgccactaactgacaatgtgcctctaagaagattatgttccctccttgagactcagtttacccatctgtaaaatggaaatgtagccatgagctccctgaagacacagccgtgggcttctgtatttactgattgattccatggtcatttcttccaggcctgcctcgtgtcaggcatcaaaggattgaggatccaggagtgcaaagtgaggagaaaacactccttacctgagttgctcatagactaatgcggaagggaggcaaggtaacaagtgctgtagtttgacttttgttaattgaattttaaatagcccgtactttatgatatacccagcattcccaatgcacctttggcatgtaggttttgaacctttcaacgatcacttgacatagaaataaatccgacaaccacatggtggcgctgcatgctagcaaacaaagcttttccggcagcatctccctcttccttcccttcctcggcttttgagtatttcgaatactttgttgaggtcctgagagttcaatagatactcctcggaggatgctttgggggcaaaggggagaagcgggcagagtccagcaaccctactgcttttcaattgtagctgccgtctgctgagatcagcaaatatttaacttcaataagcccttaacggactctagccaatccattatatgtttaacagggggacggggtgggaggcgcagggaaaagaccagagggattttaaaataatatttgcccccttagaatataactccataaataatgtaaatacggttggttgtcttgtgcatattctgcttttagaacatgtttttgggacaaagtatgaatgataacacatcttgatccataccaccctagtggcacacacagggctgctgggtagggctgtacatgttgtgcactgcacaaaagcgcttggtcaggagggtcaactagatgctgaaaaagccagagcttgactctgaagcttgtcctctgcagtgttgcatcaacccgaaggaaggagcactttgtaaaatttgttctcccagagaccaagactgccccgtattctggtggtcttgcatatgtacccagggccgtgacgggtcattctgcctgtgagcaaggcttggtattagggaaagtgtcccagaggagaagacattgtaggttgtacaatcactactctatggacctatcccagggtctagcacaagctctggtggctgcaaaataaatgtttgttgaatgaatttttttaaaaagggtagagtgtgagttctttgagattcttactcagctcaagaattctctagtaatctggttactgtctcattggtgcaacgctctgcttagagccttttttagccacacaaattcctaaagctggttaactttcaaggtgagacagtctccctcactgtttggtggaaattcagttctgaatgattccttctcaggccatgaaacaaggctgaaaattaagggggagacaagagaggtctccaaatggaaagtggtatgccctggaaagtcttgtggttagcagaaggcacttcagaggagcccagagacctcattgaaatgatacccacatgaatgggtccaacatgaacttggacaatgtagtcactttctgccactcagtatgtcaggtattttattgtgaccatcacaccatgacaggttgccagcatgtctgaacttggagtggcattgggatcttccagtccaagctcctgttttacagatgagatcattgaggtttagagcctggcaaatcatggcaagaatgtccagcttcccaggtctcctgactccaagtccagtgtcatcataataacagtacctatctgtgaggctgttgtgaagactgaaatgagttaatctttgtgaggcacatacaacagcgcctgacatagagaagttgctcaaaagcattaagaattacaatgatggcatctggctcgaggtcaaaggctcaataagtagtagctgctacaatgaaggcctttgtgctgttccctaatcccctgtaagagtggaaggccctgtctggtgaatgaagaagaatgatcagttttgcacagtggatctctgtttttcttttgtgacacatgtttcaggggtgcattcccctggggagttctgcagctcagataagacggcctgaaggtcatgttccattacctccttgaaacatgttgcttgaatgcaaatgagccaaaatgactttattagagacataattttgactatgctctaatttatttttgcaataagttataaggaggtatgattgggtgttcttcgataatggggagagtttcagtaggggaagatgaaaaagtcctggaggtggacagtggcgtgattgcatatcaatgtgaatgcacttaatgctcctcagctgtacacttaaaagtggctgacatggtcaatattatgtatattttaccacaactttttaaaaagtaagtaataatcttcatgaccttattcctttaaacacaacatttcatggattaggtcattctttgtgtcaggtgttccactgagcatgccccacttgctgtctggtgacaaccctccaagactttggctatcaagacgtctcactttctagaccaggaaactgggctcagagggtgagtcacttgccagggtggcccttgcccagaagggggcgctctggcaatgaccagacccaggtgggccagtctgacttgttctggctccaataccctaaactgctaggccggtcctttgagacaaactaagcattccagtgatgactcactgcgtaactgtcacaattatcggtagtacttgcttgggaagacccttagggtggccagcttcttgctagggtgtggtgaaacacagcaggtgacagcttccttgctgcgcacaaagaaggaaagtcagtgccagctagaaggtgccagttagaagcacgtggtcagccctgggcaacagtgaggatgacccgaatctggggtacgtgcacattagggtacgtgggtgggatggtgtggggctgcttctgaaatccagtatcctcctgttggccaagtcaggcgcccttggctgtggggctgtgtctacctggaggaaagacctccttctcctaatttgcccaaggaagtctctggggctgtcgtggcctcatgggtcagtgctgtccttgtgggcctgagaaggctcccgaagcctcgccttggtctgcatgccaggtttggagtcccggctcccctagctgtctcccaatggccggtctcatggctgcatagccagtgtctgagcccaaggccttgctggtcggacagacctgggctggggtcagggagttcaggctctgccttttgcttgtcgagtgccctgagccaggcactgaagatgcctggaactcattttcctgggcagcgaaatgcatataatggtgttttttcatcagggaatcatgagggttaaatctgatgagtcttataaaggtagaacaccatctccggcacttgctcagtgctcagtgaggactgcccataattaatgctgattgtgtttccacaccaggtgccgtgggcggggtcattgcccagggctatgtaggatcctcactaatgtgaagtggtggatttttcccccgggtaaatttgccaggtgctctctgtctggcattgcatcttttcacagatcgactcagagcaatttgggttttcagctatatcctggagcaacatccaggtctcagacagccctgagagcatcccctaccaccttatcctccattcatttatctcctcatttcccctccattcttctctcccttcctccctctttccctctctccctccctcccttctaccctctctttctccctccttttcttccttcatcaatttctcctacttttcttccctcccctccttcctcctaccctttctgcctctttccatctcccaaccatttccctccctccctcactcttttcctccatccctccctctccccctccttctctttctccctcccttcttctcttctcccattcatttccctcctttacttgctgtcttcctccctccctccctcccctgctctctgtctccccaaccatcaggaataacagatgcctaccgtgaaggctccactggagtgcagctggatgaatgatgctagggtttgaacaggtgcagactgagtcctttataccccaaggaaagacccttagccatgtgatgcctctccccactcctcacatcaagaaggaagcctttcatgaatgttggcaggtgggctgcctctgaagggaggaggcatcataatactatagcttggaacaggtgaaggctccatattaagaagccccatcttgagcctcagaggagacctcaggctctaattagagaacaatgggttccctgttctaccccacctcacctcaaaggctaggaggcccatggattatgtctcagggtcatcatgggaactctcgggggatgaaagactgatgaaagtcatgtgggctctcctgcaagataaagggctggctttgtctggtctagttcattctttagcaagaggatttctagcgtgattcttcttatatgttcttggcctcagatctcggaatttgatgcctctttcctttagggcttttctgcccaagctgtggggtagggtgaggtggggctttataatcagacacccaaggttcctctgccagccctgctactgagtagcagtgtgaccttgagtttgtcactgctcagctttagcttccacatctgttaaatgggcttaatcctactaagatttcgaggctgaagaaagccatatatagtgtgtggtatgggggctcattaagtcctagttctttttgtcacttcctcatatcgccttgtgaagggttttctttcctgattttgaaaggagaactggcatttattgagtacgtgtaatgtaccacaagtgttttataggattttgccctcatcatccctgcagcagcccttccattactgctttcataatcctaggagcaaggattaattagctccactttacagatgagaaaaaatggtgtcgttgaggttgaggcattgtagaagctcacgtgtaatggaagcattctggaatgcaggttagcctggctgtgaaccctctgagttgctcactctatggccacctcctgaagaagcaggactcctcaaacgattaaatcagggcacaattgagcctctatggaagaacccattgaagggcttcttcaaataagtaattactctcattcattgaggcttgtgtttgtagatcattaactattgttcagcatctcatgtgatttgaggaatgaagatgcattgtctgtaatattttctcattagcaccaaagacccaccatctctaaaatgactaagcccgtgggcatctatggctcagtgcagcaaaatccatcttttcagcatctcaagagcatagctgagattgttattatgctcaagagcataactgagattgttattatgcctgagcaatacaaaggcataacaatctacataggtatcttgttcgagacatttcatggggttttgagaaggatcattttttcccatagcttacaacagagtatcttaatcttagcactgttgacatttgggccaaattattctttttttgtaggagttgtcccgagcattgcaatatcgcaggtctctactcagctgatgccagtagcactccctaccttgttgtgacaaccaaaaatgtctccaaacattgccaagtgtcccctgggggccaaatcacccctgattgaaaaccactggccttcaatatccctggaaccttggccttttaccattctgttggtggaataaagtgagagtgaagttttcacaagaacccagacataagaggataaagtctgttctaaatgagagtttaaaattttagatttaattatgacagcacctaaatgtctcgttcacacaaacccacagcactagtgcacctgtgcagaaatagggagccacacgcacacacacacaaacacttagaaccatgcactggggccaagaaacaaacacaatctttctcctctctcccccttctcctccccctttccctctcctctttcccttcccctccttcctttccctctccctcatcttctccctctcccttcctccttctctctgtccctctctttctttctcttgacaacttagctggtcttctgggcatgaatgaaataaacagaaatgtgcctgcacctttgctacctggaagaatgaagtttaatgacgttacgcatgtaactgcacaatctccttcatcagaacagatacagtcactgtcccatctctttcacctcatctgctaaccactggtggccacatacatacacactaattagtaggagttccagatcagtagcaccccctgaggctttcttattgccctagctatcccctaaatcaggacacattcagctcagaaggggtcaaacctcttatgaactccaggagaccagttggactccaggcagtcgtcagccactcctgggccctgttttccttctgcctttgctgctgttgtggctgatggtatgactgtagaaaactggccccaaggagtcctctgagcctttgtccttgatgctaggccagatccttgcataagcctcagaggtattccgcatttgagcttcttaaagattatagtcatgctcagtcattattcccaaagtatggggtaactgggttcacatgggtttccccacaagtgaatagccccagtcttgggcccaagccatccatccattcttcaccttccttgatgataagttactctttgaggaaccttgaggaccctctcctgtcaaaggtccttaattaatatccacgcaagggttgaatcaaattaatccatgcttaatgagtttgcttggttcctatccaggtagaagtccctttgcctcccagaaaatttgctgtatcctcttcatctttgcaaaagtcatgataatatacatttatgagcttgacatttgcataacttggagagtctttcaagtgttctggagaataaaaagaaatctttctgcatatgattagaaagagaaatgtctggctgctcctctctccaatgtccaagttctacctccccaggcaccttgtggttcacaggagtgccagggaactcaggggtcttccaatggcagaaactgttttctacttagatgatgaacagaaagtgactttcctcacccaagggcagccctaggtcacaaaagggtaaaaaaaatactcatggcccagcagagtcctaaggcaagagagtggagaaggagcaccaggcacatcaggagaaaaactgtccctggtaatgagaaataaggggtccgtgcagagccactggagagggcacacatgctctacgtggagactcagcatagatagggatactggagcaaccgcaaacctgaatatagtgaaaaaggaatctgatacaatgaaggtggcaaatcagtatgtggggatgcagaagaaagttgactccacagacgtactccacagatgagctcaaatcctgcacctttcagaggaacatctggtccttgaccagctcctggggaatcccctgtaaatccttggaatatcctgcctgatgagtgtctcttgtacctggggcttcgtgccatgacagatagtttatgttaacaatgtgatttatggtggggccttaggtcgtgcagtatcagcttgacctctagagggactggagactgaataactaagtttagccatgcaggccatgaaggcactccatacctactccatacccccaaacatcccctggacaccaaaacttggatgagctttcctggctgacaatactccacgtgttgttaaacattatttctgggaggattagatgctgactgtgcaaccccactggaagcttgtgcctgggctctcttggactctctctagaggtgcctttcacatttgctgcttttcatctgcatccttttgttgtaataaatgctaaatgtgcatataacagttttccctacctctgagttctactcatgaatcattgaacctgagggtgctcatggggacctctgaatccagtgggtcacacagaagaggaatcaagacaagatgtcaataaaaacaactctctaaacctcctcctaatgataaagttttcattatcgcccacttaagggggcttggaagattatatagatcccataccatggcaagtctgattggttgtacatgtattagttaactatcagtatatgtgaatctctctagagtatgaattcaatattaatttatgcttttgttgtctttcaaaagtctcaagtttaagtcacctcaggtattgtgtcagagtctccatggctgaactggtttgtgcaggctagagtgcccacgcatccccagtttttaaattgtcaggattgcctcaagcattgtaggagtttcttgagcatgaacactagatttccatgattttgaagcaggcaaggagtctggcattactgtattaattgtgaggcctctaacaagaaaacttctaccttcaatgttttgcacagattcacaggagagggaaaaaaaatgctacttccaagaataaccactttaaattaactgatgatctagctggagcctgggtgaattcttcaaatgtagcccacttgggacaagagccagggtggatggcgtatcccctgaagtaattgttatgccctagtgttattttcaaggcctcacacagacttgcagatattctcaaggcacttttgctttaaggctgaaaaaaaagcagaagaagaaggaactcaaggtactgagatataaacactgtagagacataaattaaaccctgatgtcgccaacaagggtctagcctcttccggagagcaagtgtggggcagtgcactggacaggggtcactaggggtgagactcagaaggtggaaaagactcatgcaagaccccattgctagttagcatcaaagtctagtgaggagcagcccgtatttctccactcactgcccaatgctcttttccaatacccagctgtatctaacgcatctctagagcttgcacgaggctaatttaaactcattaataagttgttcagtaaatgtaatacgttgtgtgcatagagattttaatgtggcaaggacgaaagaactattgaattatgtgtttgcaagatttcctatttcctctcctcataccagataaagggatttctatctttgatttctgaccaccccacccccactcctaatgcctatatattttttggtcatttaatgcaggatgaaggtgatgataggaagagaagacaggaaaagtgggatggtggacttgcataagaaataaggaccctgtgtttgggaaagaaaaaaataatggaggcagaaggaaagcaaagtaagcagaagaggtaaatggaaacacagagaagagccagagagcttgattttaggagagctactgagtgataagttgaaaaatgaaatataaagagaagttttaagatgatggtttgtgagaagtgagaaacagtggagggcagaaagaataagaagtttctaagaagtgttgacatggatcccagggatcccagctggtattcttcaggacccaaatggaaagattgggttattacactgtttcgtttgtttgttcaaatgtaaaacattgccaagactgagccctgtgagcacccagcttatatttgggttacagtaagaagggattttctatacagtttaatgtaattccatgatatttagaagaccagagtcccttgaggtagttcaggtagatggcatgataatggccaaactatgggttttgggatattttttgcagaagtgagagataaaaaaaagctgatactggaatatctggaaataaatgaagttttgaagctaccaggacaaggagagaggtaccataatgaaacataaattctagtacttcacggtgccttcttttgcatgaattatttgtttgcatttttccttcagtcaagaatttcagtatttgtgggggttgtcccccacaaactgctctgtggcccaggctggagtgcagtggtgcaatctcggcttactgcaagctctgcctcctaggttcatgccattctcctgcctcagcctcccgagtagctgggactacaggcatccaccaccacgcctggctagtttttttgtgtttttagtagagacggggtttcaccatgttagccaggatggtctcgatctcctgacctcgtgatccgcccgccttggcctcccaaagtgctgggattacaggcgtgagccaccgcgcccagccttttatttgtctttttagcttacataggagctctgtggctaagagatttgcctgagttcacgcagagagcaaaagtgcttggtcaggctgactcctgctgtgtttttcccttgaccttattgcaaacatcaagctgttagctgaaagggaataagaaaaatcctaggtgctctttgtcccaaattaatgaggactagatgagctacagcattccaaagacatctgggaatcactcagatttacaggacccttttgtgtaatgcattcactgcaaagttacaacatagtcaagctgtgtacttactgtgcgagtgggacacccttttcaccctctgacagatatatgttgcattcttcagaaaccaggaatagtattcaagacagtatctaaagatttaaatcagagcagggagcaataactatcctgtgatggctattagaagacagccagagttttaaaggtaagcacataaacatgaactatatggccaattattcacagcaaggaaccctctgcccatctttaaagggtgtcagaggagagataggaaagaaggctgggtaggaagaagttcagactttggggccagcaagacttatttttatccctagccccactgcgtactaggatgcatgacttcagggtgataacttaattctctggactttaattttctcatatataaatgaaaggcaataatggaacctatactcgcaggttatataaggataagataacatatgtaaagttcctgaacagtacccagcatacaagaggtgctcaatacatggtgacatttgtctgtttgtttgcttatttattttagtgggagatgagagagagagtggagtgctgctgtcattatcaatgcagcaaacactttgcaaaataacattctgttcttggtcagaggtggcaggagctgcatcagagggctgtggtgaaaagatcactcacccaattaataggaagaagagttctcagtgcaggctgtgtcaactagctcattgtatgactctgggaattcacttctctttcaatctctcacctgatatttgaggaggttgggctaaacattctctaatttctcttccacctctaacattctgtgattctagcacaccctagagagttgtgcaactaattgctcaagtcaagctgtggtagagattttccaagggctatcacccccaatttataggaaagtgcaaagctgaggaaactgaggcacagagattaaatgacttccccaagggatttctggtggtagtattggattttaacccaggtaacccactttatgccagatcacactgtcattctggtaggcaataagatcagtatttgagtcaggaccctgccaggaaagcaggacttggtggttctatagagggactttaatacgatgaatcagtgataaagggctgaaagaaccaacaggggaaggtgtggtaaccccgcattagcagccacagggagttgctgggcctggagggacagacagagaaagtactgtggccaaagcacagagggcaatggccttggtggcagctgggaccgcagagctgaagctgttatgaggattccttgtggaaactggaaccacagaggaaatagagccgctcctggaaatattgcccaaagcagaaagggagggggagaaattccctggcttctcccctctactgccctccaatctcctctcagtatcttctatgggatgaactcactggaatctggttggcagtgtgttctgggaaaggtagtttgcacaagtcagccccttcattaaagagtctactagagaatgggcaggaaatggaaccaagtgtgaagaagcaattgatgaacacatccaggatgctttttgtgagcttacgaacctgtactcttagggagacctttcaaaacttccagagataggatgtcaaggttggccaactcaacagctgacatggccactagatatctggttagcacagctacaacatgtcaagggtgctagcaatgggattcccacttggctgtgggagtggacaagagagcaaccacaacagtgtcttaatcagcaaccacaaagcgcagcgttccaggtaccatgcaggaacaattagatatattatttcatttcattttgttctcacaacactgtcaatatgagactgctgctacccttttacatgaatggtgctcctggagttgtgcaacatgtggtctagaatgtaggcaggactatacccattttgtagatgaacaaactgaggttcagagattgaatgactagctcaagaccacatagcttgtaagtggcagagactaaatttaaacccaggcctgcctggcttcaagccctgccctctctatagctcactaagctacctactagctttgagggccttggctgtatggtctaatcccatttcttcttagccagtgtaacaatctgcaagtcatactttcttacaacagcaacaaaacttcttagaaagcttctgtgatagaggatatcaagcaactttggggcatgatctgctttagaagttcatattgctagatggacaacatcatccaaactaaggtgctctgtgaaactgccaagtgtgaggccaggatttaagttgcagcaaccacagaaacatgacccacaccttcatgagccaaaaagctgccattctgtgcctgaattcactttgaaagtgtacaccgctctcaggatgaatgagaatcttgcctctatgatcaggagagaaactatttaaacacgacatgtcccatccccaagctttctggcagaggcatcccctacagtgataaatgaacaatagataaatgcccctggttattactttgtagggaatggagtcaaagtcctgttcataacaagaatatgtattcctcacatggactggaagaggcatttccttgcagaaaaggctagatggtcttagaagaaaatcagttgttaaggtggcctattttgtttgtcttcagttcttcagggcagatataagctgcaatgccatggctagcatgagctgaaactagtgttgaagcacccatgattacaccagccccttgtaggtatgtctatctaaattcctagcagaccagagctgctgcacgcactgtggagtcagcagcagcctgaatagaagaggagccgtggacacttcactggggcctgaggtagcaaaatttgtaagtcaccagggactgtggctttgttatcctgcaaggagtgtgccccaagaatggttctggggccccaaataagaaagatttcaagagatgaccctgggagggagatgatatgaagagaatgggagtttagattatctcagtttaaaaatggacaggatttagagggctgcacttcaataattctgtatgttttatttattctgaacaaggagggagtagaaagcttggagaagggacagatggagctctagagtgaaagtcaaagagggcatataataagttacccaggtgcttaaaaggcaaggtagtcctgaatagggacatgccagagacttggtctcccatctttgctgcagctacattgaacatctcagtggtccttagatgctccaggctacccgtgactccatctttgcagatgctatttcttttgcctggaaaactcctattcctccataaagccctattagttatttctcctgggatcccatagcatgttattcaggtctgctacagcacttgtcagctgctttgaaatttgtcttcttacattgccaactttcccaccagtgttttttttgtttgtttgtttgtttgttttttatcacagaggctacgttcagcaaacatactgagtaatgccacctcccctactctagtatttttggcctatattcagtgtatgagtttatggaaaaatgtactattcagtgaaaatacacagaaaagaaggaacaagcccaagcaaacaggtgcttcattgctctaaatttaaactcaaacttctctctgaaaatttatgcctttttcatttattcattcagctattatttgcaatatgaatgttttcaaatgtgatattttccaacgggatatgattaacaggtttttgcctggttacgtgacttggttctatgatctataatactatattttgatttgtgtttatgaccatttcttccttccaactatgaaaccctcagtttctgaggctatttctgattcacctctagatccccatggcttgggacagagcatcacagatatacagcagagactcaatacacattgttggttaagttaatgagtaaaggatctcctaaagtgggcttaacctagacaatacagaaagtattgtcgtaaaagttttctttagaataaacctgtgctttgggtctacaggtcttgcttattgccctctatgaatggaggaaatgttcgtactgacgggggggggggggagggggatgaaagattacagctggaaaggggtgatggaaaatggctgtccacctgggcttcagactcagaggcagaaattttaaaagctaatggatcttgagagtagtctttgaaaataacattaaagacaggagagattggtatccaggccactggagcaggagtattcatctatgtaatttagttctgggtttacagccaagaataaatcccagaagaaataacagtatgtcagcattgtaggacagaaaatgtccatgtctagaccttcttaatataaaacaggttctgctttttaatttaagttttccaacttattttgctggtggatgattggtagggatttatgaaagaactggaatttagatagacatatcttctaattattctctcctagcaaatctcgccaggctgagggcatttggattaaagaaccactcaagatcctctgccatgatcctctgccataattctacttaacatgacacaagttaacttaactcaacacaacatatctcaatatagcttaattaatctaaactcaacacaattcaacttatctcaactcaactcaactcatctcaactcaacacagctccactcatctcaactcattacacctcaactcaactcatctcatcacggctaaactcatcccaactcaactcaactcatctcaactcaacacaactcacctcatctcaactcaacacatctcaactcaacacaactcaactcatctcaactcatcactattcaactcatctcatcacagctcagctcatcacaactcaagtcatctcaacttaatacaactcaagtcatcccaactcaacacaactcaactcatctcaactcattacaactcaactcaactcatctcaacacaattcatctcatcacaggtcaattcatctcaactgatcacaactccagtcatctcaactcatcacagcttaagtcatctcaactcaacacaactcaactcatctcaactcaacttatctcaactcatcacaactccaattcaactcaactcaactccaattcaattcacctcaactcaattatacttattgtgtcaggcactgcacttggttctggggaccagctatgaatgagattccatcatgaatctcaaggatgtgactgtctagtaaaggactcactggagagtcccttaccttgaggtaacccctactgcctgtctgtggattgcttattgcctgactgccttgtggcctgcagaaaagccacacggaataagaaagctataaatattccatgtgaaaagaagccactgggccaagttacctcatgtcctctgtcctgggactcctctggctcatgcagaggcattgctggaagaattggcacagctccatcaggcatgggttgagcctctgaatagccagggtgagaggagcagctgccatagctcccaccttgaagagtggctgaggacaccctagaagatgcaatcccagaccaattaaatcagtaagagtttagtaagcatcttctcttcgttaagccctgcatcaggtgaatatagtcttgtcctgatctcacgtagctctgagacagacaggtaaacatagcattcctcaccattacccaataaagctactgtaacagttttaagagtcaggtggaaagaaggtggtgatcgagtttctggaggaaaacagagatagtttcactgagaagcaaatattaaacagagatctaagatgttgaacaggaatgtatcaggtggagggcatttctggtgtaagggacagggcatacaatgactctgcgtgatgtaagggggtgacttgctgagggggatttgagaaaaatttgagtacaggggatatgaggtatgtatatgcatgtgtgtgtgcagggtacatgtgtgtgcatgtgtgtgtatatgtatgtgcatgcatgtgagagcatatggatatgtgtttgcctgtgtgtgtgtgtatgcacgtgcatgtgtatgtgagtgcaaaaaggagagaaagaagggcaggttgaaagatgagactggcatgctgggtcactggagagtcaggctctgcgtttgtgagttctgtgtttccatcaagtgaaagttaattatgtttccgtgaaccagattttcagtgttgtttaatttttagtttttaggaggaaggccaaagataaaagaaggagacaacaaattatttttaataaaaattccaacttgttgggatctgctgagactcagaaagcttcctatggctacacctacatttccttatatggaaaatagggagacccatccctatcatatagaatgactggaaggattaaatgagacaatttatataaagtatattgcatattgaaaagttgtttgcatgaaacaaacaataaataatagcaattattattattatttatttattaaaaatttgttgagctccactacatgctaggcactggtgacagccactagagctacaaaatgaataagaagaaaaagggccttcctctcacagagcttactttctagggactggaaggttatattacaatattactaataatcttccaacaaaggttcactaatcactaccaggaaaaattatctaaaacctggaaataaacaaaaaggattactgaatacccttttaatgcagtttggttattgtctagcagactctctgcggattgtctgggtaaaaataattgtgcaccattaattgactttctattgaataggtgattttacaactagcaaggggtagatgttaaccgatagcaatataaataattcttgtctaagagcaatgctaattatttctgtccatagcaatgtaaatgaattttgtctggtagaggtgcaattgatttttgtccagtaaaaaaaaaaaagagtttattgctgtggaatatcaattcatttgagaattcctttgactgactacataaatcttttcccctcaaattccccattgaagcaatcttcacatctttctggtttcctcattacttaatgagttcaataatatctttgacatgttgacatgtattcattcaatattattctgtcatgtttgaacatttttcaaaattataatttagcagtaattacttatttttattttttatttcttgtagagatggggccttgctatgttgcccaggctggtctgaaactcctgggctcatgtaattctcccacctcagcctcccaaagtgctggaattacaggtgtaaaccaccacatctggccaggaatatatttatttaagtggagaaggggctcataatgttagcagaagactttttaagaaggtagaaagaaaatactttattctgagttaactagaagataaaccctccagaacactgaccaatttccatcaatgtgcccatgagtttttctctatcagttattgagcagttgctatgtatcaggcactctcctaagatctttgcatacattattttatctaattctcataaatcttgtgaagtagacatcagaagtatacagaagaggacacagagggtctgagtaactctgcagttgtatcagttatgcaacagggagtaactccagcaagatctgactctgaagcctgtgctcataaccatcacacttcactacttgaacataattaacattcccaaggcttgatttccatgtcccttgccccaacccaccccatccatccttctctaactcaggaaatgacacttgggatattcacttagttgctcaaacttgggcatgatttttggttccctcctgtgctcactccatcatcaccgcatcctcttggttctatctcctacatatcacccaagtctccctatccacacagtcacctctactttccagtcaccatcaccactggcatcattagcacaacagtcacctcgcaagtttccctgcttccagcttctcctgttcctccccattctgcagtcagaggaacctttctgcaacacaaatctgatcacattactcccacttgcctcagaagtctacactccttgacatggtgacaaagtactgttaatctcactcttggctacctctctagcctcattccacactaccctcactttcagcaccttcaacatcagtccttttaaggtatagtgcctttgcttaagctatgcctttgcttagaatgcaccttgttcaggtgccacattctcaatcaataaactcttgaaccatcccctcacctgaaggtaatctcttcctctctggatatacatagcagtttctgcttctcttaagaatctagtcactcttttttttcaattgcagcttttttttttttcacatatggcatatcttggaaaaggtgacacagaggtgtggaataagcacaagctttgaaattagatctggtgtcaaaatggagtctccttttatatgtagaactcaccataattcctgaatgagatattacctctcctgcagaattattgtgaagagaaagtaaaaataggcctagcatagtacctggcagtacagtaggagcccaataaatatttattaaatggatgaatgagtggatgccaaatactttgtggcatgattttttttttggatgagcagaataagttactgagaaatgccagaacccctttaatttgcaattatgtttttcattacataggtagatagatagatgacagatggataggcaaatatctatgatatggaagttaagaaataaagataaccagatagaaagataaataggtatggatggataggtgggttaattgaaggatggatagatagatggcttggataaatatctattagatagatgacagatgagtgggtaggttaataaatagatagatatggtaaatacctattaggtaagtagatagagctaacaagagagacagatagatatagttatacagatagctatagatacatggatatagatacagatatagatatagatatggatatagacaccaatatagacatagatatagacagagtcaacctgttttctaaaacttgatctcacaacagcctccatctgcaccactgtgtttctttagaactcctgggagaaaagggatataatttccttagtagcataatcacatctttgtgtctgaaaggttcattcactattcactgcaggctgagagtaaggctggggcagatctaaggtgacatgaaaaaggagacggggcagggtccagcagtttaataatgccctgttgacatatgactggatggtccaggagctggtgtacaaaggtattcaggagtgggaggagcctgagaattcaagcagctttgaagcactcactttgtatccatttctttcttatggaatctggctcaacagaaaagaggagagtgagatgtccaaaagcaagctggaaaaatgcagcaaaaaccacagcaactcaagtcctcagcctctgtcaggagttctgaaaacagagctacaaactcaggaaatggtcttcgtctagatccatgagaattaactgtgcagggatggaaagggtaggtatcccttaacacagaacaacaggacatcatacccagcaaatgaggttgcattttgcagtcatcttttttattccttcctccttcactgccaacaaccctgctccttgcaagagcagcttctatttgcaaagtgcccagcatgctacccaatagacattcactcgataaaagttgattgaattaagttctacagagctctatacttccaaaccacttccacaaacattatcttacttaatacaaaaccttgtgggaaagataaaaaaacaggcatttttagtgcccacaaaaattgcaattattcttgctttacagttgagggaactgaatcttggaagttgagtactggaggccacctcaccagtaaatggctgagctaagatttgaatcccatgagtctgattttctaaatttaccgaacaaacctctccttattctactacatgaccttgttcaaaataaacacaagcaatagtaatgactgggcactggtacttacttgaaacttgatggggtcttgggaccaggactggctcttcaccagcagtgaatggccactctgcagagagatcacctgcaaagggcaggaatggccttccagaagtcagaatggaattaccttaggtgatacacacagcaagaaatttataacatcttaaaggtaaatcattcttgaaatctataggaaaatgatagccaaaaaaggggaagtgatttatggaagaccacacggaaatttagcagcacggcaaggcttataaaccagttctcctgactcttttcctgcaggaaacaataaagcagagtaacctgcttagattgcttactggtaccccatcccaccacgcccttgtccccaagcttcccacccctcccatcccccacccccaaacccccacttccgtactgagtttctcacctgtttgtggatatctgggggctgttgccttggttggactcggagtctgtgtgccatgggtgaatgctcctgggaggagatgagagagaggtgagcctgcatctcttctagggggccctgagggagaagatgaccagggccacctatcctgccctatccaaccaatgttggttaagccaggtaccaggcagggctttaagaagcagagatgagtaagaaataaaacccacaatggagtgtgaaaaaggaaatgggaaaatcccatcattactgtgatacagcgtgatggttgccattggaaaccagctgacattggtaagttatggtgaccctgcacaggagagcgatggtgttgtacaggaagtgtccaatgtgggtgatccagggcaaagaaaaaatgagttaaaatgagttgcttctcattcatctgagtcttagtgtacaccttgatccatcccacccccgacaaattgcctgtcatattcatgacagagtacgggctcagttcaacaatttttgtatcatgttcatgaagccttagtttaagggtcacttcaggacttgatcgtttctgttaagattctggcctctggcgaaaccagcaatacagtctccttgtaggagaccagtgagagagaagctcaagcaggcaagttccgtcatatggataagtggaaggaaagaggtgaggctgagaaacagccgaggcacaggcgatggacacccagttcagctgggtcaggtgacatctactctaagtcaagcagcacagcagttctggaggagagtgggctttgcaggcacacaaagtgcatacataaagatcaggagatgtgggcagtaagcagtgtgctgtggctggagtctagaggaggaaggtggaggggcgaggtgaggctgagcagcaggcagggctcatgcctggaatttcaggcccgggtccatccatcacagttgtcatactgcttgttaatggacaatgaaacagggaaaaagagaagaaattggggagtgcgattacataaaagagagaggaactggggaatgaaagaaggagctttcattcatctacagatgggaagaaagaggtagggcaaaggaaaggagaaggaagagtttgcttcatgatcattatgttttcttagttttctgtgtgtgtattatattccaaaaatttagaaagtttatcaaaacagacaatgctaagtttaatttaaaacgggagaggtagaagtgagcaccctctcttacccaaggtagggctggcctgggatgggctgccaggtgtggccattatctcagcagcaggcgccaaaaggcttgtagtgttcatggcttcctcggtccctgcagtagtcggtggggaagaacgagtttcccagagagaccctccaggttttttctcactgatggtctgagcccagggcacggacgatatggaagcccacgtctgtctgtcagctgtcaaccactgagttctggccaccctcctggtagctgcacattcaaagtgacagtaacagggtacagacatgcttgaccacttcgattcagacatgaaattgctgagacattgaaacgttatcaaagggcaaatgcaatgtgaacgtgtaatataagtaatccaggggcttaaactttactctgaactgaaataaatggaccaaagtaagcattgttttcctaagcaccaataaacacacacacacacacacacacacacacacacacacacacaacctctaaatctctaggaccccaggcgtgtgtgtttgggggtcagttagctgtctctcaaatttatttaatacatttctaatcatgtgacatagtgaaaagattgtggggtctgaggtgaggagatgcaaacagattagttgtgcctctgtctcctccatggtgtgaactttggggcaaattgcttaactccttttagcttcctcatgaaaatgaggataccagtgttaataataacacctgccctccaacatcacaggattgttattagttaagaaaaatccttggctcatgctatttaatggagttgatttattttcctctacaccacccagtcatccttgtttccttgcctgctgacacctatcaattcttatctcttggggaagcctgtgtctcctctatgccggggtaggagccctctctgtgttctctgtgtacaatagttctttgtaggctggatcataactattggtttacttgcctgggctctctcctaggtctgagagctcaggaaagagagagaacatttatagagctgtgtttttaacagctagcctcctgtctggaaggaaatatgttttgatgagtaaataaatgaatgaattctgatgactgaatgaatgaattctgatgacaatattgtctcagtgggtccattgttggaacccactgagaaaatattgactcttttttgtttgtttgttttttctgcactttattttattttaagtcttaggttacatgtgcacaacttgcaggtttgttacatatgtatacatgtgccatgttggtgtgctgcacccattaactcttcatttaacattaggtatatctcctaatgctatccctaatgagctacctccattttatattatttccaaataggaaaaataagataagcatgccttttgtgtctttagtgatgacactgctgatcatgtacgcaagatgctggggaggcaggggcctcctgcaggtcattggcggcagctttcccactggtgttaagtcattaacatattcttggcatcattatgaaaccagctacagactgacctaaccatgccataaaatattgacttcacttcaccgtttcatgtgaatttcaggagcgatatcgctaaatgctttgctgaaatataaatacttttgccacaatgtactgtgattatgtaagacattagcattgaaggaggctgggtgaagggtaccagggaactctctatattatttttgcaagtcttcgtgagtcttaaactatttcagaataaaaaggcatcaaaatacatattagaaacacattatgtcaatggcattttctgcctttgtttgatcttgtagaacccataccggctcatacagatctgttatttaagtaagctgaagcgtaacttcagttggtggagaagggattaaggtaaggattttgcttatatatgtgagcctagaggtaacaggagcgagacatagcaaaggggaggaaagggtgagaatgcatcttacccaaggctgggagagtgtgggaggcactgaaccatgtggctgtggccctggctggaagctcaggaggcctgcctgtgttcagggtctcctctgtttctgaaggagcagctgtctctgtccagggagaggatctcagagcagatgcccagggtgtgctctgggtccagtgtccggggcttgtacttggtagtgttttctggcttttggttgtgggctttgaagtcctggcagttcccctggtagctgcaccttcagggaggcagtcaatggtaacattaacatgcttgacactttgggcataaaaaaactgaaaggtcactggactgcaaatatatgaatgtggagattcaaacctttctctaattaaaagtatcaggcatttgctatgtttcctgaacagattctgacccctgtcagagcctttcttccacctccagccctcagcctctccaggactctggtacatgtctttggaagaggaattagtctcctccatatgccttatggatacaactaaatttcatgtgcgtccgtgtgaagagaccaccaaacaggctttgtgtgagcaataaagctgtttatttcacctgggtgcaggcgggctgagtccgaaaagagagtcagtgaagggagataagggtggggccgttttataggatttgggtaggtaaaggaaaattacagtcaaagggggtttgttctctggcgggcaggagtgggggtctcaaggtgctcagtgggggtgctttttgagccaggatgagccaggaagaggactttcacaaggtaatgtcatcagttaaggcaaggaccagccatttacacttcttttgtggtggaatgtcatcagttaaggtggggcagggcatattcacttcttttgtgattcttcagttacttcaggccatctgggcgtataggtgcaagtcacaggggttgcgatggcttggcttgggctcagaggcctgacatgcctgccttcttatattaataagaaaaataaaatagtgttgaagtgttggggtggcgaaaatttttggggggtggtatggagagagaatgggcgatgtttctcagggctgcttcaagcgggattaggggcagcgtgggaacctagagtgggagagattaagctgaagggaggtcttgtggtaaggggtgatattgtggggatgttagaagaaacatttgttgtatagaatgataggtgatggcctggatacagttttgtatgaattgaaaaactaaatggaataagagaaggagaaaaacaggtataaaaggactaagaattgggaggacctaggacatctaattagagagtgcctaaggagattcagcatagtcctgccagcaaagattatttatttacttcaagagttaagagtggcagtttggggatagcaccagtagatatcagctgtgatggcttgcaaaaaaacagtgtaaaccggcagtgtaaacaagagcagggcatgtatgagtagttgagaacggtgaataggtgtatgactagacagaagatagtagggatgacaagttttttggggcacagtctaagttggtctggtgtctggaatgagaagcatctatacaggagcctaaatgggctgtaccctgtagcattccgaggacaggcctgaattctgagaagggaaagtggtaaaagtattgtccagtcctttttaagttggtggctgagcttggtgaggtgtgtttttaaaagacctttagtccattctacttttcttgaagacggaggaccgtaagggatataaagctttcactgaatactaagagcctgaaaaactgcttggctgatttgactaataaaggctcatctgttatcagactgtattgaggtgggaaggcttaactgaggaattatgtctgacagaacagaagaaatgactgcggtggccttctcagaccctgtaggaaaggcttctacctatccagtgaaagtgtctacctagactaagaggtattttagttatctgactcagggcatgttgagtaaagctaatttgccagtcctgggtggcgcaaatcctcgagcttcatgtgtacggaagggagggggcctgaataatccctgaggagtagtagaatagcagatgggacactgagaagttatttccttgaggatagatttccacgatggaaaggaaatgagaggttctaagaggtgggctagtggcttgtactatagtataacctgcctttgctggtgtgtggcgattaggcctggtggaactgccatcaataaatcaagcgtgatcagggtgaggaacaggaaagaaggaaatttggggaaatggggtgaatgtcaggtggatcagagagatacagtaatgggggccaggtgtggtatcaggaataatgtgggaggccggactgaagtccaggccaggaacaatggtaattgtgggagactcaacaaagagtgagtatagctgaaggagccaggaagcagaaagtatatgcatcagctatgaggaagaaaatagattttggaagttatgagaactgtagagagtgagttgagcatagtttgtgattttgagggcctctaaaagcattaaagcagcagcagccgctgcacgcagacatgagggctaggctaaaacagtaaggtcaagttgtttggacagaaaggctacagggtgtggtcctggctcttgtgtaagaattctgaccacactaaccatgcctaggaaagaaaggagttgttgttttgtagaagatgctggggtttgagagatcagtcggacatgattggcagggagagcacgtgtgtttttatgagaattatgccgagataggtaacagatgaggaagaaatttgggcttgattgaagtaatgggggctgtctgtgaagctttgcagcagtacagccttggtaatttgctgagcctaatgggtgtcagagtcagtctaagtgaaagcaaagagaggctgggacgaggggtgcaggggaatagtgaaaaaagcatctttaagatcaagcacggaatagtgagttgtggaggaaggtattgaggacaaaagagggtacgggttgggcaccacagggtggacaggcaaaacaatttcgttgataaggtgcagatcctgaactaacttgtaaggcttgtctggttttaggacaggtaaaatgagggaattgtaaggagagtttatagggtttaaaaggccatgctgtagcaggtgagtgataacaggctttaatctttttaaagcgtgctgtgggatgggatattggcattgagtggggtaagggtgattaggttttaatgagatggtaaggggtgcatgattggtcgccaaggagggagtagaggtatcttatacttgtgggttaaggtgggggggatacaagaggaggacgcaaaggaggctttggattgggaagaagggtggcaatgagatatagctgtagtccaggaatagtcagggaagcagataatttagttaaagtatctcagcctaataagggaaatgcacaggtggggataactaaaaaggagtgcttaaaagagtattgtctaagttggcaccagagttggggagttttaagaggtttagaagcctggccgtcaatacccacaacagttatggaggcaagggaaacaggcccttgaaaagaaggtaatgtggagtgggtagcctccgtattgattaagaaggggacgggcttaccttccactgtgagagttacccgaagcttggcgtccgtgatggtctagggggcttccgaggcaatcgggcagtgtcagtcttcagccgctaagccaagaagatctgggaaggagtcagtcagagagccttgggccagcgttccaagggctctgggagtggctgccaggtgagttgaacagtccgattttcagtggggtcccacacagatgggacgcggcttaggaggaatcccaggctgcgggcattccttggcccagtggccagatttccagcacatgtagcaagctcctgtgggaggaggttctggaggaacgcctggccgctgcggttcgggcgtttggaagttcttgtgtgctggagatgtggctggggtttgtctcacagtggaggcaaggaattgcaacttttttctattattgtacaccttgaaggcaaggttaattaaatcctgttgtggggtttgagggccggaatttaatttttggagttttatttaatgtcgggagcagattgggtgataaaatgtattttgagaataagacggccttttgaccttttagggtctagggctataaagtgtctcagggttgctgccaaacaagtcatgaactgggctggatttttatatttgatgaaaaagagcctaaatgctatctgatttgggataaagaaaaaggagcattaaccttgactatgcctttggctccagccacctttttaagagtaaattgctgggcaggtgggggagggctagtcagggaacgaaactgtaagccggaccaggtgtgaggaggggaggtgataaaaagattatagggtggaggagcggaggctgagggagaattgggacctagctcggcctggcgaggagcagcctggggaggaagggagaggtcagatgggtctgtagaaaaggaagattagaaagactcagcgacgcttggggttggtactgaggggacaggctggagggaaagaaggaagatttgggacgagttgcactgggcacagggactaggaaggactgagtgtgtaaaagaatgcctggacgtcaggcacctcagacagtttgcctattttacgacaagaattatttagattttgcaggatggagaaattcaaagtgccattttctggctatttggaactactgtcgagtttgtactggggtcaagcggcattgcagaagaaaataaggcatttaggttttaggtcaggtgtgagttgaagaggttttaaatttttgagaacacaggctaagggataagaaggaggaatggaaggtggaagcttacccatagtgaaggaggcaagcctagagaaaagacttgagacacagagaaggggtgggggtggtggttcttgccctccagaaaagtagagaaggggttggggcacagaaataagggattggggcacagagataagaggttggggtgtggaaataagcgattggggggttcttgccgcctaggaaagcaggacttgccgctaagggtgaaggagaaggggttgaggggtacttgcccctgccccaggaaagtgggatgtgccgctaagggtgaaggagaaggggttgaggggtatttgcccctgccccaggaaagcagagaaggggtagagacaaggagagaaggggttggagtacttgccccttccccagaaaagtgggacttgccgctaagggtgaaggaccaaggcaggcatccctgcgtggtctgacacccttgaaacgtgagtgtataatcagagaggcatccctgcaatgattaaacaccaagggaaggctgccttcccagtccgtgaccggcgctggagttttgggttcacggataaaacatatctcctttgcctctaccagaaaatgaaaggaattgaaattaagagaagggagagattgaagtgtggtgccaagattgaaaggagaaagaggttgagggatagtgagggaggttggagatgagagtaaaaagaggccgcttaccggatttgaaattggtgagatgtttcttgggctggtctgtctgaggacctgaggtcgtaggtggatctttctcatggagcaaagaacaggaggacggggattgatttcccaagggaggtcccccgatccgagtgacggcaccaaatttcatgcgcgtccgtgtgaagagaccaccaaacaggctttgtgtgagcaataaagctgtttatttcacctgggtgcaggcgggctgagtccgaaaagagagtcagtgaagggagataagggtggggccgttttataggatttgggtaggtaaaggaaaattacagtcaaagggggtttgttctctggcgggcaggagtgggggtctcaaggtgctcagtgggggtgctttttgagccaggatgagccaggaagaggactttcacaaggtaatgtcatcagttaaggcaaggaccggccatttacacttcttttgtggtggaatgtcatcagttaaggtggggcagggcatattcacttcttttgtgattcttcagttacttcaggccatctgggcgtataggtgcaagtcacaggggttgcgatggcttggcttgggctcagaggcctgacactaaaaaccaaaagtgaaatttgggggaataagaagctcccagacagaaacagcttgaaaaacaggctcccatagagttagcaggagggcttaattcagagggaaatctacccagcatccttggttgctccatgaagaaaagaaggaagtgggctcagatattagtttcactttatatcttccctagggaaggctcaagaggtggcagcagtggatatgacgatgacaggctcctattagcccatagagtcccgttgggaagacaaagactggcctatagtctggtgggcagagcctgtgttggattttggccctcacctcccccagaatgcatgtagaatgtgaactgtctaattgcaggagtggccttgcagcctcatttcctctctctgtgctacttggcccattaacaccctgcgtgcaacaccagcctgagccttgctcccagatcaaccagacatggggctaagtacatccagccaaactatcactcagccaggtacttgctttcaggcacatcaactttagcatcagcttgtaaatatgttctctcttcctaagcatttgttcttgttgaaggagcacaaagtgggacaattaattgagactggcgtgtgtgtgtgttgaggggggtgtggtgggaatgtcatggaaagactctccaaataaatgagattgactcaaccctaagatctgtcctgagaatcgggcaggtgaaggtaatctaccaactgcccatttcatcttcttatcaagtagtcttgtcccatacaaactgaggttccttccatagcataatcaaaagatgccctacaataaatataaagcaaagtagcccctgaagtggagttgcagatttattgtatccttcattgcacttcaagagaaagtaatgaagagcccactatgtgcaaggcaccaaggggaaggcaggctgtgaggctgaaggaaactacttattctaccttaaagttcctttcattgaggagacacacatagaaatgtcaactgtgttgcaacctggagtaacgtagagtccaaatgacatacaagtgctgtgttatgggaccgtagaagacaaagactgacttttattgggggatatttaaaaaagaggaggtagtgtctaagctggcaattgacaggcaaatagggttctgaaatatggaaaagtagggaaggagttccagatggagacaacagcatgcccaatgtggcacagaggggcatgagggtggggcatggttaggtagggactgggtattacagagtggccaggtctccagcatggggtcacaaattcaaatatctacaaaacccaagtagttaatatgagaaaaaagtggaaggcaacagggggcagacaggactatggaaaagaaggaaaaggtgcccttcctagtgggggaatgagagtggcagttactcagctccagttgcttattgccatgtgttggggggaagatctagaattgccagaccttttgcaagagaatccagaaattcatttttttaaaatgacgaagttctctgatttttaatacagtatagcgaaacacaacacctgagggcagctttcagtcattagtgtgccaggtgggaccctggtctggagagtctatgggaggagtttgggaggttggtactcacccagaacaccagacaagccgctggtaaatgtgtaattgataatgggatatttttcttctatctcccagaagtcagaaagattccttcctgcaaaaatcacaccaacagggtcagatcttgggtttcatctgctggacagttttgtctatttctcagcccagagagcttgtcaaagaatgtctagtggtttacctgactttcctgtcatcacacagatgaagatgcaatctgattcattgctttgattcactgtaagacaaacgagtatacaataagacttagccatgggtctgaagctgtaagtctttgattgaaatgaaaattaagttgaaagtacctaacttaggataatataggaaaataagcctgactctttactcttaaggcagtcctcattttccaatttctttattttatttggctgggtttgggggatggtgtagtggctaaggcagagtctctgaggtcagcttctagggttacatcagctctgccacttactagctatgcaaacatgaatgaattgcttgacctcttcaggactcagtttgcccctttataaaatggtgaaaattcaaatcccacaatccctcatttgtaattcccaaatgcccacatctctgtaagacaacatgttttgtgtgagcttggtacccaaatttgaaaaatgtgaagctatgatagtttttatctatcctacttagtgtaaagagctgaacgttttaccaaattactactaacgtgtttgattacactgggcttctctggatgctgttggagaagttacatcaggtaggatgtgcacactttattagatcttcaaagcctgaaaacttctgaattcctaaacatacttttccctgacgatgctgcctaaagaactgtggacatggggttgcatgtctctcataggataacaggaaaatccaaggagataatgggagtgatgcactaggtatggtgcccagcagatagaagagcacagtagccagagtaatcattattgttattattaatgtattactatgattatatatattaggatagtgacataagagcacaggctaagagccagatcacctggtttagaattctgcttctgcagagtgattttgagcaatttacttaacccctctgtgacagtttcctcacctgtcaagtagggataaatatagtagctgccccaaaaggttgtagtgaggatccaatgagagaatacatggaaagcccttacaagattatctgacacctaatgatagctgttactatttctctttctccctccctcctgcttttaaaacagtagctgctccacagtgcatcacaccgtctttctgctgagaccagttgttggactgttgaatagatccttgcttttcaaagtgggtccctaaggaaatgtgattttcctgggggttacatgaaaccaccttataaatgaggtgtatcattcttaactgtactccaaacatgggcttaggctccatatagatttaaacctgaattctggcaccaaacttaagcaaaatattcaacctctctggatctcagttttgtcctccttaaaatgggggtatatctactcaccagatgttttcttgagtctgcctacgttccttaaacttttgagctttggattcttcctccaactttgtggatcccagaaatatgaagacttaaatattggtgcatttactggtgtggaaactggaccacactgattatctaaataaagattacatgaacgtttctggcagcctggcctatagcaggggttggggtgaggaacataatatgcttcattatctaccacaacagatgctaaacctcaaagttgccaaagcgttttcaagatgaacacacctgagaggatggaggttgacttaaaaatctctgtgaggtagctggtagaattgccgatgatatctaccagtagagctgtaaaatctgcaagacaaatttaggaggtttaaatttctgcttcagagacaaaaggagaaaagtatactgtgtttggatttttcagagtaaattttacaagtttgatttagtttagtgtattgaggaatatcaacaatgatacagagaaatactactaattctaaaaaatgaaaagtcttgatttcaggccggaaggaaactttggatccactgttaaaatcccctttcttcttctatcatcctctctcccaaccccttctctgctaacatctaagagctatgaagggccacattgttgaataatggtctttctggcggcttttccccattgtttcgtctaattgtttttagaatgccaactccatctgataggaagcagccccacagatatgtaatgtaaatttacagacttgggggttggaaaatgaaaatgcaatgtggaatttagtggtgagttataaactatgaacaatagagacaagacaagacactttctaaaattatcaccaggaacctttccttctgccccctttttaaaggaactgatggtggtgctggtggggagtggtgtctgagataactttttcagcatttcaggttatagaatcattgtgaactttctttctcagctgggtttgaacagggccattgcctgacatggtgggcactattgactgtgaaaattcctaatattcttctagtcaaacagctgtcatctccttctttgttcatttatggctttccacgatccagcaaattcttgacttctaaacaaggcgaatgtgtttgaggagccccagtcattttctaaagaggaagcttcttgggtccagtggatgatttcccatcatgctgtgtaaatgctcctttcactcattaatgggatcaggagtttgtgcaggagcaagctaacttgaaaatacagaaccaagagaggtaggaaaatgttatgtgacaattagtggaatgtgtagccaagaccaacagatgatgggcactactatgcaagaaccaaagccatgaggtggtcttactatatccgcacaggcacattttaacctcctggagggagaacacctatgcagtatgtagtatacctatgcaggtatagacacctgtatatgcagaaggtaggcaaacatgggcaccctctttctctctctttttttttttttttttttttgagatggagtctcgctctgtggcccaggctggcgtgcagtggcgcaatctcggctcactgcaagttccgcctcccaggttcacgccattctcctacctcagcctcccaagtatctgggactacaggcgcccgccaacacgcccagctaatttttttgtatttttagtagagacagggtttcaccgtgttagccaggatggtctcgatctcctgaccttgtgctccacccgcctcggcctccttctctgtctctttagaccagtctgttcttagtcatttgtggaaactgaggaagggtattatctcttagttattccatatattttctttctgcttctaaaaattctcctttgtttattaatccatttatctaataaatattcatcgagcatttgcagtgtgccatacacttaccaggtactggagagttgtgtaagacataaagaagaagctgaggttagtggtgaagcaagattcataaacagaccaataaaatgcaacttggaagttgttatcaattttgcatagaagctatgagcatggctttgaagccacacacaaaacatggagtccaggttttgcccaaggctcagaaagatgatgtaacttgtccaaagttccagagtacgtggcagagcttagatttcagctgaggtctgtcttgggccaaagcttccactattcccggtattcttaccaccactcatgaaacctcttgctcaatgcattttgtcaattctccctttagaactgttttcacaatctgctttttaggcttggaagaaagccagttattattaactcaaagatattctttgcatttatgaaagtttgaattatctaatttactagcaatgatcaaaatgaatgattcatgactaccttcatacataaatagaaggtagaagtacaaaaccctttttacctatccatttggtaatgatttaagactgtctcttaacactcagtgttggcacatgtgagtgggaaaacataggcattcaaacattgttggtaagactgtaaacgtatgcaagacttttagaagacattttggcaattcaaatcattataatattgtttccctgtcagccaacaattctattttgcgggctttattctgcagaggtaattgggcaagtactctaagtgattttacacaggtgttcatcacagcattgtttataattgcaaaaatatgaggaattagccaagtttccaacaagaggagacaaattaaataaatcatggtacattctttcaatgaatactaagaattcatgtgaaggatgaggcatacttgcattcactggaatgaaatggtttttttaagacatatgggttagtttgagagtagattataaaattgcataaaaaatgaaagatactgatggttgtctttcaatatcgattctcccaccttcctttagtaagacagtatcaaattttaattgggaacgtgatggtttaaagcaacgactataatttcaagcttgctttgtgggtgagtgtagccatgagattaaattctgggaaataggatgtaagagggagaaatgtgaatggtttccaggctgtgagttcatagtgaatgtgcatgccttctgggatggtttggatctgtgtccttgcccaaatctcatgtcaaattgcaatccacaatgctggaggtgaggcgtggtgggaggtgattggatcatgggggcagacttctcatgaatgggttagcaccacgtccttggtgctgttctcatgatagtgagtgagttattgtgagatctgcttgtttaaaacacctttccccttgttctcccttgctcctgctctagtcatgtaagatctgcctgcttctacttcccctctgccatgattgtaagtttcctgaggtctccccagaagctgagcagaagcactcatgcttcctgtacagcctgcagaaccatgagctgattagacctcttctctttataaattacccagtttcaggtatttctttatagcaatgtgagaatggactaatacactttccaaatcctttctcctcttcattgtagctggagtgtgaatgtgctggcaacctaccttaggctatgagaacacagatcattatgaaaggcaaagcaacaagctagaaggagcctgggccccaaaaccatcaaaccattagagcagccctggcatgtctacatgcccagactatcagacatgaaagcaattaacttctaccttctttaaactaggctattttggagtcatttgttatagtagcccaccctgtattctaaataatgcagagaatgggttcctatatgtagagcaggatgtctacactgatgggtagatattagctggggtacataggtagataggtaggttagataagtagcttagatgacaggttggatagataagtgggttatataggtagtgtagacagccttggatagcatttattagcatgttaatagtgactaccagtggatggtgggatatcagctatttctggtttcttagttttcctgtccatgttgcatagcatcttgatatcacagtacattatttttaaaattaagaaaacttaagccagtttcatttaggaagaaaaaaaagggaaaatatgcaaatataccctcaaaggagggctttagatcagagttgaaaacattaaaaagaaaatgatccagatctaaagatcatccacaaagtcaccttccccgaaggtgttgagcaacggcaagttgttggcctacgtgtacagcctctcatggtagcgctgtgacttcttgtcaaacctggcaaaagttgctatgacctaccagaggaacaccaggcaattttatcagtagataatgatccatggatcattcttttcatataattgttgcaagtgtgtgaaaatatgatttgactcatgaaataaaattataaactttctccaggcagatttaactttttaaatcttatttttatctttcacacaatcaggatagggactcacccagcacagacttctatcctcagcaggggttaatgaaatggtgagaaaggcatgattatgaagaatttttttcctcacaaatgtatttgttgaagagattcacagggtaaattacataagataatagacattaagcactcattgtagcatctcgcatatagtacttattatatattagtaattctttcctttaatattttcacaatatttgttttagaaattctactcagtgggggaaataatctgaaacagggaagagtttacgcacaaagatgtccaaggcaaaattatttatattaagaaaaacctagatccactcaaaagtctaagaacagcatagtggctaagagaatgcttgcattcactggttacatagtcattacaagtgtttaagaagagtttgtaatatggtggaagggttaatattagactaagtggggaaaagtacaagaggctttggttgcctttagtgcttgcttcccttacaatctcgtatttaacaagcagccagaaggattcttttaaaatgtcagatcatgtcccttccctgtgcagacaatcacaatggcttccttcttattccgggtgaaaattagaatcctcgcagcagaccttaaacttctatatgatctgcaacaccctccccactttgccctcaccaatcagagctaatttcctatagctctgtcctggctaacctgctccagttatgctggcttctctggtcctcatgctgctgcttaggcctttgtctggaccactgtccccaggaacaaccacagggctggcccctcacatcctgaaggtcacccctcaaccctcacttcatttggagaagactttcctgaccaccctacacacagcaatatcccccactgtgacttttccctccggctttcccctgcttcatggttcccatggtcaggctgtcagcccacacaccatgtacttgcagtacatttatttactccctgctccccgctaggaatctatgtcccacgggagcaggaggtgtgttcactttgctctgtgtgctttccccacacctagaagagtgcatgggtcatagtgcacgctcagatggaattactgaatgggtgaattaacggtgcaaaaattgtcacttttatttttcagtattctctaaaatagggaacacataatactttaaagttttatttataaaactagcatatgaacatactctccttgggaaaaaaaagttcagataatctaaggctcttttaaccaacattcttccactccaaaacacatactccaatcttcttttcaaagggaatcacctatctggtgcccacatatgtgttcttatcaataacattgtgctctttgtatttttgtgtaactttctttttctaattaaaaatctcttggaagcttttccatgcacaggccaaatttgagcactctcactcatttatgtattatctgtggctgtttttgaaagtttaattatgtgggagactacttaaccattttccctcatgatggatatttaggttgttcctcacttttgcaattaaaacaatgcttcatgaacattttcctcctgttgcttttgcacacacaactgtttttcactgggataatgaatgagcacatggttaagggtagtggataggaccatattttcctgtcttacattgaggaaaaacaaaaacttatctaaaaggacacaagcaaaattggaaaagctgcaagcctcatggtaagctgtacccacctgataagtcattggtccgattgtttaaacagtagcaataccttgtggggaatttggcggggtctactgtcttcaggttagaaactgtgaagagaaaggattcaacagagatcctaacacacagaccagccctgcaacacatccctaccaccaccaccatcaagaagagccagccagcactcactgttgtaaacagctacagaaaacttgtggaaggcgaaggaactgtaggaagtgacactcagcaaggagaagaacttcttgctatctgccaaaacataccagataaagagacagacagatgcctactaacgtaacaataatacaaataagtaaacagtccctagaaattaacaatgccaataaagaaaccaatgatgaaaataaagaaacaaacaaaaaagtaatacaaataaaaaataataaacaaagtaaaactaaaccaaaaagctgaagagtatgtatcatttgtctatatttcctgatccatcctcaagctgtggcaactctctaagaaagattcccaaggcatattaggcccctgaggctgctgtaacaaattatcacaaacttggtggcctaaaacagcagaaatttgttctctcacagttctgggggctagaagtctgaaactcagtagggcaccctcccttcaagggctctaaaggagaatctgttcttgcctctctcagctctggcggctgcctgtgtcccttggcttgtggccacatctctctctgttctatcatcacatggtcttttcttctgtgtgtgtctcctttgcctctttcttgtaaggacccttgtggtggcatccggcacaacacaaataatcctggacaatggcctcatctcaaaatctttaactcaatcaatctgttatcacatcaggtgatattcactcttttgacatataaagtagtattcacagattctggtaattacaaagtggatatctttggccattttcagtctactacacaaagaaaggggatttttttttaaaccaggaatactaacataaacaatcaggtttaggaatacatgcatgagcatcttatctactctccatcagtgcctgttcttagtttatatctggagcacagtgcagcagagggagatctttccagacctggtctgagaatcacccacctgcatcaacacacctttaataggtgcacctattaaaagggcacattcctggatgtgatcccaggtctactaaatcagatggtgaagaaaggggaaaggtgggtatgcccagggttctgaattttaacaaagttctcaggtgaatttttttcttttttagacagggtcttgctctgtcactcaggctggaatgcaatcgtgcagtcacagctcactgcggcctccacctctcaggctccagtaatcctcccacctcagcctcccaaagcgctgggattataggcatgacccactgtgcccggtctcaggtgatttttattcatataaatttgttacccactgtgatggtggatggaatggcagaatcattaaactttggtgcacgcaaatcaccagtggtgcccattccccctcggctctctggctattgccctctggagaccccaatgccatacactgagcgggaaccagactctgtatttaataagctccttagaagaatggggtatacaccaaaatgtgaatactccagtactcagtttttttgatgataaaaatcacaaatagcacttggtaaaaatatacatttgggccccttcaaagatcagctggatcagaatctccagagcaggaacctggggagatgaatatttaataagtacatcaggtccttcttattgtgcaacaagtttgggaaactttgggctagatcacaaacctggattaaacagttctgaattccaactctatgtggcaatgggcacgcctcaacgtctgcatctagaaaatgggagtgagagtaccaccacgtggaggttatacaaggattaaattagtcattggaagagttttgagctcctcgaagttatgacatttgagtttctgcaaaatttcacagagcccagtgcagggccttgcagaggaaaggacaggattaacagttcagggctggaagggctaaatgttttgcagcccatccctccatctgataattcagtctctccacagcaccctctacaggtagatcactgccttcttatcgtgcacctcccaaggcagccctatcctcggcaactctcatgagagcaaattggaggcaggagctatatctctgtaatttacagcaacaggcattgggacagggatggataataatgttgatgggtcaatgtctaagtgaacaagtgtagagccaccaagcacagttgtgcagtgtgtgtattgcacaatcctagggaatgccatttacatagacctcaacactgaattggcattctccatcccccttattctaaataacatgtaccattacctatttattatgtttattgtttgtttttggttactccttcacccaatagaatggaagctctcccaggccaaagatttttggtttttatttactgccttatcctccaatatttggaatagtgcctggcatgtgataggtgttctatgaatattgattgaagaatgtgagaataaatagtgaaggcagtgaggaatgtacactagggatcattcaggctttgctgtcattcactaagtatgtgttacatacaaatcattttgctagacgctaaagaacacagaaagaagattacaataatattgtcttaaaaaaagaaatccagaatcatattaacttaatcaatgagtaattattgttagtttatggcaacagagttacaattacaaacataccatgtctgtattgcaattcgctctcaaaattcttgtaaaccttctacgtcattttgagtaaggtaggacaatgaattctgcattctgcttttgtttttgttttttgaggcaaagtctcactctttctctcaggctggaatgcagtggcattaccacagctcactacagcctcgaccttctgggctccagcaatcctcccacctcagcctcccgagtagctgggaccacaggcatgcaccactgcacgcagctaatttttgtattttttgtaaagatggggttttgtcatgttgcctaggctggtctcaaactcctgagctcaagcagcttcccaaagtgctgggattacaggcgtgagacactgagctcagcctattctccactctgaacaccgagaaactgacacacagaagtgaagtgtcttgtcaaggcaacagaaaggttcagaaaagacactggtacaagaaaaaaaaatggacctggattgtgcttttaaaatatgattaccttttaacgctctactgagcatcccattcacaagctctgtcaggttaagcgcggacagatcgattgaccttgcgggcagctctgaaagagattcagagataatgtcactggcaggcttttctcggcaagagaaaaagtttctcatttccaaacttcaggcagatattcaacttctttgcacatgtgctgaaaacaaaccaagtgccaggcacgaggggttccaagttaagttaagaggagggtctcgacctcaggaggtaacagtcaacaaagtgacaaatatagataagagaaattccaatgaagcacaggaaacatgaacttttagtgacaattgcatctctgcatttggagagactcgggactcatataaaaggtatctcaagcataatgggaagcccaagggcattgcaggagttctcagcagctctgtgtttgaaaataagtaccccatgtaaacgtggggtccacacaccctggctgagcattgacatcacctggaaatatacatttttttaattacacaattccagtacctggccttcatcttcagaaattcagaattaataggtccaggatgtgacccagaatggagggagaaatctcccaggtgtagcaggagtggcagtgtgcagtgagtggagttaaaattcccacttcccccggctattccttaatgatttggaaactggccaggagcagtggctcacgcctgtaatcccagcactttgggaggctgaggtgggtggatcacatgggatcaggagtttgagaccagcctggccaacatggtgaaaccccatagctactaaaaatataaaaattagctgtgtgtggtggccggtgcctgtaatcccagctacttgggaggctgaggcaggagaatcgcttgagcctgggaggcggaggttgcagtgagccaagatcgtgctattgcactccagcctgggcaacaagggtgaaactccatctaaaaaaaaaaaaagatttggaaacaacagttgacaagatggaaatagttgttgatgctcctctttattcaaacattgtcttcttcaacttttacttaatgatcagataatgattaaagagatgacgttctcgccactgtgggaaagaacagaggtctagagaagtggaggaattgctctgcaacccagacaacccaagggatttgggggagcggcactcacccgtcgtagcaagaaatgccacccccttctccttcctctcttcttctctaaggccagacactgggaaggagacagttttcatgagaaaaatgtgagtggtccagttcctacccccaggaatggaagaagccttggaagcctctttggcctctgctctccgtgtgagagtcactggcggacactgaggaaaggcagatcacagactcttctgcatggccctaatacagtttctaatctctgtggggtggctgtggcctagaaatgcacattttaataaatcagcttggcgattctgatacagggggtctaatttaccccactctttaaccaagtaggaaaactgaggcccagagaggagaaaaaccttgcttaaatttgcacaactgagcaatggtaataaagggttcagaacctactttctagaactgtagagcagtgcccaaaatattaggtctcttgcctacttaacaaatttcatgcgaccaaaggagaaaatgtcaaagaaaaatttgaaatttcaaaggcattcattcgttcttcattggttcattcaacaaatattcactgagggccaagctatatgctaggcaatgtgaactggtaaaggggaatgaacccggtctccagctccatggggcctcctgtgtgtgatttggtcagggaggaggagactcacatgtcgtgacaagagaggagtgtcaacacggtgtgggagcacagggcactagcctggtgtcaggggatggagaaggccttcttgtggaagtgacattaagcagagaccagaaggatcagttattttggatcacatgctatagaatcacactgcacttgtaactacgactgttggagtggtcatttttcatgttatacctggatggagaaggtgcattattttacattttattttattaaattaaatgagacaagcaacatatcttattggggtcacagagaggttcaggaaagaagctggtacaaaaaaagctagtatcacatgtaacatgaaaaatgaccattgcaacaataggtaccagcaggatggagaaggtgctgtattttacattttcattttgcaaatagaaaacaacaagccacagagtctgaaaaatgtgccctgcattacagagtaagtcaggatttagggctgcccgggaatgtcccatctttttccttcttaacatctttatttccagacactgaaataaaagttttcatgaataaaatagtaacctgaaagtacagcctcaaacagttcataacatccttagaaatcatcttgtccaacactttgaagaatggataaactgaaatccccaaacttgatttgatgtacctaaatttgatgtaccatattgggctagtggctaagataaaatcagaaaagagcaatctcaactctttgtccatgttcttctccctcacttctttctattttactttttaatttattaaaattcatttataattttattgaaaaacacttaacatgaaatctaccctattaattttttttttttttgagatggagtctcactctgttgcccaggctggagtgcagtggcgtgatctcactgcaacctctgtctcctggggtcaagcgattctcctgcctcagcctcctgagtagctgggattacaggtgtgcaccaccacgcctggctattatttgtatttttagtagatctggggtttcaccatgttggtcaggctggtctcaaactcctgacctcatgatctgcctgcctcggcttcccaaagtgttgggattacaggcgtgagccactgcactcatcccaattaacagatttttaagtgcacaatatagtattgttacctattgttattatgttgtacagcagctctttagaacttattcatcttgtgtaaccgaaatttcatactcattgaacaacgaacttcccatttccctctcctaagcccctggtgagcacctttctattctctgcttctgtgagtttgactattttagatacctcatgaaagtggtatcatgcagtgtttgttcttctgtgactggttcatttcacttagcatactgtcctccaggttcactcatgttattgcatatggcaccgttttcttatttaagaccgaataatattgcattgtatgtatataccacatttccttttcctgtttatccgtcaatggacatttaggttgctatcttctgcttttctgataccatgttgaaactgagtcaagtcagctgttgcctgcttaagccaatttggccttttgccaattgccaagctttttggttaaatatcaaagcttctggggttagaggtaggtggggactctaactcccctttgccataacagggctactggaggcaacgggcttatgtcacaaaaggaagtctgagacagacatacttaagcagggcaaatgctgccaactagccagctgtctagcttgagtgaggccactgcaccccctcaggcacccgtgtggcactttacctgcgcagtctactctgcaccagctgctataagatctgcaggaggcacaatcaaccttgcttgtgacctgccaaggcaacagagtcctccatgcaggtgagggaccaggattatgtcagttcggggtctccaggcagcagggatgacccaggctaccaggctggcaggattcctacacctttccagccagaatggtgggccctgtgtcatcttcttcactgagcaccattttttttttttttttttttttttttagcactattcctttcaccaaggaaagaatatttctaccactccttgggttgattttgataacaatatttgaagtctgaactaaacaccagtggagcacagatttttgaacaggatgtaatacagagcatttttcaaataagttccctgacagattacaaaaagatatttttctatttttggataaaaagaccaccataataaaagctttaaaatagaaggttcctggcattttttgtacttcttaatgcttagtattttatcaccttttatccttcatgattacctgtggggcaggaattgttgttcccatttaaggagaagaaaattgaaagctcaactaagaaaaatatttatggaaagttatcctacttacacacaataaattggtacaattggtgctttcaggaaagggaagctgttggctgggggagcattttaactttatacgcttttgtaacttttgctatttgaactatgtgaatgtactaatttaaaataaatgagaatataaaaatcgtccaggtctggtggctcatgcctgtaatcctagcactttggaaggccgaggcaagtggatcccctgagctcaggagttcatgacctgcctgggcaacatggtgaaaccctgtttctaacaaaagtacaaaatattatccaggtgtggtggtacatgcctgtggtcctagctatcgggaggctgaggtgggagaatctcttgagcctttggggtggaggttgcagtgagttgagatcgtgccacctgactcccatctgggtgacagagtaagaccccatctcaaaataaataaataaataaataaataagtataaaaagtaacacaatttccatcaaaactaaaaccgaaaccaaaccaaaaaattgtactgcttgtttactaaaatttagaaacctgttcccagactcctcttgtcctttctgttgcaatgtagtcaccattgtactgcaaagtgaatttcatagcatcaagaaggaaaggcaaagaaagatgaaattattacaaaggtgatgatggcaagtaacggtaccccccattataaaatcttctatcaccaatggaatgttacagtctttgtggatttattaagatcacaaggattcaatgaaccataaagaaatgagtagctcacaggaaccattgttgtgactattagagtgttagatgcggatataatcgctgtcttatcagtagagaaagtcaggcttggggaagtcaaggaatttgcccaagaacagtgagctgaaaaggagcccttatcacccacttacctgttgtaggtaaaaaggtcattcccttctctttcttggcttctcctttgatgccagacactaaagtaaaaaaggtttaatcacagaaattataaccaagacataataccggagataatttgattcaacatacccattttacagatgagagaattatgatctggggttgtgaagtgacatttagtcccgcatctagtttgttcattcatttaatcatttcgtcatcaaatatttgttgaccaactactaatgatggatggtgctattgagtgccgagtgtttgctaggcattgtgtgtgctggaaaggcagacacataggaaaacaacattgaataccttgaagggaacggtgagggtgtgtaacagggggctgatctagtctcgggaatctggaagccttccctgagagggggcgttgagctgagctctgaagaatgaatagcatttgagtaggatttatctaattgggggaatatttgatgtatacagaacagcacaagcccagtgtctgaggtaggcatgagtattactttaaagaatggaaagaaggccaacagtgatgggggcataaagaatgaaggaggtggaggtggtaggaagcagggatggtgagggaggcagggtcacgcagagcctgaaaaggacctggtacactacacagagagaagtaggaactccgcaaataatttggggcagagggagagaaaagagggaggaacttggaagatgacctaacaagatttatgaaaagatcactcaggctgattggtggtgaatgggtcatggagagacaagaggagaagctggtttggaaagctgtctcatggtctgggaaagggaaaataatgttttggacaaaggtggtaacagtgaaggtgaagagaattcttttgcatagatgggacttaaatgcaaaaagtcaagggaacaacagaagtagggaagttattcttgccattttgaacaaattcatttggattctgggcagaaatgttggctgctgaggttctctaaagcacagtgaatactccaacttattatacagaacagacacttgcagaactccagctagacccacctgtgttccaaagggacaagacacatgccaggcccatgcacagcttcattgaaggaccacgtgacaggaagcccagcatgcttgtgatactctggcccacctggaatgaagcaagcaaacacttaggaaactaagcacatggatgcagaccttcctttgagaacagtgcgtgaggaatcattaaaatcacaatctgaaaaaggctttgtactaggttaggagaggttaggaagctaaggtaacaaattaactccacaatttaattggcttaacaagaaaagttgatttcttgtttgcagtatatattcactgcattttggtggggagctctgctccacatagtcactcagggaccccagttaatggagttccactatcctatagttctccatgtggcaactgtagcctccttagactttgattcactgtggcactggaagggagagagctgtgggattatgcaccaactcttaagtgctttggcaggaagcaacacatttgcttttgcttgcagcccataccaaaattacttatatgaccctgcattattgcaggaaaactgggaatgtagagggggatgcatacatattcagggagcatttatatttcttccatggattttctggctgttgagatgggaaatatccctcaaatacagcatcacttactctcataaataggaaaatatattaccataggaatatgtgtgttcacagtctctttccttctctgcagcactctgtgcctgccattctcattttgcatttgccatgcaaaatcaccatgatagttacagttgcaaagctgagactaaattgaataatggctaacatttattgagcactttctgtgtgccagatgccacattgaacactatctgatgcatacttgataactgtacagtctttctgtgtaggtattaatgtaatgacaatttaaatacaggctgcacagatacgatttgaaaagaggagaaatttaccattggtagagggttggatttatctggaaggtgaattgggcgggaagaaaagtcaagagaatgtgcgcttggattggagaaaaatgttgcaggaagggaacatcctcagggcaaattgcatgttgagaagttctgctgtccattaggaatgggaaggccctgtggaattttcttaaaagatcccagaaacatttaaatagctttttgattattgatagctatgtgactggcatttgttctagatgccaccccaaagagacaggtcttacaaggaagcaagaagctggggctttcgtggaattcacctattctcataagtctggttactctctgaataccaattttcctcatctgcaaaacagttaagagaactctcacagggtctttgtgaggatttaaaataatgaatccagagcatttaacccagtgcctggcacatcgttatgctcacaatccataccaggcatcaagctcaggctggagacacagggacagagtagacactgccacttcccttgagctgtttagtaacttagaagatgtagacacatgcccaactaattcacactcaaggctggctgtgctaaattctccactgagagagagatggacagggagagagagagtgagcacctggtgaagccaggacttagcgttctctccagcaaagggtgactgggggtggaagtgggcaggcaattcctgctgagtgaggattctttcctttggcacctcaagcactgcgcaaacattatctcccctttctgagcctcgtgtgtagaatggacagagtcataactactttgaaggtatttttgtgaagattaaatgagaatgaaatatggtgcttagtacaatctctggaatatggtagatattaggaatcttaatttctttctcattctcttatctttctctcttttctcctctttcccccttcttcttctgtatctatttcagaactcagcatcataccagagaaatacggaattaaaagataaaaacttatagtagaaaaacaattatttacatttatataatagctacaaatttctctctttttgactcttgcttcattatgttaaccaccctcttccatttactaagagtaaacattctaagatttatttatttttatttttatttttttgagaagagtttcgctcttgttgcccaggctggagtgcagtggtacgatgtcagctcactgcaacctccgcctcccaggttcaaatggttctcatgccttagactcccgagcagctgggattacaggtatgtgccatcatgcccggctaatttttgtattattagtagagacagggtttctccatgttggtcaggctggtctcgactcccgacctcagatgacctacctgcctcagcctcccaaattgctgggattacaggcatgagccaccgcgcttggccgacatgctaagatttattactcgaagactacaattcctactgatactgttcttggagaaaaatgcatttttacttctccctggttttttaattctttgtcattggccaagtcaactacatgtttggattaaggttgttagatttaacaagtaaaaaacacaggacaccatttgaatttgagataacagtgaataatatcttagtgtatctcatgcaatatttggaacatccatagtttggagagagttttataaaggaacacttcagactggctagacttcacctgacagtaaaactgagactatctggccttacgcttccctttgcaagtctggaccactctgggacagtctaggacagggagagagcctgggtttaaggtcagacagatcttacttgaatcttgagttggccattgtgatctgggctaacttacttaaccttcactcagtcaaaaagcatttgtggagctcatggcatagttgttacaagcacagaccctggagttggaggactgcctaggtttatttcctggctctgcagtgcaacttactaaacctctctgtgcctcacccaagactgtcctgaggattaaaatatgttacatttgtagactgtagcggacagtgtctggcatggagcaagtgctatataaatgtataacaaataagtgatagaattaaagtaatggcgaaaacctcaattacttctgcaccaacaatatagtgataataatgaaaataagaaaagatgttggtactccaatagtaagcaagacagataatgttcttcatatgatagagtgagtcctcaaagttatatctttaaaagactgaattagatatccaaaattttaccgtaaaattaagttgtcattatttcactacatttctcatgactattattggataattatttataaatctttgtttcttccgtgacacttgcagaaaagcaagtcatgtgatgacaggacaataataattatttattgaaggggatcaaaatatgccagccaaaatatactatcccctaataagcgactttgggcacatggattattttgagtggaaggccatcgagaatcaacagatgcaggaagaagccttcttggagcttctcctacctgactatgtgcagacacttctgagaaattctctctccctgggagagttttatggccaagaagaagacggaaagtgggtgctgagtgggcacacaaacctcacaaaaataaaccttagcatccattggtttccccatatatttccttcccacattatactgcctctagaagctcaaagctcttttcctttgtcttgtcacttctccacaaatcggtcaccttattgttaaagtgatatataagtccccaagtctgatggcccttttagggtatttttgtttgtttttgcttgtttgttttgagacgaagtctcgctctgtcgcccaggctggagtgcagtggcacgatctcggcatactgcaacctccgcctcctgggttcaagcaactctctgcctcagcctcccgagtagctgggattacaggtgcccaccaccatgccaggccaatttttgtatttttagtagaggcagggtttcaccatcttggccaggctggtcttgaactcagtcaaaaagcatttgtggagctcatggcatagttgttacaagcacagaccctggcgttggaggactgcctaggtttacttcctggtcttgatctaccggcctcggtctcccaaagtgcttggattgcaggcgtgagccactgtgcccagctccctttagggtttctatttcatttttgtgaggactcctataagcatatgaaaaaaaacatttttctcctgtaatctaacttttgttagtttaatttgcaggacctcataaactaaacctaagagagtacaggaaacaggggtttcatcccctagagtacaatgtattgatcctcccattttgagcacagtctgttatttcaacttctcaagcatcccatgggacaggcattgatctttgagttacagaagataaaggcccagggttctgacgtgcagcccccagctcccagtcacatgatttctaagcaggagaactgggatttgaaccatggtctctcagatactaaagtcccttgtccacagtgttctgttttactcacctgctttcaaaataccatcttggagtgccagtaaaaagcctgatattgaacacaaacagaatggccatttcaaagcattttgggaggtggaagggttagaaatccagtcacactcccgggagaagggctgagaagtgttgctatgactgatgggccaagaagtcacccaagagtcttgtgaaattaatttgcaatatgtctgtgtgttttgtcacagcagccctggagccgggagtgtggacggtgaaggcaaatgctccagagtcttaattaaacagtgtgttaccgccaatcaacaagggtcccaaatcaaccaaggttagttcagcttgatgagcaaacctgcctttctcactgaactctgcagccaaaaggtgctggatcagatatagcataattacagtgcgctgtggagaggtggaaatacacactggagtgtcagcgggtgtgggctagccacagtgagtgacacatgttctgaggcattacgacggtccgttcagccagtaaccttccatatagatatcagctctccctccttgaagagaaaccagaagcctggggattctagacagtttgcccaaggtcattaagctggcaggggcagccctggacttcaaacttaggtagtttggttccatgggctgtgagagaacaccaaaaggtgaggggctggcttttaaaaaatacctaatgacagaaaagaattgaagatcctaatatttgttgaccatctgatgtgctaggagttttacatatagtaacccattggtccttcaagcaacactactaatggcttgttataattcttattttatagctttaggaaaccaagcctcagacaaacataaggcccaagggaatgcatctgggagaaccaggattggaagtccagtctgtttgctgccaaaatccattgcaggttgaacactgagtaaggaaatataactttttagctgttgcctcactgccagccattttttagcgcactcttttatcctcattccaatgttttggaggctttggattaaagattatgtttcttttatcatagatgtgaggacaaaccatcctcccagctggtgactgccttaagattctaatcaatagcttaaaatgaacaacggcaagaaatagactgtgatcaagcgatcaagggatggagtcttagctaaatgcaatgtagatattggatgatagaaactgctctcatatctttcagtatccaggagcccagagctcaggattatagcagatgaattctttagctggcttcatgctttgaagaggaggaggaaccaaaagagttttaaaatacctaccatatactacttacctatcttctgagcaggataaattgacttcaaagaacttaaacagctagaaagattaacagctaggtcagagccatggggtttatatgagttcttagtgaaagatacagtgggtgggggtggggaaagtcttgataacattgctttaaatccccttgggaaaaaatggaataataatcctaccaaaaacttagtgtggtgaaagaagatcaactccaagggagaaaaatgcctgtgcaatgagctgaatagcatccgtgaacagaatgacaatgcactgattcagcatgggatgggaaaacctttctgacagtaattacgtgtattgaaggagtagaacaggagtccctaggtgatgagttctgtcttcctgttgcaaaggtttgtgggccaagcatcacctggcatgcaggcccgtgggagtcttctgaagaaagggctgggggctgtagttgatttcagcaggtgcctacagcgggatacttgtttttatacagcttgcaacctcaggatggttatatatttttaaaatgattgaaaaaaattaaagaagaatgatatttcataacatgtaaaaaccgtttgaaattcaaatttcagtgtccacaaataaaatgttattggaacattgccacactcattcatttatgtattgtctgtggctgtatttgggctacaacggcataactgagtagctgtgacagagaccatatggcctacaaagctagaatatttattactatctggccctttatagaaaaagtttgccaactcctggcctagaagaacttggccccttctgctcctagggcccacattcaacactgatggtttttatctagacactggagttgccatttgcttgtcaccttgctactactcatacttccctaaataagtctccattatatcagccatagtaatctttaaaagaaccttattaaggtataattcaccctttttaagtataaaattcaatgtaaatttagtgcaaccgtcactgcattctaatttgaaaacatttctattaccccagaaagattcttcctgcccatgtatggttaatccccattccctctacccacaccaggccaaggaaaccactaatctactttcaatctctgtatgcttttattttctgaacatttcctgtcactgaaatcatgcaatatatagtcagagaaatagttttaaaacatgaatcaagatcatgtcactacgttgcctaaaatccttccatagctttcgtcagcacctagaataatatcccaattctttaccctggctacaaagactcttgcagttgccctcttcctaagtctctagccatatcacatgcggccttttcccttgttcacagccacagcgactttgcactggctgctccttattggaagcctcttccccaggcttctcaacgactggctccttcctattcttcagttattctttctatttaaacaaaatcaaagccctcttgaccttacatctcctacagctcctccttcatttctctttcccttcatggcaaaactcctcaatgagttgtctctacttgatgccccagagcatctccatctcctctctctcaagcccactcccatcaggcatgacatcagcatccctccagtgcagctcttgtcaaggccatgctgattcttacatggctaactccacttctcactaggcactacagcacttgatgccattagccaatcactccctccttgagaccctctcagcacgtggcttccccacaccacagtctcctattttccctcctatttcattagctgctcctgctcagcctcctttgctcgttccttctcatttccttggcctcttgatgttgtagggcttagtctttgattgtcttctcttttctctctatacttgttcacttggggatctcattctgtctcatggttttaaatgttacctacaagtcgttgaatagatagatacaaagagtcaatcattgcttaatagtggagatacgttctgagaaatgtgtcattaggcaattttgtcatgcaaacatcaatagagtatgcttacacaaacctagatggtatagcctactacacgcctaggctatacggtgtagccgattgctgctaggctacaaacctgtacagacattactatactgaatactgtactcaactgtaatgcaatggtaagcatttgtgtatctaaacatagaaaaggtacaataaaaatacagtataaaagattaaaaaatggtacacctgtgtagaagaacacttcatcatgaaaggaacttacaggactagaagttgctctggatgtcagtgagtgagtggtgagtgaatgtgaaggcctcgaaattactgcacaggactgaagactttataaacactgtacacttaggctatgctaaatttattgaaaatattaaccttgctataagtagtaaacttagcttacggtagtttttttcctttataaacaaaatttttttaaaaccttttgactcttttgtaataacttagcttaaaacacaaacatattgcacggctgtacaaaaattttctttatatccttattttaaaagctttttcctgtttttaatttttcttttttactttttacttttcttttcttcttttaaacttttctgttaaaaactaggacacaaacaaacacattagcctaggtttacgcaagttcagtatcatccctatcactgtctttcacctccccaccttgtcttgctggaaggtcttcagggacaatcacacacatggagctgtcatctcctaggaaaacaatgcctttgtctagaatacctcctgaaagaccgctctaagactttatagtatttttttttttttttttgagacagagtctcgctccgtcacccaggctggagtgcagtggtgtgatctcggctcactgcaacttctgtgtcccaggttcaagcaattctcctgtctcagcctcccaagtagctgggactacaggcacctgccaccatgcctggttatttttttgtatttttagtagagacagggtttcaccttgttggtcaggctggtctcgaactcctgacctcaggtgatccaactgcctcatcctcccaaagtgctgggattacaggcatgagccaccatgcctggccagtaaattttttttataagtagaaggagtacactctaaaataagtattgtataataagtacataaaccagtagcatagtcattaattatcattatcaattattatgtactgtacataattgtatgtgctatatttgtatccaactgatagtgcagtaggtttgtttatgccaggatcaccataaatagatgagtaatgcactgtactgtgacactgggatggctacaatatcactaggcgataggaatttttcagctccagtataatcttaggggaccatcatcctatatgcggtcgcttgttgactgatacatcattatgcggtgcatgactgcataaatttgggtatggatatagatatagacagagatatcttcagccctgaacagtctccttaactccatacttaaatacctgctggatatttccaccaaatgtttcatatgcatttgaaatttaacgcataaaaaaccaaactcatgttctgcttgcaaaacctgactcttccccaatctaccctatctgcatgataccctccctcattatggtactcaaaaaactttgcaataatcctattctcctctcatatctcacatttactccatcagtaaattttgtcatctctgctcttaaaatgtatccagatttctaacatttctcacttcctccactgctctcacttgggtccaagcctctcttgactctcattgaaccaattactgcaaatgcagtattgctcctaactaatctcttgttctctgctcttgcctccttcagtgtattctcaatacagcagccagagaaactctttttaacataaatcagatcttgctatctctctactcaaagctttgcaatgacttccttcttgattcaaagtaaaaacaaataacctcataatggcccatgagggccttcatggcctgacccctgatacttctcaaatctcctctgtcattgctcttccttctgctaactcccttgccctgcactggcctgaggtagacctacccagaaccagaagataaactgctgggaatattttcatatctgatgtggggacaggacatgggacaattggacttttctcaggggagacaccttacatgtcttcagtgaggagaaatcactgggtccttggtcaactggatgcccatgctcattgttggaagctgaaattatcagaaacaagaacaagttcccagaatatctgggatgtaaggctatgaatgggcttttgctgcatttgtgcagatgaaggaaatttttaggctttttcactgtcatacagtcacctgaggcttgaagtgggtaagttttatttggacagaaatatgcagttgcagagtgggtacacttaccataaccacaaagttgtccatacgcctttgtgatgctagacatggactttgcagccagattgttgtgttctagaagagcagtctaccctggttctcagaggctacaggcaatgtttccacagtgaattgtgtccatgagtgtatcacttccagcttgcccagtgttagccatctttgtcctttgatttacagagtaaattgtggcaaccaggatgggtttttgtttatttttgtgtatgtctggttttcaagtaggcttatgtgccagatgctgatggtatcctgcaagtgtccttcctccctacttctgagttcacctgcagttcatagataattctcagcatgctggcagctctccaccttcattggcagtgcttctgtttctctatcagagacttttcttccccctgtttcatctctccactctctaagttgtgttttctgggaatgctttctgaataaaccacctacactcaacatctcatctctatttttgtggacgcccaaattaagacaacttgttgtgtgggcatgggatggagtcaaaagctatcactcactgaacattttctgtgtcctagatcctttgctaagtactctactgacatcattccagataatgacctttcaagataggtataattctccccattttacagatgcttaccataaaagggttaagtaaccctctcaaggttctttctctcacatgaagaaacattccttcccactgtactgacagtagagagaggaaaacttatataagctttactaatttcctctcccatcaactcacagtgtttttgattcacttattctttcttcagcccacgtggtctgagaagtgaagtttctccaaaggaagtaacctgagactcaggggtaggtggtcctggttaagttcaaattgtgactctttcccttattagtgtttgaccttgagcagatttctttatgtttccgagcctcaatttccccatgtataaaatgggaattacaatgacttaaaattttagtgactgaataacactttaattaggctgggcatggtagctcacgcctgtaatcccagcactttgggatgccgaggcaggtggatcactttgagaccagcctggccaacatggtgagaccccatctctactaaaaatacaaaaattagcctggtgtggttgcatgcacctgtaatcccagctacttgggaggctgaggcaggagaattgcttgaacccaggaggtggaggttgcagtgagccaaggttgcaccactgcactccagcctgggtgacagagtgagactccgtctcaaaaataaaataaaataaaaacattttaaaacactttaattaaacctgtgtagtggctggtaccaaggaggtgttcaattcccattgtgttgtctttgcggctatgggttttattttgtatttgattattttcttcccctgggtctttatgccatcgattacctattttctttcttttgtctttattctttccctctcaacccttcttttctcacattttataaatatactcaagcctcccctatcattccctcctgcaaccgataacaaactctctttacttgtcattgatttcgtcaacacttggaccttcatttttcctgcagctctttgcctttcttttaaaagagttcctcaactttacctatattacataaattcagtgggctctttcctatccatatcctgcgttacctcaacatcttggtattactctatatgatggccaccccctctcatttctcccccttttaattccatgaccctcttttcatttctatctacattgatcatggtttcttggacgctccttacattttcctcttcctctgcatacctttcaaggcagatctttcccaaagttcatgcttttccaagctttgcttttttatcacagagccccttgggtggtgagctcactcgtgccatgatttcatcctagttacttttctgtttcctaaagctatgtagcagtcttccttcctccctggctttctgttgggttcaggcaacagtagataccagtagaggatcagaagagggaaagagagtgaggttgcagtctttattcccttagctccctccctgctggttcactaccagattgctgtgtccaatgcccaaggtcatagtccctatcacatggccctctccataaagctccatctgggttctggcccttgtcgggctgttgctagctttgcaggactgtaccatgccaatttgatttaactaaactatgaccaaatctttgtaagcaaaccctttattaaaatctcctccacttacgtagtatgtgtatgactttcttatttctccacctggatcttaactgagacaaccacacattatataacccctggagaagcatgcaggaaaggaacttgagtgagaaaacttaggtaaattaaatgaatataaacatatttatcccctgcttagctagcaaggacttttattcaaaataaaagattctaatggattagtagctaagaataaatgctctggagtcagacttcctgctctgaatcctggctgtgccatttactagctatgtgaccctggacaaatgactaaattctctatagttcagtgtccctatctataaagtagggatgataatagttctgagtgccaagatttattgtgaagagtaaatgagatgattcacagagaatactaagtaaaatgtctggcacatgtaagtgctcagtaaatattagctattattcttatgtctcacagaactgagcttgggagaggctgataaagcctaggtccagagattaagcaatcacccttattagctggagggaggtttctgagctctaatatttgcttcatcagtccctaaagacaataccatgttcatgctaactttgagaatgccctagttacctagtgtggcagacaggttttaagatggtctcaattatatcttatgtagtaataccctccccttgagcataggcaaaatctgtgaattcccgttaatcaataaaatatggcaaaggtaatgggctgtcagttttatgatatgttacataagattgcaactttcatcttgccagcagactctcccttgctcattttgatgaagcaagctgctatgttgtgagctgacctatggagaaggctatgtgacaaggaactgagagcaatctctgcccaacagccagcaaaaaactgatgtcctcagtctaacaaccctcaaagaactaaattgttcctaatgtgagaacagaagtggattcttccccagttgagcctcagataagaccacagctccagccaacaccttggttgaatcttgtaagaccctaaagcagaggacccagttaagctgttcttggattcctggtcagcagaaactatgagataataaatttgtattgtttttaaccactaagtttgtggcaatatttgtgtagcaatagataacacacctaaaatgtagcgcatttactcattgtttcattctttagcacttacagtcaccatgatagtttctgaggacaaagattagatcgctatgttggccggtgctgcaagtccccaaaatgggaccatgggagggagtggaattgtagggagtggaagtaggagggtaggacatgagttcttgggcgatggcattagttaccaataaaaaaggcagtatgattctttctcctaatgagaggaactgagaccaggcagctgagagaaccttgtcagtctctagatacgagaggaatagtttctttgaaaggaaaagttcaaaattgaattctgagaaccatgatcaggatggaaggcagaagtcagaagggagggtgcagtttcagaccgtggatctgggaagagatttcctcactgcaccgtgatctccttaacttaaggacagaaagttccaaagagccctaatataccttcaaggcctcatctgcctggatcagtctccttcttgctttctgcattccaagcacactgccttcttactacatctggaatgctccctgtttacccagggcataaatcccactgagttgcttgccagaggcaatggagaccataagaacaagaatgtaaaaatgcaactttgctttgaacagaacttctaaactgagcctgtctcatgcctctttgcacatcctcttcccaatgtctggcaggcctgtcttctctacttccttcttcatttttggctttgtgaagtcatccttaaatattccattcatacctcatgctctgcatgaagcgttttctatacctctaatatgagccagtgccatgctttgtgtttccacagaacccttgattatctcattcatcacgcttctcacagaactgaaatgatcttaaggccaataccacattaaattgtaaaatactgcatctccgatgcttggtatatggtagatatccaattaatatttcttaaatacatgtttcttgaagtgaactgaatagaagaaaaaaaacaagaattctaatcaatacccaaatgtttaaggaaacagagttggcaaactggcctatgaagggattatacccaccaatggtccaattgataatagagaaatatttaggacatgttcttgtttgctgcatttgaaatgtgtaccactagtagataactcttgccaacactttatgctattaaactgctgccacatatatatttgtaactccaatgactcccaggggaactttgtggttcccttactttattccctgtgcaacaactacacttgggttagacgtagtcattactcaatactaacaaggagatctacaggatcaatcatagtattcttgtggtgagcaaagaaatatattcaacacatttcctcatgctgggcattcatgaagactacatttcccagcctcccttgcagattgccccataccatgtgactgagtttaagcaatgagtctgtgagtgggaatgatgagtcactttgagtccaaggcaggaagatgtgggtatggcttttctagagcctctcctcctggcaatctggctagaagtgaaggactccagctcttcagtagcccaaatccttgcatcactctttgaaactcagacaatctacactggactgtgatttgagcaagaaataaatctttgttgcattaggccgttgatatttagagcttttccgttagcaacagtcagtgttgtttatgataaataatacaatctcataaaagttgttctgaagcaaataacattcctccacataatcactgaaatgtctatttattaataagtggtgcagtatttatttaagatagaacataaaaaatcaggtaagaattatttgcataatatatatggaaatcaacctattaaagttgttcaaatattggacagagccagaatataaattacacatacagtttaaaaattacaggaaatcatattataaagagcactaaaggccacttgtggaatagttgtgttcccctgtaagccagaatgtgaagaaagcctactcgtagcacagagacaaacccagggagcaaggcaccagaattctatgccataaaaaggggttaaaaaatacaatccaaaatcaaatataatggattcatgtcactgggaacctccatttgttcttcaaagactggtgttttccatcactgctacattagaaaaagaaggaaaaaaaatagccatatcctatctgaagcaaggcagagatcatttttctctttaaaacaaggaaggactgacaaatctgatgaagtccacctatctttctaagttgatttttcaggatggcacagagccgacttcacctctccaacagcaaatctctgatccttggcaaaatggctgccccaaatcttgcaacttggccaccacctggactcttaaccctcaggggaactatgggggtgggaactgccccctccttggaaaagtattcagaggggtatagaagggcccaattaccagatcaggttggcttccatgggaaaccttgatcttttaatactgagaaagagagacacaactgttctgagcttttggcctcccacagaatgtttttatagactcacctgtttctgcagaatttcagagctcagaaataaaggtacagtctagagacaaaagaccagagtcagcagagacgtggagatatttgaacttgtttggttaaaaacaatgacaggagctccttgggggcagagaacaaggggcacttgtggatgatctgcagccacaccaagccccgagagctaatggattccaatgtgaaccaggtgaataatttatgaggcattattttctgccactgacagctggattgtgaagccactcaaaggacctgctgaacagacaagaggaacagctgacccaggcatcactctactctccagggaactcatgggtctgtgatcattttactgtaaagctgagtgaacgtagaagcccgaagcattgtggaggagctagggcctgatttgagaggaagaagggggagagagaatgagctttccaggtacacgggctttggttgaatatgcagctctacttcttacaatgtgtgacctttgacaaggtcttgtcttcagtttcctcatctataagatggagacaataacatctccctcatggggttgcgatgaggattgcatgaactttagagagagctatcatgtcacaagcaactcataaggagtgcttaggataagtttctctccccttccctgacctcacttttgtgggcagaggaagagaagggaacattaacataaaatatggtctcattttttattactcttaggtatcaaattgcagtccattcgacaagtctaagaggaattacaaatacattcctgagcactgagctgggcttttgactacctcattgtaagccattctcaagttattcttatatacagccgatttttggcggtgtcaaagaaattcattatttcccttgaagcttattttaagcccataacagtgctatctggaggatgaaggcttaggtagaatgacaatctttaaaattactgggccaactattgttgttctagaacttgaccatccttctttctgaatatctcttaggagctttcacttcagcctgactcaggagaactgaaacatcctaccacagggattcacaaggactgctggggagccctgccaaattctggccgatttatcccgagaacaatgtaagtggaaactaacaatgaagatgttcatttgagccaacagaggcctgtttacttcctaaaataatctcttagttacataaccataagatgtcctaagttgagtcctgtctgcttctttgtagcttttggtgaaggccctagagtatctgcttatgtcctgacaacatggggattctctgggacaaggcaagaggacccaagctgtgttcaaatgagacccagagatcccagtgcaaagggacacctttggtaaaactggccactttgggtcatatagatcccaaaagaaatcatgcctgacttcctaaaatcaaaaccatagggatttatcaagaagaaaccctgtttaattggcctatagatgaggatgcaacctaaggcatgactgttgctgaatgagcccaagtgatgggccaaaaaggtgaaccagtccaaacatggcaggaccaagaaggaacaaggtaggagagggcagaggggtggagggcagagaggtggaggggaggagtgcctgcatggccatgggattgttcttcattccctttctcaactgcacccagcagcagtggtttttgctaggccttttgaagacagcaggaatctgggagaaggaacatcatcgtctttcacagcatggaaacatcaatcttattccagagaagggaagaagtccccaaaggtatgtccggtttaacagtttctcaagtgggccaggaagttctttttcatgttctgggctcaatcgtgatgctttgatttcacttggtattttacttttagttttgtctctaacatctggagcaaagatcagaagacctgagttccagatttattctgtgactcaatcactgcttcaatcactgagggacgtggtgtgatgttctttgctctgagcttctgcttcttgatctaaaaagacaggtggttgttctgggtgatcaaaatctcttcttgctctgtgtatcttagaatccatgaaattatgtctctttgcatctatgataacaaatcaaaatatgaatcacaattttcttacagggttgctgaggttaaagccgtgaaagcctgggaacaaatctccccagggaaggtttaagcaagcattaactctttcttctgcaagttcccccattcattgctagggtgttgtctgatagccaccatgcacagttgactttccttgggaaatcatgagtcttttatttttgcaatttggctttatctcctacacatcatccagatggtgcaacatctggtactatatgtccaatggaattgccacaactcagccactgagatccaacattcctgcctctcttcattccaagattccttctgttctttattgtggtagacaagagcaagcatttttatttgcagagatgtacttatttaaaaagcaattgtgaaagcatttgatttaataattaggattgtttctccacgattctttttctgaaaaaaaatttttttggtgtaaatttagagtttataagtaacaccaaagactgagacaatgaagtccatgcatactgtaaaaaaataacataaataacagtccctagtatgcatcagtcattgtactagaggctttgtgtgtacattgattgtctcacttgaaaatcaaaacacggcagaagagtcaaatgcttttgaatgtgctgaaactcaaattttcttccaggtcatctctgtatgattcctcttttacactgccattactggcttctaaaaccactaaagctatgcagatagcctggtcctaacatgttctaaactgggcattagtgaggggctgcctgctggcttgtttaggggccagttggctcagtttggaatggtttaatatcagcaacaatagcattgggttgatttgaatataaacaacttagactttaaaagttcatcctgaaaaacaagccttcaaggacaagtgaggacatgaagcaaattcctaaggatgcctggggttcaggaagcaaagaagaatctttggttattcatgaaaaccaaataccagctatgtggcatcttctgggaaagcacaggggtgggagaatcctggggtgtgtttggcagcactgtccaaagtacagttgacttcttaggctgctgaacaaatttcttctcttgccccaggagaatttgatctgcaggttcccataagtagagtaacatctttctcttgaaataggtgctgtgtcatagtctgtatcataagcttctcttggtcaacataatgaaatgaaagtaactcagctccttgactggtatcaatcttagcagggattaggagacaagaagaaagctggtttctccaacaatcttcctcagattccctagaatcccccctttttgcacagcttctctggagctatgaagttcaaagcattgaatccaaactcgaagtccccattcaacccactcagccttgactgggctgattggatcattgccattttgatgtagaacctttgctctcattttcatgttcaattttctatagtttccaagctaggaagcatttctgtctgtatagagttgtttgcagggatgaaatcaggtattttattttcctggtagacatagtttgaagggacctctgatgtttgagacctctagattcctagcattttgatggcagaaactgtggttttcttatctctgaacttccagtgcactgcaaagttctgggcatatattggtgcttaataaaccctgttaaatgaataaacaaatgattttttctttattacacccccatttcagtagcatggaattatgaggatgattttgccaaattatgagtaggtcctattagattaggtttttctacaggaaattattctttcaaactacagtgtctaggaagtaccttgcacaaaatagacacaggatgcataattgttgattaattagttcattttaacctatacaccttggggttatctagttatgttttcatgaaaactagggctttgtaatatcaatgtcatgtctaataagcctaaatttccagttctaaaaacaaagttattcaacaacactttctcaatttgatgtgtttttcttttcctgaaataatttttttcttaagcttttgatatttgcttttgtgctgacatttgatagtcaaaactcaaacgatctcaagtacttctttctcagctcaataggatcatgtttcgcttaacatttgtgaagttgatttatctaattcattttgatcttgctaaaatatgacctttaaaatttttcatgtcacattgtgacccagaaagaccaaatacatacatgagatcatagttttacctgataatatcactcctagtttgatgatggaattaatttgtcaatatttcatggagttctgctgaaacgattgcaatttcttgggcaagtttcagaacgactagtatatctgtggtcatgtcatcttgggtcatgttaaccagttccattggattatcatgctgttcccttgtctgacaaaaggatctttgtgaaagcacacactgtttcttcccataactcacattcaattccagaccctgaatttctgcttgctcactgtgagccatattgttgagattgtctgtgcataggcaacttgagcaggtttggtttgcctttctggaatatattttttggggaacaaaaggagaatatcaaagcagagttattccagaagatcatttttctcttttcctttttgttttcttaaatgggaaaaaaacagctcttaaggttcttattatttattcattcataaaacatgtattttgtctatgtctatactgataaagacaacttactagatgttagacaatcaatacttccctactactgatcaatacaaacccaatctctgctgtcctggtcttatgatccagcagggaagctagactgcctgcctatggtttaggaaataatacaagctccaatcctttctaggttattaaaagaatctaaggtagttcctccaattttggatcaatggtaaatactttaaggtagaactccattgagttgctggaaaatgcatcatctattggtaaaatacttaagatagtaagttcttcaagaatactaataagtaagttaaaaaattaattggcgcatatgtggagagtatgactcactctcctttattgttggtttccagtaactcatgtacataaagaagagcatagtaacatgttggatgttaggcaggtgatcatctcaagtgaaagattccaggaagatcaggattcaataatcaagccctcaggaaggggcatagggtttcttaggttgtaatccccactggcactttcagccatattgaaggagtggggaaaaggcccaaataacccatgccctttgtccttcagattgccagcatgtcaaatggaagcccgtggggcttcatgtctgatgcattgagcattctggtatatttcccatctcagacttcaaggtctacacccatccatttatggggctctccataaaaaacacccttctgctgctcacaatgtctgcccttggcctgggactttgggctttggttctaagttcctaaagtcaggggtcaagctctaatcattactgtattctgagctttttgtacagtaactgttgacatgtgagttgaaaggaacctatctctgaggaagtgattcctctctccaacatagtgccagactctcgtattaggaaatgatgaaaccatcaaccaaatgtctgccatagc

SEQ ID NO: 6 - DNA, 77 bp, Homo sapiens
  misc_feature: intron 9
gcataagttgccaagagcacacagctaattacaagggaagccaggttttgaagtctagtagtctgactctggactct